(19) United States
(12) Patent Application Publication
(10) Pub. No.: US 2011/0201603 A1
(43) Pub. Date: Aug. 18, 2011

(54) Novel Acylaminobenzamide Derivatives

(75) Inventors: Jun Mihara, Tochigi (JP); Tetsuya Murata, Tochigi (JP); Kei Domon, Tochigi (JP); Yukiyoshi Watanabe, Tochigi (JP); Yasushi Yoneta, Saitama (JP); Takuma Mori, Tochigi (JP); Eiichi Shimojo, Tochigi (JP); Katsuhiko Shibuya, Tochigi (JP); Teruyuki Ichihara, Tochigi (JP); Masashi Ataka, Saitama (JP); Ulrich Görgens, Ratingen (DE)

(73) Assignee: Bayer CropScience AG, Monheim (DE)

(21) Appl. No.: 13/057,934

(22) PCT Filed: Jul. 30, 2009
(86) PCT No.: PCT/EP09/05506

§ 371 Date: May 2, 2011

(30) Foreign Application Priority Data
Aug. 8, 2008 (JP) 2008-205273

(51) Int. Cl. A01N 43/84; C07D 401/12; C07C 237/42; C07D 213/56; C07D 241/24; C07D 285/06; C07D 413/12; C07D 417/12; A01N 43/48; A01N 37/24; A01N 43/40; A01N 43/60; A01N 43/828; A01N 43/58; A01P 7/00

(52) U.S. Cl. 514/227.8; 546/275.4; 564/168; 546/316; 544/406; 548/127; 546/261; 544/238; 544/131; 544/58.6; 514/341; 514/619; 514/355; 514/255.06; 514/361; 514/335; 514/252.03; 514/235.5

(57) ABSTRACT

The present invention relates to novel Acylaminobenzamide derivatives represented by the following formula (I) and use thereof as pesticides: wherein V represents an aromatic ring group of V1 to V5 described in the detailed description, Q represents an aromatic ring group of Q1 to Q7 described in the detailed description, G¹ and G² represent O or S, R¹ represents hydrogen, alkyl, haloalkyl, alkoxy, haloalkoxy, phenyl or a heterocyclic group and R² and R³ represent hydrogen, alkyl, haloalkyl, alkylcarbonyl, haloalkylcarbonyl, alkoxycarbonyl or haloalkoxycarbonyl.

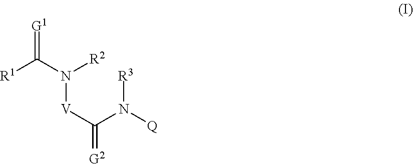

The present invention relates to novel acylaminobenzamide derivatives and use of the same as pesticides.

From WO2005/021488, WO2005/073165 and its English equivalent EP-A-1714958, WO2006/137376, WO2006/137395, WO2007/128410 WO2008/000438, WO2009/049845 and JP2006-306771A, JP2006-225340A and JP2006-302617A it is known that certain benzamide compounds can be used as pesticides.

Since ecological and economic demands on modern plant treatment agents are continually increasing, particularly in respect to the amount applied, residue formation, selectivity, toxicity and favourable production methodology, and also because, for example, resistance problems can occur, there is the on-going task to develop new plant treatment agents that at least in certain areas are able to demonstrate advantages over known agents.

The inventors of the present invention devotedly conducted research to create a novel compound exhibiting higher effects and having a wide spectrum as an insecticide. As a result they found novel acylaminobenzamides, which exhibit an excellent pesticidal effect and are highly safe to use.

As a result, the inventors found that novel amides represented by the following formula (I).

Thus, the invention is directed to acylbenzamide compounds of formula (I)

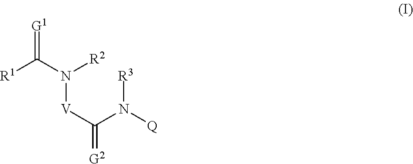

wherein:
R¹ represents hydrogen, or optionally substituted alkyl, haloalkyl, alkoxy, haloalkoxy, phenyl or a 5- or 6-membered heterocyclic group, preferably R¹ represents hydrogen, optionally substituted C₁-C₈ or C₁-C₄-alkyl, C₁-C₈ or C₁-C₄ haloalkyl, C₁-C₈ or C₁-C₄ alkoxy, C₁-C₈ or C₁-C₄ haloalkoxy, phenyl or a 5-to 6-membered heterocyclic group comprising at least one heteroatom selected among N, O, and S;
R² and R³ independently represent hydrogen, or optionally substituted alkyl, haloalkyl, alkylcarbonyl, haloalkylcarbonyl, alkoxycarbonyl or haloalkoxycarbonyl, preferably R² and R³ independently represent hydrogen, optionally substituted C₁-C₆ or C₁-C₄ alkyl, C₁-C₆ or C₁-C₄ haloalkyl, C₂-C₇ or C₂-C₅ alkylcarbonyl, C₂-C₇ or C₂-C₅ haloalkylcarbonyl, C₂-C₇ or C₂-C₅ alkoxycarbonyl, or C₂-C₇ or C₂-C₅ haloalkoxycarbonyl;
G¹ and G² independently represent oxygen or sulfur, preferably oxygen;
V is selected among the cyclic groups V1 to V5:

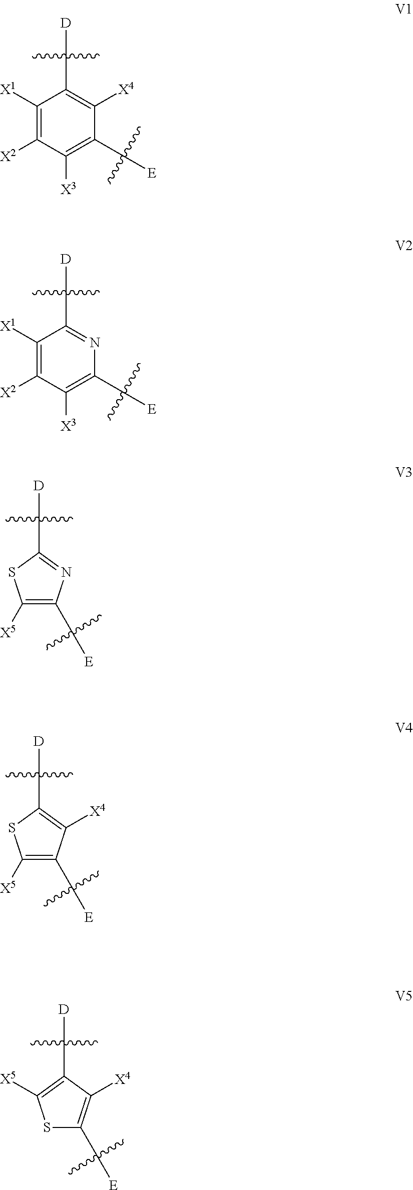

wherein
D stands for the bonding site to the nitrogen atom, i.e. to the moiety

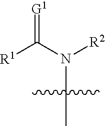

of formula (I), and
E stands for the bonding site to the carbon atom, i.e. to moiety:

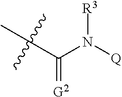

of formula (I), and
X¹ to X⁵ independently represent hydrogen, halogen, or optionally substituted alkyl, haloalkyl, alkoxy, haloalkoxy, or cyano or nitro, preferably X¹ to X⁵ independently represent hydrogen, halogen, optionally substituted C₁-C₆ or C₁-C₄alkyl, C₁-C₆ or C₁-C₄haloalkyl, C₁-C₆ or C₁-C₄alkoxy, C₁-C₆ or C₁-C₄haloalkoxy, or cyano or nitro;
Q is selected aming the groups Q1 to Q7

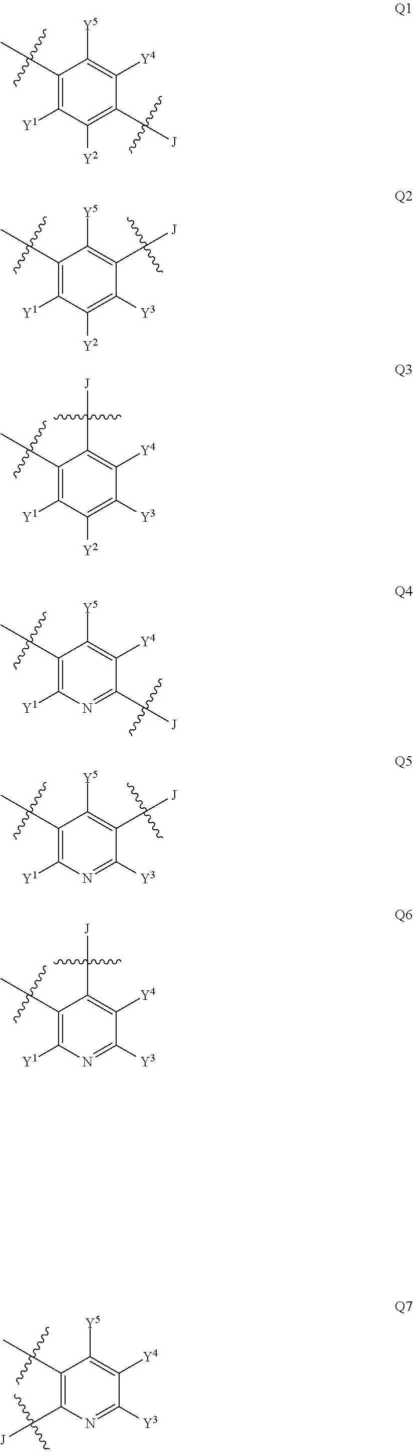

wherein
Y² to Y⁴ independently represent hydrogen, halogen, or optionally substituted alkyl, haloalkyl, alkoxy, haloalkoxy, or cyano or nitro, preferably Y² to Y⁴ independently represent hydrogen, halogen, optionally substituted C₁-C₆ or C₁-C₄alkyl, C₁-C₆ or C₁-C₄haloalkyl, C₁-C₆ or C₁-C₄alkoxy, C₁-C₆ or C₁-C₄haloalkoxy, cyano or nitro;
Y¹ and Y⁵ independently represent halogen, or optionally substituted alkyl, haloalkyl, alkoxy, haloalkoxy, alkylthio, alkylsulfinyl, alkylsulfonyl, haloalkylthio, haloalkylsulfinyl, haloalkylsulfonyl, or cyano or nitro, preferably Y¹ and Y⁵ independently represent halogen, optionally substituted C₁-C₆ or C₁-C₄alkyl, C₁-C₆ or C₁-C₄haloalkyl, C₁-C₆ or C₁-C₄alkoxy, C₁-C₆ or C₁-C₄haloalkoxy, C₁-C₆ or C₁-C₄alkylthio, C₁-C₆ or C₁-C₄alkylsulfinyl, C₁-C₆ or C₁-C₄alkylsulfonyl, C₁-C₆ or C₁-C₄haloalkylthio, C₁-C₆ or C₁-C₄haloalkylsulfinyl, C₁-C₆ or C₁-C₄haloalkylsulfonyl, or cyano or nitro;
and
J represents a chemical grouping having the following formula:

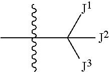

wherein
J¹ represents C₁-C₆ haloalkyl preferably C₁-C₆ fluoroalkyl more preferred C₁-C₆ perfluoroalkyl;
J² represents hydrogen, halogen, or optionally substituted alkyl, haloalkyl, phenyl or a heterocyclic group, preferably J² represents hydrogen, halogen, optionally substituted C₁-C₆ or C₁-C₄alkyl, C₁-C₆ or C₁-C₄haloalkyl, such as C₁-C₆ or C₁-C₄fluoroalkyl or C₁-C₆ perfluoroalkyl, phenyl or a 5- or 6-membered heterocyclic group comprising at least one hetero atom selected among N, O and S; and
J³ represents hydroxyl, cyano, azide, halogen, or optionally substituted alkyl, preferably C₁-C₆ or C₁-C₄alkyl, or haloalkyl, preferably C₁-C₆ or C₁-C₄haloalkyl, or OR⁴, SR⁵, NR⁶R⁷, N(R⁸)NR⁶R⁷, N(R⁸)OR⁶, an optionally substituted heterocyclic group, or a chemical group having the following formulae:

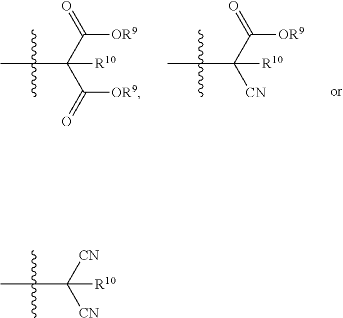

R⁴ represents optionally substituted alkyl, haloalkyl, alkoxyalkyl, alkylthioalkyl, haloalkylthioalkyl alkenyl, alkynyl, cycloalkyl, cycloalkenyl, phenyl, aralkyl, iminyl, alkylcarbonyl, haloalkylcarbonyl, phenylcarbonyl, alkylsulfonyl, haloalkylsulfonyl, phenylsulfonyl, or a heterocyclic group, heterocyclic group-alkylene or a heterocyclic group-carbonyl, preferably R⁴ represents optionally substituted C₁-C₆ or C₁-C₄alkyl, C₁-C₆ or C₁-C₄haloalkyl, C₁-C₆alkoxyC₁-C₆alkyl or C₁-C₄alkoxyC₁-C₄alkyl; C₁-C₆alkylthioC₁-C₆alkyl or C₁-C₄ alkylthioC₁-C₄alkyl, C₁-C₆haloalkylthioC₁-C₆alkyl or C₁-C₄haloalkylthioC₁-C₄alkyl, C₂-C₆alkenyl, C₂-C₆ or C₂-C₄alkynyl, C₃-C₁₀ cycloalkyl, C₃-C₁₀ cycloalkenyl, phenyl, C₇-C₁₂aralkyl or C₇-C₁₀ aralkyl, C₁-C₆iminyl or C₁-C₄iminyl, C₂-C₇ or C₂-C₅alkylcarbonyl, C₂-C₇ or C₂-C₅haloalkylcarbonyl, phenylcarbonyl, C₁-C₆ or C₁-C₄ alkylsulfonyl, C₁-C₆ or C₁-C₄haloalkylsulfonyl, phenylsulfonyl, 5- to 6-membered heterocyclic group comprising at least one hetero atom selected among N, O and S, a 5- or 6-membered heterocyclic group-C₁-C₆ alkylene comprising at least one hetero atom selected among N, O and S, or a 5- or 6-membered heterocyclic group-carbonyl comprising at least one hetero atom selected among N, O and S;
R⁵ represents optionally substituted alkyl, haloalkyl, alkoxyalkyl, haloalkoxyalkyl, alkylthioalkyl, haloalkylthioalkyl, alkenyl, alkynyl, phenyl, aralkyl, or a 5- or 6-membered heterocyclic group comprising at least one hetero atom selected among N, O and S, or a 5- or 6-membered heterocyclic group-alkylene comprising at least one hetero atom selected among N, O and S, preferably R⁵ represents optionally substituted C₁-C₆ or C₁-C₄alkyl, optionally substituted C₁-C₆ or C₁-C₄haloalkyl, C₁-C₆alkoxyC₁-C₆alkyl or C₁-C₄alkoxyC₁-C₄alkyl, C₁-C₆haloalkoxyc₁-C₆ alkyl or C₁-C₄haloalkoxyC₁-C₄alkyl, C₁-C₆ or C₁-C₄alkyl substituted with optionally substituted C₁-C₆ alkylthio, such as C₁-C₄ alkylthioC₁-C₄alkyl, C₁-C₆ haloalkylthioC₁-C₆ alkyl or C₁-C₄ haloalkylthioC₁-C₄ alkyl; optionally substituted C₂-C₆ or C₂-C₄alkenyl, C₂-C₆ or C₂-C₄alkynyl, phenyl, C₇-C₁₂ or C₇-C₁₀aralkyl, a 5- or 6-membered heterocyclic group comprising at least one hetero atom selected among N, O and S, or a 5- or 6-membered heterocyclic group-C₁-C₆ alkylene comprising at least one hetero atom selected among N, O and S;
R⁶, R⁷ and R⁸ independently represent hydrogen, optionally substituted alkyl, haloalkyl, alkoxyalkyl, haloalkoxyalkyl, alkylthioalkyl, haloalkylthioalkyl, alkenyl, alkynyl, phenyl, aralkyl, alkylsulfonyl, haloalkylsulfonyl, phenylsulfonyl, alkylcarbonyl, haloalkylcarbonyl, phenylcarbonyl, a 5- or 6-membered heterocyclic group comprising at least one hetero atom selected among N, O and S, a 5- or 6-membered heterocyclic group-alkylene comprising at least one hetero atom selected among N, O and S, or a 5- or 6-membered heterocyclic group-carbonyl and comprising at least one hetero atom selected among N, O and S, preferably R⁶, R⁷ and R⁸ independently represent hydrogen, optionally substituted C₁-C₆ or C₁-C₄alkyl, C₁-C₆ or C₁-C₄haloalkyl, C₁-C₆ alkoxyC₁-C₆ alkyl or C₁-C₄ alkoxyC₁-C₄ alkyl C₁-C₆ haloalkoxyC₁-C₆ alkyl or C₁-C₄ haloalkoxyC₁-C₄ alkyl, C₁-C₆ alkylthioC₁-C₆ alkyl or C₁-C₄ alkylthioC₁-C₄ alkyl, C₁-C₆ haloalkylthioC₁-C₆ alkyl or C₁-C₄ haloalkylthioC₁-C₄ alkyl, C₂-C₆ or C₂-C₄ alkenyl, C₂-C₆ or C₂-C₄ alkynyl, phenyl, C₇-C₁₂ or C₇-C₁₀ aralkyl, C₁-C₆ or C₁-C₄alkylsulfonyl, C₁-C₆ or C₁-C₄ haloalkylsulfonyl, phenylsulfonyl, C₂-C₇ or C₂-C₅alkylcarbonyl, C₂-C₇ or C₂-C₇ haloalkylcarbonyl, phenylcarbonyl, a 5- or 6-membered heterocyclic group comprising at least one hetero atom selected among N, O and S, a 5- or 6-membered heterocyclic group-C₁-C₆ alkylene comprising at least one hetero atom selected among N, O and S, or a 5- or 6-membered heterocyclic group-carbonyl comprising at least one hetero atom selected among N, O and S; or
or
R⁶ and R⁷ may form a cyclic amino group together with the nitrogen atom to which they are bonded, preferably a 3- to 7-membered cyclic amino group, and said cycle may comprise an oxygen atom, a sulfur atom or a carbonyl group;
R⁹ represents optionally substituted alkyl or haloalkyl, preferably R⁹ represents C₁-C₆ or C₁-C₄ alkyl or C₁-C₆ or C₁-C₄ haloalkyl; and
R¹⁰ represents hydrogen, optionally substituted alkyl or haloalkyl, preferably R⁹ represents optionally substituted C₁-C₆ or C₁-C₄ alkyl or C₁-C₆ or C₁-C₄ haloalkyl;

provided that compounds are excluded wherein J¹ and J² are perfluoroalkyl and J³ is hydroxyl or halogen.

In an embodiment A, acylaminobenzamide compounds of the following structures (I-a), (I-b), (I-c), (I-d) and (I-e), wherein the chemical groups R¹, R², R³, G¹, G², X¹, X⁴, X⁵, Y¹, Y⁵, J¹, J² and J³ are as defined herein, are preferred.

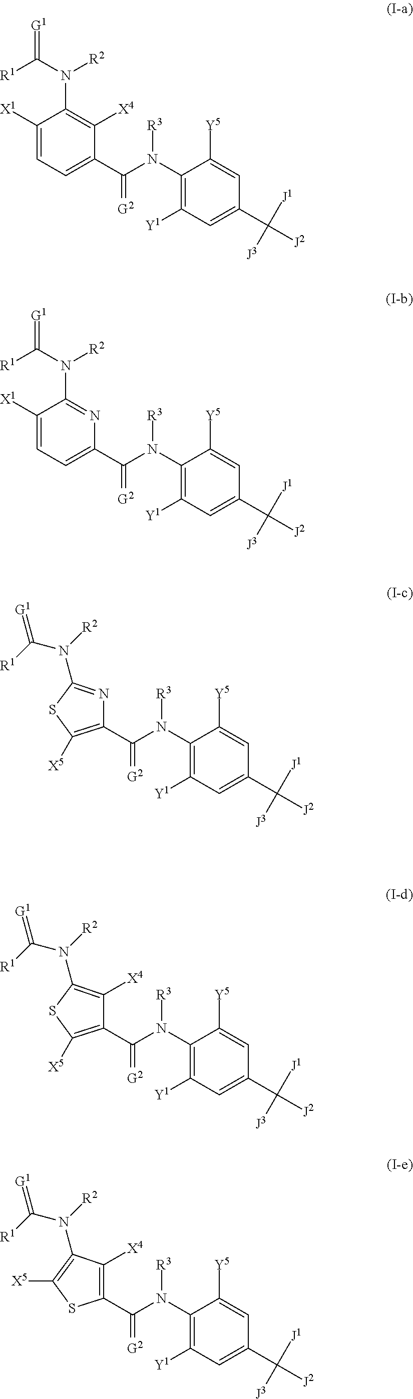

In an embodiment B, the invention is directed to compounds as defined in embodiment A, wherein J¹ and J² independently of each other stands for C₁-C₆ haloalkyl, preferably C₁-C₆ fluoroalkyl, more preferred C₁-C₁ perfluoroalkyl.

In an embodiment C, the invention is directed to compounds as defined in embodiment A, wherein J¹ stands for C₁-C₆ haloalkyl, preferably C₁-C₆ fluoroalkyl, more preferred C₁-C₁ perfluoroalkyl and J² stands for optionally substituted phenyl or a 5- to 6-membered heterocyclic group comprising at least one heteroatom selected among N, O and S.

In an embodiment D, the invention directed to compounds as defined in any one of the embodiments A to C, wherein J³ stands for a group OR⁴ wherein R⁴ preferably represents C₂-C₆ alkyl, C₂-C₆ alkenyl, C₂-C₆ alkynyl, C₃-C₈ cycloalkyl, C₃-C₈ cycloalkenyl, C₁-C₆alkoxyC₁-C₆alkyl, C₁-C₆alkylC₁-C₆thioalkyl, C₁-C₆haloalkylC₁-C₆thioalkyl, phenyl, aralkyl (preferably benzyl), pyridine, which groups may be substituted with halogen, C₁-C₆ haloalkyl, C₁-C₆alkyl, C₁-C₆alkoxy, C₁-C₆haloalkylsulfan, CN, acetamido, amino, diC₁-C₆ alkylamino and sulfamoylC₁-C₆alkyl, C₃-C₈ cycloalkyl, substituted phenyl, or aralkyl or represents iminyl which may be substituted with C₁-C₆alkyl, C₃-C₈ cycloalkyl, substituted phenyl, or aralkyl. Preferred OR⁴ groups are optionally substituted C₁-C₄ alkoxy, C₁-C₄ haloalkoxy, C₁-C₄ alkylthio, phenoxy, C₇-C₁₀ aralkoxy and pridyloxy which may be substituted with the aforementioned substitutents.

In an embodiment E, the invention is directed to compounds as defined in any one of the embodiments A to C, wherein J³ stands for an optionally substituted heterocyclic group selected among benzimidazol (i.e. indazol), benzotriazol, pyrrolidin, piperidin, morpholino and thiomorpholino which groups may be substituted with halogen, C₁-C₆ haloalkyl, C₁-C₆alkyl, C₁-C₆alkoxy, C₁-C₆ haloalkylsulfan, CN, acetamido, amino, diC₁-C₆alkylamino and sulfamoylC₁-C₆alkyl, C₃-C₈ cycloalkyl, substituted phenyl, or aralkyl; or stands for one group selected among the groups T1 to T9

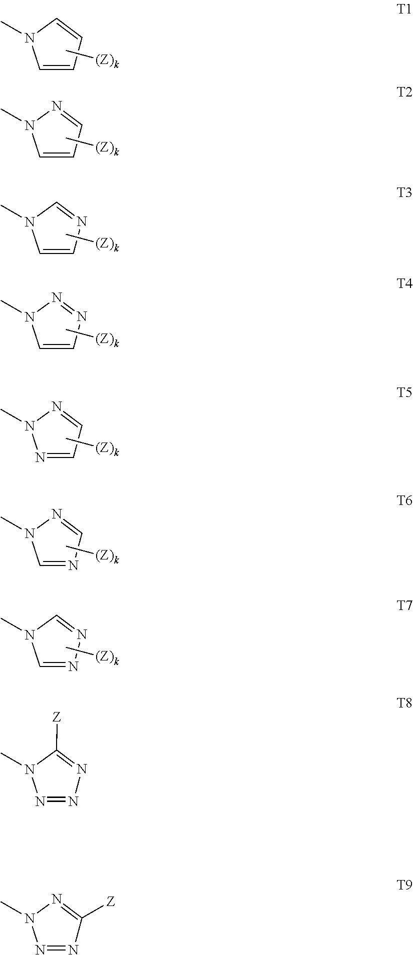

wherein k stands for 0, 1, 2, 3 or 4 and Z independently represents halogen, CN, nitro, hydroxyl, thiol, C₁-C₄ haloalkyl, C₁-C₄ alkoxy, C₁-C₄ haloalkoxy, C₁-C₄ alkylsulfenyl, C₁-C₄ alkylsulfinyl, C₁-C₄ alkylsulfonyl, C₁-C₄ haloalkylsulfenyl, C₁-C₄ haloalkylsulfinyl, C₁-C₄ haloalkylsulfonyl, C₁-C₆-alkyl-O—CO— which groups may be substituted with halogen, C₁-C₆ haloalkyl, C₁-C₆alkyl, C₁-C₆alkoxy, C₁-C₆ haloalkylsulfan, CN, acetamido, amino, diC₁-C₆ alkylamino and sulfamoyl C₁-C₆alkyl, C₃-C₈ cycloalkyl, substituted phenyl, or aralkyl, or iminyl which may be substituted with C₁-C₆alkyl, C₃-C₈ cycloalkyl, substituted phenyl, or aralkyl. Preferably Z represents halogen and optionally substituted C₁₋₄ haloalkyl.

According to the present invention, the amides of the above formula (I) show a strong pesticidal activity.

In the description of the present invention, the term “halogen” stands for fluoro, chloro, bromo, or iodo.

The term “alkyl” used either alone or combined with other terms such as “aminoalkyl” or “haloalkyl”, “haloalkoxy”, “haloalkylthio”, “haloalkylsulfinyl”, “haloalkylsulfonyl”, “alkoxy”, “alkylthio”, “alkylsulfinyl”, and “alkylsulfonyl”includes straight-chained or branched alkyl containing up to 12 carbon atoms, such as methyl, ethyl, n- or iso-propyl; n-, iso-, secondary- or tertiary-butyl; n-pentyl, n-hexyl, n-heptyl, n-octyl, n-nonyl, n-decyl, n-undecyl or n-dodecyl, and preferably represents alkyl having 1 to 6 carbon atoms, more referably represents alkyl having 1 to 6 carbon atoms. Alkyl may be substituted by at least one suitable substituent.

The term “alkylene” indicates a divalent group wherein one hydrogen atom is removed from the above “alkyl”.

The term “haloalkyl” used either alone or combined with other terms refers to alkyl groups which are partially or fully substituted with halogen atoms which may be the same or different. Examples of “haloalkyl” includes among others chemical groups like CF₃, CH₂F, CHF₂, CH₂CHF₂, CCl₃, CH₂Cl, CHCl₂, CF₂CF₃, CH₂CF₃, CH₂CH₂Cl, CH₂CH₂F, CHClCH₃, CHFCH₃, CH₂CHFCl, CHCl₂, CF₂CF₂H, CH₂CF₃. Preferred haloalkyl groups are CF₃, CH₂F, CHF₂, CCl₃, CH₂Cl, CHCl₂, CF₂CF₃, CHFCF₃. Haloalkyl groups may be substituted by at least one suitable substituent.

The term “alkenyl” used either alone or combined with other terms preferably stands for alkenyl having 2 to 6 or 2 to 5 carbon atoms. Examples include vinyl, allyl, 1-propenyl, 1-, 2-, or 3-butenyl or 1-pentenyl. More preferred it stands for alkenyl having 2 to 4 carbon atoms.

The term “alkynyl” used either alone or combined with other terms preferably stands for alkynyl having 2 to 6 or 2 to 5 carbon atoms. Examples include ethynyl, propargyl, 1-propynyl, but-3-ynyl or pent-4-ynyl. More preferred it stands for alkynyl having 2 to 4 carbon atoms.

The term “heterocyclic group” or “heterocycles” stands for heterocycles comprising at least one hetero atom selected among N, O and S. Examples thereof include, thienyl, furyl, pyrrolyl, pyrrolidinyl, pyrazolyl, imidazolyl, oxazolyl, isoxazolyl, thiadiazolyl, triazolyl, oxadiazolyl, pyridyl, piperidinyl, morpholinyl, pyrimidinyl, pyrazinyl, triazinyl, pyrrolyl, pyrrolidinyl, pyrazolyl, imidazolyl, triazolyl, piperidinyl, and morpholinyl, benzimidazolyl (indazolyl) and benzotriazolyl. The heterocycles may be substituted with at least one suitable substituent, which are preferably selected among the following groups nitro, cyano, fluoro, chloro, bromo, iodo and C₁-C₆ haloalkyl, for example, trifluoromethyl, difluoromethyl, difluorochloromethyl, 1,1,2,2-tetrafluoroethyl, pentafluoroethyl, heptafluoropropyl and heptafluoroisopropyl, C₁-C₆alkyl, C₁-C₆alkoxy, C₁-C₆ haloalkylsulfan, acetamido, amino, diC₁-C₆ alkylamino and sulfamoyl.

The term “optionally substituted” means unsubstituted or substituted with at least one substituent which is selected among C₁-C₆ haloalkyl, C₁-C₆alkyl, Cᵢ-C₆alkoxy, C₁-C₆ haloalkylsulfan, CN, acetamido, amino, diC₁-C₆ alkylamino and sulfamoyl, C₂₋₆Alkenyl, C₂₋₆Alkynyl, C₃₋₆Cycloalkyl, Chloro, Fluoro, Bromo, Iodo, NO₂, NRₓR_{y}, N₃, CN, SCN, ORₓ, SH, SF₅, COORₓ, C(O)Rₓ, CONRₓR_{y}, N═C(Rₓ)OR_{y}, SO₂NRₓR_{y}, Phenyl, heterocycles, whereas Rₓ and R_{y} independently of each other stands for H, C₁₋₆alkyl or C₁₋₆haloalkyl. The substituent preferably stands for methyl, ethyl, i-propyl, C₃₋Cycloalkyl, Chloro, Fluoro, Bromo, Iodo, NO₂, NH₂, NMe₂, NHMe, CN, SCN, OH, OMe, SH, SF₅, COOH, COOMe, C(O)H, COMe, CONH₂ COMe₂, N═CHOH. N═CHOMe, N═CMeOH, SO₂NHMe, SO₂NH₂, SO₂NMe₂, phenyl, or pyridine.

The compounds represented by the formula (I) of the present invention can be obtained according to a method of the following preparation methods:

Preparation Method (a):

A method of reacting compounds of formula (a-I):

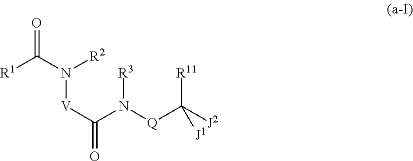

in which R¹¹ represents halogen or a group —O-L¹,

wherein L¹ represents alkylsulfonyl or phenylsulfonyl; R¹ to R³, V, Q, J¹ and J² have the same meaning as defined herein and wherein D in the chemical group V stands for the bonding site to the following moiety:

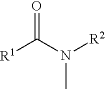

of formula (a-I), and E in the chemical group V stands for the bonding site to the following moiety:

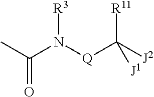

of formula (a-I)] with the compounds represented by the following formula:

M¹-J³ (r-I)

wherein M¹ represents hydrogen, an alkaline metal, an alkaline earth metal or salts thereof, for example, lithium, potassium, sodium, magnesium, and magnesium bromide and the like, and J³ has the same meaning as defined herein.

Preparation Method (b):

A method of reacting the compounds of fomula (b-I):

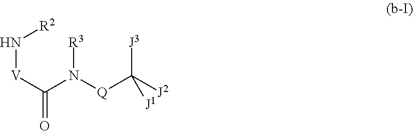

wherein
R², R³, V, J¹, J², J³ and Q have the same meaning as defined herein and wherein D in the chemical group V stands for the bonding site to the following moiety:

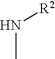

of formula (b-I), and wherein E in the chemical group stands for the bonding site to the following moiety:

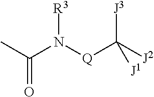

of formula (b-I) with compounds represented by the formula (r-III):

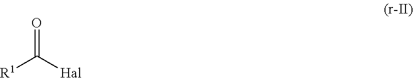

wherein R¹ has the same meaning as defined herein and Hal stands for halogen.

Preparation Method (c):

A method of reacting the compounds represented by the formula (c-I):

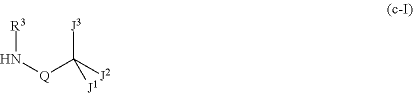

wherein
R³, J¹, J², J³ and Q have the same meaning as defined above, with the compounds represented by the following formula:

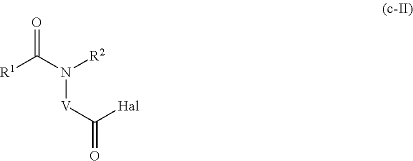

wherein
R¹, R², V and Hal have the same meaning as defined herein and wherein D in the chemical group V stands for the bonding site to the following moiety:

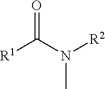

of formula (c-II), and wherein E in the chemical group V stands for the bonding site to the following moiety:

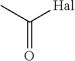

of formula (c-II)].

The above described Preparation method (a) can be represented by the following reaction scheme when, for example, 2-(4-{[(3-{[(2-chloropyridin-3-yl)-carbonyl]amino}phenyl)carbonyl]-amino}-3,5-dimethylphenyl)-1,1,1,3,3,3-hexafluoropropan-2-yl methanesulfonate and 4-bromo-1H-pyrazole are used as starting materials.

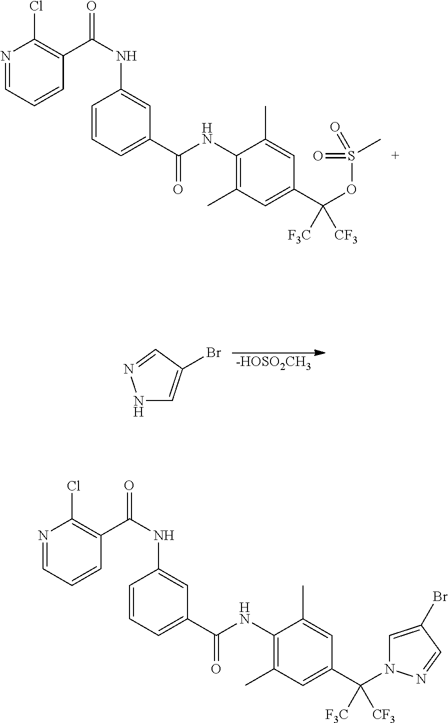

The above described Preparation method (b) can be represented by the following reaction scheme when, for example, 2-chloropyridine-3-carbonyl chloride and 3-amino-N-{4-[2-(4-bromo-1H-pyrazol-1-yl)-1,1,1,3,3 ,3-hexafluoropropan-2-yl]-2,6-dimethylphenyl}benzamide are used as starting materials.

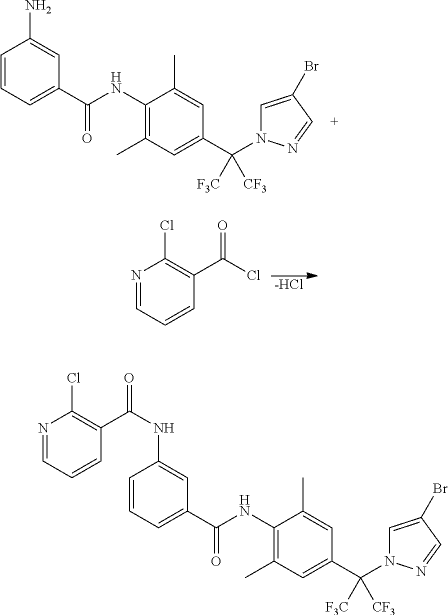

The above described Preparation method (c) can be represented by the following reaction scheme when, for example, 3-{[(2-chloropyridin-3-yl)carbonyl]-amino}benzoyl chloride and 4-[2-(4-bromo-1H-pyrazol-1-yl)-1,1,1,3,3,3-hexafluoro-propan-2-yl]-2,6-dimethylaniline are used as starting materials.

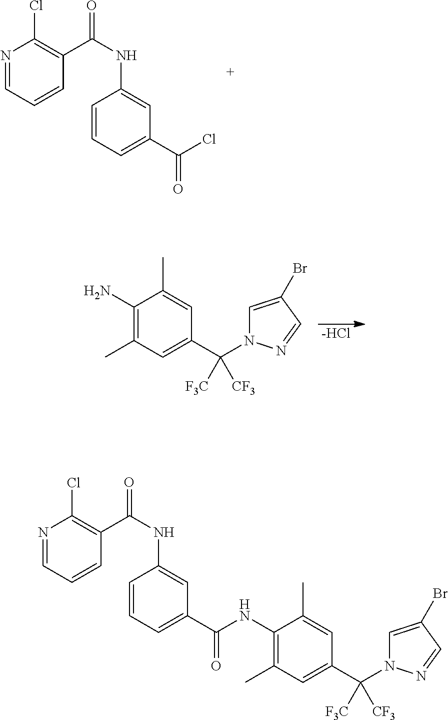

The above described Preparation method (a) can be performed according to a method that is described in JP-A No 8-311036, Journal of Fluorine Chemistry, 121, (2003) pp. 141-146 or Journal of the American Chemical Society, III, (1989) pp. 1455-1465.

The compounds of formula (a-I), which are used as reaction materials for the above Preparation method (a) are novel compounds and representative examples thereof for example include: 2-(4-{[(3-{[(2-chloropyridin-3-yl)carbonyl]amino}phenyl)carbonyl]amino}-3,5-dimethylphenyl)-1,1,1,3,3,3-hexafluoropropan-2-yl methanesulfonate, 2-(3,5-dibromo-4-{[(3-{[(2-chloro-pyridin-3-yl)carbonyl]amino}phenyl)carbonyl]-amino}phenyl)-1,1,1,3,3,3-hexafluoropropan-2-yl methanesulfonate, 1-(4-{[(3-{[(2-chloropyridin-3-yl)carbonyl]amino}phenyl)carbonyl]amino}-3,5-dimethylphenyl)-2,2,2-trifluoroethyl methanesulfonate, 1-(4-{[(3-{[(2-chloropyridin-3-yl)-carbonyl]amino}phenyl)carbonyl]amino}-3,5-dimethylphenyl)-2,2,2-trifluoro-1-phenylethyl methanesulfonate, 1-(4-{[(3-{[(2-chloropyridin-3-yl)carbonyl]amino}phenyl)carbonyl]amino}-3,5-dimethylphenyl)-1,1,1-trifluoropropan-2-yl methanesulfonate, 2-(4-{[(3-{[(2-chloropyridin-3-yl)carbonyl]amino}-2-fluorophenyl)carbonyl]amino}-3,5-dimethylphenyl)-1,1,1,3,3,3-hexafluoropropan-2-yl methanesulfonate, 2-(4-{[(3-{[(2-fluoropyridin-3-yl)carbonyl]amino}phenyl)-carbonyl]amino}-3,5-dimethylphenyl)-1,1,1,3,3,3-hexafluoropropan-2-yl methanesulfonate, 2-(4-{[(3-{[(2-chlorophenyl)carbonyl]amino}phenyl)carbonyl]amino}-3,5-dimethylphenyl)-1,1,1,-3,3,3-hexafluoropropan-2-yl methanesulfonate, 2-(4-{[(3-{[(2-fluorophenyl)carbonyl]amino}-phenyl)carbonyl]amino}-3,5-dimethylphenyl)-1,1,1,3,3,3-hexafluoropropan-2-yl methanesulfonate, 2-(4-{[(3-{[(3-chlorophenyl)carbonyl]amino}phenyl)carbonyl]amino}-3,5-dimethylphenyl)-1,1,1,-3,3,3-hexafluoropropan-2-yl methanesulfonate, 2-(4-{[(3-{[3-(fluorophenyl)carbonyl]amino}-phenyl)carbonyl]amino}-3,5-dimethylphenyl)-1,1,1,3,3,3-hexafluoropropan-2-yl methanesulfonate, 2-(4-{[(3-{[(4-chlorophenyl)carbonyl]amino}phenyl)carbonyl]amino}-3,5-dimethylphenyl)-1,1,1,-3,3,3-hexafluoropropan-2-yl methanesulfonate, 2-(4-{[(3-{[(4-fluorophenyl)carbonyl]amino}-phenyl)carbonyl]amino}-3,5-dimethylphenyl)-1,1,1,3,3,3-hexafluoropropan-2-yl methanesulfonate, 2-(4-{[(3-{[(2,6-dichlorophenyl)carbonyl]amino}phenyl)carbonyl]amino}-3,5-dimethylphenyl-1,1,1,3,3,3-hexafluoropropan-2-yl methanesulfonate, 2-(4-{[(3-{[(2,6-difluorophenyl-)carbonyl]-amino}phenyl)carbonyl]amino}-3,5-dimethylphenyl)-1,1,1,3,3,3-hexafluoropropan-2-yl methanesulfonate.

Representative examples of the compound of formula (r-I) which are used as reaction materials for the above Preparation method (a) include for example sodium cyanide, sodium azide, methylmagnesium bromide, ethylmagnesium bromide, sodium methoxide, methanol, sodium ethoxide, ethanol, sodium ethenolate, sodium phenoxide, phenol, sodium thiomethoxide, thiomethanol, sodium thioethoxide, thioethanol, sodium thiophenoixde, thiphenol, sodium dimethylazanide, dimethylamine, sodium bis(methoxycarbonyl)methanide, sodium cyano(methoxycarbonyl)methanide, sodium dicyanomethanide, sodium pyrazol-1-ide, pyrazole, sodium 4-chloropyrazol-1-ide, 4-chloropyrazole, sodium 4-bromopyrazol-1-ide, 4-bromopyrazole, sodium 3,5-bistrifluoromethylpyrazol-1-ide, 3,5-bistrifluoromethylpyrazole, sodium pyrrolidin-1-ide, pyrrolidine, sodium piperidin-1-ide, piperidine, sodium morpholin-4-ide, morpholine.

The reaction of the above Preparation method (a) can be carried out in an appropriate diluent. Examples of the diluent which may be used during the process include for example:

ethers such as ethyl ether, methyl ethyl ether, isopropyl ether, butyl ether, dioxane, dimethoxyethane (DME), tetrahydrofuran (THF), diethylene glycol dimethyl ether (DGM) and the like; nitriles such as acetonitrile, propionitrile and the like; acid amides such as dimethylformamide (DMF), dimethylacetamide (DMA), N-methylpyrrolidone, 1,3-dimethyl-2-imidazolidinone, hexamethylphosphoric triamide (HMPA) and the like; sulfones and sulfoxides such as dimethyl sulfoxide (DMSO), sulfolane and the like; and bases such as pyridine and the like.

Preparation method (a) can be carried out in the presence of an acid coupling agent, and such acid coupling agent includes inorganic bases such as hydride, hydroxide, carbonate and bicarbonate of an alkaline metal and an alkaline earth metal, for example, sodium hydride, lithium hydride, sodium bicarbonate, potassium bicarbonate, sodium carbonate, potassium carbonate, lithium hydroxide, sodium hydroxide, potassium hydroxide, calcium hydroxide and the like and; inorganic alkaline metal amides, for example, lithium amide, sodium amide, potassium amide and the like.

Preparation method (a) can be carried out within a substantially broad range of temperatures.

Generally, it can be carried out at a temperature of between approximately −10 and approximately 100° C., preferably between approximately 0 and approximately 30° C.

In addition, although the above reaction is preferably carried out at atmospheric pressure, it can be also carried out under reduced or elevated pressure.

For carrying out Preparation method (a), for example, relative to 1 mole of the compound of formula (a-I), 1.0 to 1.2 moles of the compound of formula (r-I) can be reacted in a diluent, for example DMF, to obtain the desired compound.

The compounds of formula (a-I) that are used as reaction materials for the above Preparation method (a) can be obtained by reacting the compounds represented by the following formula:

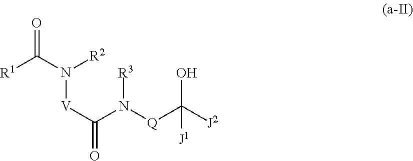

wherein
R¹ to R³, V, Q, J¹ and J² have the same meaning as defined herein and wherein D in the chemical group V stands for the bonding site to the following moiety:

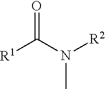

of formula (a-II), and wherein E in the chemical group V stands for the bonding site to the following moiety:

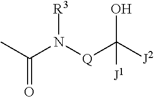

of formula (a-II) with the compounds represented by the following formula:

Hal-L¹ (r-III)

wherein
L¹ and Hal have the same meaning as defined above or halogenating agents according to conventional methods.

Representative examples of the compound of formula (a-II) are as follows and include known compounds:

2-chloro-N-(3-{[4-(1,1,1,3,3,3-hexafluoro-2-hydroxypropan-2-yl)-2,6-dimethylphenyl]-carbamoyl}phenyl)pyridine-3-carboxyamide, 2-chloro-N-(3-{[2,6-dibromo-4-(1,1,1,3,3,3-hexafluoro-2-hydroxypropan-2-yl)phenyl]-carbamoyl}phenyl)pyridine-3-carboxyamide, 2-chloro-N-(3-{[2,6-dimethyl-4-(2,2,2-trifluoro-1-hydroxyethyl)phenyl]carbamoyl}-phenyl)pyridine-3-carboxyamide, 2-chloro-N-(3-{[2,6-dimethyl-4-(2,2,2-trifluoro-1-hydroxy-1-phenylethyl)phenyl]-carbamoyl}-phenyl)pyridine-3-carboxyamide, 2-chloro-N-(3-{[2,6-dimethyl-4-(1,1,1-trifluoro-2-hydroxy-propan-2-yl)phenyl]-carbamoyl}phenyl)pyridine-3-carboxyamide, 2-chloro-N-(2-fluoro-3-{[4-(1,1,1,3,3,3-hexafluoro-2-hydroxypropan-2-yl)-2,6-dimethylphenyl]carbamoyl}phenyl)pyridine-3-carboxyamide, 2-fluoro-N-(3-{[4-(1,1,1,3,3,3-hexafluoro-2-hydroxypropan-2-yl)-2,6-dimethyl-phenyl]-carbamoyl}phenyl)pyridine-3-carboxyamide, 2-chloro-N-(3-{[4-(1,1,1,3,3,3-hexafluoro-2-hydroxypropan-2-yl)-2,6-dimethylphenyl]-carbamoyl}phenyl)benzamide, 2-fluoro-N-(3-{[4-(1,1,1,3,3,3-hexafluoro-2-hydroxypropan-2-yl)-2,6-dimethylphenyl]-carbamoyl}phenyl)benzamide, 3-chloro-N-(3-{[4-(1,1,1,3,3,3-hexafluoro-2-hydroxypropan-2-yl)-2,6-dimethylphenyl]-carbamoyl}phenyl)benzamide, 3-fluoro-N-(3-{[4-(1,1,1,3,3,3-hexafluoro-2-hydroxypropan-2-yl)-2,6-dimethylphenyl]-carbamoyl}phenyl)benzamide, 4-chloro-N-(3-{[4-(1,1,1,3,3 ,3-hexafluoro-2-hydroxypropan-2-yl)-2,6-dimethylphenyl]-carbamoyl}phenyl)benzamide, 4-fluoro-N-(3-{[4-(1,1,1,3,3,3-hexafluoro-2-hydroxypropan-2-yl)-2,6-dimethylphenyl]-carbamoyl}phenyl)benzamide, 2,6-difluoro-N-(3-{[4-(1,1,1,3,3,3-hexafluoro-2-hydroxypropan-2-yl)-2,6- dimethyl-phenyl]carbamoyl}phenyl)benzamide, and the like.

Representative examples of the compound of formula (r-III) include: methanesulfonyl chloride, trifluoromethanesulfonyl chloride, p-toluenesulfonyl chloride, and the like.

The reaction from the above compounds of formula (a-II) to the compounds of formula (a-I) can be carried out in the presence of an appropriate diluent. Examples of the diluent which may be used during the process include: aliphatic, alicyclic, and aromatic hydrocarbons (which may be chlorinated in some cases) such as pentane, hexane, cyclohexane, petroleum ether, ligroin, benzene, toluene, xylene, dichloromethane, chloroform, carbon tetrachloride, 1,2-dichloroethane, chlorobenzene, dichlorobenzene and the like; ethers such as ethyl ether, methyl ethyl ether, isopropyl ether, butyl ether, dioxane, dimethoxyethane (DME), tetrahydrofuran (THF), diethylene glycol dimethyl ether (DGM) and the like; ketones such as acetone, methyl ethyl ketone (MEK), methyl isopropyl ketone, methyl isobutyl ketone (MIBK) and the like; nitriles such as acetonitrile, propionitrile, acrylonitrile and the like; esters such as ethyl acetate, amyl acetate and the like; acid amides such as dimethylformamide (DMF), dimethylacetamide (DMA), N-methylpyrrolidone, 1,3-dimethyl-2-imidazolidinone, hexamethylphosphoric triamide (HMPA) and the like; sulfones and sulfoxides such as dimethyl sulfoxide (DMSO), sulfolane and the like; and bases such as pyridine and the like.

The reaction from the above compounds of formula (a-II) to the compounds of formula (a-I) can be carried out in the presence of an acid coupling agent, and such acid coupling agent is for example an inorganic base such as hydride, hydroxide, carbonate and bicarbonate of an alkaline metal and an alkaline earth metal, for example sodium hydride, lithium hydride, sodium bicarbonate, potassium bicarbonate, sodium carbonate, potassium carbonate, lithium hydroxide, sodium hydroxide, potassium hydroxide, calcium hydroxide and the like; inorganic alkaline metal amides, for example, lithium amide, sodium amide, potassium amide and the like; tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2.2.2]octane (DABCO), 1,8-diazabicyclo[5.4.0]undec-7-ene (DBU) and the like, and; organic lithium compounds, for example, methyl lithium, n-butyllithium, sec-butyllithium, tert-butyllithium, phenyl lithium, dimethyl copper lithium, lithium diisopropylamide, lithium cyclohexylisopropylamide, lithium dicyclohexylamide, n-butyllitium-DABCO, n-butyllithium-DBU, n-butyllithium-TMEDA and the like.

The reaction from the above compounds of formula (a-II) to the compounds of formula (a-I) can be carried out within a substantially broad range of temperatures.

Generally, it can be carried out at a temperature of between approximately −20 and approximately 100° C., preferably between approximately −10 and approximately 50° C.

In addition, although the above reaction is preferably carried out at atmospheric pressure, it can be also carried out under reduced or elevated pressure.

For carrying out the reaction from the above compounds of formula (a-II) to the compounds of formula (a-I), for example, relative to 1 mole of the compound of formula (a-II), 2 to 3 moles of the compound of formula (r-III) can be reacted in a diluent, for example dichloromethane, to obtain the desired compound in the presence of triethylamine.

The compounds of formula (a-II) can be obtained by reacting the compounds represented by the following formula:

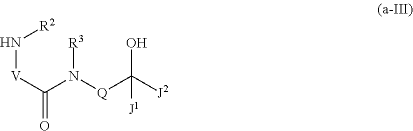

wherein

R², R³, V, Q, J¹ and J² have the same meaning as described herein and wherein D in the chemical group V stands for the bonding site to the following moiety:

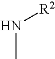

of formula (a-III), and wherein E in the chemical group V stands for the bonding site to the following moiety:

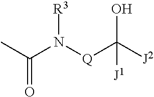

of formula (a-III) with the compounds of the above formula (r-II).

Representative examples of the compound of formula (a-III) are as follows and include known compounds:

3-amino-N-[4-(1,1,1,3,3,3-hexafluoro-2-hydroxypropan-2-yl)-2,6-dimethylphenyl]-benzamide,-3-amino-N-[2,6-dibromo-4-(1,1,1,3,3,3-hexafluoro-2-hydroxypropan-2-yl)phenyl]-benzamide, 3-amino-2-fluoro-N-[4-(1,1,1,3,3,3-hexafluoro-2-hydroxypropan-2-yl)-2,6-dimethylphenyl]-benzamide, 3-amino-N-[2,6-dimethyl-4-(2,2,2-trifluoro-2-hydroxyethyl)phenyl]benzamide, 3-amino-N-[2,6-dimethyl-4-(2,2,2-trifluoro-1-hydroxy-1-phenylethyl)phenyl]-benzamide, 3-amino-N-[2,6-dimethyl-4-(1,1,1-trifluoro-2-hydroxypropan-2-yl)phenyl]benzamide, and the like.

The compounds of formula (r-II) are known and representative examples may include:

2-chloropyridine-3-carbonyl chloride, 2-fluoropyridine-3-carbonyl chloride, 2-chlorobenzoyl chloride, 2-fluorobenzoyl chloride, 3-chlorobenzoyl chloride, 3-fluorobenzoyl chloride, 4-chlorobenzoyl chloride, 4-fluorobenzoyl chloride, 2,6-difluorobenzoyl chloride and the like.

The reaction from the above compounds of formula (a-III) to the compounds of formula (a-II) can be carried out in an appropriate diluent. Examples of the diluent which may be used during the process may include: aliphatic, alicyclic, and aromatic hydrocarbons (which may be chlorinated in some cases) such as pentane, hexane, cyclohexane, petroleum ether, ligroin, benzene, toluene, xylene, dichloromethane, chloroform, carbon tetrachloride, 1,2-dichloroethane, chlorobenzene, dichlorobenzene and the like; ethers such as ethyl ether, methyl ethyl ether, isopropyl ether, butyl ether, dioxane, dimethoxyethane (DME), tetrahydrofuran (THF), diethylene glycol dimethyl ether (DGM) and the like; ketones such as acetone, methyl ethyl ketone (MEK), methyl isopropyl ketone, methyl isobutyl ketone (MIBK) and the like; nitriles such as acetonitrile, propionitrile, acrylonitrile and the like; esters such as ethyl acetate, amyl acetate and the like; acid amides such as dimethylformamide (DMF), dimethylacetamide (DMA), N-methylpyrrolidone, 1,3-dimethyl-2-imidazolidinone, hexamethylphosphoric triamide (RMPA) and the like; sulfones and sulfoxides, for example, dimethyl sulfoxide (DMSO), sulfolane and the like; and bases such as pyridine and the like.

The reaction from the above compounds of formula (a-III) to the compounds of formula (a-II) can be carried out in the presence of an acid coupling agent, and such acid coupling agent may includes inorganic bases such as hydride, hydroxide, carbonate and bicarbonate of an alkaline metal and an alkaline earth metal, for example, sodium hydride, lithium hydride, sodium bicarbonate, potassium bicarbonate, sodium carbonate, potassium carbonate, lithium hydroxide, sodium hydroxide, potassium hydroxide, calcium hydroxide and the like; inorganic alkaline metal amides, for example, lithium amide, sodium amide, potassium amide and the like; tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2.2.2]octane (DABCO), 1,8-diazabicyclo[5.4.0]undec-7-ene (DBU) and the like; and organic lithium compounds, for example, methyl lithium, n-butyllithium, sec-butyllithium, tert-butyllithium, phenyl lithium, dimethyl copper lithium, lithium diisopropylamide, lithium cyclohexylisopropylamide, lithium dicyclohexylamide, n-butyllitium-DABCO, n-butyllithium-DBU, n-butyllithium-TMEDA and the like.

The reaction from the above compounds of formula (a-III) to the compounds of formula (a-II) can be also carried out based on a method which uses a phase-transfer catalyst. Examples of the diluent which may be used during the process may include water; aliphatic, alicyclic, and aromatic hydrocarbons (which may be chlorinated in some cases) such as pentane, hexane, cyclohexane, benzene, toluene, xylene, and the like; ethers such as ethyl ether, methyl ethyl ether, methyl butyl ether, isopropyl ether, butyl ether, and the like.

Examples of a phase-transfer catalyst may include quaternary ions such as tetramethylammonium bromide, tetrapropylammonium bromide, tetrabutylammonium bromide, tetrabutylammonium bisulfate, tetrabutylammonium iodide, trioctylmethylammonium chloride, benzyltriethylammonium bromide, butylpyridinium bromide, heptylpyridinium bromide, benzyltriethylammonium chloride and the like; crown ethers such as dibenzo-18-crown-6, dicyclohexyl-18-crown-6, 18-crown-6 and the like; cryptands such as [2.2.2]-cryptate, [2.1.1]-cryptate, [2.2.1]-cryptate, [2.2.B]-cryptate, [2O2O2S]-cryptate, [3.2.2]-cryptate and the like.

The reaction from the above compounds of formula (a-III) to the compounds of formula (a-II) can be carried out within a substantially broad range of temperature. Generally, it can be carried out at a temperature of between approximately −20 and approximately 100° C., preferably between approximately −10 and approximately 50° C.

In addition, although the above reaction is preferably carried out at atmospheric pressure, it can be also carried out under reduced or elevated pressure.

For carrying out the reaction from the above compounds of formula (a-III) to the compounds of formula (a-II), for example, relative to 1 mole of the compound of formula (a-III), 1 to 1.5 moles of the compound of formula (r-II) can be reacted in a diluent, for example tetrahydrofuran, to obtain the desired compound in the presence of pyridine, for example.

The compounds of formula (a-III) can be obtained by reacting the compounds represented by the following formula:

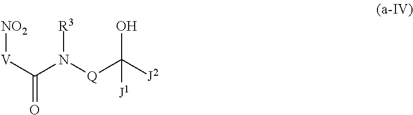

wherein
R³, V, Q, J¹ and J² have the same meaning as described herein and wherein D in the chemical group V stands for the bonding site to the following moiety:

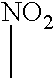

of formula (a-IV), and wherein E in the chemical group V stands for the bonding site to the following moiety:

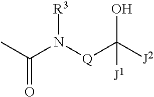

of formula (a-IV) with appropriate reducing agents.

Representative examples of the compound of formula (a-IV) are as follows and include known compounds:

N-[4-(1,1,1,3,3,3-hexafluoro-2-hydroxypropan-2-yl)-2,6-dimethylphenyl]-3-nitrobenzamide, N-[2,6-dibromo-4-(1,1,1,3,3,3-hexafluoro-2-hydroxypropan-2-yl)phenyl]-3-nitrobenzamide, 2-fluoro-N-[4-(1,1,1,3,3,3-hexafluoro-2-hydroxypropan-2-yl)-2,6-dimethylphenyl]-3-nitrobenzamide, N-[2,6-dimethyl-4-(2,2,2-trifluoro-2-hydroxyethyl)phenyl]-3-nitrobenzamide, N-[2,6-dimethyl-4-(2,2,2-trifluoro-1-hydroxy-1-phenylethyl)phenyl]-3-nitrobenzamide, N-[2,6-dimethyl-4-(1,1,1-trifluoro-2-hydroxypropan-2-yl)phenyl]-3-nitrobenzamide, and the like.

The reaction from the above compounds of formula (a-IV) to the compounds of formula (a-III) can be carried out in an appropriate diluent. Examples of the diluent which may be used during the process may include water; ethers such as dioxane, dimethoxyethane (DME), tetrahydrofuran (THF), diethylene glycol dimethyl ether (DGM) and the like; and alcohols such as methanol, ethanol, isopropanol, butanol, ethylene glycol and the like;

The reaction from the above compounds of formula (a-IV) to the compounds of formula (a-III) can be carried out in the presence of an acid catalyst. Examples of the acid catalyst may include organic acids, for example, formic acid, acetic acid, trifluoroacetic acid, propionic acid, methanesulfonic acid, benzenesulfonic acid, p-toluenesulfonic acid, and the like.

The reaction from the above compounds of formula (a-IV) to the compounds of formula (a-III) can be carried out in the presence of an appropriate reducing agent. Examples of the reducing agent may include lithium aluminum hydride, sodium boron hydride, nickel chloride, iron and acetic acid, hydrochloric acid and stannic chloride and the like.

The reaction from the above compounds of formula (a-IV) to the compounds of formula (a-III) can be carried out within a substantially broad range of temperature.

Generally, it can be carried out at a temperature of between approximately −20 and approximately 150° C., preferably between approximately 0 and approximately 100° C.

In addition, although the above reaction is preferably carried out at atmospheric pressure, it can be also carried out under reduced or elevated pressure.

For carrying out the reaction from the above compounds of formula (a-IV) to the compounds of formula (a-III), for example, relative to 1 mole of the compound of formula (a-IV), 3 to 4 moles of stannic chloride in a diluent, for example ethanol, can be reacted in the presence of concentrated hydrochloric acid to obtain the desired compound.

The compounds of formula (a-IV) can be obtained by reacting the compounds represented by the following formula:

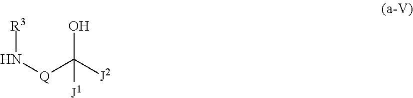

wherein
R³, Q, J¹ and J² have the same meaning as described above, with the compounds represented by the following formula:

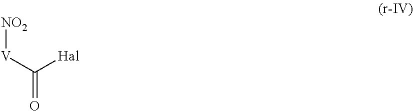

wherein
V and Hal have the same meaning as defined herein and wherein D in the chemical group V stands for the bonding site to the following moiety:

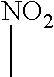

of formula (r-IV), and wherein E in the chemical group V stands for the bonding site to the following moiety:

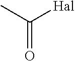

of formula (r-IV).

Representative examples of the compound of formula (a-V) are as follows and include known compounds:

2-(4-amino-phenyl)-1,1,1,3,3,3-hexafluoropropan-2-ol, 2-(4-amino-3,5-dimethylphenyl)-1,1,1,3,3,-3-hexafluoropropan-2-ol, 2-(4-amino-3,5-dibromophenyl)-1,1,1,3,3,3-hexafluoropropan-2-ol, 1-(4-amino-phenyl)-2,2,2-trifluoroethanol, 1-(4-amino-3,5-dimethylphenyl)-2,2,2-trifluoroethanol, 1-(4-amino-3,5-dibromophenyl)-2,2,2-trifluoroethanol, 1-(4-amino-phenyl)-2,2,2-trifluoro-1-phenylethanol, 1-(4-amino-3,5-dimethylphenyl)-2,2,2-trifluoro-1-phenylethanol, 1-(4-amino-3,5-dibromophenyl)-2,2,2-trifluoro-1-phenylethanol, 2-(4-amino-phenyl)-1,1,1-trifluoropropan-2-ol, 2-(4-amino-3,5-dimethylphenyl)-1,1,1-trifluoropropan-2-ol, 2-(4-amino-3,5-dibromo-phenyl)-1,1,1-trifluoropropan-2-ol, and the like.

Representative examples of the compound of formula (r-IV) may include 3-nitrobenzoyl chloride and the like.

The reaction from the above compounds of formula (a-V) to the compounds of formula (a-IV) can be carried out in an appropriate diluent. Examples of the diluent which may be used during the process may include: aliphatic, alicyclic, and aromatic hydrocarbons (which may be chlorinated in some cases) such as pentane, hexane, cyclohexane, petroleum ether, ligroin, benzene, toluene, xylene, dichloromethane, chloroform, carbon tetrachloride, 1,2-dichloroethane, chlorobenzene, dichlorobenzene and the like; ethers such as ethyl ether, methyl ethyl ether, isopropyl ether, butyl ether, dioxane, dimethoxyethane (DME), tetrahydrofuran (THF), diethylene glycol dimethyl ether (DGM) and the like; ketones such as acetone, methyl ethyl ketone (MEK), methyl isopropyl ketone, methyl isobutyl ketone (MIBK) and the like; nitriles such as acetonitrile, propionitrile, acrylonitrile and the like; esters such as ethyl acetate, amyl acetate and the like; acid amides such as dimethylformamide (DMF), dimethylacetamide (DMA), N-methylpyrrolidone, 1,3-dimethyl-2-imidazolidinone, hexamethylphosphoric triamide (HMPA) and the like; sulfones and sulfoxides, for example, dimethyl sulfoxide (DMSO), sulfolane and the like; and bases such as pyridine and the like.

The reaction from the above compounds of formula (a-V) to the compounds of formula (a-IV) can be carried out in the presence of an acid coupling agent, and such acid coupling agent includes inorganic bases such as hydride, hydroxide, carbonate and bicarbonate of an alkaline metal and an alkaline earth metal, for example, sodium hydride, lithium hydride, sodium bicarbonate, potassium bicarbonate, sodium carbonate, potassium carbonate, lithium hydroxide, sodium hydroxide, potassium hydroxide, calcium hydroxide and the like; inorganic alkaline metal amides, for example, lithium amide, sodium amide, potassium amide and the like; tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2.2.2]octane (DABCO), 1,8-diazabicyclo[5.4.0]undec-7-ene (DBU) and the like, organic lithium compounds, for example, methyl lithium, n-butyllithium, sec-butyllithium, tert-butyllithium, phenyl lithium, dimethyl copper lithium, lithium diisopropylamide, lithium cyclohexylisopropylamide, lithium dicyclohexylamide n-butyllitium-DABCO, n-butyllithium-DBU, n-butyllithium-TMEDA and the like.

The reaction from the above compounds of formula (a-V) to the compounds of formula (a-IV) can be also carried out by a method which uses a phase-transfer catalyst. Examples of the diluent which may be used during the process may include water; aliphatic, alicyclic, and aromatic hydrocarbons (which may be chlorinated in some cases) such as pentane, hexane, cyclohexane, benzene, toluene, xylene, and the like; ethers such as ethyl ether, methyl ethyl ether, methyl butyl ether, isopropyl ether, butyl ether, and the like.

Examples of the a phase-transfer catalyst may include quaternary ions such as tetramethylammonium bromide, tetrapropylammonium bromide, tetrabutylammonium bromide, tetrabutylammonium bisulfate, tetrabutylammonium iodide, trioctylmethylammonium chloride, benzyltriethylammonium bromide, butylpyridinium bromide, heptylpyridinium bromide, benzyltriethylammonium chloride and the like; crown ethers such as dibenzo-18-crown-6, dicyclohexyl-18-crown-6, 18-crown-6 and the like; cryptands such as [2.2.2]-cryptate, [2.1.1]-cryptate, [2.2.1]-cryptate, [2.2.B]-cryptate, [2O2O2S]-cryptate, [3.2.2]-cryptate and the like.

The reaction from the above compounds of formula (a-V) to the compounds of formula (a-IV) can be carried out within a substantially broad range of temperature. Generally, it can be carried out at a temperature of between approximately −20 and approximately 100° C., preferably between approximately −10 and approximately 50° C.

In addition, although the above reaction is preferably carried out at atmospheric pressure, it can be also carried out under reduced or elevated pressure.

For carrying out the reaction from the above compounds of formula (a-V) to the compounds of formula (a-IV), for example, relative to 1 mole of the compound of formula (a-V), 1 to 1.5 moles of the compound of formula (r-IV) in a diluent, for example tetrahydrofuran, can be reacted in the presence of pyridine, for example, to obtain the desired compound.

The compounds of formula (a-V) can be obtained according to the following Preparation methods (d) and (e).

Preparation Method (d):

A method of reacting the compounds represented by the following formula:

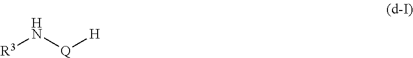

wherein
R³ and Q have the same meaning as described above, with the compounds represented by the following formula:

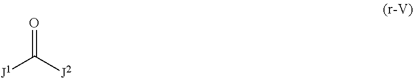

wherein
J¹ and J² have the same meaning as defined above, if necessary, in the presence of an acid catalyst.

Preparation Method (e):

A method of reacting the compounds represented by the following formula:

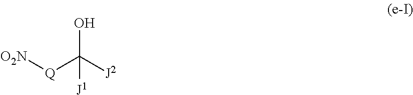

wherein
Q, J¹ and J² have the same meaning as described above, with appropriate reducing agents.

The compounds of formula (d-I) as reacting materials for Preparation method (d) are publicly known and the representative examples thereof may include:

aniline, 2,6-dimethylaniline, 2,6-dibromoaniline, and the like.

The compounds of formula (r-V) as reacting materials for Preparation method (d) are publicly known and the representative examples thereof may include:

1,1,1,3,3,3-hexafluoropropan-2-one, 1,1,1,3,3,4,4,4-octafluorobutan-2-one, 1,1,1,2,2,4,4,5,5,5-decafluoropentan-3-one, and the like.

Preparation method (d) can be carried out according to the methods described in WO2005/073165 and WO2006/137395.

The reaction for Preparation method (d) described above can be carried out in an appropriate diluent. Examples of the diluent which may be used during the process may include aliphatic, alicyclic, and aromatic hydrocarbons (which may be chlorinated in some cases) such as benzene, toluene, xylene, chlorobenzene, dichlorobenzene and the like; ethers such as dioxane, dimethoxyethane (DME), tetrahydrofuran (THY), diethylene glycol dimethyl ether (DGM) and the like, and; nitriles such as acetonitrile and propionitrile, and the like.

Preparation method (d) can be carried out in the presence of an acid catalyst. Examples of the acid catalyst may include mineral acid, for example, hydrochloric acid, sulfuric acid, nitric acid, hydrobromic acid, sodium hydrogen sulfite and the like; organic acids, for example, formic acid, acetic acid, trifluoroacetic acid, propionic acid, methanesulfonic acid, benzenesulfonic acid, p-toluenesulfonic acid, and the like; hydrochlorides of organic amines, for example, pyridine hydrochloride, triethylamine hydrochloride and the like; sulfonates of amines, for example, pyridine p-toluenesulfonate, triethylamine p-toluenesulfonate and the like.

Preparation method (d) can be carried out within a substantially broad range of temperature.

Generally, it can be carried out at a temperature of between approximately 50 and approximately 140° C., preferably between approximately 60 and approximately 120° C.

In addition, although the above reaction is preferably carried out at atmospheric pressure, it can be also carried out under reduced or elevated pressure.

For carrying out Preparation method (d), for example, relative to 1 mole of the compound of formula (d-I), 1 to 1.5 moles of the compound of formula (r-V) in a diluent, for example toluene, can be reacted in the presence of an acid catalyst to obtain the desired compound.

Representative compounds of formula (e-I) for Preparation method (e) are as follows that include publicly known compounds:

1-(4-nitrophenyl)-2,2,2-trifluoroethanol, 1-(3,5-dimethyl-4-nitrophenyl)-2,2,2-trifluoroethanol, 2-(4-nitrophenyl)-1,1,1-trifluoropropan-2-ol, 2-(3,5-dimethyl-4-nitrophenyl)-1,1,1-trifluoro-propan-2-ol, 1-(4-nitrophenyl)-2,2,2-trifluoro-1-phenylethanol, 1-(3,5-dimethyl-4-nitrophenyl)-2,2,2-trifluoro-1-phenylethanol and the like.

The above described Preparation method (e) can be carried out in reference to the method for synthesizing the compounds of formula (a-III) from the compounds of formula (a-IV) as described above.

The compounds of formula (e-I) as reacting materials for Preparation method (e) described above can be obtained by reacting the compounds represented by the following formula:

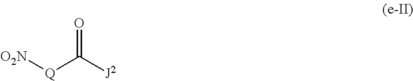

wherein
Q and J² have the same meaning as described above, with the compounds represented by the following formula:

J¹-SiMe₃ (r-VI)

wherein
Me represents methyl and J¹ has the same meaning as defined above.

The representative examples of the compound of formula (e-II) are as follows and include known compounds:

4-nitrobenzaldehyde, 3,5-dimethyl-4-nitrobenzaldehyde, 1-(4-nitrophenyl)ethanone, 1-(3,5-di-methyl-4-nitrophenyl)ethanone, (4-nitrophenyl)(phenyl)methanone, (3,5-dimethyl-4-nitrophenyl)(phenyl)methanone and the like.

The representative examples of the compound of formula (r-VI) may include: trimethyl(trifluoromethyl)silane, trifluoromethylsulfinylbenzene, trifluoromethylsulfonylbenzene and the like.

The above reaction from the compounds of formula (e-II) to the compounds of formula (e-I) can be carried out according to the methods described in Tetrahedron, 56 (2000) pp. 7613-7632, Journal of Fluorine Chemistry, 112 (2001) pp. 123-131, Synlett, 2006, pp. 112-114, Organic Letters, 2003 (5) pp. 3253-3256.

The reaction from the above compounds of formula (e-II) to the compounds of formula (e-I) can be carried out in an appropriate diluent. Examples of the diluent which may be used during the process include aliphatic, alicyclic, and aromatic hydrocarbons (which may be chlorinated in some cases) such as pentane, hexane, cyclohexane, petroleum ether, ligroin, benzene, toluene, xylene, dichloromethane, chloroform, carbon tetrachloride, 1,2-dichloroethane, chlorobenzene, dichlorobenzene and the like; ethers such as ethyl ether, methyl ethyl ether, isopropyl ether, butyl ether, dioxane, dimethoxyethane (DME), tetrahydrofuran (THF), diethylene glycol dimethyl ether (DGM) and the like; nitriles such as acetonitrile, propionitrile, acrylonitrile and the like; esters such as ethyl acetate, amyl acetate and the like; acid amides such as dimethylformamide (DMF), dimethylacetamide (DMA), N-methylpyrrolidone, 1,3-dimethyl-2-imidazolidinone, hexamethylphosphoric triamide (HMPA) and the like; sulfones and sulfoxides, for example, dimethyl sulfoxide (DMSO), sulfolane and the like.

The reaction from the above compounds of formula (e-II) to the compounds of formula (e-I) can be carried out in the presence of a catalyst. Examples of the catalyst may include tetrabutylammonium fluoride, tetraethylammonium fluoride, potassium fluoride, and the like.

The reaction from the above compounds of formula (e-II) to the compounds of formula (e-I) can be carried out within a substantially broad range of temperature.

Generally, it can be carried out at a temperature of between approximately −20 and approximately 100° C., preferably between approximately 0 and approximately 50° C.

In addition, although the above reaction is preferably carried out at atmospheric pressure, it can be also carried out under reduced or elevated pressure.

For carrying out the reaction from the above compounds of formula (e-II) to the compounds of formula (e-I), for example, relative to 1 mole of the compound of formula (e-II), 1 to 2 moles of the compound of formula (r-VI) in a diluent, for example, dichloromethane can be reacted in the presence of the above catalyst to obtain the desired compound.

The compounds of formula (e-II) can be obtained according to the following Preparation methods (f), (g) and (o).

Preparation Method (1):

A method of reacting the compounds represented by the following formula:

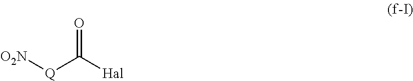

wherein
Q and Hal have the same meaning as described above, with the compounds represented by the following formula:

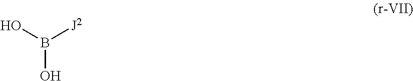

wherein
J² has the same meaning as defined above.

Preparation Method (g):

A method of oxidizing the compounds represented by the following formula:

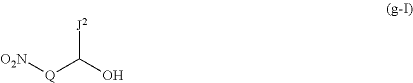

wherein
Q and J² have the same meaning as described above.

The compounds of formula (f-I) in the above Preparation example (f) are publicly known and representative examples thereof may include: 4-nitrobenzoyl chloride, 4-nitrobenzoyl bromide, 3,5-dimethyl-4-nitrobenzoyl chloride, 3,5-dimethyl-4-nitrobenzoyl bromide and the like.

Representative examples of the compound of formula (r-VII) in the above Preparation example (f) may include: phenylboronic acid, 4-chloro-phenylboronic acid, and the like.

The above described Preparation method (f) can be carried out according to the methods described in Tetrahedron Letters, 44 (2003) pp. 271-273, ibid., 40 (1999) 3057-3060 and Tetrahedron, 62 (2006) pp. 11675-11678.

Reaction of Preparation method (f) can be carried out in an appropriate diluent. Examples of the diluent which may be used during the process include water; aliphatic, alicyclic, and aromatic hydrocarbons (which may be chlorinated in some cases) such as pentane, hexane, cyclohexane, petroleum ether, ligroin, benzene, toluene, xylene, dichloromethane, chloroform, carbon tetrachloride, 1,2-dichloroethane, chlorobenzene, dichlorobenzene and the like; ethers such as ethyl ether, methyl ethyl ether, isopropyl ether, butyl ether, dioxane, dimethoxyethane (DME), tetrahydrofuran (THF), diethylene glycol dimethyl ether (DGM) and the like; ketones such as acetone, methyl ethyl ketone (MEK), methyl isopropyl ketone, methyl isobutyl ketone (MIBK) and the like; nitriles such as acetonitrile, propionitrile, acrylonitrile and the like; alcohols such as methanol, ethanol, isopropanol, butanol, ethylene glycol and the like; esters such as ethyl acetate, amyl acetate and the like; acid amides such as dimethylformamide (DMF), dimethylacetamide (DMA), N-methylpyrrolidone, 1,3-dimethyl-2-imidazolidinone, hexamethylphosphoric triamide (HMPA) and the like; sulfones and sulfoxides, for example, dimethyl sulfoxide (DMSO), sulfolane and the like; and bases such as pyridine and the like.

Preparation method (f) can be carried out in the presence of a pH buffer. Examples of such pH buffer may include phosphate salt and sulfate salt of an alkaline metal and an alkaline earth metal, as an inorganic base, for example, tripotassium phosphate and the like.

Preparation method (f) can be carried out in the presence of a catalyst. Examples of the catalyst may include dichlorobis(triphenylphosphine)palladium (II), and the like.

Preparation method (f) can be carried out within a substantially broad range of temperature.

Generally, it can be carried out at a temperature of between approximately 0 and approximately 150° C., preferably between approximately 50 and approximately 120° C.

In addition, although the above reaction is preferably carried out at atmospheric pressure, it can be also carried out under reduced or elevated pressure.

For carrying out Preparation method (f), for example, relative to 1 mole of the compound of formula (r-VII), 1 to 1.2 moles of the compound of formula (f-I) in a diluent, for example toluene, can be reacted in the presence of a pH buffer and a catalyst that are described above to obtain the desired compound.

Representative examples of the compound of formula (g-I) in the above Preparation example (g) are known and may include the following: (4-nitrophenyl)methanol, 1-(4-nitrophenyl)ethanol, (4-nitrophenyl)(phenyl)methanol and the like.

The reaction for Preparation method (g) can be carried out in an appropriate diluent. Examples of the diluent which may be used during the process may include water; aliphatic, alicyclic, and aromatic hydrocarbons (which may be chlorinated in some cases) such as pentane, hexane, cyclohexane, petroleum ether, ligroin, benzene, toluene, xylene, dichloromethane, chloroform, carbon tetrachloride, 1,2-dichloroethane, chlorobenzene, dichlorobenzene and the like; ketones such as acetone, methyl ethyl ketone (MEK), methyl isopropyl ketone, methyl isobutyl ketone (MIBK) and the like; nitriles such as acetonitrile, propionitrile, acrylonitrile and the like; esters such as ethyl acetate, amyl acetate and the like; acid amides such as dimethylformamide (DMF), dimethylacetamide (DMA), N-methylpyrrolidone, 1 ,3-dimethyl-2-imidazolidinone, hexamethylphosphoric triamide (HMPA) and the like; sulfones and sulfoxides, for example, dimethyl sulfoxide (DMSO), sulfolane and the like; and bases such as pyridine and the like.

Preparation method (g) can be carried out in the presence of an oxidizing agent. Examples of the oxidizing agent may include chromic acid, pyridium chlorochromate, periodic acid, manganese dioxide, potassium permanganate and the like.

Preparation method (g) can be carried out within a substantially broad range of temperature.

Generally, Preparation method (g) can be carried out at a temperature of between approximately −60 and approximately 100° C., preferably between approximately −20 and approximately 50° C.

In addition, although the above reaction is preferably carried out at atmospheric pressure, it can be also carried out under reduced or elevated pressure.

For carrying out Preparation method (g), for example, relative to 1 mole of the compound of formula (g-I), 1 to 2 moles of an oxidizing agent in a diluent, for example, dichloromethane can be reacted to obtain the desired compound.

The compounds of formula (g-I) as reacting materials for Preparation method (g) can be obtained according to Preparation method (h) and Preparation method (i).

Preparation Method (o):

A method of reacting the compounds represented by the following formula:

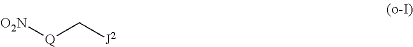

wherein
J² and Q have the same meaning as described above, with appropriate oxidizing agents, for example, chromic acid to obtain the desired products.

Preparation Method (h):

A method of reacting the compounds represented by the following formula:

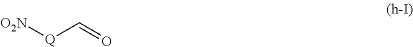

wherein
Q has the same meaning as described above with the compounds represented by the following formula:

M¹-J² (r-VIII)

wherein
M¹ and J² have the same meaning as defined above.

Preparation Method (i):

A method of reducing the compounds represented by the following formula:

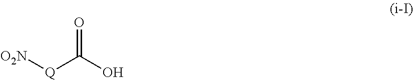

wherein
Q has the same meaning as described above.

Representative examples of the compound of formula (h-I) as reacting materials in the above Preparation example (h) may include: 4-nitrobenzaldehyde, 3,5-dimethyl-4-nitrobenzaldehyde and the like.

The compounds of formula (r-VIII) as reacting materials for the above Preparation example (h) are publicly known and representative examples thereof may include: methylmagnesium bromide, ethylmagnesium bromide, phenylmagnesium bromide and the like.

The above described Preparation method (h) can be carried out according to Grignard reaction, which is a common reaction in organic synthesis.

Representative examples of the compound of formula (i-I) as reacting materials in the above Preparation example (i) may include: 4-nitrobenzenecarboxylic acid, 3,5-dimethyl-4-nitrobenzenecarboxylic acid and the like.

Reaction for the above Preparation method (i) can be carried out in an appropriate diluent. Examples of the diluent which may be used during the process include ethers such as ethyl ether, methyl ethyl ether, isopropyl ether, butyl ether, dioxane, dimethoxyethane (DME), tetrahydrofuran (THF), diethylene glycol dimethyl ether (DGM) and the like.

Reaction for the above Preparation method (i) can be carried out in the presence of a reducing agent. Examples of the reducing agent may include diborane and the like.

Preparation method (i) can be carried out within a substantially broad range of temperature.

Generally, it can be carried out at a temperature of between approximately −20 and approximately 100° C., preferably between approximately 0 and approximately 50° C.

In addition, although the above reaction is preferably carried out at atmospheric pressure, it can be also carried out under reduced or elevated pressure.

For carrying out Preparation method (i), for example, relative to 1 mole of the compound of formula (i-I), 1 to 1.2 moles of a reducing agent in a diluent, for example, tetrahydrofuran can be reacted to obtain the desired compound.

The compounds of formula (i-I) as reacting materials for Preparation method (i) can be obtained by oxidizing the compounds represented by the following formula:

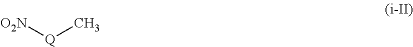

wherein
Q has the same meaning as defined above.

The compounds of formula (i-II) are publicly known and representative examples thereof may include: 1-methyl-4-nitrobenzene, 1,3,5-trimethyl-4-nitrobenzene and the like.

The above reaction from the compounds of formula (i-II) to the compounds of formula (i-I) can be carried out according to the method described in U.S. Pat. No. 6,455,528 B1.

The reaction from the compounds of formula (i-II) to the compounds of formula (i-I) can be carried out in the presence of an oxidizing agent. Examples of the oxidizing agent include chromic acid, pyridium chlorochromate, periodic acid, potassium permanganate and the like.

The reaction from the compounds of formula (i-II) to the compounds of formula (i-I) can be carried out in an appropriate diluent. Examples of the diluent which may be used during the process may include, water; alcohols such as methanol, ethanol, isopropanol, butanol, ethylene glycol and the like.

The reaction from the compounds of formula (i-II) to the compounds of formula (i-I) can be carried out in the presence of an acid catalyst. Examples of the acid catalyst include organic acids, for example, formic acid, acetic acid, trifluoroacetic acid, propionic acid and the like.

The reaction from the compounds of formula (i-II) to the compounds of formula (i-I) can be carried out within a substantially broad range of temperature. Generally, it can be carried out at a temperature of between approximately 50 and approximately 100° C., preferably between approximately 60 and approximately 80° C.

In addition, although the above reaction is preferably carried out at atmospheric pressure, it can be also carried out under reduced or elevated pressure.

For carrying out the reaction from the compounds of formula (i-II) to the compounds of formula (i-I), for example, relative to 1 mole of the compound of formula (i-II), 3 to 3.5 moles of the above oxidizing agents in a diluent, for example, acetic acid and isopropanol can be reacted to obtain the desired compound.

The compounds of formula (b-I) as reacting materials for the above Preparation example (b) are novel and representative examples thereof may include: 3-amino-N-{4-[1,1,1,3,3,3-hexafluoro-2-(1H-pyrazol-1-yl)propan-2-yl]-2,6-dimethyl-phenyl}benzamide, 3-amino-N-{2,6-dibromo-[1,1,1,3,3,3-hexafluoro-2-(1H-pyrazol-1-yl)propan-2-yl]-phenyl}-benzamide, 3-amino-N-{2,6-dimethyl-[2,2,2-trifluoro-1-(1H-pyrazol-1-yl)ethyl]phenyl}benzamide, 3-amino-N-{2,6-dimethyl-4-[2,2,2-trifluoro-1-phenyl-1-(1H-pyrazol-1-yl)ethyl]phenyl}-benzamide and the like.

The above described Preparation method (b) can be carried out in reference to the method for synthesizing the compounds of formula (a-II) from the compounds of formula (a-III) as described above.

The compounds of formula (b-I) as reacting materials for Preparation method (b) can be obtained by reacting the compounds represented by the following formula:

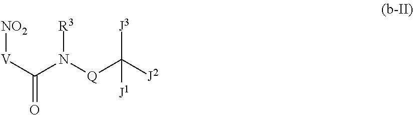

wherein
R³, V, Q, J¹, J² and J³ have the same meaning as described above [provided that, in V, D represents a bonding site to the following moiety:

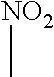

of formula (b-II), and E represents a bonding site to the following moiety:

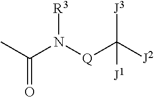

of formula (b-II)] with an appropriate reducing agent.

The compounds of formula (b-II) as described above are novel and representative examples thereof may include:

3-nitro-N-{4-[1,1,1,3,3,3-hexafluoro-2-(1H-pyrazol-1-yl)propan-2-yl]-2,6-dimethyl-phenyl}benzamide, 3-nitro-N-{2,6-dibromo-[1,1,1,3,3,3-hexafluoro-2-(1H-pyrazol-1-yl)propan-2-yl]-phenyl}-benzamide, 3-nitro-N-{2,6-dimethyl-[2,2,2-trifluoro-1-(1H-pyrazol-1-yl)ethyl]phenyl}benzamide, 3-nitro-N-{2,6-dimethyl-4-[2,2,2-trifluoro-1-phenyl-1-(1H-pyrazol-1-yl)ethyl]phenyl}-benzamide and the like.

The reaction from the compounds of formula (b-II) to the compounds of formula (b-I) as described above can be carried out in accordance with the method for the reaction from the compounds of formula (a-IV) to the compounds of formula (a-III) as described above.

The compounds of formula (b-II) can be obtained according to Preparation method (j) and Preparation method (k).

Preparation Method (j):

A method of reacting the compounds represented by the following formula:

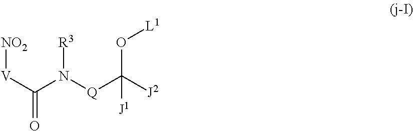

wherein
R³, V, Q, J¹, J² and L¹ have the same meaning as described above [provided that, in V, D represents a bonding site to the following moiety:

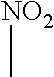

of formula (j-I), and E represents a bonding site to the following moiety:

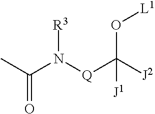

of formula (j-I)] with the compounds of formula (r-I) as described above.

Preparation Method (k):

A method of reacting the compounds represented by the following formula:

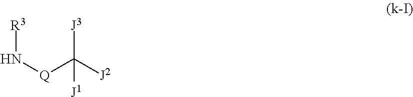

wherein
R³, Q, J¹, J² and J³ have the same meaning as described above, with the compounds of formula (r-IV) as described above.

The compounds of formula (j-I) as reacting materials for Preparation method (j) as described above are novel and representative examples thereof may include:

2-(3,5-dimethyl-4-{[(3-nitrophenyl)carbonyl]amino}phenyl)-1,1,1,3,3,3-hexafluoro-propan-2-yl methanesulfonate, 2-(3,5-dimethyl-4-{[(3-nitrophenyl)carbonyl]amino}phenyl)-1,1,1,3,3,3-hexafluoro-propan-2-yl methanesulfonate, 1-(3,5-dimethyl-4-{[(3-nitro-phenyl)carbonyl]amino}phenyl)-2,2,2-trifluoroethyl methanesulfonate, 2-(3,5-dimethyl-4-{[(3-nitrophenyl)carbonyl]amino}phenyl)-1,1,1-trifluoropropan-2-yl methanesulfonate, 1-(3,5-di-methyl-4-{[(3-nitrophenyl)carbonyl]amino}phenyl)-2,2,2-trifluoro-1-phenylethyl methanesulfonate, and the like.

Preparation method (j) as described above can be carried out in reference to Preparation method (a) as described above.

The compounds of formula (k-I) as reacting materials for Preparation method (k) as described above are novel and representative examples thereof may include:

4-[1,1,1,3,3,3-hexafluoro-2-(1H-pyrazol-1-yl)propan-2-yl]-2,6-dimethylaniline, 2,6-dibromo-4-[1,1,1,3,3,3-hexafluoro-2-(1H-pyrazol-1-yl)propan-2-yl]aniline, 2,6-dimethyl-4-[2,2,2-trifluoro-1-(1H-pyrazol-1-yl)ethyl]aniline, 2,6-dimethyl-4-[1,1,1-trifluoro-2-(1H-pyrazol-1-yl)propan-2-yl]-aniline, 2,6-dimethyl-4-[2,2,2-trifluoro-1-phenyl-1-(1H-pyrazol-1-yl)ethyl]aniline, 4-(1,1,1,3,3,3-hexafluoro-2-methoxypropan-2-yl)-2,6-dimethylaniline, and the like.

Preparation method (k) as described above can be carried out in reference to the method for synthesizing the compounds of formula (a-IV) from the compounds of formula (a-V) as described above.

The compounds of formula (k-I) as reacting materials for the above Preparation method (k) can be obtained according to Preparation method (l) and Preparation method (m).

Preparation Method (1):

A method of deprotecting the t-butoxycarbonyl group of the compounds represented by the following formula:

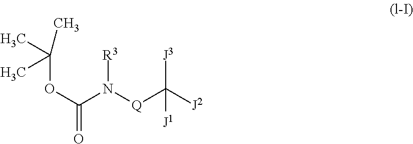

wherein
R³, Q, J¹, J² and J³ have the same meaning as described above.

Preparation Method (m):

A method of reacting the compounds represented by the following formula:

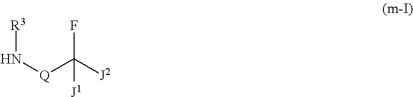

wherein
R³, Q, J¹ and J² have the same meaning as described above, with the compounds of formula (r-I) as described above.

The compounds of formula (l-I) for Preparation method (l) as described above are novel and representative examples thereof may include:

t-butyl {4-[1,1,1,3,3,3-hexafluoro-2-(1H-pyrazol-1-yppropan-2-yl]-2,6-dimethylphenyl}-carbamate, t-butyl 4-[1,1,1,3,3,3-hexafluoro-2-(1H-pyrazol-1-yl)propan-2-yl]phenyl}carbamate, t-butyl 2,6-dimethyl-4-[2,2,2-trifluoro-1-(1H-pyrazol-1-yl) ethyl]phenyl}carbamate, t-butyl {4-[2,2,2-trifluoro-1-(1H-pyrazol-1-yl)ethyl]phenyl}carbamate, t-butyl {2,6-dimethyl-4-[1,1,1-trifluoro-2-(1H-pyrazol-1-yl)propan-2-yl]phenyl}-carbamate, t-butyl {4-[1,1,1-trifluoro-2-(1H-pyrazol-1-yl)propan-2-yl]phenyl}carbamate, t-butyl {2,6-dimethyl-4-[2,2,2-trifluoro-1-phenyl-1-(1H-pyrazol-1-yl)ethyl]phenyl}-carbamate, t-butyl {4-[2,2,2-trifluoro-1-phenyl-1-(1H-pyrazol-1-yl)ethyl]phenyl}carbamate, and the like.

The reaction for Preparation method (l) can be carried out in an appropriate diluent. Examples of the diluent which may be used during the process include water; ethers such as ethyl ether, methyl ethyl ether, isopropyl ether, butyl ether, dioxane, dimethoxyethane (DME), tetrahydrofuran (THF), diethylene glycol dimethyl ether (DGM) and the like; ketones such as acetone and the like; nitriles such as acetonitrile, propionitrile, acrylonitrile and the like; alcohols such as methanol, ethanol, isopropanol, butanol, ethylene glycol and the like; acid amides such as dimethylformamide (DMF), dimethylacetamide (DMA), N-methylpyrrolidone, 1,3-dimethyl-2-imidazolidinone, hexamethylphosphoric triamide (HMPA) and the like; sulfones and sulfoxides, for example, dimethyl sulfoxide (DMSO), sulfolane and the like.

The above described Preparation method (l) can be carried out in the presence of an acid catalyst. Examples of the acid catalyst may include mineral acid, for example, hydrochloric acid, sulfuric acid, nitric acid, hydrobromic acid, sodium hydrogen sulfite and the like; organic acids, for example, formic acid, acetic acid, trifluoroacetic acid, propionic acid, methanesulfonic acid, benzenesulfonic acid, p-toluenesulfonic acid, and the like; hydrochlorides of organic amines, for example, pyridine hydrochloride, triethylamine hydrochloride and the like; sulfonates of amines, for example, pyridine p-toluenesulfonate, triethylamine p-toluenesulfonate and the like.

The above described Preparation method (l) can be carried out within a substantially broad range of temperature.

Generally, it can be carried out at a temperature of between approximately 0 and approximately 100° C., preferably between approximately 30 and approximately 80° C.

In addition, although the above reaction is preferably carried out at atmospheric pressure, it can be also carried out under reduced or elevated pressure.

For carrying out the above Preparation method (l), for example, relative to 1 mole of the compound of formula (l-I), 4 to 5 moles of aqueous hydrochloric acid in a diluent, for example, tetrahydrofuran can be reacted to obtain the desired compound.

The compounds of formula (m-I) as reacting materials for the above Preparation example (m) are publicly known and representative examples thereof may include:

4-(1,1,1,2,3,3,3-heptafluoropropan-2-yl)-2,6-dimethylaniline, 4-(1,1,1,2,3,3,3-heptafluoropropan-2-yl)aniline, and the like.

The compounds of formula (r-I) as reacting materials for the above Preparation example (m) are publicly known and representative examples thereof may include: sodium methoxide, sodium ethoxide and the like.

The reaction for Preparation method (m) can be carried out in an appropriate diluent. Examples of the diluent which may be used during the process include water; ethers such as dioxane, dimethoxyethane (DME), tetrahydrofuran (THF), diethylene glycol dimethyl ether (DGM) and the like; nitriles such as acetonitrile, propionitrile, acrylonitrile and the like; alcohols such as methanol, ethanol, isopropanol, butanol, ethylene glycol and the like; acid amides such as dimethylformamide (DMF), dimethylacetamide (DMA), N-methylpyrrolidone, 1,3-dimethyl-2-imidazolidinone, hexamethylphosphoric triamide (HMPA) and the like; sulfones and sulfoxides, for example, dimethyl sulfoxide (DMSO), sulfolane; and bases such as pyridine and the like.

Preparation method (m) can be carried out in the presence of an acid coupling agent, and such acid coupling agent may includes inorganic bases such as hydride, hydroxide, carbonate and bicarbonate of an alkaline metal and an alkaline earth metal, for example, sodium hydride, lithium hydride, sodium bicarbonate, potassium bicarbonate, sodium carbonate, potassium carbonate, lithium hydroxide, sodium hydroxide, potassium hydroxide, calcium hydroxide and the like and; inorganic alkaline metal amides, for example, lithium amide, sodium amide, potassium amide and the like.

Preparation method (m) can be carried out within a substantially broad range of temperatures.

Generally, it can be carried out at a temperature of between approximately 0 and approximately 100° C., preferably between approximately 20 and approximately 80° C.

In addition, although the above reaction is preferably carried out at atmospheric pressure, it can be also carried out under reduced or elevated pressure.

For carrying out Preparation method (m), for example, relative to 1 mole of the compound of formula (m-I), 4 to 5 moles of the compound of formula (r-I) in a diluent, for example a corresponding alcohol, can be reacted in the presence of a base as described above to obtain the desired compound.

By reacting the compounds of formula (k-I) as described above with appropriate halogenating agents, hydrogen represented by Y¹ and Y⁵ on Q can be replaced with halogen.

The halogenating reaction for the compounds of formula (k-I) as described above can be carried out in an appropriate diluent. Examples of the diluent which may be used during the process include acid amides such as dimethylformamide (DMF), dimethylacetamide (DMA), N-methylpyrrolidone, 1,3-dimethyl-2-imidazolidinone, hexamethylphosphoric triamide (RMPA) and the like; sulfones and sulfoxides, for example, dimethyl sulfoxide (DMSO), sulfolane; and organic acids, for example, acetic acid and the like.

The halogenating reaction for the compounds of formula (k-I) as described above can be carried out within a substantially broad range of temperatures.

Generally, it can be carried out at a temperature of between approximately 0 and approximately 100° C., preferably between approximately 20 and approximately 80° C.

In addition, although the above reaction is preferably carried out at atmospheric pressure, it can be also carried out under reduced or elevated pressure.

For carrying out the halogenating reaction for the compounds of formula (k-I) as described above, for example, relative to 1 mole of the compound of formula (k-I), 2 moles of a halogenating agent, for example, N-bromosuccinimide or N-iodosuccinimide can be reacted in a diluent, for example acetic acid, to obtain the desired compound.

The compounds of formula (l-I) as reacting materials for the above Preparation example (1) can be obtained by reacting the compounds represented by the following formula:

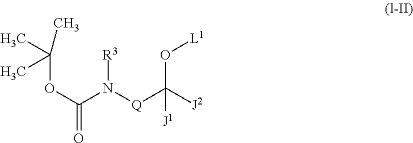

wherein
R³, Q, J¹, J² and L¹ have the same meaning as described above, with the compounds of formula (r-I) as described above.

The compounds of formula (I-II) as described above are novel and representative examples thereof may include:

2-{4-[(t-butoxycabonyl)amino]-3,5-dimethylphenyl}-1,1,1,3,3,3-hexafluoropropan-2-yl methane-sulfonate, 2-{4-[(t-butoxycabonyl)amino]phenyl}-1,1,1,3,3,3-hexafluoropropan-2-yl methane-sulfonate, 1-{4-[(t-butoxycabonyl)amino]-3,5-dimethylphenyl}-2,2,2-trifluoroethyl methane-sulfonate, 1-{4-[(t-butoxycabonyl)amino]phenyl}-2,2,2-trifluoroethyl methanesulfonate, 2-{4-[(t-butoxycabonyl)amino]-3,5-dimethylphenyl}-1,1,1-trifluoropropan-2-yl methanesulfonate, 2-{4-[(t-butoxycabonyl)amino]phenyl}-1,1,1-trifluoropropan-2-yl methanesulfonate, 1-{4-[(t-butoxycabonyl)amino]-3,5-dimethylphenyl}-2,2,2-trifluoro-1-phenylethyl methanesulfonate, 1-{4-[(t-butoxycabonyl)amino]phenyl}-2,2,2-trifluoro-1-phenylethyl methanesulfonate, and the like.

The synthesis of the compounds of formula (l-1) from the compounds of formula (l-II) as described above can be carried out in reference to Preparation method (a) as described above.

The compounds of formula (l-II) can be obtained by reacting the compounds represented by the following formula:

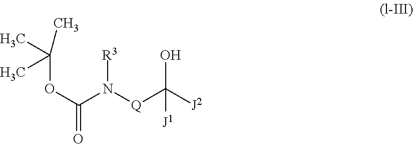

wherein
R³, Q, J¹ and J² have the same meaning as described above, with the compounds of formula (r-III) as described above.

The compounds of formula (l-III) as described above are novel and representative examples thereof may include:

t-butyl [4-(1,1,1,3,3,3-hexafluoro-2-hydroxypropan-2-yl)-2,6-dimethylphenyl]carbamate, t-butyl [4-(1,1,1,3,3,3-hexafluoro-2-hydroxypropan-2-yl)phenyl]carbamate, t-butyl [2,6-dimethyl-4-(2,2,2-trifluoro-1-hydroxyethyl)phenyl]carbamate, t-butyl [4-(2,2,2-trifluoro-1-hydroxyethyl)-phenyl]carbamate, t-butyl [2,6-dimethyl-4-(1,1,1-trifluoro-2-hydroxypropan-2-yl)phenyl]-carbamate, t-butyl [4-(1,1,1-trifluoro-2-hydroxypropan-2-yl)phenyl]carbamate, t-butyl [2,6-dimethyl-4-(2,2,2-trifluoro-1-hydroxy-1-phenylethyl)phenyl]carbamate, t-butyl [4-(2,2,2-trifluoro-1-hydroxy-1-phenylethyl)phenyl]carbamate and the like.

The synthesis of the compounds of formula (l-II) from the compounds of formula (l-III) as described above can be carried out in reference to the method for synthesizing the compounds of formula (a-I) from the compounds of formula (a-II).

The compounds of formula (l-III) can be obtained by reacting the compounds of formula (a-V) as described above with di-t-butoxycarbonate or t-butoxycarbonylchloride.

The compounds of formula (m-I) as reacting materials for the above Preparation example (m) can be obtained by reacting the compounds of formula (d-I) as described above with the compounds represented by the following formula:

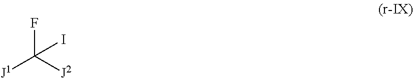

wherein
J¹ and J² have the same meaning as described above.

The compounds of formula (r-IX) as described above are publicly known and representative examples thereof may include:

1,1,1,2,3,3,3-heptafluoro-2-iodopropane and the like.

The synthesis of the compounds of formula (m-I) from the compounds of formula (d-I) and formula (r-IX) can be carried out according to the methods described in JP-A No. 2001-288129A.

The compounds of formula (c-II) that are used as reaction materials for the above Preparation method (c) can be obtained by reacting the compounds represented by the following formula:

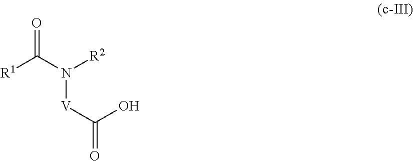

wherein
R¹, R² and V have the same meaning as defined above [provided that, in V, D represents a bonding site to the following moiety:

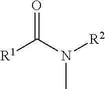

of formula (c-III), and E represents a bonding site to the following moiety:

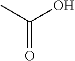

of formula (c-III)] with halogenating agents.

The compounds of formula (c-III) described above are publicly known and representative examples thereof may include:

3-{[(2-chloropyridin-3-yl)carbonyl]amino}benzene carboxylic acid, 3-{[(2-fluoropyridin-3-yl)carbonyl]amino}benzene carboxylic acid, 3-{[(2-chlorophenyl)carbonyl]amino}benzene carboxylic acid, 3-{[(2-fluorophenyl)carbonyl]amino}benzene carboxylic acid, 3-{[(3-chlorophenyl)carbonyl]amino}benzene carboxylic acid, 3-{[(3-fluoro-phenyl)carbonyl]amino}benzene carboxylic acid, 3-{[(4-chlorophenyl)carbonyl]amino}benzene carboxylic acid, 3-{[(4-fluorophenyl)carbonyl]amino}benzene carboxylic acid, and the like.

The reparation method for obtaining the compounds of formula (c-II) from the compounds of formula (c-III) can be carried out in an appropriate diluent. Examples of the diluent which may be used during the process include aliphatic, alicyclic, and aromatic hydrocarbons (which may be chlorinated in some cases) such as hexane, cyclohexane, benzene, toluene, xylene, chlorobenzene, dichlorobenzene, dichloromethane, dichloroethane and the like.

The above described reaction can be carried out by using a halogenating agent such as thionyl chloride, thionyl bromide and the like and by adding DMF and the like as a catalyst.

The above described reaction can be carried out within a substantially broad range of temperatures. Generally, it can be carried out at a temperature of between approximately 0 and approximately 200° C., preferably between room temperature and approximately 150° C. In addition, although the above reaction is preferably carried out at atmospheric pressure, it can be also carried out under reduced or elevated pressure.

For carrying out the above reaction, for example, relative to 1 mole of the compound of formula (c-III), a catalytic amount of DMF in a diluent, for example, 1,2-dichloroethane is added and then reacted with thionyl chloride to obtain the desired compound of formula (c-II).

The compounds of formula (c-III) as described above can be easily obtained by hydrolyzing the compounds represented by the following formula:

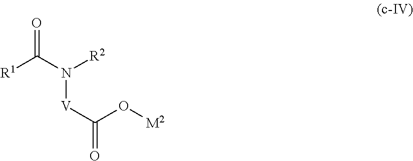

wherein
R¹, R² and V have the same meaning as defined above [provided that, in V, D represents a bonding site to the following moiety:

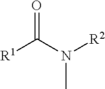

of formula (c-IV), and E represents a bonding site to the following moiety:

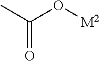

of formula (c-IV)] and M² represents C₁₋₄ alkyl, according to a conventional method.

The compounds of formula (c-IV) described above are publicly known and representative examples thereof may include:

methyl 3-{[(2-chloropyridin-3-yl)carbonyl]amino}benzoate, methyl 3-{[(2-fluoro-pyridin-3-yl)carbonyl]amino}benzoate, methyl 3-{[(2-chlorophenyl)carbonyl]amino}benzoate methyl 3-{[(2-fluorophenyl)carbonyl]amino}benzoate, methyl 3-{[(3-chlorophenyl)-carbonyl]amino}benzoate, methyl 3-{[(3-fluorophenyl)carbonyl]amino}benzoate, methyl 3-{[(4-chlorophenyl)carbonyl]amino}benzoate, methyl 3-{[(4-fluorophenyl)carbonyl]-amino}benzoate, and the like.

The preparation from the above compounds of formula (c-IV) to the compounds of formula (c-III) by hydrolysis can be carried out in an appropriate diluent. Examples of the diluent which may be used during the process include water; ethers such as ethyl ether, methyl ethyl ether, isopropyl ether, butyl ether, dioxane, tetrahydrofuran (THF) and the like; alcohols such as methanol, ethanol, isopropanol, butanol, ethylene glycol and the like; etc.

The above described reaction is can⁻led out by using inorganic bases such as hydroxides of an alkaline metal and an alkaline earth metal including sodium hydroxide, potassium hydroxide, calcium hydroxide and the like or inorganic acids such as hydrochloric acid, sulfuric acid and the like.

The above described reaction can be carried out within a substantially broad range of temperatures. Generally, it can be carried out at a temperature of between approximately 0 and approximately 200° C., preferably between room temperature and approximately 150° C. In addition, although the above reaction is preferably carried out at atmospheric pressure, it can be also carried out under reduced or elevated pressure.

For carrying out the above reaction, for example, 1 mole of the compound of formula (c-IV) is reacted with potassium hydroxide in a diluent, for example, a mixed solvent of ethanol and water to obtain the desired compound of formula (c-III).

The compounds of formula (c-IV) as described above can be easily obtained by reacting the compounds represented by the following formula

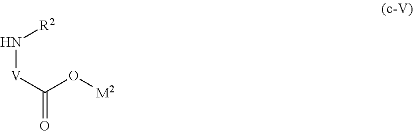

wherein
R², V and M² have the same meaning as defined above [provided that, in V, D represents a bonding site to the following moiety:

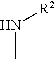

of formula (c-V), and E represents a bonding site to the following moiety:

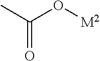

of formula (c-V)] with the compounds of the formula (r-II) as described above according to a conventional method.

The compounds of formula (c-V) described above are publicly known and representative examples thereof may include: methyl 3-aminobenzoate, and the like.

The reaction between the compounds of formula (c-V) and the compounds of formula (r-II) can be carried out in an appropriate diluent. Examples of the diluent which may be used during the process may include aliphatic, alicyclic, and aromatic hydrocarbons (which may be chlorinated in some cases) such as pentane, hexane, cyclohexane, petroleum ether, benzene, toluene, xylene, dichloromethane, dichloroethane, and the like; ethers such as ethyl ether, methyl ethyl ether, isopropyl ether, butyl ether, dioxane, dimethoxyethane (DME), tetrahydrofuran (THF), diethylene glycol dimethyl ether (DGM) and the like; ketones such as acetone, methyl ethyl ketone (MEK), methyl isopropyl ketone, methyl isobutyl ketone (MIBK) and the like; nitriles such as acetonitrile, propionitrile, acrylonitrile and the like; esters such as ethyl acetate, amyl acetate and the like.

The above described reaction can be carried out in the presence of a base. Examples of such base includes inorganic bases such as a hydroxide, a carbonate and a bicarbonate of alkaline metal for example, sodium bicarbonate, potassium bicarbonate, sodium carbonate, potassium carbonate, lithium hydroxide, sodium hydroxide, potassium hydroxide, and the like; and organic bases such as alcoholates, tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2.2.2]octane (DABCO), 1,8-diazabicyclo[5.4.0]undec-7-ene (DBU) and the like.

The above described reaction can be also carried out based on a method which uses a phase-transfer catalyst. Examples of the diluent which may be used during the process may include water; aliphatic, alicyclic, and aromatic hydrocarbons (which may be chlorinated in some cases) such as pentane, hexane, cyclohexane, benzene, toluene, xylene, and the like; ethers such as ethyl ether, methyl ethyl ether, methyl butyl ether, isopropyl ether, butyl ether, and the like.

Examples of the phase-transfer catalyst may include quaternary ions such as tetramethylammonium bromide, tetrapropylammonium bromide, tetrabutylammonium bromide, tetrabutylammonium bisulfate, tetrabutylammonium iodide, trioctylmethylammonium chloride, benzyltriethylammonium bromide, butylpyridinium bromide, heptylpyridinium bromide, benzyltriethylammonium chloride and the like; crown ethers such as dibenzo-18-crown-6, dicyclohexyl-18-crown-6, 18-crown-6 and the like; cryptands such as [2.2.2]-cryptate, [2.1.1]-cryptate, [2.2.1]-cryptate, [2.2.B]-cryptate, [2O2O2S]-cryptate, [3.2.2]-cryptate and the like.

The above described reaction can be carried out within a substantially broad range of temperatures. Generally, it can be carried out at a temperature of between approximately −40 and approximately 200° C., preferably between −20 and approximately 110° C. In addition, although the above reaction is preferably carried out at atmospheric pressure, it can be also carried out under reduced or elevated pressure. For carrying out the above reaction, for example, relative to 1 mole of the compound of formula (c-V), 1 mole or slightly excessive amount of the compound of formula (r-II) in a diluent, for example, THF is reacted in the presence of pyridine to obtain the desired compound.

For substituting G¹ and G² in the compounds of formula (I) with a sulfur atom, it can be achieved by reacting the above compounds of formula (I), (b-II), (b-I) or (c-IV) with Lawesson's reagent (XX).

In order to introduce R² and R³ that are not hydrogen to the above descried compounds of formula (I), the above compounds of formulae (I), (b-II), (l-1) or (c-IV), etc. and the compounds that are represented by the following formula:

Hal-L² (r-X)

wherein
L² represents R² or R³, and Hal has the same meaning as defined above, can be reacted in the presence of appropriate bases, or alternatively, they can be also obtained by reductive alkylating the compounds represented by the following formula:

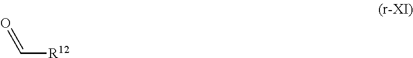

wherein
R¹² represents hydrogen or alkyl, using appropriate reducing agents such as sodium borohydride, sodium borocyanohydride and the like in appropriate diluents such as acetic acid and formic acid.

When V in the compounds of formula (I) of the present invention is any one of V2 to V5, corresponding compounds can be synthesized according to the methods described below. Each of the reactions is designated as Scheme 1 to 5.

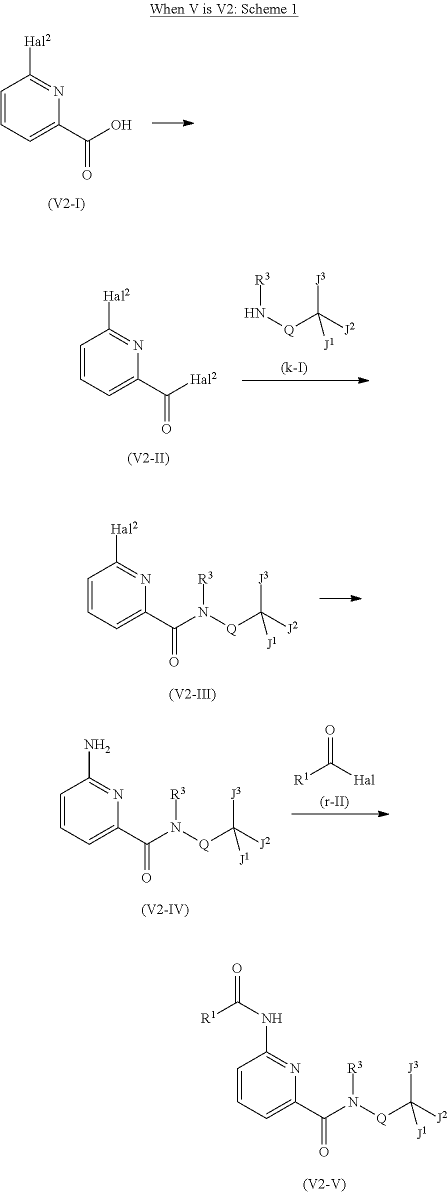

(wherein Hal² represents halogen, and bromo and chloro are preferable. In addition, Q, J¹ to J³, Hal, R¹ and R³ have the same meaning as defined above).

Specifically, the above compounds of formula (V2-I) such as 6-chloropyridine-2-carboxylic acid, 6-bromopyridine-2-caboxylic acid and the like, are treated with appropriate halogenating agents to give the above compounds of formula (V2-II), which are then reacted with the compounds of formula (k-I) to give the above compounds of formula (V2-III). The halogen in the compounds of formula (V2-III) can be substituted with an amine to give the compounds of formula (V2-IV). Subsequently, in accordance with the above Preparation method (b), the compounds of formula (V2-V), which are encompassed by the compounds of formula (I) of the present invention, can be synthesized.

When V is V3: Scheme 2 and Scheme 3

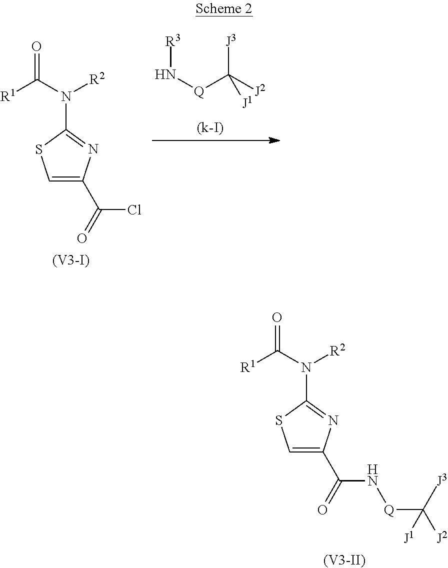

(wherein Q, J¹ to J³ and R¹ to R³ have the same meaning as defined above).

The compounds of formula (V3-I) which are disclosed in Patent document (WO2007-051560 Publication Pamphlet) are reacted with the compounds of formula (k-I) in accordance with Preparation method (c) described above. As a result, the compounds of formula (V3-II), which are encompassed by the compounds of formula (I) of the present invention, can be obtained.

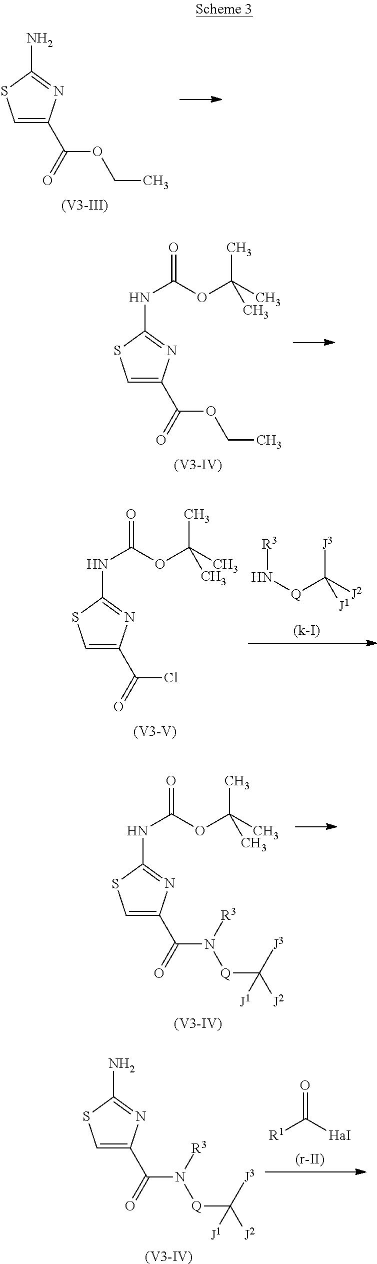

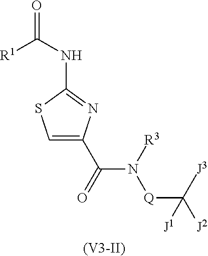

(wherein Q, J¹ to J³, Hal, R¹ and R³ have the same meaning as defined above).

The compounds of formula (V3-III) are converted to the compounds of formula (V3-V) in accordance with the method disclosed in Patent document (WO2007-051560 Publication Pamphlet). Then, they are reacted with the compounds of formula (k-I) in accordance with

Preparation method (c) described above, yielding the compounds of formula (V3-IV) after deprotection of the t-butoxide group. Finally, in accordance with the above Preparation method (b), the compounds of formula (V3-IV) are reacted with the compounds of formula (r-II) to obtain the compounds of formula (V3-II), which are encompassed by the compounds of formula (I) of the present invention.

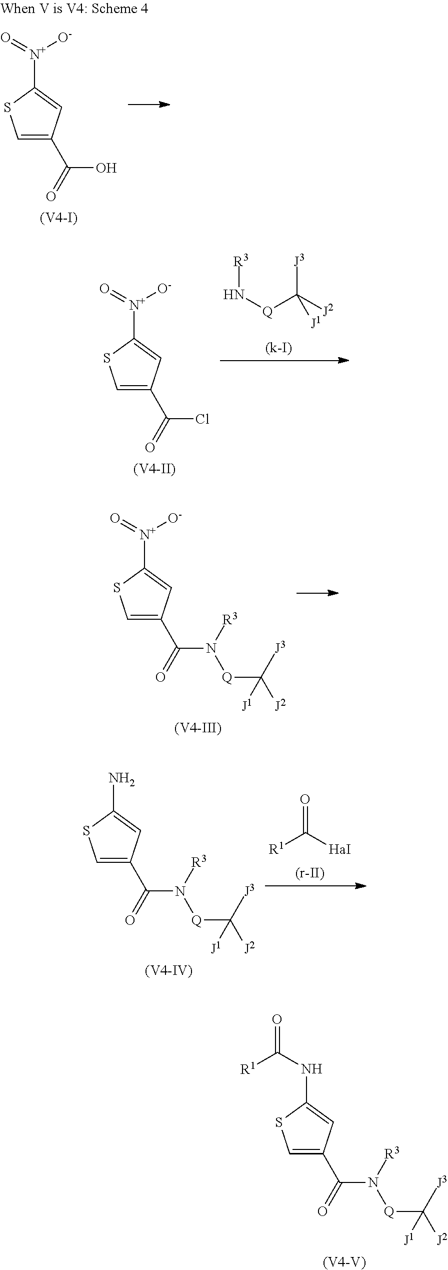

(wherein Q, J¹ to J³, Hal, R¹ and R³ have the same meaning as defined above).

After converting the compounds of formula (V4-I), which are publicly known compounds, to the compounds of formula (V4-II) according to a conventional method, by following the above Preparation method (k) and (b), the compounds of formula (V4-V), which are encompassed by the compounds of formula (I) of the present invention, can be obtained.

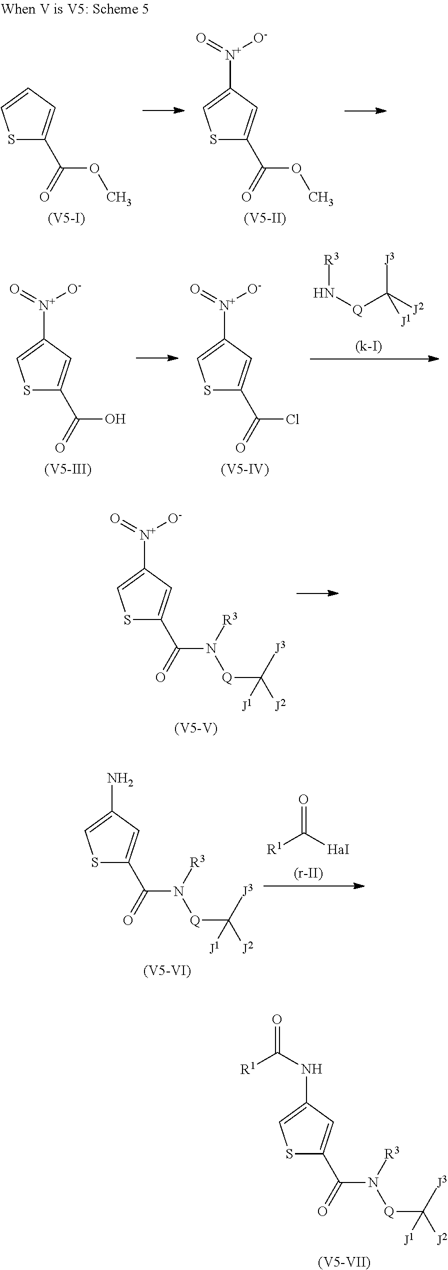

(wherein Q, J¹ to J³, Hal, R¹ and R³ have the same meaning as defined above).

After converting the compounds of formula (V5-I), which are publicly known compounds, to the compounds of formula (V5-III), by nitration and ester hydrolysis according to conventional methods, the compounds of formula (V5-VII), which are encompassed by the compounds of formula (I) of the present invention, can be obtained by following Preparation method (k) and Preparation method (b) that are described above.

For substituting the substituent X⁴ in V in the compounds of formula (I) of the present invention with fluoro, the following processes can be carried out. Specifically, the compounds of formula (a-III) and the compounds of formula (b-II) of the above Preparation method (a) and (b) in which X⁴ corresponds to chloro are reacted with appropriate fluorinating agents such as potassium fluoride in appropriate solvents for example, DMF and the like, thereby chloro can be replaced by fluoro. Subsequently, according to the above Preparation method (b), the compounds of formula (I) of the present invention can be obtained. Specific examples of this reaction are illustrated in Scheme 6 and Scheme 7.

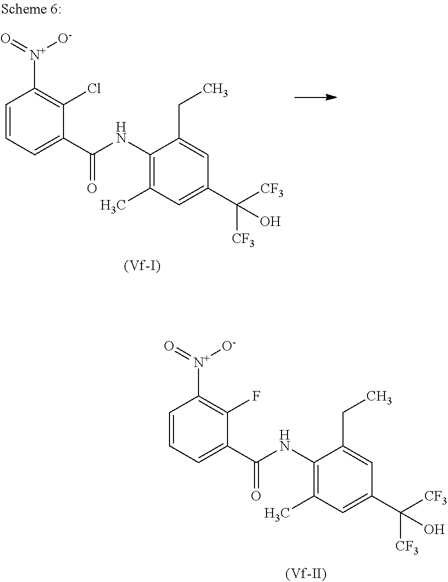

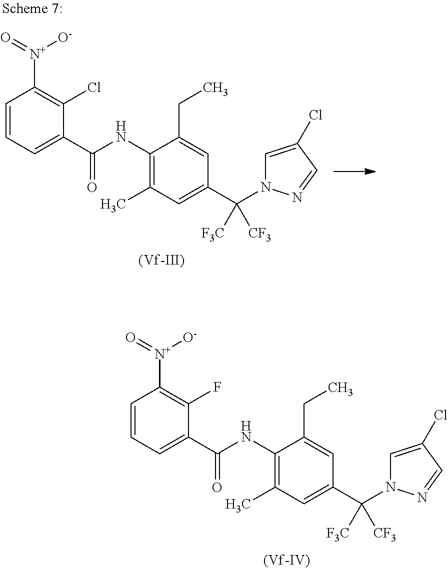

Intermediates that are useful for the synthesis of the compounds of formula (I) of the present invention, i.e., the compounds that are included in any one of formulae (a-I), (a-III), (a-IV), (a-V), (b-I), (c-I), (j-I), (k-I), (l-I), (l-II) and (l-III), are summarized in formula (II) below.

Compounds represented by following formula (II).

Formula (II)

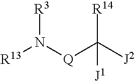

wherein
R¹³ represents hydrogen, alkyloxycarbonyl, phenyloxycarbonyl, aralkyloxycarbonyl, a group of the following formula:

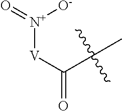

[wherein V and X¹ to X⁴ have the same meaning as defined above (provided that, in V, D represents a bonding site to the following moiety:

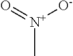

of the group, and E represents a bonding site to the following moiety:

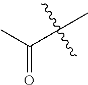

of the group),

or

a group of the following formula:

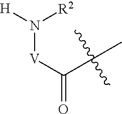

(wherein V, X¹ to X⁴ and R² have the same meaning as defined above),
R¹⁴ represents J³ or a group of the following formula:

—O-L¹

(wherein L¹ has the same meaning as defined above), and
R³, Q and J¹ to J³ have the same meaning as defined above, provided that the cases wherein J¹ and J² are perfluoroalkyl and J³ is hydroxyl or halogen are excluded.

The compounds of formula (I) of the present invention exhibit a very strong pesticidal activity. Thus, the compounds of formula (I) of the present invention can be used as pesticides. In addition, the active compounds of formula (I) of the present invention have an efficacy that is specific for harmful insects without exhibiting any phytotoxicity to crop plants. Further, the compounds of the present invention can be used for controlling various kinds of harmful pests, for instances, harmful sucking insects, mastication insects, plant-parasitic insects, storage insects and hygienic insects, etc. and also for combating and extermination thereof.

Harmful animal pest are for example:

As for insects, coleopterans, for example, Callosobruchus chinensis, Sitophilus zeamais, Tribolium castaneum, Epilachna vigintioctomaculata, Agriotes fuscicollis, Anomala rufocuprea, Leptinotarsa decemlineata, Diabrotica spp., Monochamus alternatus, Lissorhoptrus oryzophilus, Lyctus bruneus, Aulacophora femoralis; lepidopterans, for example, Lymantria dispar, Malacosoma neustria), Pieris rapae, Spodoptera litura, Mamestra brassicae, Chilo suppressalis), Pyrausta nubilalis, Ephestia cautella, Adoxophyes orana, Carpocapsa pomonella, Agrotisfucosa, Galleria mellonella, Plutella maculipennis, Heliothis virescens, Phyllocnistis citrella; hemipterans, for example, Nephotettix cincticeps, Nilaparvata lugens, Pseudococcus comstocki, Unaspis yanonensis, Myzus persicas, Aphis pomi, Aphis gossypii, Rhopalosiphum pseudobrassicas, Stephanitis nashi, Nezara spp., Trialeurodes vaporariorm, Psylla spp.; thysanopterans, for example, Thrips palmi, Franklinella occidental; orthopterans, for example, Blatella germanica, Periplaneta americana, Gryllotalpa Africana, Locusta migratoria migratoriodes; isopterans, for example, Reticulitermes speratus, Coptotermes formosanus; dipterans, for example, Musca domestica, Aedes aegypti, Hylemia platura, Culex pipiens, Anopheles sinensis, Culex tritaeniorhynchus, Liriomyza trifolii.

As for acari, for example, Tetranychus cinnabarinus, Tetranychus urticae, Panonychus citri, Aculops pelekassi, Tarsonemus spp.

As for nematodes, for example, Meloidogyne incognita, Bursaphelenchus lignicolus Mamiya et Kiyohara, Aphelenchoides besseyi, Heterodera glycines, Pratylenchus spp.

Additionally, the compounds according to the present invention show a good plant tolerance and favourable toxicity to warm-blooded animals and being tolerated well by the environment, and thus are suitable for protecting plants and plant parts.

Application of the compounds of the invention may result in increasing the harvest yields, improving the quality of the harvested material. Additionally, the compounds can be used for controlling animal pests, in particular insects, arachnids, helminths, nematodes and molluscs, which are encountered in agriculture, in horticulture, the field of veterinary medicine, in forests, in gardens and leisure facilities, in the protection of stored products and of materials, and in the hygiene sector. They may be preferably employed as plant protection agents. They are active against normally sensitive and resistant species and against all or some stages of development. These pests include inter alia:

From the order of the Anoplura (Phthiraptera), for example, Damalinia spp., Haematopinus spp., Linognathus spp., Pediculus spp., Trichodectes spp.

From the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example, Dreissena spp.

From the order of the Chilopoda, for example, Geophilus spp., Scutigera spp.

From the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Stemechus spp., Symphyletes spp., Tenebrio molitor, Tribolium spp., Trogoderma spp., Tychius spp., Xylotrechus spp., Zabrus spp.

From the order of the Collembola, for example, Onychiurus armatus.

From the order of the Dermaptera, for example, Forficula auricularia.

From the order of the Diplopoda, for example, Blaniulus guttulatus.

From the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tarmia spp., Tipula paludosa, Wohlfahrtia spp.

From the class of the Gastropoda, for example, Arion spp., Biomphalaria spp., Bulinus spp., Deroceras spp., Galba spp., Lymnaea spp., Oncomelania spp., Succinea spp.

From the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

It is furthermore possible to control protozoa, such as Eimeria.

From the order of the Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma pin, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Cameocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example, Diprion spp., Hoplocampa spp., Lasius spp., Monomorium pharaonis, Vespa spp.

From the order of the Isopoda, for example, Armadillidium vulgare, Oniscus asellus, Porcellio scaber.

From the order of the Isoptera, for example, Reticulitermes spp., Odontotermes spp.

From the order of the Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Cheimatobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example, Ceratophyllus spp., Xenopsylla cheopis.

From the order of the Symphyla, for example, Scutigerella immaculata.

From the order of the Thysanoptera, for example, Baliothrips biformis, Enneothrips flavens, Franldiniella spp., Heliothrips spp., Hercinothrips femoralis, Kakothrips spp., Rhipiphorothrips cruentatus, Scirtothrips spp., Taeniothrips cardamoni, Thrips spp.

From the order of the Thysanura, for example, Lepisma saccharina.

The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on their surroundings, habitat or storage space by the customary treatment methods, for example by immersion, spraying, evaporation, fogging, scattering, painting on, injecting and, in the case of propagation material, in particular in the case of seed, also by applying one or more coats.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetically Modified Organisms), and parts thereof, are treated. The terms “parts”, “parts of plants” and “plant parts” have been explained above.

Particularly preferably, plants of the plant cultivars which are in each case commercially available or in use are treated according to the invention. Plant cultivars are understood as meaning plants having novel properties (“traits”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, diet), the treatment according to the invention may also result in superadditive “synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars (obtained by genetic engineering) which are to be treated according to the invention include all plants which, by virtue of the genetic modification, received genetic material which imparts particularly advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, sugar beet, tomatoes, peas and other vegetable varieties, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized in particular are the increased defence of the plants against insects, arachnids, nematodes and slugs and snails by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to hereinbelow as “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya beans), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plant cultivars will be developed and/or marketed in the future.

The plants listed can be treated according to the invention in a particularly advantageous manner with the compounds according to the invention at a suitable concentration.

Furthermore, in the field of veterinary medicine, the novel compounds of the present invention can be effectively used against various harmful animal parasitic pests (endoparasites and ectoparasites), for example, insects and helminthes. Examples of such animal parasitic pests include the pests as described below. Examples of the insects include Gasterophilus spp., Stomoxys spp., Trichodectes spp., Rhodnius spp., Ctenocephalides canis, Cimx lecturius, Ctenocephalides felis, Lucilia cuprina, and the like. Examples of acari include Ornithodoros spp., Ixodes spp., Boophilus spp., and the like.

In the veterinary fields, i.e. in the field of veterinary medicine, the active compounds according to the present invention are active against animal parasites, in particular ectoparasites or endoparasites. The term endoparasites includes in particular helminths, such as cestodes, nematodes or trematodes, and protozoae, such as coccidia. Ectoparasites are typically and preferably arthropods, in particular insects such as flies (stinging and licking), parasitic fly larvae, lice, hair lice, bird lice, fleas and the like; or acarids, such as ticks, for examples hard ticks or soft ticks, or mites, such as scab mites, harvest mites, bird mites and the like.

These parasites include:

From the order of the Anoplurida, for example Haematopinus spp., Linognathus spp., Pediculus spp., Phtirus spp., Solenopotes spp.; particular examples are: Linognathus setosus, Linognathus vituli, Linognathus ovillus, Linognathus oviformis, Linognathus pedalis, Linognathus stenopsis, Haematopinus asini macrocephalus, Haematopinus eurysternus, Haematopinus suis, Pediculus humanus capitis, Pediculus humanus corporis, Phylloera vastatrix, Phthirus pubis, Solenopotes capillatus;

from the order of the Mallophagida and the suborders Amblycerina and Ischnocerina, for example Trimenopon spp., Menopon spp., Trinoton spp., Bovicola spp., Werneckiella spp., Lepikentron spp., Damalina spp., Trichodectes spp., Felicola spp.; particular examples are: Bovicola bovis, Bovicola ovis, Bovicola limbata, Damalina bovis, Trichodectes canis, Felicola subrostratus, Bovicola caprae, Lepikentron ovis, Werneckiella equi;

from the order of the Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; particular examples are: Aedes aegypti, Aedes albopictus, Aedes taeniorhynchus, Anopheles gambiae, Anopheles maculipennis, Calliphora erythrocephala, Chrysozona pluvialis, Culex quinquefasciatus, Culex pipiens, Culex tarsalis, Fannia canicularis, Sarcophaga carnaria, Stomoxys calcitrans, Tipula paludosa, Lucilia cuprina, Lucilia sericata, Simulium reptans, Phlebotomus papatasi, Phlebotomus longipalpis, Odagmia ornata, Wilhelmia equina, Boophthora erythrocephala, Tabanus bromius, Tabanus spodopterus, Tabanus atratus, Tabanus sudeticus, Hybomitra ciurea, Chrysops caecutiens, Chrysops relictus, Haematopota pluvialis, Haematopota italica, Musca autumnalis, Musca domestica, Haematobia irritans irritans, Haematobia irritans exigua, Haematobia stimulans, Hydrotaea irritans, Hydrotaea albipuncta, Chrysomya chloropyga, Chrysomya bezziana, Oestrus ovis, Hypoderma bovis, Hypoderma lineatum, Przhevalskiana silenus, Dermatobia hominis, Melophagus ovinus, Lipoptena capreoli, Lipoptena cervi, Hippobosca variegata, Hippobosca equina, Gasterophilus intestinalis, Gasterophilus haemorroidalis, Gasterophilus inermis, Gasterophilus nasalis, Gasterophilus nigricomis, Gasterophilus pecorum, Braula coeca;

from the order of the Siphonapterida, for example Pulex spp., Ctenocephalides spp., Tunga spp., Xenopsylla spp., Ceratophyllus spp.; particular examples are: Ctenocephalides canis, Ctenocephalides felis, Pulex irritans, Tunga penetrans, Xenopsylla cheopis;

from the order of the Heteropterida, for example Cimex spp., Triatoma spp., Rhodnius spp., Panstrongylus spp.

From the order of the Blattarida, for example Blatta orientalis, Periplaneta americana, Blattela germanica, Supella spp. (e.g. Suppella longipalpa);

From the subclass of the Acari (Acarina) and the orders of the Meta- and Mesostigmata, for example Argas spp., Ornithodorus spp., Otobius spp., Ixodes spp., Amblyomma spp., Rhipicephalus (Boophilus) spp Dermacentor spp., Haemophysalis spp., Hyalomma spp., Dermanyssus spp., Rhipicephalus spp. (the original genus of multi host ticks) Ornithonyssus spp., Pneumonyssus spp., Raillietia spp., Pneumonyssus spp., Stemostoma spp., Varroa spp., Acarapis spp.; particular examples are: Argas persicus, Argas reflexus, Ornithodorus moubata, Otobius megnini, Rhipicephalus (Boophilus) microplus, Rhipicephalus (Boophilus) decoloratus, Rhipicephalus (Boophilus) annulatus, Rhipicephalus (Boophilus) calceratus, Hyalomma anatolicum, Hyalomma aegypticum, Hyalomma marginatum, Hyalomma transiens, Rhipicephalus evertsi, Ixodes ricinus, Ixodes hexagonus, Ixodes canisuga, Ixodes pilosus, Ixodes rubicundus, Ixodes scapularis, Ixodes holocyclus, Haemaphysalis concinna, Haemaphysalis punctata, Haemaphysalis cinnabarina, Haemaphysalis otophila, Haemaphysalis leachi, Haemaphysalis longicorni, Dermacentor marginatus, Dermacentor reticulatus, Dermacentor pictus, Dermacentor albipictus, Dermacentor andersoni, Dermacentor variabilis, Hyalomma mauritanicum, Rhipicephalus sanguineus, Rhipicephalus bursa, Rhipicephalus appendiculatus, Rhipicephalus capensis, Rhipicephalus turanicus, Rhipicephalus zambeziensis, Amblyomma americanum, Amblyomma variegatum, Amblyomma maculatum, Amblyomma hebraeum, Amblyomma cajennense, Dermanyssus gallinae, Ornithonyssus bursa, Ornithonyssus sylviarum, Varroa jacobsoni;

from the order of the Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.; particular examples are: Cheyletiella yasguri, Cheyletiella blakei, Demodex canis, Demodex bovis, Demodex ovis, Demodex caprae, Demodex equi, Demodex caballi, Demodex suis, Neotrombicula autumnalis, Neotrombicula desaleri, Neoschongastia xerothermobia, Trombicula akamushi, Otodectes cynotis, Notoedres cati, Sarcoptis canis, Sarcoptes bovis, Sarcoptes ovis, Sarcoptes rupicaprae (=S. caprae), Sarcoptes equi, Sarcoptes suis, Psoroptes ovis, Psoroptes cuniculi, Psoroptes equi, Chorioptes bovis, Psoergates ovis, Pneumonyssoidic mange, Pneumonyssoides caninum, Acarapis woodi.

The active compounds according to the invention are also suitable for controlling arthropods, helminths and protozoae, which attack animals. Animals include agricultural livestock such as, for example, cattle, sheep, goats, horses, pigs, donkeys, camels, buffaloes, rabbits, chickens, turkeys, ducks, geese, cultured fish, honeybees. Moreover, animals include domestic animals—also referred to as companion animals—such as, for example, dogs, cats, cage birds, aquarium fish and what are known as experimental animals such as, for example, hamsters, guinea pigs, rats and mice.

By controlling these arthropods, helminths and/or protozoae, it is intended to reduce deaths and improve performance (in the case of meat, milk, wool, hides, eggs, honey and the like) and health of the host animal, so that more economical and simpler animal keeping is made possible by the use of the active compounds according to the invention.

For example, it is desirable to prevent or interrupt the uptake of blood by the parasites from the hosts (when applicable). Also, controlling the parasites may help to prevent the transmittance of infectious agents.

The term “controlling” as used herein with regard to the veterinary field, means that the active compounds are effective in reducing the incidence of the respective parasite in an animal infected with such parasites to innocuous levels. More specifically, “controlling”, as used herein, means that the active compound is effective in killing the respective parasite, inhibiting its growth, or inhibiting its proliferation.

Generally, when used for the treatment of animals the active compounds according to the invention can be applied directly. Preferably they are applied as pharmaceutical compositions which may contain pharmaceutically acceptable excipients and/or auxiliaries which are known in the art.

In the veterinary field and in animal keeping, the active compounds are applied (=administered) in the known manner by enteral administration in the form of, for example, tablets, capsules, drinks, drenches, granules, pastes, boluses, the feed-through method, suppositories; by parenteral administration, such as, for example, by injections (intramuscular, subcutaneous, intravenous, intraperitoneal and the like), implants, by nasal application, by dermal application in the form of, for example, bathing or dipping, spraying, pouring-on and spotting-on, washing, dusting, and with the aid of active-compound-comprising shaped articles such as collars, ear tags, tail tags, limb bands, halters, marking devices and the like. The active compounds may be formulated as shampoo or as suitable formulations usable in aerosols, unpressurized sprays, for example pump sprays and atomizer sprays.

When used for livestock, poultry, domestic animals and the like, the active compounds according to the invention can be applied as formulations (for example powders, wettable powders [“WP”], emulsions, emulsifiable concentrates [“EC”], flowables, homogeneous solutions, and suspension concentrates [“SC”]) which comprise the active compounds in an amount of from 1 to 80% by weight, either directly or after dilution (e.g. 100- to 10 000-fold dilution), or else as a chemical bath.

When used in the veterinary field the active compounds according to the invention may be used in combination with suitable synergists or other active compounds, such as for example, acaricides, insecticides, anthelmintics, anti-protozoal drugs.

In the present invention, a substance having an insecticidal action against pests including all of these is referred to as an insecticide.

When it is used as a pesticide, the active compounds of the present invention can be prepared in a form of a common preparation. Such preparation form may includes, for example, liquids, emulsions, wettable powders, granulated wettable powders, suspensions, powders, foams, pastes, tablets, granules, aerosols, natural or synthetic agents impregnated with the active compounds, microcapsules, coating agents for seeds, formulations equipped with a combustion devise (the combustion devise can be a smoke or fog cartridge, a can or a coil, etc.) and ULV (cold mist, warm mist), etc.

These formulations can be produced by known methods per se. For example, they can be prepared by mixing the active compounds with extenders, namely, liquid diluents or carriers; liquefied gas diluents or carriers; solid diluents or carriers and, optionally, with surfactants, namely, emulsifiers and/or dispersants and/or foam formers and the like.

In case of using water as an extender, for example, organic solvents can be used as auxiliary solvents.

Examples of the liquid diluents or carriers may include aromatic hydrocarbons (for example, xylene, toluene, alkylnaphthalene and the like), chlorinated aromatic hydrocarbons or chlorinated aliphatic hydrocarbons (for example, chlorobenzenes, ethylene chlorides, methylene chloride and the like), aliphatic hydrocarbons (for example, cyclohexane and the like, paraffins (mineral oil fractions and the like)), alcohols (for example, butanol, glycol and the like, and ethers and esters thereof), ketones (for example, acetone, methyl ethyl ketone, methyl isobutyl ketone, cyclohexanone and the like), strongly polar solvents (for example, dimethylformamide, dimethyl sulfoxide and the like), water, etc.

Liquefied gas diluent or carrier may includes those present as gas at atmospheric pressure and temperature, for example, bulan, propane, nitrogen gas, carbon dioxide, and aerosol propellant such as halogenated hydrocarbons.

Examples of the solid diluents may include ground natural minerals (for example, kaolins, clay, talc, chalk, quartz, attapulgite, montmorillonite or diatomaceous earth) and ground synthetic minerals (for example, highly dispersed silicic acid, alumina and silicate) and the like.

Examples of the solid carriers for granules may include crushed and fractionated rocks (for example, calcite, marble, pumice, sepiolite and dolomite), synthetic granules of inorganic or organic powders, and fine granules of organic materials (for example, sawdust, coconut shells, maize cobs and tobacco stalks) and the like.

Examples of the emulsifiers and/or foam formers may include nonionic and anionic emulsifiers [for example, polyoxyethylene fatty acid esters, polyoxyethylene fatty acid alcohol ethers (for example, alkylaryl polyglycol ether), alkyl sulfonates, alkyl sulfates and aryl sulfonates] and albumin hydrolysates and the like.

The dispersants include lignin sulfite waste liquor and methylcellulose.

Binders may also be used in the formulations (powders, granules and emulsion). Examples of the binders may include carboxymethyl cellulose, natural or synthetic polymers (for example, gum arabic, polyvinyl alcohol and polyvinyl acetate).

Colorants may also be used. Examples of the colorants may include inorganic pigments (for example, iron oxide, titanium oxide and Prussian blue), organic colorants such as Alizarin colorants, azo colorants or metal phthalocyanine colorants, and further, trace nutrients such as salts of iron, manganese, boron, copper, cobalt, molybdenum or zinc.

The formulation may comprise the above active component in an amount of 0.1 to 95% by weight, preferably 0.5 to 90% by weight.

The active compounds of formula (I) of the present invention can be provided as a mixture with other active compounds such as a pesticide, a poison bait, a sterilizing agent, an acaricidal agent, a nematocide, a fungicide, a growth regulating agent, a herbicide, etc. in a form of commercially useful formulation or an application form prepared from formulation thereof. The pesticide may include, for example, an organic phosphorous agent, carbamate agent, carboxylate agent, chlorinated hydrocarbon agent, and pesticidal substance produced by microorganisms, etc.

Further, the active compounds of formula (I) of the present invention can be provided as a mixture with a synergist. Such formulation and application form may include those that are commercially useful. The synergist is not necessarily active by itself. Rather, it is the compound which enhances the activity of the active compounds.

The amount of the active compounds of formula (I) of the present invention that is comprised in a commercially useful form may vary over a broad range.

The concentration of the active compounds of formula (I) of the present invention for actual use can be, for example, between 0.0000001 and 100% by weight, preferably between 0.00001 and 1% by weight.

The active compounds of formula (I) of the present invention can be used according to any common method that is appropriate for an application form.

The active compounds of formula (I) the present invention have stability that is effective for alkaline substances present in lime materials when the compounds are used against hygienic pests and storage pests. In addition, it exhibits excellent residual effectiveness in woods and soils.

The active compounds of formula (I) of the present invention have low toxicity and can be safely used for warm-blooded animals.

Next, the present invention is exemplified by way of the following examples, but the invention is not intended to be limited thereto.

### SYNTHETIC EXAMPLE 1

### Synthesis of N-[3-({4-[2-(3-bromo-1H-pyrazol-1-yl)-1,1,1,3,3,3-hexa-fluoropropan-2-yl]-2,6-dimethylphenyl}carbamoyflphenyl]-2-chloropyridine-3-carboxamide (No. 2-76)

### Step 1. Synthesis of

N-[4-(1,1,1,3,3,3-hexafluoro-2-hydroxypropan-2-yl)-2,6-dimethylphenyl]-3-nitrobenzamide

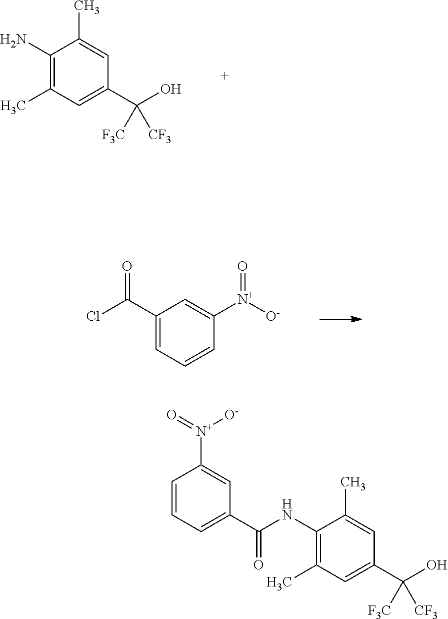

2-(4-amino-3,5-dimethylphenyl)-1,1,1,3,3,3-hexafluoropropan-2-ol (5.39 g) and pyridine (2.0 ml) were dissolved in THF (150 ml), and 3-nitrobenzoyl chloride (2.53 g) was slowly added thereto at room temperature. After stirring overnight, water and tBuOMe were added thereto. The organic layer was separated and the aqueous layer was extracted with tBuOMe. The organic layer was washed with dilute hydrochloric acid, and the solvent was evaporated off under reduced pressure. To the residuals, THF and an aqueous solution of sodium hydroxide (10%) were added followed by stirring for 1 hour. The mixture was extracted with tBuOMe and the organic layer was dried over magnesium sulfate. After the filtration, the solvent was evaporated off under reduced pressure to obtain the title compound as a crude product (8.00 g).

¹H-NMR (CDCl₃) δ: 1.60 (1H, s), 2.36 (6H, s), 3.49 (1H, s), 7.49 (2H, s), 7.75 (1H, t), 8.30 (1H, d), 8.46 (1H, d), 8.75 (1H, s).

### Step 2. Synthesis of 3-amino-N-[4-(1,1,1,3,3,3-hexafluoro-2-hydroxypropan-2-yl)-2,6-dimethyl-phenyl]benzamide

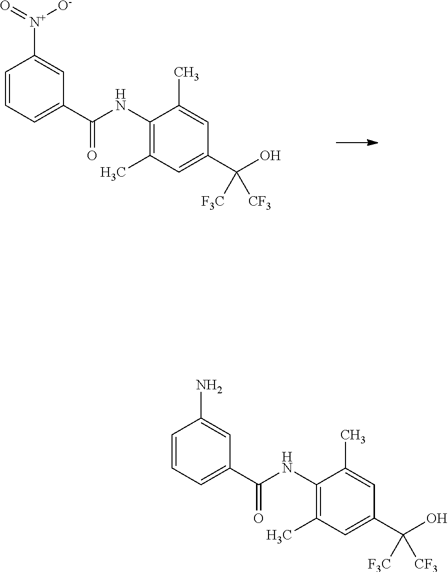

N-[4-(1,1,1,3,3,3-hexafluoro-2-hydroxypropan-2-yl)-2,6-dimethylphenyl]-3-nitrobenzamide (8.00 g) was dissolved in ethanol (150 ml), and stannic chloride dihydrate (16.5 g) and concentrated hydrochloric acid (15 ml) were added thereto at room temperature. The reaction solution was refluxed under heat for 4 hours. The solvent was evaporated off under reduced pressure, and an aqueous solution of sodium carbonate and ethyl acetate were added thereto. Insoluble matters were filtered using Celite and the filtrate was extracted with ethyl acetate. The organic layer was dried over magnesium sulfate. After the filtration, the solvent was evaporated off under reduced pressure. With the purification on silica gel chromatography, the title compound was obtained (4.50 g, 60%).

¹H-NMR (CDCl₃) δ: 2.29 (6H, s), 5.10 (1H, br s), 6.87 (1H, d), 7.10-7.52 (9H, m).

### Step 3. Synthesis of 2-chloro-N-(3-{[4-(1,1,1,3,3,3-hexafluoro-2-hydroxy-propan-2-yl)-2,6-dimethylphenyl]carbamoyl}phenyl)pyridine-3-carboxamide

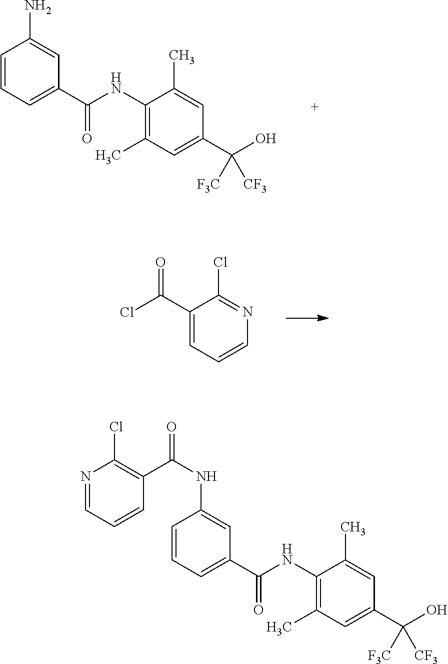

3-amino-N-[4-(1,1,1,3,3,3-hexafluoro-2-hydroxypropan-2-yl)-2,6-dimethylphenyl]benzamide (4.50 g) was dissolved in THF (50 ml), and pyridine (2.0 ml) and 2-chloronicotnic acid chloride (2.53 g) were added thereto under ice cooling. After stirring overnight, the solvent was evaporated off under reduced pressure. The resulting residue was purified with silica gel chromatography to obtain the title compound (5.57 g, 92%).

¹H-NMR (CDCl₃) δ: 2.05 (1H, br s), 2.31 (6H, s), 7.26-7.56 (5H, m), 7.76 (1H, d), 7.94 (1H, dd), 8.05 (1H, d), 8.26 (1H, s), 8.48 (1H, dd), 8.77 (1H, s), 10.14 (1H, s).

### Step 4. Synthesis of 2-(4-{[(3-{[(2-chloropyridin-3-yl)carbonyl]amino}-phenyl)carbonyl]-amino}-3,5-dimethylphenyl)-1,1,1,3,3,3-hexafluoro-2-yl methanesulfonate

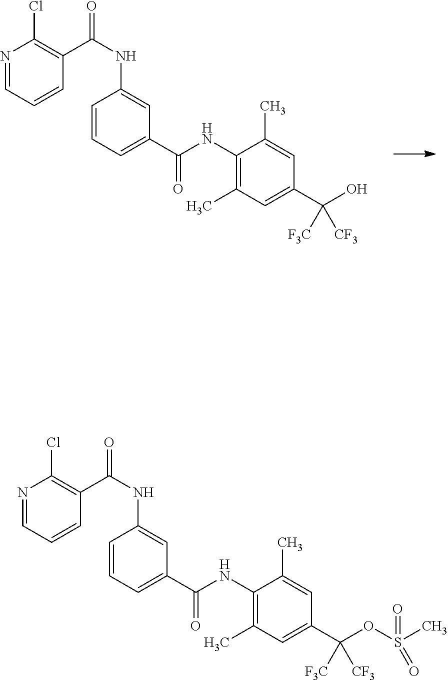

2-chloro-N-(3-{[4-(1,1,1,3,3,3-hexafluoro-2-hydroxypropan-2-yl)-2,6-dimethylphenyl]carbamoyl}phenyl)pyridine-3-carboxamide (4.50 g) and triethylamine (3.45 ml) were dissolved in methylene chloride (50 ml), and methanesulfonyl chloride (1.60 ml) was added thereto at room temperature. After stirring overnight, water was added. The resulting mixture was extracted with ethyl acetate and dried over magnesium sulfate. After the filtration, the solvent was evaporated off under reduced pressure. The resulting residue was purified with silica gel chromatography to obtain the title compound (3.74 g, 72%).

¹H-NMR (CDCl₃) δ: 2.28 (6H, s), 3.27 (3H, s), 7.30-7.37 (1H, m),7.41-7.52 (3H, m), 7.65-7.74 (2H, m), 7.86 (1H, d), 8.04 (1H, d), 8.20 (1H, s), 8.43-8.49 (1H, m), 8.65 (1H, s).

### Step 5. Synthesis of N-[3-({4-[2-(3-bromo-1H-pyrazol-1-yl)-1,1,1,3,3,3-hexafluoropropan-2-yl]-2,6-dimethylphenyl}carbamoyl)phenyl]-2-chloropyridine-3-carboxamide

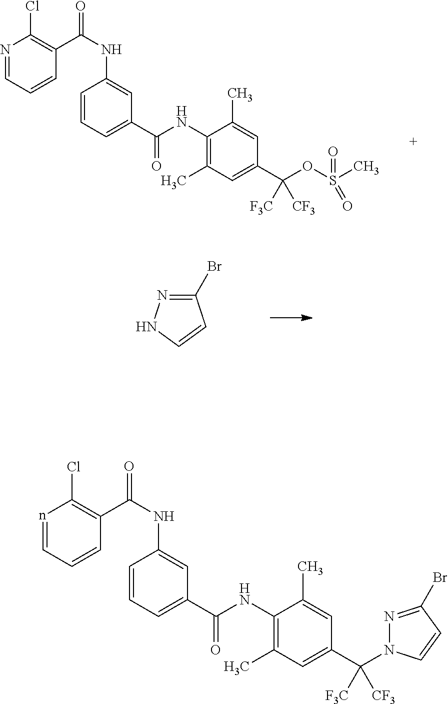

2-(4-{[(3-{[(2-chloropyridin-3-yl)carbonyl]amino}phenyl)carbonyl]amino}-3,5-dimethylphenyl)-1,1,1,3,3,3-hexafluoro-2-yl methanesulfonate (0.10 g) and 3-bromopyridine (28 mg) were dissolved in DMF (1.5 ml), and sodium hydride (in oil, 10 mg) was added thereto under ice cooling. After stirring for 4 hours, water was added. The resulting mixture was extracted with ethyl acetate, washed with water, and dried over magnesium sulfate. After the filtration, the solvent was evaporated off under reduced pressure. The resulting residue was purified with silica gel chromatography to obtain the title compound (76 mg, 70%).

¹H-NMR (CDCl₃) δ: 2.31(6H, s), 6.45 (1H, d), 7.16 (2H, s), 7.36-7.64 (4H, m), 7.74 (1H, d), 7.86 (1H, d), 8.14-8.22 (1H, m), 8.29 (1H, s), 8.44-8.55 (2H, m).

### SYNTHETIC EXAMPLE 2

### Synthesis of 2-chloro-N-{3-[(2-ethyl-4-{1,1,1,3,3,3-hexafluoro-2-[4-(trifluoromethyl)-1H-pyrazol-1-yl]propan-2-yl}-6-methylphenyl)carbamoyl]-phenyl}pyridine-3-carboxamide (No. 2-105)

### Step 1. Synthesis of 2-(4-amino-3-ethyl-5-methylphenyl)-1,1,1,3,3,3-hexafluoropropan-2-ol

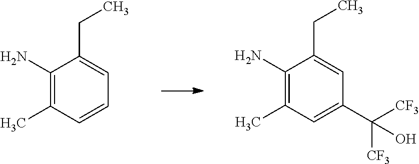

To a mixture of 2-ethyl-6-methylaniline (25.45 g) and 1,1,1,3,3,3-hexafluoro-propan-2-one hydrate (50 g), p-toluenesulfonic acid monohydrate (0.54 g) was added and the resulting mixture was refluxed under heating for 4 hours. After cooling the reaction solution to room temperature, it was dissolved in ethyl acetate. This ethyl acetate solution was washed with water and a saturated solution of sodium bicarbonate followed by drying over magnesium sulfate. After separating the drying agent by filtration, the solvent was evaporated off under reduced pressure to obtain a crude product. This resulting crude product was washed with a mixed solvent comprising hexane-ethyl acetate to obtain 2-(4-amino-3-ethyl-5-methylphenyl)-1,1,1,3,3,3-hexafluoropropan-2-ol (36.7 g).

¹H-NMR (CDCl₃): 1.26 (3H, t), 2.20 (3H, s), 2.54 (2H, q), 3.32 (1H, s), 3.78 (2H, s), 7.25 (2H, s).

### Step 2. Synthesis of N-[2-ethyl-4-(1,1,1,3,3,3-hexafluoro-2-hydroxypropan-2-yl)-6-methylphenyl]-3-nitrobenzamide

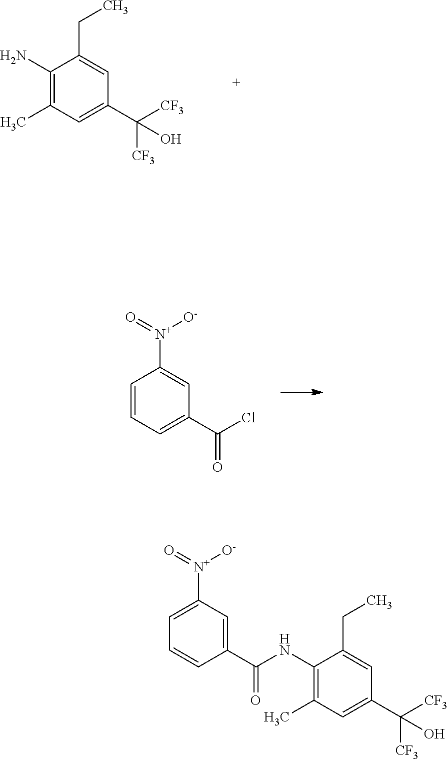

2-(4-amino-3-ethyl-5-methylphenyl)-1,1,1,3,3,3-hexafluoropropan-2-ol (10.2 g) and pyridine (5.6 g) were dissolved in THF. To the resulting solution, THF solution in which 3-nitrobenzoyl chloride (12.8 g) had been dissolved was added dropwise under ice cooling. After the completion of dropwise addition, the mixture was brought back to room temperature and stirred for 6 hours. The reaction solution was poured over water and extracted twice with TBME. The organic layers were combined, washed with 2N hydrochloric acid, and dried over magnesium sulfate. After separating the drying agent by filtration, the solvent was evaporated off under reduced pressure. Thus obtained residue was dissolved in THF and an aqueous solution of 2N sodium hydroxide (60 ml) was added thereto. The mixture was stirred at room temperature for 1 hour. After diluting the reaction solution with water, it was extracted twice with TBME. The organic layers were combined, washed with water, and dried over magnesium sulfate. After the flirtation, the solvent was evaporated off under reduced pressure to obtain N-[2-ethyl-4-(1,1,1,3,3,3-hexafluoro-2-hydroxy-propan-2-yl)-6-methylphenyl]-3-nitrobenzamide (15.0 g).

¹H-NMR (CDCl₃) δ: 1.20 (3H, t), 2.30 (3H, s), 2.66 (2H, q), 4.19 (1H, s), 7.49 (2H, s), 7.69-7.74 (2H, m), 8.27 (1H, d), 8.44 (1H, d), 8.74 (1H, dd).

### Step 3. Synthesis of 2-(3-ethyl-5-methyl-4-{[(3-nitrophenyl)carbonyl]amino}phenyl)-1,1,1,3,3,3-hexafluoropropan-2-yl methanesulfonate

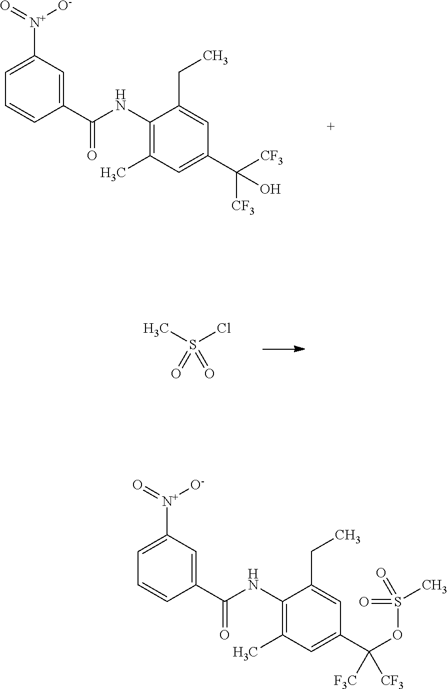

N-[2-ethyl-4-(1,1,1,3,3,3-hexafluoro-2-hydroxypropan-2-yl)-6-methylphenyl]-3-nitrobenzamide (9.40 g) and triethylamine (4.65 g) were dissolved in dichloromethane. To this solution, methanesulfonyl chloride (5.02 g) was added dropwise. After the completion of dropwise addition, the mixture was stirred at room temperature for 2 hours. The reaction solution was evaporated off under reduced pressure to remove the solvent. To thus obtained residue, ethyl acetate was added. The ethyl acetate solution was washed with 2N hydrochloric acid followed by drying over magnesium sulfate. After separating the drying agent by filtration, the solvent was evaporated off under reduced pressure to obtain the target compound of 2-(3-ethyl-5-methyl-4-{[(3-nitrophenyl)carbonyl]amino}phenyl)-1,1,1,3,3,3-hexafluoropropan-2-yl methanesulfonate (11.8 g).

¹H-NMR (CDCl₃) δ: 1.18 (3H, t), 2.26 (3H, s), 2.64 (2H, q), 3.26 (3H, s), 7.44 (1H, s), 7.52 (1H, s), 7.64 (1H, dd), 7.99 (1H, s), 8.21 (1H, d), 8.39 (1H, d), 8.73 (1H, s).

### Step 4. Synthesis of N-(2-ethyl-4-{1,1,1,3,3,3-hexafluoro-2-[4-(trifluoromethyl)-1H-pyrazol-1-yl]propan-2-yl}-6-methylphenyl)-3-nitrobenzamide.

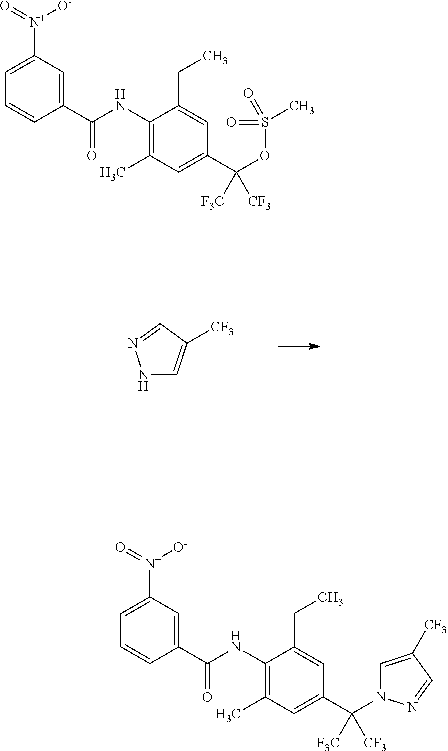

2-(3-ethyl-5-methyl-4-{[(3-nitrophenyl)carbonyl]amino}phenyl)-1,1,1,3,3,3-hexafluoropropan-2-yl methanesulfonate (0.40 g) was dissolved in acetonitrile. To this solution, 4-(trifluoromethyl)-1H-pyrazole (0.11 g) and potassium carbonate (0.13 g) were added, and then stirred under heating at 70° C. for 1 hour. After cooling the reaction solution to room temperature, a saturated aqueous solution of ammonium chloride was added and the resulting solution was extracted twice with ethyl acetate. The organic layers were combined, washed with water, and dried over magnesium sulfate. After separating the drying agent by filtration, the solvent was evaporated off under reduced pressure to obtain a crude product. Thus obtained crude product was purified with silica gel chromatography to obtain N-(2-ethyl-4-{1,1,1,3,3,3-hexafluoro-2-[4-(trifluoromethyl)-1H-pyrazol-1-yl]propan-2-yl}-6-methylphenyl)-3-nitrobenzamide (0.35 g).

¹H-NMR (CDCl₃) δ: 1.19 (3H, t), 2.34 (3H, s), 2.68 (2H, q), 7.16 (2H, s), 7.50 (1H, s), 7.77 (1H, dd), 7.87 (1H, s), 7.95 (1H, s), 8.30 (1H, d), 8.48 (2H, d), 8.75 (1H, s).

### Step 5. Synthesis of 3-amino-N-(2-ethyl-4-{1,1,1,3,3,3-hexafluoro-2-[4-(trifluoromethyl)-1H-pyrazol-1-yl]-propan-2-yl}-6-methylphenyl)benzamide

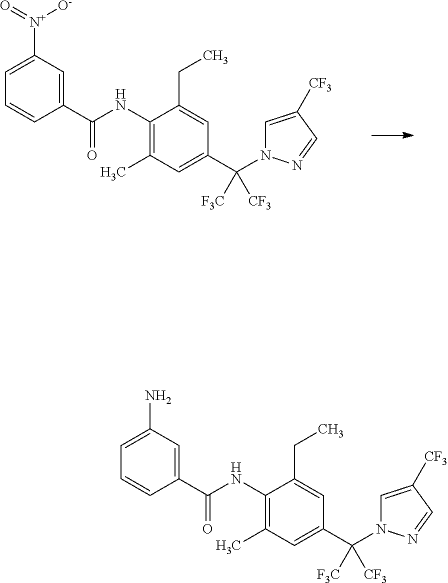

N-(2-ethyl-4-{1,1,1,3,3,3-hexafluoro-2-[4-(trifluoromethyl)-1H-pyrazol-1-yl]-propan-2-yl}-6-methylphenyl)-3-nitrobenzamide (0.34 g) was dissolved in ethanol. To this solution, stannic chloride dihydrate (0.42 g) and concentrated hydrochloric acid (1 ml) were added and stirred under heating at 70° C. for 4 hours. After cooling down to room temperature, ethyl acetate and water were added. With vigorous stirring, potassium carbonate was added for neutralization. The resulting mixture was filtered using Celite to separate the organic layer. The aqueous layer was extracted with ethyl acetate. The organic layers were combined, washed with water, and dried over magnesium sulfate. After separating the drying agent by filtration, the solvent was evaporated off under reduced pressure to obtain 3-amino-N-(2-ethyl-4-{1,1,1,3,3,3-hexafluoro-2-[4-(trifluoromethyl)-1H-pyrazol-1-yl]propan-2-yl}-6-methylphenyl)-benzamide (0.26 g).

¹H-NMR (CDCl₃) δ: 1.16 (5H, t), 2.31 (3H, s), 2.65 (2H, q), 3.87 (2H, br s), 6.88 (1H, d), 7.13 (2H, s), 7.19-7.30 (3H, m), 7.39 (1H, s), 7.85 (1H, s), 7.93 (1H, s).

### Step 6. Synthesis of 2-chloro-N-{3-[(2-ethyl-4-{1,1,1,3,3,3-hexafluoro-2-[4-(trifluoromethyl)-1H-pyrazol-1-yl]propan-2-yl}-6-methylphenyl)carbamoyl]-phenyl}pyridine-3-carboxamide

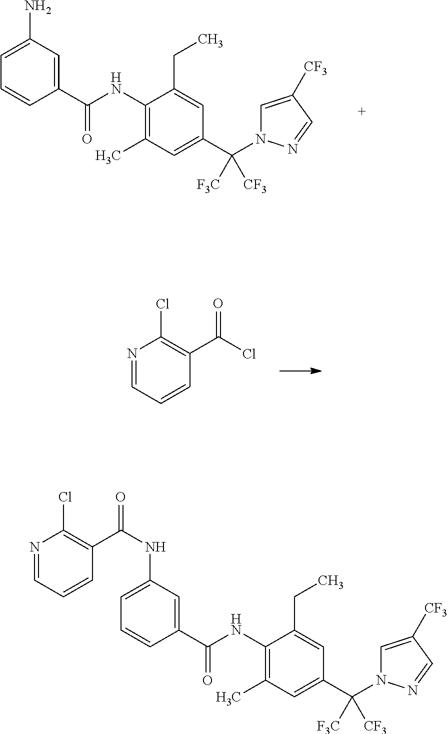

3-amino-N-(2-ethyl-4-{1,1,1,3,3,3-hexafluoro-2-[4-(trifluoromethyl)-1H-pyrazol-1-yl]propan-2-yl}-6-methylphenyl)benzamide (0.20 g) and pyridine (0.04 g) were dissolved in THF. To this solution, 2-chloropyridine-3-carbonyl chloride (0.07 g) was added followed by stirring at room temperature for 1 hour. The reaction mixture was poured over water, and then extracted twice with ethyl acetate. The combined organic layers were washed with water, and dried over magnesium sulfate. After separating the drying agent by filtration, the solvent was evaporated off under reduced pressure to obtain a crude product. The resulting crude product was purified with silica gel chromatography to obtain 2-chloro-N-{3-[(2-ethyl-4-{1,1,1,3,3,3-hexafluoro-2-[4-(trifluoromethyl)-1H-pyrazol-1-yl]propan-2-yl}-6-methylphenyl)-carbamoyl]phenyl}pyridine-3-carboxamide (0.25 g).

¹H-NMR (CDCl₃) δ: 1.14 (3H, t), 2.27 (3H, s), 2.64 (2H, q), 7.12 (2H, s), 7.31-7.37 (1H, m), 7.47 (1H, t), 7.69 (1H, d), 7.81-7.94 (4H, m), 8.03 (1H, d), 8.29 (1H, s), 8.44 (1H, d), 8.86 (1H, br s).

### SYNTHETIC EXAMPLE 3

### Synthesis of N-{3-[(4-{1-[3,5-bis(trifluoromethyl)-1H-pyrazol-1-yl)-2,2,2-trifluoroethyl}-2,6-dimethylphenyl)carbamoyl]phenyl}-2-chloropyridine-3-carboxamide (No. 2-123)

### Step 1. Synthesis of 3,5-dimethyl-4-nitrobenzoic acid

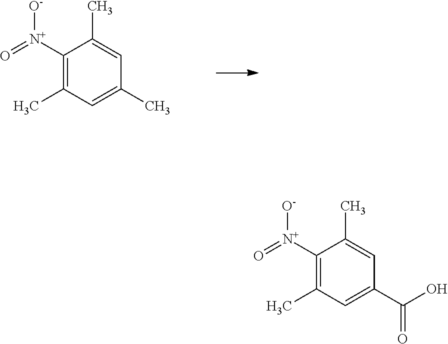

To an acetic acid solution to which chromium oxide (VI) (10.0 g) had been dissolved, an acetic acid solution of 1,3,5-trimethyl-2-nitrobenzene (5.00 g) was added dropwise at 70° C. After the completion of dropwise addition, the mixture was stirred under heating at the same temperature for 30 min. Isopropanol (11 ml) was added thereto, and then again the mixture was stirred under heating at 50° C. for 30 min. Water was poured to this solution, which was then cooled down in ice bath. Crystals obtained therefrom were filtered and taken, dissolved in ethyl acetate, and dried over magnesium sulfate. After separating the drying agent by filtration, the solvent was evaporated off under reduced pressure to obtain a crude product. The resulting crude product was washed with hexane to obtain 3,5-dimethyl-4-nitrobenzoic acid (1.40 g).

¹H-NMR (CDCl₃) δ: 2.37 (3H, s), 7.89 (1H, s).

### Step 2. Synthesis of (3,5-dimethyl-4-nitrophenyl)methanol

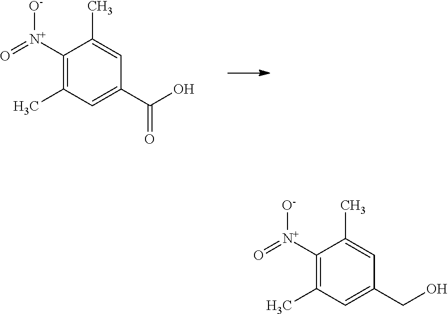

Under the argon atmosphere, 3,5-dimethyl-4-nitrobenzoic acid (1.39 g) was dissolved in THF. Under ice cooling, 0.9 N THF solution of diborane was added dropwise thereto. After the completion of dropwise addition, the mixture was stirred at the same temperature for 30 min. The mixture was then brought back to room temperature and stirred again overnight. A mixed solvent comprising water and THF was added slowly under ice cooling until foaming stops. The mixture was then poured over water and extracted twice with ethyl acetate. The organic layers were combined, and dried over magnesium sulfate. After separating the drying agent by filtration, the solvent was evaporated off under reduced pressure to obtain (3,5-dimethyl-4-nitrophenyl)methanol (1.17 g).

¹H-NMR (CDCl₃) δ: 1.79 (1H, t), 2.32 (3H, s), 4.68 (1H, d), 7.13 (1H, s).

### Step 3. Synthesis of 3,5-dimethyl-4-nitrobenzaldehyde

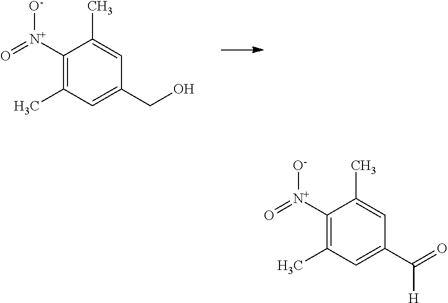

(3,5-dimethyl-4-nitrophenyl)methanol (1.10 g) was dissolved in dichloromethane. To this solution, manganese dioxide (5.28 g) was added followed by reflux under heating for 6 hours. After cooling down to room temperature, the precipitates were filtered. The solvent was evaporated off under reduced pressure to obtain a crude product. The resulting crude product was purified with silica gel chromatography to obtain 3,5-dimethyl-4-nitrobenzaldehyde (0.79 g).

¹H NMR (CDCl₃) δ: 2.39 (6H, s), 7.66 (2H, s), 10.00 (1H, s).

### Step 4. Synthesis of (3,5-dimethyl-4-nitrophenyl)-2,2,2-trifluoroethanol

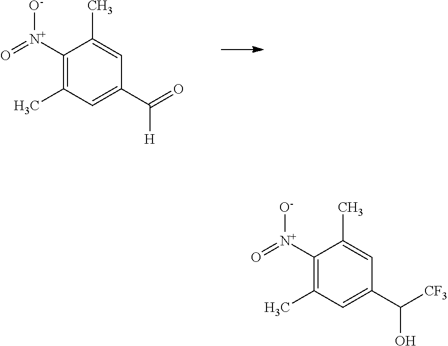

3,5-dimethyl-4-nitrobenzaldehyde (2.33 g) and trimethyl(trifluoromethyl)silane (2.22 g) were dissolved in THF. To the resulting solution, THF solution of 1N TBAF (1.3 ml) was added dropwise under ice cooling. After the completion of dropwise addition, the mixture was brought back to room temperature and stirred overnight. To this solution, 6N hydrochloric acid (6 ml) was added followed by stirring for 2 hours. Potassium hydrocarbonate was added thereto for neutralization, until foaming stops. Water was added to this solution, which was then extracted twice with ethyl acetate. The organic layers were combined, washed with water and dried over magnesium sulfate. After separating the drying agent by filtration, the solvent was evaporated off under reduced pressure. Thus obtained crude product was purified by silica gel chromatography to obtain (3,5-dimethyl-4-nitrophenyl)-2,2,2-trifluoroethanol (3.36 g).

¹H-NMR (CDCl₃) δ: 2.34 (6H, s), 4.08-4.16 (1H, m), 5.05-4.97 (1H, m), 7.26 (2H, s).

### Step 5. Synthesis of 1-(4-amino-3,5-dimethylphenyl)-2,2,2-trifluoroethanol

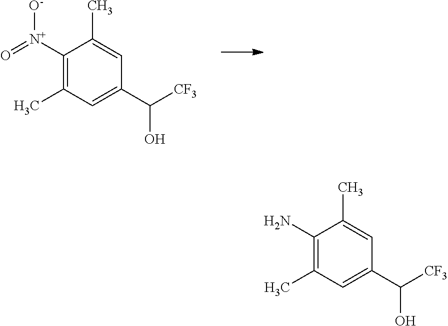

(3,5-dimethyl-4-nitrophenyl)-2,2,2-trifluoroethanol (3.36 g) was dissolved in methanol and nickel chloride (II) hexahydrate (4.81 g) was added thereto. Under ice cooling, sodium tetrahydroborate (1.53 g) was slowly added thereto. After the completion of the addition, the mixture was brought back to room temperature and stirred for 3 hours. To the reaction mixture, ethyl acetate and water were added. With vigorous stirring, ammonia water was added thereto until the precipitates disappear. The organic layer was separated and the aqueous layer was extracted with ethyl acetate. The organic layers were combined, washed with water and dried over magnesium sulfate. After separating the drying agent by filtration, the solvent was evaporated off under reduced pressure to obtain 1-(4-amino-3,5-dimethylphenyl)-2,2,2-trifluoroethanol (2.49 g).

¹H-NMR (CDCl₃) δ: 2.19 (6H, s), 2.44 (1H, d), 3.69 (2H, s), 4.88-4.80 (1H, m), 7.03 (2H, s).

### Step 6. Synthesis of N-[2,6-dimethyl-4-(2,2,2-trifluoro-1-hydroxyethyl)-phenyl]-3-nitro-benzamide

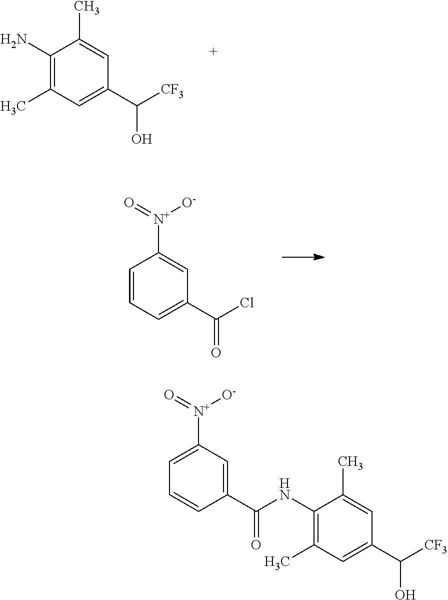

1-(4-amino-3,5-dimethylphenyl)-2,2,2-trifluoroethanol (2.49 g) and pyridine (1.80 g) were dissolved in THF. To the resulting solution, THF solution in which 3-nitrobenzoyl chloride (2.11 g) had been dissolved was added dropwise under ice cooling. After the completion of dropwise addition, the mixture was brought back to room temperature and stirred for 2 hours. This solution was poured over water and extracted twice with ethyl acetate. The organic layers were combined, washed with 2N hydrochloric acid, and dried over magnesium sulfate. After separating the drying agent by filtration, the solvent was evaporated off under reduced pressure. Thus obtained crude product was purified by silica gel chromatography to obtain N-[2,6-dimethyl-4-(2,2,2-trifluoro-1-hydroxyethypphenyl]-3-nitrobenzamide (3.60 g).

¹H-NMR (DMSO-d6) δ: 2.21 (6H, s), 5.06-5.15 (1H, m), 6.82 (1H, d), 7.26 (2H, s), 7.86 (1H, t), 8.43-8.48 (2H, m), 8.81 (1H, dd), 10.18 (1H, s).

### Step 7. Synthesis of N-[4-(1-chloro-2,2,2-trifluoroethyl)-2,6-dimethylphenyl]-3-nitrobenzamide

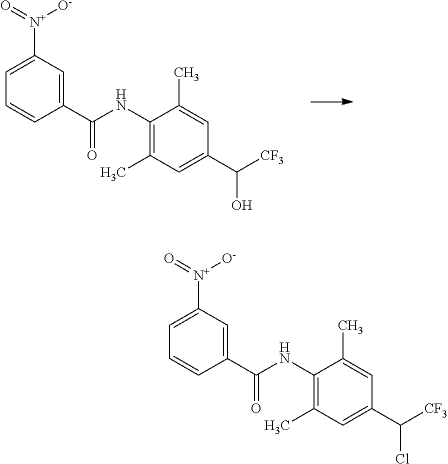

N-[2,6-dimethyl-4-(2,2,2-trifluoro-1-hydroxyethyl)phenyl]-3-nitrobenzamide (1.00 g) and pyridine (0.22 g) were dissolved in dichloroethane. To this solution, thionyl chloride (0.65 g) was added, and then stirred under heating at 70° C. for 6 hours. After cooling the reaction solution to room temperature, the solvent was evaporated off under reduced pressure. To the residue, ethyl acetate was added, washed with 2N hydrochloric acid, and dried over magnesium sulfate. After separating the drying agent by filtration, the solvent was evaporated off under reduced pressure to obtain a crude product of N-[4-(1-chloro-2,2,2-trifluoroethyl)-2,6-dimethyl-phenyl]-3-nitrobenzamide (0.93 g). This crude product was used for the next reaction without further purification.

### Step 8. Synthesis N-(4-{1-[3,5-bis(trifluoromethyl)-1H-pyrazol-1-yl]-2,2,2-trifluoroethyl}-2,6-dimethyl-phenyl)-3-nitrobenzamide

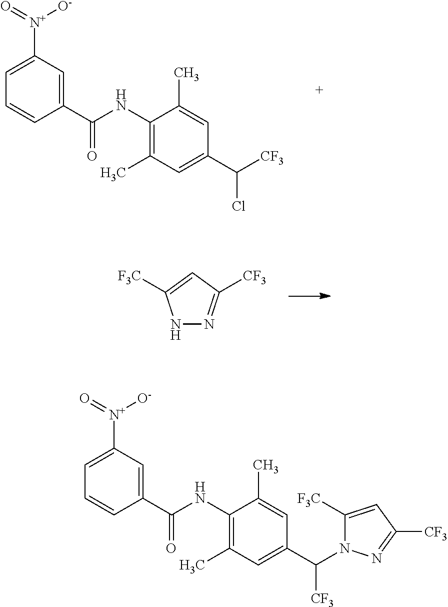

N-[4-(1-chloro-2,2,2-trifluoroethyl)-2,6-dimethylphenyl]-3-nitrobenzamide (0.50 g) and 3,5-bis(trifluoromethyl)-1H-pyrazole (0.29 g) were dissolved in acetonitrile. To this solution, potassium carbonate (0.21 g) was added, and then stirred under heating at 60° C. for 1.5 hours. After cooling the reaction solution to room temperature, a saturated aqueous solution of ammonium chloride was added and the resulting solution was extracted twice with ethyl acetate. The organic layers were combined, washed with water, and dried over magnesium sulfate. After separating the drying agent by filtration, the solvent was evaporated off under reduced pressure to obtain a crude product. Thus obtained crude product was purified with silica gel chromatography to obtain N-(4-{1-[3,5-bis(trifluoromethyl)-1H-pyrazol-1-yl]-2,2,2-trifluoroethyl}-2,6-dimethylphenyl)-3-nitrobenzamide (0.49 g).

¹H-NMR (CDCl₃) δ: 2.33 (6H, s), 5.80 (1H, q), 6.98 (1H, s), 7.46 (3H, s), 7.49 (3H, s), 7.74 (1H, dd), 8.29 (1H, d), 8.45 (1H, d), 8.74 (1H, s).

### Step 9. Synthesis of 3-amino-N-(4-{1-[3,5-bis(trifluoromethyl)-1H-pyrazol-1-yl]-2,2,2-ethyl}-2,6-dimethylphenyl)benzamide

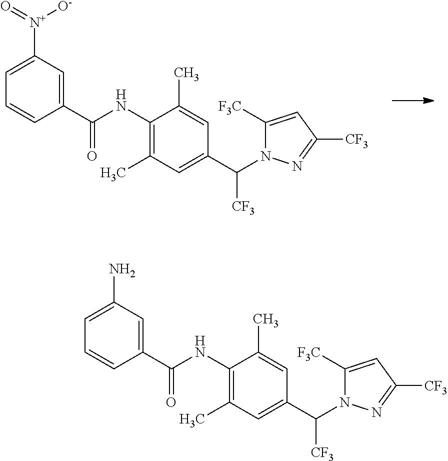

N-(4-{1-[3,5-bis(trifluoromethyl)-1H-pyrazol-1-yl]-2,2,2-trifluoroethyl}-2,6-dimethylphenyl)-3-nitrobenzamide (0.45 g) was dissolved in ethanol. To this solution, stannic chloride dihydrate (0.57 g) and concentrated hydrochloric acid (1 ml) were added and stirred under heating at 60° C. for 4 hours. After cooling down to room temperature, ethyl acetate and water were added. With vigorous stirring, potassium carbonate was added for neutralization. The resulting mixture was filtered using

Celite to separate the organic layer. The aqueous layer was extracted with ethyl acetate. The organic layers were combined, washed with water, and dried over magnesium sulfate. After separating the drying agent by filtration, the solvent was evaporated off under reduced pressure to obtain 3-amino-N-(4-{1-[3,5-bis(trifluoromethyl)-1H-pyrazol-1-yl]-2,2,2-trifluoroethyl}-2,6-dimethylphenyl)benzamide (0.39 g).

¹H-NMR (CDCl₃) δ: 2.30 (6H, s), 3.85 (2H, s), 5.78 (1H, q), 6.86 (1H, d), 6.96 (1H, s), 7.19-7.33 (4H, m), 7.43 (2H, s).

### Step 10. Synthesis of N-{3-[(4-{1-[3,5-bis(trifluoromethyl)-1H-pyrazol-1-yl)-2,2,2-trifluoroethyl}-2,6-dimethylphenyl)carbamoyl]phenyl}-2-chloropyridine-3-carboxamide

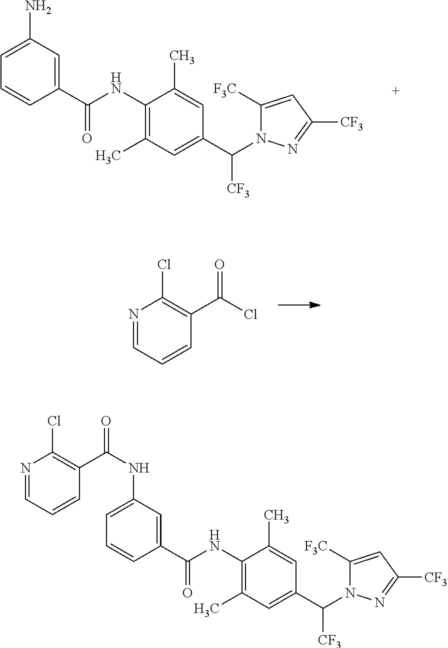

3-amino-N-(4-{1-[3,5-bis(trifluoromethyl)-1H-pyrazol-1-yl]-2,2,2-trifluoro-ethyl}-2,6-dimethylphenyl)benzamide (0.12 g) and pyridine (0.03 g) were dissolved in THF. To this solution, 2-chloropyridine-3-carbonyl chloride (0.04 g) was added, and then stirred at room temperature for 1 hour. The reaction mixture was poured over water and extracted twice with ethyl acetate. The organic layers were combined, washed with water and dried over magnesium sulfate. After separating the drying agent by filtration, the solvent was evaporated off under reduced pressure to obtain a crude product. Thus obtained crude produce was purified with silica gel chromatography to obtain N-{3-[(4-{1-[3,5-bis(trifluoromethyl)-1H-pyrazol-1-yl]-2,2,2-trifluoroethyl}-2,6-dimethylphenyl)carbamoyl)phenyl)-2-chloropyridine-3-carboxamide (0.13 g).

¹H-NMR (CDCl₃) δ: 2.28 (6H, s), 5.79 (1H, q), 6.97 (1H, s), 7.35-7.50 (4H, m), 7.68-7.81 (3H, m), 8.11(1H, d), 8.26 (1H, s), 8.47-8.59 (2H, m).

### SYNTHETIC EXAMPLE 4

### Synthesis of 2-chloro-N-[3-({2,6-dibromo-4-[1-(4-chlorophenyl)-1-(4-chloro-1H-pyrazol-1-yl)-2,2,2-trifluoro-ethyl]phenyl}carbamoyl)phenyl]pyridine-3-carboxamide (No. 2-73)

### Step 1. Synthesis of (4-chlorophenyl)(4-nitrophenyl)methanone

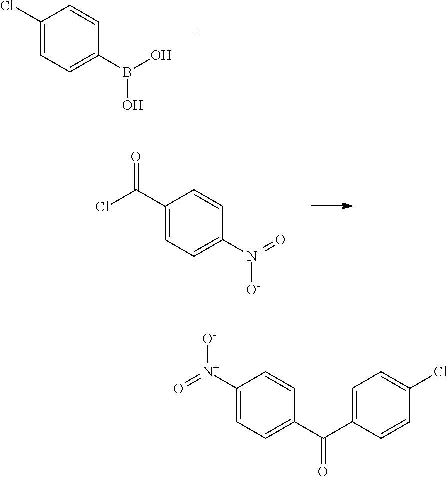

4-chlorophenylboric acid (3.00 g), 4-nitrobenzoyl chloride (4.27 g), potassium phosphate hydrate (6.52 g), dichlorobis(triphenylphophine)palladium (II) (0.27 g) and toluene (60 ml) were mixed and heated at 100° C. for 6 hours under the argon atmosphere. Water and ethyl acetate were added thereto, the organic layer was separated and extracted from the aqueous layer. The resulting solution was dried over magnesium sulfate. After the filtration, the solvent was evaporated off under reduced pressure. Thus obtained residue was purified with silica gel chromatography to obtain the title compound (3.52 g, 70%).

¹H-NMR (CDCl₃) δ: 7.51 (2H, d), 7.76 (2H, d), 7.92 (2H, d), 8.35 (2H, d)

### Step 2. Synthesis of [1-(4-chlorophenyl)-2,2,2-trifluoro-1-(4-nitrophenyl)-ethoxy]-(trimethyl)silane

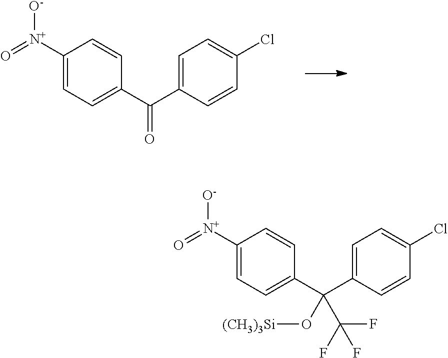

(4-chlorophenyl)(4-nitrophenyl)methanone (3.03 g) was dissolved in DMF (40 ml), and lithium acetate (76 mg) and trifluoromethyltrimethylsilane (2.57 ml) were added thereto under ice cooling. The resulting solution was stirred at room temperature overnight and then water was added thereto. The solution was extracted with ethyl acetate, washed with water, and dried over magnesium sulfate. After the filtration, the solvent was evaporated off under reduced pressure. Thus obtained residue was purified with silica gel chromatography to obtain the title compound (4.11 g, 88%).

¹H-NMR (CDCl₃) δ: −0.01 (9H, s), 7.29 (2H, d), 7.34 (2H, d), 7.58 (2H, d), 8.19 (2H, d).

### Step 3. Synthesis of 4-{1-(4-chlorophenyl)-2,2,2-trifluoro-1-[(trimethylsilyl)-oxy]ethyl}aniline

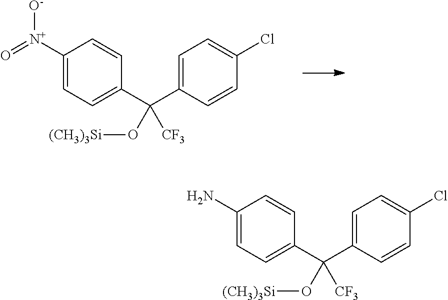

Nickel chloride (II) hexahydrate (1.19 g) was dissolved in methanol (70 ml) and sodium borohydride (0.57 g) was added thereto at room temperature. After stirring for 30 min, [1-(4-chlorophenyl)-2,2,2-trifluoro-1-(4-nitrophenyl)ethoxy]-(trimethyl)silane (4.04 g) was added thereto followed by cooling in ice water bath. Then, sodium borohydride (1.51 g) was added thereto in small amount followed by stirring for 1 hour. The solvent was removed under reduced pressure, ammonia water and ethyl acetate were added, and insoluble matters were filtered using Celite. The organic layer was separated from the filtrate and the aqueous layer was extracted by ethyl acetate. The combined extracts were dried over magnesium sulfate. After the filtration, the solvent was evaporated off under reduced pressure to obtain the title compound as a crude product (3.20 g).

¹H-NMR (CDCl₃) δ: −0.06 (9H, s), 3.74 (2H, s), 6.61 (2H, d), 7.11 (2H, d), 7.28 (2H, d), 7.36 (2H, d).

### Step 4. Synthesis of tert-butyl {4-[1-(4-chlorophenyl)-2,2,2-trifluoro-1-hydroxyethyl]-phenyl}carbamate

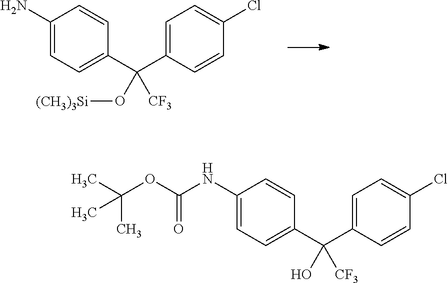

4-{1-(4-chlorophenyl)-2,2,2-trifluoro-1-[(trimethylsilyl)oxy]ethyl}aniline (0.67 g) was dissolved in THF (25 ml), and triethylamine (0.28 ml), di-tert-butyl bicarbonate (0.62 ml) and 4-dimethylaminopyridine (22 mg) were added thereto at room temperature. The reaction solution was stirred overnight, and then tetrabutylammonium fluoride (1.0M/THF, 2.51 ml) was added thereto followed by stirring for 5 hours. An aqueous solution of ammonium chloride was added, and the mixture was extracted with ethyl acetate and dried over magnesium sulfate. After the filtration, the solvent was evaporated off under reduced pressure. Thus obtained residue was purified with silica gel chromatography to obtain the title compound (0.34 g, 47%).

¹H-NMR (CDCl₃) δ: 1.44 (9H, s), 2.92 (1H, s), 6.51 (1H, br s), 7.23 (2H, s), 7.31 (2H, d), 7.38 (2H, d), 7.45 (2H, d).

### Step 5. Synthesis of tert-butyl {4-[1-chloro-1-(4-chlorophenyl)-2,2,2-trifluoroethyl]-phenyl}carbamate

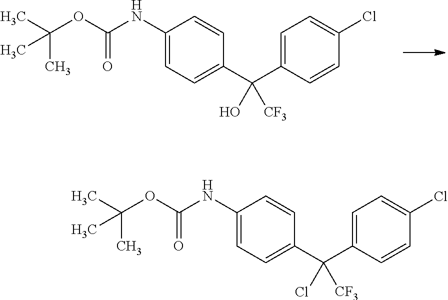

tert-butyl {4-[1-(4-chlorophenyl)-2,2,2-trifluoro-1-hydroxyethyl]phenyl}-carbamate (0.34 g) was dissolved in toluene (20 ml), and pyridine (0.14 ml) and thionyl chloride (0.12 ml) were added thereto. The reaction solution was heated at 70° C. for 8 hours. After adding an aqueous solution of sodium bicarbonate and ethyl acetate, the organic layer was separated. The aqueous layer was extracted with ethyl acetate, and dried over magnesium sulfate. After the filtration, the solvent was evaporated off under reduced pressure to obtain the title compound as a crude product (0.25 g).

¹H-NMR (CDCl₃) δ: 1.53 (9H, s), 6.54 (2H, s), 7.32 (2H, d), 7.36 (4H, s), 7.42 (2H, d).

### Step 6. Synthesis of tert-butyl {4-[1-(4-chlorophenyl)-1-(4-chloro-1H-pyrazol-1-yl)-2,2,2-trifluoroethyl]phenyl}carbamate

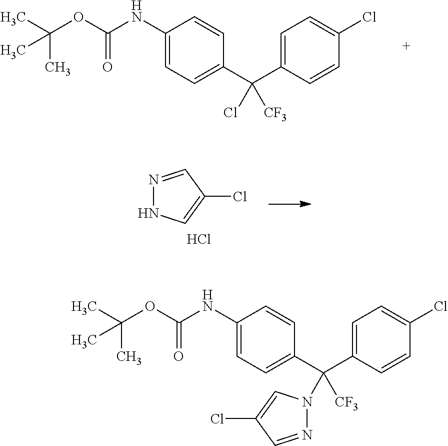

tert-butyl {4-[1 -chloro-1-(4-chlorophenyl)-2,2,2-trifluoroethyl]phenyl}-carbamate (0.25 g), 4-chloropyrazole hydrochloride (99 mg), potassium carbonate (0.20 g) and potassium iodide (10 mg) were mixed in acetonitrile (5 ml), and the resulting mixture was heated at 80° C. for 3 hours. After cooling down to room temperature, insoluble matters were filtered off and the solvent was evaporated off under reduced pressure. The residue was purified with silica gel chromatography to obtain the title compound (0.25 g, 86%).

¹H-NMR (CDCl₃) δ: 1.52 (9H, s), 6.57 (1H, s), 6.96-7.04 (4H, m), 7.11 (1H, s), 7.33-7.42 (4H, m), 7.64 (1H, s).

### Step 7. Synthesis of 4-[1-(4-chlorophenyl)-1-(4-chloro-1H-pyrazol-1-yl)-2,2,2-trifluoroethyl]aniline

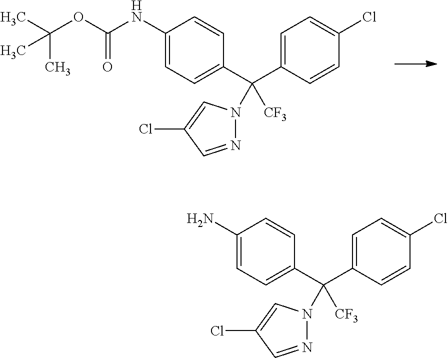

tert-butyl {4-[1-(4-chlorophenyl)-1-(4-chloro-1H-pyrazol-1-yl)-2,2,2-trifluoroethyl]phenyl}carbamate (0.25 g) was dissolved in ethanol (5 ml), and concentrated hydrochloric acid (1 ml) was added thereto. The reaction mixture was heated at 50° C. for 4 hours. The solvent was evaporated off under reduced pressure, and an aqueous solution of sodium carbonate and ethyl acetate were added thereto to separate off the organic layer. The aqueous layer was extracted with ethyl acetate and dried over magnesium sulfate. After the filtration, the solvent was evaporated off under reduced pressure. The residue was purified with silica gel chromatography to obtain the title compound (0.15 g, 75%).

¹H-NMR (CDCl₃) δ: 6.40 (2H, br s), 6.65 (2H, d), 6.88 (2H, d), 6.96 (2H, d), 7.11 (1H, s), 7.34 (2H, d), 7.64 (1H, s).

### Step 8. Synthesis of 2,6-dibromo-4-[1-(4-chlorophenyl)-1-(4-chloro-1H-pyrazol-1-yl)-2,2,2-trifluoroethyl]aniline

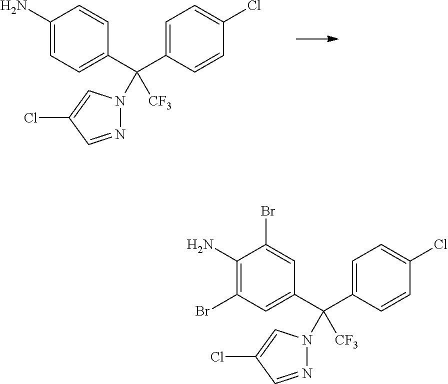

### Synthesis of 2,6-dibromo-4-[1-(4-chlorophenyl)-1-(4-chloro-1H-pyrazol-1-yl)-2,2,2-trifluoroethyl]aniline

4-[1-(4-chlorophenyl)-1-(4-chloro-1H-pyrazol-1-yl)-2,2,2-trifluoroethyl]aniline (0.15 g) was dissolved in DMF (3 ml), and N-bromosuccinimide (0.15 g) was added thereto. The reaction mixture was heated at 60° C. for 1 hour. After adding water, the mixture was extracted with ethyl acetate and washed with water. After drying over magnesium sulfate and the filtration, the solvent was evaporated off under reduced pressure. The residue was purified with silica gel chromatography to obtain the title compound (0.18 g, 87%).

¹H-NMR (CDCl₃) δ: 4.81 (2H, s), 6.98 (2H, d), 7.11 (2H, s), 7.17 (1H, s), 7.39 (2H, d), 7.65 (1H, s).

### Step 9. Synthesis of N-{2,6-dibromo-4-[1-(4-chlorophenyl)-1-(4-chloro-1H-pyrazol-1-yl)-2,2,2-trifluoroethyl]-phenyl}-3-nitrobenzamide

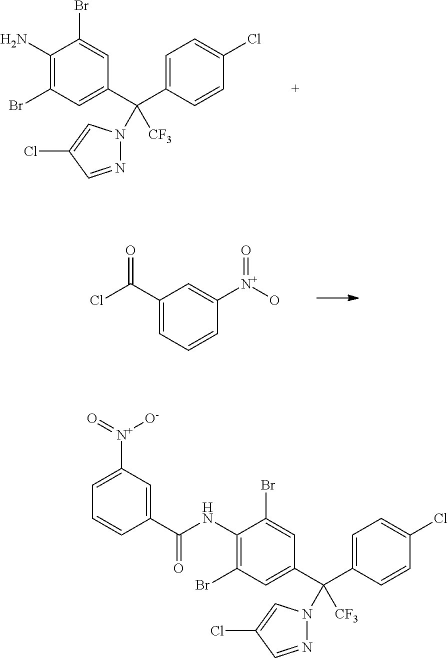

To a mixture of 2,6-dibromo-4-[1-(4-chlorophenyl)-1-(4-chloro-1H-pyrazol-1-yl)-2,2,2-trifluoroethyl]aniline (0.17 g) and 3-nitrobenzoyl chloride (0.13 g), pyridine (0.25 ml) was added. The reaction mixture was heated at 100° C. for 5 hours. After adding water, the mixture was extracted with ethyl acetate. After drying over magnesium sulfate and the filtration, the solvent was evaporated off under reduced pressure. The residue was purified with silica gel chromatography to obtain the title compound (0.096 g, 44%).

¹H-NMR (CDCl₃) δ: 7.09 (2H, d), 7.23 (1H, s), 7.35 (2H, s), 7.45 (2H, d), 7.68 (1H, s), 7.69 (1H, s), 7.76 (1H, t), 8.29-8.34 (1H, m), 8.45-8.51 (1H, m), 8.79 (1H, s).

### Step 10. Synthesis of 3-amino-N-{2,6-dibromo-4-[1-(4-chlorophenyl)-1-(4-chloro-1H-pyrazol-1-yl)-2,2,2-trifluoroethyl]phenyl}benzamide

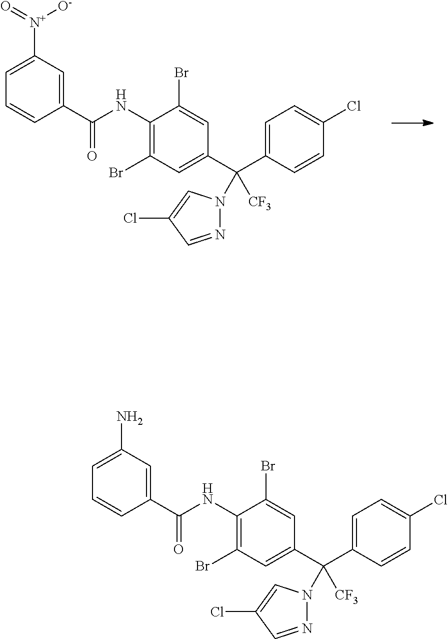

N-{2,6-dibromo-4-[1-(4-chlorophenyl)-1-(4-chloro-1H-pyrazol-1-yl)-2,2,2-trifluoroethyl]phenyl}-3-nitrobenzamide (96 mg) was dissolved in ethanol (3 ml). To this solution, stannic chloride dihydrate (0.13 g) and concentrated hydrochloric acid (0.5 ml) were added and the reaction mixture was refluxed under heating at for 4 hours. The solvent was evaporated off under reduced pressure, and an aqueous solution of sodium carbonate and ethyl acetate were added. Insoluble matters were filtered using Celite and filtrate was extracted with ethyl acetate. The organic layer was dried over magnesium sulfate. After the filtration, the solvent was evaporated off under reduced pressure and purified with silica gel chromatography, and the title compound was obtained (90 mg, 98%).

¹H-NMR (CDCl₃) δ: 3.88 (2H, br s), 6.86-6.92 (1H, m), 7.07 (2H, d), 7.22 (1H, s), 7.24-7.30 (3H, m), 7.32 (2H, s), 7.43 (2H, d), 7.58 (1H, s), 7.66 (1H, s).

### Step 11. Synthesis of 2-chloro-N-[3-({2,6-dibromo-4-[1-(4-chlorophenyl)-1-(4-chloro-1H-pyrazol-1-yl)-2,2,2-trifluoro-ethyl]phenyl}carbamoyl)phenyl]pyridine-3-carboxamide

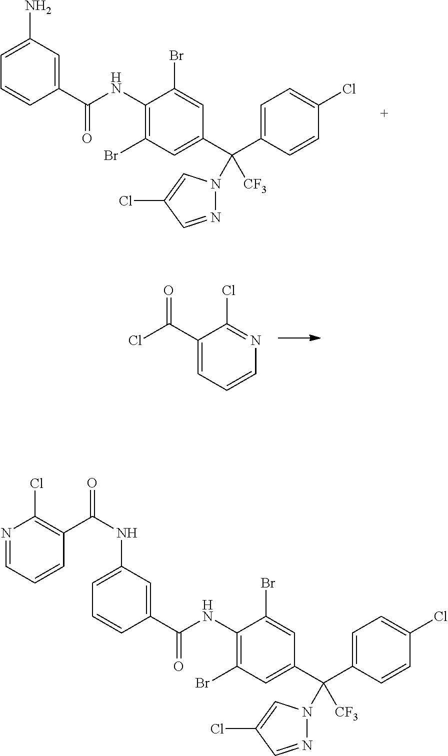

3-amino-N-{2,6-dibromo-4-[1-(4-chlorophenyl)-1-(4-chloro-1H-pyrazol-1-yl)-2,2,2-trifluoroethyl]phenyl}benzamide (90 mg) and pyridine (0.016 ml) were dissolved in THF (2 ml), and then 2-chloronicotinic acid chloride (31 mg) was added thereto at room temperature. After stirring overnight, water was added and the mixture was extracted with ethyl acetate. The organic layer was dried over magnesium sulfate. After the filtration, the solvent was evaporated off under reduced pressure. After the purification with silica gel chromatography, the title compound was obtained (90 mg, 83%).

¹H-NMR (CDCl₃) δ: 7.08 (2H, d), 7.22 (1H, s), 7.34 (2H, s), 7.42-7.45 (3H, m), 7.57 (1H, t), 7.67 (1H, s), 7.72-7.82 (2H, m), 7.93 (1H, d), 8.20-8.30 (2H, m), 8.37 (1H, s), 8.55 (1H, ).

### SYNTHETIC EXAMPLE 5

### Synthesis of N-[4-(1,1,1,3,3,3-hexafluoro-2-methoxypropan-2-yl)-2,6-diiodophenyl]-3-[(phenylcarbonyl)amino]benzamide. (No. 1-1)

### Step 1. Synthesis of 4-(1,1,1,2,3,3,3-heptafluoropropan-2-yl)aniline

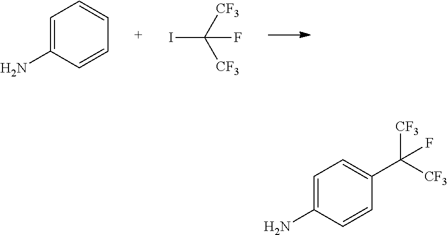

To a mixed solution comprising TBME (75 ml) and water (100 ml), aniline (6.96 g), Na₂S₂O₄ (15.61 g), NaHCO₃ (7.53 g) and tetrabutylammonium bisulfate (3 g) were added. To this mixed solution, heptafluoroisopropyl iodide (26.53 g) was added dropwise and the mixture was stirred at room temperature for 2 hours. The organic layer was washed with 0.75N hydrochloric acid (65 ml) and saturated NaHCO₃. After evaporating off the solvent, the residue was purified with silica gel chromatography (hexane:ethyl acetate=4:1) to obtain the desired 4-(1,1,1,2,3,3,3-heptafluoropropan-2-yl)aniline (18 g). Yield 92%, ND₂₀ 1.4167.

### Step 2. Synthesis of 4-(1,1,1,2,3,3,3-heptafluoropropan-2-yl)-2,6-diiodoaniline

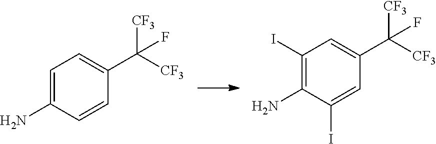

4-(1,1,1,2,3,3,3-heptafluoropropan-2-yl)aniline (22.91 g) was dissolved in acetic acid, and N-iodosuccinimide (39.48 g) was added thereto in small amount. After stirring at 60° C. for 1 hour, the solvent was evaporated off under reduced pressure and water was added. Hexane was used for further extraction, and the solvent was evaporated off. The residue was purified with silica gel chromatography (hexane:ethyl acetate=14:1) to obtain the desired compound of 4-(1,1,1,2,3,3,3-heptafluoro-propan-2-yl)-2,6-diiodoaniline (33.17 g). Yield 74%, mp: 72-75° C.

### Step 3. Synthesis of 4-(1,1,1,3,3,3-hexafluoro-2-methoxypropan-2-yl)-2,6-diiodoaniline

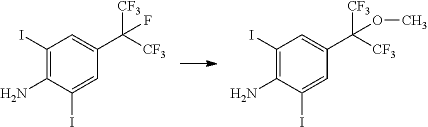

To a methanol solution of 4-(1,1,1,2,3,3,3-heptafluoropropan-2-yl)-2,6-diiodoaniline (2 g), sodium methoxide (0.84 g) was added. After stirring at room temperature overnight, water was added and the mixture was extracted with ethyl acetate. Then, the solvent was evaporated off. The residue was purified with silica gel chromatography (hexane:ethyl acetate=18:1) to obtain the desired compound of 4-(1,1,1,3,3,3-hexafluoro-2-methoxypropan-2-yl)-2,6-diiodoaniline (1.88 g). Yield 92%, mp: 57-59° C.

### Step 4. Synthesis of N-[4-(1,1,1,3,3,3-hexafluoro-2-methoxypropan-2-yl)-2,6-diiodo-phenyl]-3-nitrobenzamide

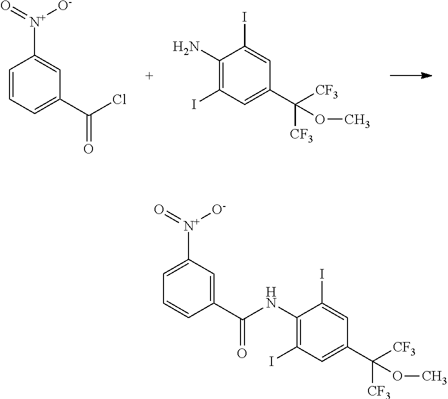

4-(1,1,1,3,3,3-hexafluoro-2-methoxypropan-2-yl)-2,6-diiodoaniline (1.95 g) and 3-nitrobenzoyl chloride (1.38 g) were dissolved in pyridine followed by reflux under heating for 3 hours. After cooling down to room temperature, and water was added followed by extraction using ethyl acetate. Then, the solvent was evaporated off, ethanol was added and then 30% NaOH (2 ml) was added thereto. The mixture was stirred at room temperature for 1 hour. After adding water, the mixture was extracted with ethyl acetate. Then, the solvent was evaporated. The residue was purified with silica gel chromatography (hexane:ethyl acetate=5:2) to obtain the desired compound of N-[4-(1,1,1,3,3,3-hexafluoro-2-methoxypropan-2-yl)-2,6-diiodophenyl]-3-nitrobenzamide (1.86 g). Yield 74%, mp: 214-218° C.

### Step 5. Synthesis of 3-amino-N-[4-(1,1,1,3,3,3-hexafluoro-2-methoxypropan-2-yl)-2,6-diiodophenyl]benzamide

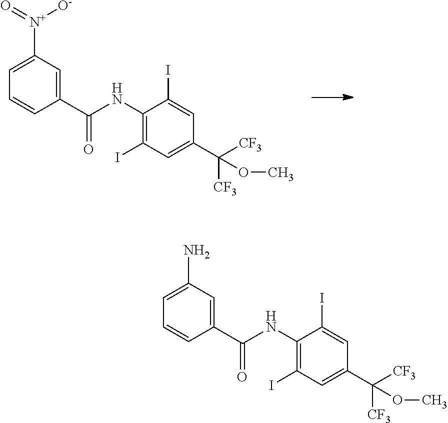

N-[4-(1,1,1,3,3,3-hexafluoro-2-methoxypropan-2-yl)-2,6-diiodophenyl]-3-nitrobenzamide (1.86 g) and stannous chloride (2.09 g) were dissolved in ethanol followed by addition of concentrated hydrochloric acid (3 ml). The mixture was then heated at 80° C. for 1 hour. After cooling down to room temperature, water was added, K₂CO₃ was added to obtain an alkaline solution followed by addition of ethyl acetate. The precipitated insoluble matters were removed by filtration using Celite, the organic layer was separated off and dried over Na₂SO₄. Then, the solvent was evaporated off under reduced pressure. The residue was purified with silica gel chromatography (hexane:ethyl acetate=1:1) to obtain the desired compound of 3-amino-N-[4-(1,1,1,3,3,3-hexafluoro-2-methoxypropan-2-yl)-2,6-diiodophenyl]benzamide (1.7 g).

Yield 96%, mp: 189-191° C.

### Step 6. Synthesis of N-[4-(1,1,1,3,3,3-hexafluoro-2-methoxypropan-2-yl)-2,6-diiodo-phenyl]-3-[(phenylcarbonyl)amino]benzamide

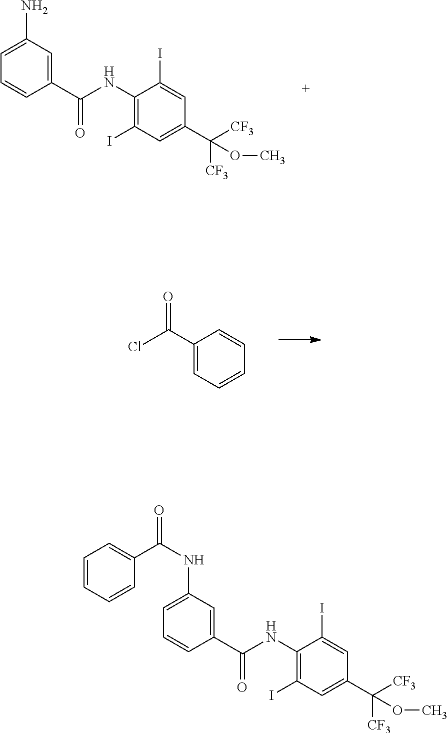

3-amino-N-[4-(1,1,1,3,3,3-hexafluoro-2-methoxypropan-2-yl)-2,6-diiodophenyl]benzamide (100 mg) and pyridine (25 mg) were dissolved in THF, and then benzoyl chloride (44 mg) was added thereto dropwise. The mixture was stirred at room temperature for 1 hour and water was added followed by extraction with ethyl acetate. Then, the solvent was evaporated off. The residue was purified with silica gel chromatography (hexane:ethyl acetate=3:2) to obtain the desired compound of N-[4-(1,1,1,3,3,3-hexafluoro-2-methoxypropan-2-yl)-2,6-diiodophenyl]-3-[(phenyl-carbonyl)amino]benzamide (100 mg). Yield 86%, mp: 128-141° C.

The compounds of the present invention and the intermediates for manufacturing compounds according to the invention which are prepared or can be prepared according to the methods described hereing or according to similar methods known to the skilled person are exemplified in the following tables

TABLE 1

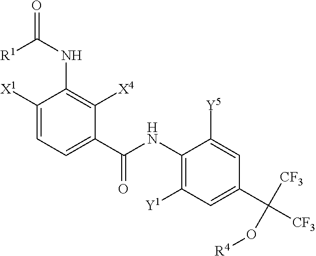

| | R1 | X1 | X4 | Y1 | Y5 | OR4 |
|---|---|---|---|---|---|---|
| 1-1 | phenyl | H | H | I | I | methoxy |
| 1-2 | 2-fluorophenyl | H | H | I | I | methoxy |
| 1-3 | 4-fluorophenyl | H | H | I | I | methoxy |
| 1-4 | 2-methylpyridin-3-yl | H | H | I | I | methoxy |
| 1-5 | 2-fluoropyridin-3-yl | H | H | I | I | methoxy |
| 1-6 | 2-chloropyridin-3-yl | H | H | I | I | methoxy |
| 1-7 | 6-chloropyridin-3-yl | H | H | I | I | methoxy |
| 1-8 | 2-chloropyridin-3-yl | H | H | CH3 | CH3 | methoxy |
| 1-9 | 2-chloropyridin-3-yl | H | H | CH3 | ethyl | methoxy |
| 1-10 | 2-chloropyridin-3-yl | H | H | CH3 | Br | methoxy |
| 1-11 | 2-chloropyridin-3-yl | H | H | Cl | Cl | methoxy |
| 1-12 | 2-chloropyridin-3-yl | H | H | Br | Br | methoxy |
| 1-13 | 2-chloropyridin-3-yl | H | H | I | I | methoxy |
| 1-14 | 2-chloropyridin-3-yl | H | H | CH3 | ethyl | methoxy |
| 1-15 | 2-chloropyridin-3-yl | H | H | CH3 | ethyl | methoxy |
| 1-16 | 2-chloropyridin-3-yl | H | H | CH3 | ethyl | methoxy |
| 1-17 | 2-chloropyridin-3-yl | H | H | CH3 | ethyl | methoxy |
| 1-18 | 2-chloro-6-methylpyridin-3-yl | H | H | I | I | methoxy |
| 1-19 | 2,6-dichloropyridin-3-yl | H | H | I | I | methoxy |
| 1-20 | pyrazin-2-yl | H | H | I | I | methoxy |
| 1-21 | 4-methyl-1,2,3-thiadiazol-5-yl | H | H | I | I | methoxy |
| 1-22 | isopropyl | H | H | I | I | ethoxy |
| 1-23 | 2-chloroethyl | H | H | I | I | ethoxy |
| 1-24 | ethoxy | H | H | I | I | ethoxy |
| 1-25 | phenyl | H | H | I | I | ethoxy |
| 1-26 | 2-fluorophenyl | H | H | I | I | ethoxy |
| 1-27 | 3-fluorophenyl | H | H | I | I | ethoxy |
| 1-28 | 4-fluorophenyl | H | H | I | I | ethoxy |
| 1-29 | 2-chloropyridin-3-yl | H | H | CH3 | CH3 | ethoxy |
| 1-30 | 2-chloropyridin-3-yl | F | H | CH3 | CH3 | ethoxy |
| 1-31 | 2-chloropyridin-3-yl | H | F | CH3 | CH3 | ethoxy |
| 1-32 | 2-chloropyridin-3-yl | H | Cl | CH3 | CH3 | ethoxy |
| 1-33 | 2-chloropyridin-3-yl | H | H | CH3 | ethyl | ethoxy |
| 1-34 | 2-chloropyridin-3-yl | F | H | CH3 | ethyl | ethoxy |
| 1-35 | 2-chloropyridin-3-yl | H | F | CH3 | ethyl | ethoxy |
| 1-36 | 2-chloropyridin-3-yl | H | Cl | CH3 | ethyl | ethoxy |
| 1-37 | 2-chloropyridin-3-yl | H | H | CH3 | Br | ethoxy |
| 1-38 | 2-chloropyridin-3-yl | H | H | Cl | Cl | ethoxy |
| 1-39 | 2-chloropyridin-3-yl | H | H | Br | Br | ethoxy |
| 1-40 | 2-chloropyridin-3-yl | H | H | I | I | ethoxy |
| 1-41 | 2,2,2-trichloroethoxy | H | H | CH3 | ethyl | ethoxy |
| 1-42 | 2,2,2-trichloroethoxy | H | H | Br | Br | ethoxy |
| 1-43 | 2-chloropyridin-3-yl | H | H | CH3 | CH3 | ethoxy |
| 1-44 | 2-chloropyridin-3-yl | H | F | CH3 | CH3 | ethoxy |
| 1-45 | 2-chloropyridin-3-yl | H | Cl | CH3 | CH3 | ethoxy |
| 1-46 | 2-chloropyridin-3-yl | H | H | CH3 | ethyl | ethoxy |
| 1-47 | 2-chloropyridin-3-yl | H | F | CH3 | ethyl | ethoxy |
| 1-48 | 2-chloropyridin-3-yl | H | Cl | CH3 | ethyl | ethoxy |
| 1-49 | 2-chloropyridin-3-yl | H | H | CH3 | Br | ethoxy |
| 1-50 | 2-chloropyridin-3-yl | H | H | Br | Br | ethoxy |
| 1-51 | 2-chloropyridin-3-yl | H | H | I | I | ethoxy |
| 1-52 | 2-chloropyridin-3-yl | H | H | CH3 | ethyl | ethoxy |
| 1-53 | 2-chloropyridin-3-yl | H | H | Br | Br | ethoxy |
| 1-54 | 2-chloropyridin-3-yl | H | H | CH3 | ethyl | ethoxy |
| 1-55 | 2-chloropyridin-3-yl | H | H | Br | Br | ethoxy |
| 1-56 | 2-chloropyridin-3-yl | H | H | CH3 | ethyl | ethoxy |
| 1-57 | 2-chloropyridin-3-yl | H | H | Br | Br | ethoxy |
| 1-58 | 2-chloropyridin-3-yl | H | H | CH3 | ethyl | ethoxy |
| 1-59 | 2-fluoropyridin-3-yl | H | H | I | I | ethoxy |
| 1-60 | 6-chloropyridin-3-yl | H | H | I | I | ethoxy |
| 1-61 | 2,6-dichloropyridin-3-yl | H | H | I | I | ethoxy |
| 1-62 | 2,6-dichloro-5-fluoropyridin-3-yl | H | H | I | I | 3-propoxy |
| 1-63 | 2-chloro-6-methylpyridin-3-yl | H | H | I | I | 3-propoxy |
| 1-64 | 2,6-dichloropyridin-3-yl | H | H | I | I | 3-propoxy |
| 1-66 | 2-chloropyridin-3-yl | H | H | CH3 | ethyl | cyclopropylmethoxy |
| 1-67 | 2-chloropyridin-3-yl | H | H | Br | Br | cyclopropylmethoxy |
| 1-68 | 2-chloropyridin-3-yl | H | H | CH3 | ethyl | cyclopentyloxy |
| 1-69 | 2-chloropyridin-3-yl | H | H | Br | Br | cyclopentyloxy |
| 1-70 | 2-chloropyridin-3-yl | H | H | CH3 | ethyl | cyclohexyloxy |
| 1-71 | 2-chloropyridin-3-yl | H | H | Br | Br | cyclohexyloxy |
| 1-72 | 2-chloropyridin-3-yl | H | H | CH3 | ethyl | prop-2-en-1-yloxy |
| 1-73 | 2-chloropyridin-3-yl | H | H | Br | Br | prop-2-en-1-yloxy |
| 1-74 | 2-chloropyridin-3-yl | H | H | I | I | prop-2-en-1-yloxy |
| 1-75 | 2-chloropyridin-3-yl | H | H | CH3 | ethyl | (3-methylbut-2-en-1-yl) |
| | | | | | | oxy |
| 1-76 | 2-chloropyridin-3-yl | H | H | Br | Br | (3-methylbut-2-en-1-yl) |
| | | | | | | oxy |
| 1-77 | 2-chloropyridin-3-yl | H | H | CH3 | ethyl | cyclohex-2-en-1-yloxy |
| 1-78 | 2-chloropyridin-3-yl | H | H | Br | Br | cyclohex-2-en-1-yloxy |
| 1-79 | phenyl | H | H | I | I | prop-2-yn-1-yloxy |
| 1-80 | 2-chloropyridin-3-yl | H | H | I | I | prop-2-yn-1-yloxy |
| 1-81 | 2-chloro-6-methylpyridin-3-yl | H | H | I | I | prop-2-yn-1-yloxy |
| 1-82 | 2,6-dichloropyridin-3-yl | H | H | I | I | prop-2-yn-1-yloxy |
| 1-83 | phenyl | H | H | I | I | but-2-yn-1-yloxy |
| 1-84 | 4-fluorophenyl | H | H | I | I | but-2-yn-1-yloxy |
| 1-85 | 2-chloropyridin-3-yl | H | H | I | I | but-2-yn-1-yloxy |
| 1-86 | 2,6-dichloropyridin-3-yl | H | H | I | I | but-2-yn-1-yloxy |
| 1-87 | 2,6-dichloro-5-fluoropyridin-3-yl | H | H | I | I | but-2-yn-1-yloxy |
| 1-88 | 4-fluorophenyl | H | H | I | I | 2-methoxyethoxy |
| 1-89 | 2,5-difluorophenyl | H | H | I | I | 2-methoxyethoxy |
| 1-90 | 2,4-dichlorophenyl | H | H | I | I | 2-methoxyethoxy |
| 1-91 | 2-chloro-4-nitrophenyl | H | H | I | I | 2-methoxyethoxy |
| 1-92 | 2,4-dichloro-5-fluorophenyl | H | H | I | I | 2-methoxyethoxy |
| 1-93 | 2-fluoropyridin-3-yl | H | H | I | I | 2-methoxyethoxy |
| 1-94 | 2-chloropyridin-3-yl | H | H | CH3 | ethyl | 2-methoxyethoxy |
| 1-95 | 2-chloropyridin-3-yl | H | H | Br | Br | 2-methoxyethoxy |
| 1-96 | 2-chloropyridin-3-yl | H | H | I | I | 2-methoxyethoxy |
| 1-97 | 6-chloropyridin-3-yl | H | H | I | I | 2-methoxyethoxy |
| 1-98 | 2-chloro-6-methylpyridin-3-yl | H | H | I | I | 2-methoxyethoxy |
| 1-99 | 2,6-dichloropyridin-3-yl | H | H | I | I | 2-methoxyethoxy |
| 1-100 | 2,6-dichloro-5-fluoropyridin-3-yl | H | H | I | I | 2-methoxyethoxy |
| 1-101 | phenyl | H | H | I | I | 2-(methylsulfanyl)ethoxy |
| 1-102 | 4-fluorophenyl | H | H | I | I | 2-(methylsulfanyl)ethoxy |
| 1-103 | 2-fluoropyridin-3-yl | H | H | I | I | 2-(methylsulfanyl)ethoxy |
| 1-104 | 2-chloropyridin-3-yl | H | H | I | I | 2-(methylsulfanyl)ethoxy |
| 1-105 | 2-chloropyridin-3-yl | H | H | CH3 | ethyl | phenoxy |
| 1-106 | 2-chloropyridin-3-yl | H | H | Br | Br | phenoxy |
| 1-107 | 2-chloropyridin-3-yl | H | H | CH3 | ethyl | 2-chlorophenoxy |
| 1-108 | 2-chloropyridin-3-yl | H | H | CH3 | ethyl | 3-chlorophenoxy |
| 1-109 | 2-chloropyridin-3-yl | H | H | CH3 | ethyl | 4-chlorophenoxy |
| 1-110 | 2-chloropyridin-3-yl | H | H | CH3 | ethyl | 3-(trifluoromethyl) |
| | | | | | | phenoxy |
| 1-111 | 2-chloropyridin-3-yl | H | H | CH3 | CH3 | [2-(trifluoromethyl) |
| | | | | | | pyridin-4-yl]oxy |
| 1-112 | 2-chloropyridin-3-yl | H | H | CH3 | ethyl | [2-(trifluoromethyl) |
| | | | | | | pyridin-4-yl]oxy |
| 1-113 | 2-chloropyridin-3-yl | H | H | Br | Br | [2-(trifluoromethyl) |
| | | | | | | pyridin-4-yl]oxy |
| 1-114 | 2-chloropyridin-3-yl | H | H | CH3 | ethyl | benzyloxy |
| 1-115 | 2-chloropyridin-3-yl | H | H | CH3 | ethyl | 2-chlorobenzyloxy |
| 1-116 | 2-chloropyridin-3-yl | H | H | CH3 | ethyl | 3-chlorobenzyloxy |
| 1-117 | 2-chloropyridin-3-yl | H | H | CH3 | ethyl | 4-chlorobenzyloxy |
| 1-118 | 2-chloropyridin-3-yl | H | H | CH3 | ethyl | (ethylideneamino)oxy |
| 1-119 | 2-chloropyridin-3-yl | H | H | CH3 | ethyl | (propan-2-ylideneamino) |
| | | | | | | oxy |
| 1-120 | 2-chloropyridin-3-yl | H | H | Br | Br | (propan-2-ylideneamino) |
| | | | | | | oxy) |
| 1-121 | 2-chloropyridin-3-yl | H | H | CH3 | ethyl | (butan-2-ylideneamino) |
| | | | | | | oxy |
| 1-122 | 2-chloropyridin-3-yl | H | H | Br | Br | (butan-2-ylideneamino) |
| | | | | | | oxy |
| 1-123 | 2-chloropyridin-3-yl | H | H | Br | Br | (cyclopentylideneamino) |
| | | | | | | oxy |
| 1-124 | 2-chloropyridin-3-yl | H | H | Br | Br | (cyclohexylideneamino) |
| | | | | | | oxy |
| 1-125 | 2-chloropyridin-3-yl | H | H | CH3 | ethyl | {(phenylmethylidene) |
| | | | | | | aminooxy |
| 1-126 | 2-chloropyridin-3-yl | H | H | CH3 | ethyl | {(1-phenylethylidene) |
| | | | | | | aminooxy |
| 1-127 | 2-chloropyridin-3-yl | H | H | CH3 | CH3 | 4-chlorobenzyloxy |
| 1-128 | 2-chloropyridin-3-yl | H | H | CH3 | ethyl | 4-fluorophenoxy |
| 1-129 | 2-chloropyridin-3-yl | H | H | CH3 | ethyl | 4-bromophenoxy |
| 1-130 | 2-chloropyridin-3-yl | H | H | CH3 | ethyl | 4-(trifluoromethyl) |
| | | | | | | phenoxy |
| 1-131 | 2-chloropyridin-3-yl | H | H | CH3 | ethyl | 4-(trifluoromethoxy) |
| | | | | | | phenoxy |
| 1-132 | 2-chloropyridin-3-yl | H | H | CH3 | ethyl | 4-(trifluoromethylthio) |
| | | | | | | phenoxy |
| 1-133 | 2-chloropyridin-3-yl | H | H | CH3 | ethyl | 4-methylphenyloxy |
| 1-134 | 2-chloropyridin-3-yl | H | H | CH3 | ethyl | 4-methoxyphenyloxy |
| 1-135 | 2-chloropyridin-3-yl | H | H | CH3 | ethyl | {[(4-fluorophenyl) |
| | | | | | | methylidene]amino}oxy |
| 1-136 | 2-chloropyridin-3-yl | H | H | CH3 | ethyl | {[(2-chlorophenyl) |
| | | | | | | methylidene]amino}oxy |
| 1-137 | 2-chloropyridin-3-yl | H | H | CH3 | ethyl | {[(3-chlorophenyl) |
| | | | | | | methylidene]amino}oxy |
| 1-138 | 2-chloropyridin-3-yl | H | H | CH3 | ethyl | {[(4-chlorophenyl) |
| | | | | | | methylidene]amino}oxy |
| 1-139 | 2-chloropyridin-3-yl | H | H | CH3 | ethyl | {[(4-bromophenyl) |
| | | | | | | methylidene]amino}oxy |
| 1-140 | 2-chloropyridin-3-yl | H | H | CH3 | ethyl | {[(4-iodophenyl) |
| | | | | | | methylidene]amino}oxy |
| 1-142 | 2-chloropyridin-3-yl | H | H | CH3 | ethyl | [2-(pentafluoroethyl) |
| | | | | | | pyridin-4-yl]oxy |
| 1-143 | 2-chloropyridin-3-yl | H | H | CH3 | ethyl | (pyridin-4-yl)oxy |
| 1-144 | phenyl | H | H | I | I | 2-methoxyethoxy |
| 1-145 | 2-fluorophenyl | H | H | I | I | 2-methoxyethoxy |
| 1-146 | 2-chloropyridin-3-yl | H | H | CH3 | ethyl | 2,2,2-trifluoroethoxy |
| 1-147 | 2-chloropyridin-3-yl | H | H | CH3 | ethyl | (2-phenylethyl)oxy |
| 1-148 | 2-chloropyridin-3-yl | H | H | CH3 | ethyl | [1-methyl-3-(trifluoro- |
| | | | | | | methyl)-1H-pyrazol-5-yl]oxy |
| 1-149 | 2-chloropyridin-3-yl | H | H | CH3 | ethyl | {[(4-methylphenyl) |
| | | | | | | methylidene]amino}oxy |
| 1-150 | 2-chloropyridin-3-yl | H | H | CH3 | ethyl | {[(4-methoxyphenyl) |
| | | | | | | methylidene]amino}oxy |
| 1-151 | 2-chloropyridin-3-yl | H | H | CH3 | ethyl | 3,4-dichlorophenoxy |
| 1-152 | 2-chloropyridin-3-yl | H | H | CH3 | ethyl | 4-iodophenoxy |
| 1-153 | 2-chloropyridin-3-yl | H | H | CH3 | ethyl | 4-ethylphenoxy |
| 1-154 | 2-chloropyridin-3-yl | H | H | CH3 | ethyl | 4-butylphenoxy |
| 1-155 | 2-fluorophenyl | H | H | CH3 | CH3 | 4-chlorophenoxy |
| 1-156 | 2-chlorophenyl | H | H | CH3 | CH3 | 4-chlorophenoxy |
| 1-157 | 4-chlorophenyl | H | H | CH3 | CH3 | 4-chlorophenoxy |
| 1-158 | 4-cyanophenyl | H | H | CH3 | CH3 | 4-chlorophenoxy |
| 1-159 | 2-fluoropyridin-3-yl | H | H | CH3 | CH3 | 4-chlorophenoxy |
| 1-160 | 2-chloropyridin-3-yl | H | H | CH3 | CH3 | 4-chlorophenoxy |
| 1-161 | 2-fluorophenyl | H | H | CH3 | ethyl | 4-chlorophenoxy |
| 1-162 | 2-chlorophenyl | H | H | CH3 | ethyl | 4-chlorophenoxy |
| 1-163 | 4-chlorophenyl | H | H | CH3 | ethyl | 4-chlorophenoxy |
| 1-164 | 4-cyanophenyl | H | H | CH3 | ethyl | 4-chlorophenoxy |
| 1-165 | 2-fluoropyridin-3-yl | H | H | CH3 | ethyl | 4-chlorophenoxy |
| 1-166 | 2-chloropyridin-3-yl | H | H | CH3 | CH3 | 4-bromophenoxy |
| 1-167 | 2-chloropyridin-3-yl | H | H | CH3 | CH3 | 3-(trifluoromethyl) |
| | | | | | | phenoxy |
| 1-168 | 2-chloropyridin-3-yl | H | H | CH3 | ethyl | 4-cyanophenoxy |
| 1-169 | 2-chloropyridin-3-yl | H | H | CH3 | ethyl | 4-acetamidophenoxy |
| 1-170 | 2-chloropyridin-3-yl | H | H | CH3 | ethyl | 4-aminophenoxy |
| 1-171 | 2-chloropyridin-3-yl | H | H | CH3 | ethyl | (pyridin-3-yl)oxy |
| 1-172 | 2-chloropyridin-3-yl | H | H | CH3 | ethyl | (pyridin-2-yl)oxy |
| 1-173 | 2-chloropyridin-3-yl | H | H | CH3 | ethyl | (5-chloropyridin-2-yl)oxy |
| 1-174 | 2-chloropyridin-3-yl | H | H | CH3 | ethyl | 4-sulfamoylphenoxy |
| 1-175 | 2-chloropyridin-3-yl | H | H | CH3 | ethyl | (6-chloropyridazin- |
| | | | | | | 3-yl)-oxy |
| 1-176 | 2-chloropyridin-3-yl | H | H | ethyl | ethyl | 4-chlorophenoxy |
| 1-177 | 2-chloropyridin-3-yl | H | H | ethyl | ethyl | 4-bromophenoxy |
| 1-178 | 2-chloropyridin-3-yl | H | H | H | H | 4-chlorophenoxy |
| 1-179 | 2-chloropyridin-3-yl | H | H | H | H | 4-bromophenoxy |
| 1-180 | 2-chloropyridin-3-yl | H | H | CH3 | CH3 | 4-cyanophenoxy |
| 1-181 | 2-chloropyridin-3-yl | H | H | CH3 | ethyl | (6-chloropyridin-3-yl) |
| | | | | | | oxy |
| 1-182 | 2-chloropyridin-3-yl | H | H | CH3 | CH3 | (6-chloropyridin-3-yl) |
| | | | | | | oxy |
| 1-183 | 2-chloropyridin-3-yl | H | H | CH3 | Cl | 4-chlorophenoxy |
| 1-184 | 2-chloropyridin-3-yl | H | H | CH3 | Br | 4-chlorophenoxy |
| 1-185 | 2-chloropyridin-3-yl | H | H | CH3 | I | 4-chlorophenoxy |
| 1-186 | 2-chloropyridin-3-yl | H | H | CH3 | SCF3 | 4-chlorophenoxy |
| 1-187 | 2-chloropyridin-3-yl | H | H | ethyl | Cl | 4-chlorophenoxy |
| 1-188 | 2-chloropyridin-3-yl | H | H | ethyl | Br | 4-chlorophenoxy |
| 1-189 | 2-chloropyridin-3-yl | H | H | ethyl | I | 4-chlorophenoxy |
| 1-190 | 2-chloropyridin-3-yl | H | H | ethyl | SCF3 | 4-chlorophenoxy |
| 1-191 | 2-chloropyridin-3-yl | H | H | n-propyl | Cl | 4-chlorophenoxy |
| 1-192 | 2-chloropyridin-3-yl | H | H | n-propyl | Br | 4-chlorophenoxy |
| 1-193 | 2-chloropyridin-3-yl | H | H | n-propyl | I | 4-chlorophenoxy |
| 1-194 | 2-chloropyridin-3-yl | H | H | Cl | Cl | 4-chlorophenoxy |
| 1-195 | 2-chloropyridin-3-yl | H | H | Cl | Br | 4-chlorophenoxy |
| 1-196 | 2-chloropyridin-3-yl | H | H | Cl | SCF3 | 4-chlorophenoxy |
| 1-197 | 2-chloropyridin-3-yl | H | H | Br | Br | 4-chlorophenoxy |
| 1-198 | 2-chloropyridin-3-yl | H | H | Br | SCF3 | 4-chlorophenoxy |
| 1-199 | 2-chloropyridin-3-yl | H | H | I | I | 4-chlorophenoxy |
| 1-200 | 2-chloropyridin-3-yl | H | H | I | SCF3 | 4-chlorophenoxy |
| 1-201 | 2-chloropyridin-3-yl | H | H | CH3 | H | 4-chlorophenoxy |
| 1-202 | 2-chloropyridin-3-yl | H | H | Br | Br | 4-bromophenoxy |
| 1-203 | 2-chloropyridin-3-yl | H | H | CH3 | ethyl | ({[4-(dimethylamino) |
| | | | | | | phenyl]methylidene |
| | | | | | | amino)oxy |
| 1-204 | 2-chloropyridin-3-yl | H | H | CH3 | CH3 | ethoxy |
| 1-205 | 2-chloropyridin-3-yl | H | H | CH3 | ethyl | ethoxy |
| 1-206 | 2-chloropyridin-3-yl | H | H | CH3 | Br | ethoxy |
| 1-207 | 2-chloropyridin-3-yl | H | H | Br | Br | ethoxy |
| 1-208 | 2-chloropyridin-3-yl | H | H | CH3 | CH3 | 4-chlorophenoxy |
| 1-209 | 2-chloropyridin-3-yl | H | H | CH3 | ethyl | 4-chlorophenoxy |
| 1-210 | 2-chloropyridin-3-yl | H | H | CH3 | Br | 4-chlorophenoxy |
| 1-211 | 2-chloropyridin-3-yl | H | H | Br | Br | 4-chlorophenoxy |

TABLE 2

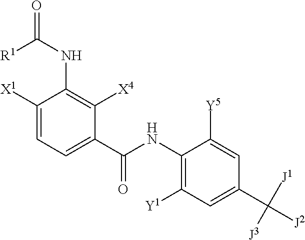

| | R1 | X1 | X4 | Y1 | Y5 | J1 | J2 | J3 |
|---|---|---|---|---|---|---|---|---|
| 2-1 | 2-chloropyridin-3-yl | H | H | CH₃ | ethyl | CF₃ | CF₃ | 1H-pyrrol-1-yl |
| 2-2 | 2-chloropyridin-3-yl | H | H | CH₃ | CH₃ | CF₃ | CF₃ | 2-bromo-1H-pyrrol-1-yl |
| 2-3 | 2-chloropyridin-3-yl | H | H | CH₃ | CH₃ | CF₃ | CF₃ | 3-bromo-1H-pyrrol-1-yl |
| 2-4 | 2-chloropyridin-3-yl | H | H | CH₃ | CH₃ | CF₃ | CF₃ | 2-trifluoroacetyl-1H-pyrrol-1-yl |
| 2-5 | 2-chloropyridin-3-yl | H | H | CH₃ | CH₃ | CF₃ | CF₃ | 1H-pyrazol-1-yl |
| 2-6 | 2-chloropyridin-3-yl | H | H | CH₃ | CH₃ | CF₃ | CF₃ | 4-fluoro-1H-pyrazol-1-yl |
| 2-7 | 2-chloropyridin-3-yl | H | H | CH₃ | CH₃ | CF₃ | CF₃ | 3-chloro-1H-pyrazol-1-yl |
| 2-8 | ethoxy | H | H | CH₃ | CH₃ | CF₃ | H | 4-chloro-1H-pyrazol-1-yl |
| 2-9 | ethoxy | H | H | Br | Br | CF₃ | H | 4-chloro-1H-pyrazol-1-yl |
| 2-10 | 2,2,2-trichloroethoxy | H | H | CH₃ | ethyl | CF₃ | H | 4-chloro-1H-pyrazol-1-yl |
| 2-11 | 2,2,2-trichloroethoxy | H | H | Br | Br | CF₃ | H | 4-chloro-1H-pyrazol-1-yl |
| 2-12 | 2-fluorophenyl | H | H | CH₃ | CH₃ | CF₃ | H | 4-chloro-1H-pyrazol-1-yl |
| 2-13 | 2-chloropyridin-3-yl | H | H | CH₃ | CH₃ | CF₃ | H | 4-chloro-1H-pyrazol-1-yl |
| 2-14 | 2-chloropyridin-3-yl | H | H | CH₃ | CH₃ | CF₃ | CH₃ | 4-chloro-1H-pyrazol-1-yl |
| 2-15 | 2-chloropyridin-3-yl | H | H | CH₃ | ethyl | CF₃ | CH₃ | 4-chloro-1H-pyrazol-1-yl |
| 2-16 | 2-chloropyridin-3-yl | H | H | CH₃ | Br | CF₃ | CH₃ | 4-chloro-1H-pyrazol-1-yl |
| 2-17 | 2-chloropyridin-3-yl | H | H | Cl | Cl | CF₃ | CH₃ | 4-chloro-1H-pyrazol-1-yl |
| 2-18 | 2-chloropyridin-3-yl | H | H | Br | Br | CF₃ | CH₃ | 4-chloro-1H-pyrazol-1-yl |
| 2-19 | 2-chloropyridin-3-yl | H | H | I | I | CF₃ | CH₃ | 4-chloro-1H-pyrazol-1-yl |
| 2-20 | 2-chloropyridin-3-yl | H | H | CH₃ | CH₃ | CF₃ | CH₂F | 4-chloro-1H-pyrazol-1-yl |
| 2-21 | 2-chloropyridin-3-yl | H | H | CH₃ | ethyl | CF₃ | CH₂F | 4-chloro-1H-pyrazol-1-yl |
| 2-22 | 2-chloropyridin-3-yl | H | H | CH₃ | Br | CF₃ | CH₂F | 4-chloro-1H-pyrazol-1-yl |
| 2-23 | 2-chloropyridin-3-yl | H | H | Cl | Cl | CF₃ | CH₂F | 4-chloro-1H-pyrazol-1-yl |
| 2-24 | 2-chloropyridin-3-yl | H | H | Br | Br | CF₃ | CH₂F | 4-chloro-1H-pyrazol-1-yl |
| 2-25 | 2-chloropyridin-3-yl | H | H | I | I | CF₃ | CH₂F | 4-chloro-1H-pyrazol-1-yl |
| 2-26 | 2,2,2-trichloroethoxy | H | H | CH₃ | ethyl | CF₃ | CF₃ | 4-chloro-1H-pyrazol-1-yl |
| 2-27 | phenyl | H | H | CH₃ | ethyl | CF₃ | CF₃ | 4-chloro-1H-pyrazol-1-yl |
| 2-28 | 2-fluorophenyl | H | H | H | CH₃ | CF₃ | CF₃ | 4-chloro-1H-pyrazol-1-yl |
| 2-29 | 2-fluorophenyl | H | H | CH₃ | CH₃ | CF₃ | CF₃ | 4-chloro-1H-pyrazol-1-yl |
| 2-30 | 2-fluorophenyl | H | H | CH₃ | ethyl | CF₃ | CF₃ | 4-chloro-1H-pyrazol-1-yl |
| 2-31 | 2-fluorophenyl | H | H | CH₃ | Br | CF₃ | CF₃ | 4-chloro-1H-pyrazol-1-yl |
| 2-32 | 2-fluorophenyl | H | H | Cl | Cl | CF₃ | CF₃ | 4-chloro-1H-pyrazol-1-yl |
| 2-33 | 2-fluorophenyl | H | H | Br | Br | CF₃ | CF₃ | 4-chloro-1H-pyrazol-1-yl |
| 2-34 | 2-fluorophenyl | H | H | I | I | CF₃ | CF₃ | 4-chloro-1H-pyrazol-1-yl |
| 2-35 | 3-fluorophenyl | H | H | CH₃ | ethyl | CF₃ | CF₃ | 4-chloro-1H-pyrazol-1-yl |
| 2-36 | 4-fluorophenyl | H | H | CH₃ | ethyl | CF₃ | CF₃ | 4-chloro-1H-pyrazol-1-yl |
| 2-37 | 2,3-difluorophenyl | H | H | CH₃ | ethyl | CF₃ | CF₃ | 4-chloro-1H-pyrazol-1-yl |
| 2-38 | 2,4-difluorophenyl | H | H | CH₃ | ethyl | CF₃ | CF₃ | 4-chloro-1H-pyrazol-1-yl |
| 2-39 | 2,5-difluorophenyl | H | H | CH₃ | ethyl | CF₃ | CF₃ | 4-chloro-1H-pyrazol-1-yl |
| 2-40 | 2,6-difluorophenyl | H | H | CH₃ | ethyl | CF₃ | CF₃ | 4-chloro-1H-pyrazol-1-yl |
| 2-41 | 2,6-difluorophenyl | H | H | CH₃ | Br | CF₃ | CF₃ | 4-chloro-1H-pyrazol-1-yl |
| 2-42 | pyridin-3-yl | H | H | CH₃ | ethyl | CF₃ | CF₃ | 4-chloro-1H-pyrazol-1-yl |
| 2-43 | 2-fluoropyridin-3-yl | H | H | CH₃ | CH₃ | CF₃ | CF₃ | 4-chloro-1H-pyrazol-1-yl |
| 2-44 | 2-fluoropyridin-3-yl | H | H | CH₃ | ethyl | CF₃ | CF₃ | 4-chloro-1H-pyrazol-1-yl |
| 2-45 | 2-fluoropyridin-3-yl | H | H | CH₃ | Br | CF₃ | CF₃ | 4-chloro-1H-pyrazol-1-yl |
| 2-46 | 2-fluoropyridin-3-yl | H | H | n-propyl | I | CF₃ | CF₃ | 4-chloro-1H-pyrazol-1-yl |
| 2-47 | 2-fluoropyridin-3-yl | H | H | Cl | Cl | CF₃ | CF₃ | 4-chloro-1H-pyrazol-1-yl |
| 2-48 | 2-fluoropyridin-3-yl | H | H | Br | Br | CF₃ | CF₃ | 4-chloro-1H-pyrazol-1-yl |
| 2-49 | 2-fluoropyridin-3-yl | H | H | I | I | CF₃ | CF₃ | 4-chloro-1H-pyrazol-1-yl |
| 2-50 | 2-chloropyridin-3-yl | H | H | H | CH₃ | CF₃ | CF₃ | 4-chloro-1H-pyrazol-1-yl |
| 2-51 | 2-chloropyridin-3-yl | H | H | CH₃ | CH₃ | CF₃ | CF₃ | 4-chloro-1H-pyrazol-1-yl |
| 2-52 | 2-chloropyridin-3-yl | F | H | CH₃ | CH₃ | CF₃ | CF₃ | 4-chloro-1H-pyrazol-1-yl |
| 2-53 | 2-chloropyridin-3-yl | H | F | CH₃ | CH₃ | CF₃ | CF₃ | 4-chloro-1H-pyrazol-1-yl |
| 2-54 | 2-chloropyridin-3-yl | H | Cl | CH₃ | CH₃ | CF₃ | CF₃ | 4-chloro-1H-pyrazol-1-yl |
| 2-55 | 2-chloropyridin-3-yl | H | H | CH₃ | ethyl | CF₃ | CF₃ | 4-chloro-1H-pyrazol-1-yl |
| 2-56 | 2-chloropyridin-3-yl | F | H | CH₃ | ethyl | CF₃ | CF₃ | 4-chloro-1H-pyrazol-1-yl |
| 2-57 | 2-chloropyridin-3-yl | H | F | CH₃ | ethyl | CF₃ | CF₃ | 4-chloro-1H-pyrazol-1-yl |
| 2-58 | 2-chloropyridin-3-yl | H | Cl | CH₃ | ethyl | CF₃ | CF₃ | 4-chloro-1H-pyrazol-1-yl |
| 2-59 | 2-chloropyridin-3-yl | H | H | CH₃ | Br | CF₃ | CF₃ | 4-chloro-1H-pyrazol-1-yl |
| 2-60 | 2-chloropyridin-3-yl | H | H | n-propyl | I | CF₃ | CF₃ | 4-chloro-1H-pyrazol-1-yl |
| 2-61 | 2-chloropyridin-3-yl | H | H | Cl | Cl | CF₃ | CF₃ | 4-chloro-1H-pyrazol-1-yl |
| 2-62 | 2-chloropyridin-3-yl | H | H | Br | Br | CF₃ | CF₃ | 4-chloro-1H-pyrazol-1-yl |
| 2-63 | 2-chloropyridin-3-yl | H | H | I | I | CF₃ | CF₃ | 4-chloro-1H-pyrazol-1-yl |
| 2-64 | 2-chloropyridin-3-yl | H | H | CF₃ | Br | CF₃ | CF₃ | 4-chloro-1H-pyrazol-1-yl |
| 2-65 | 2-chloropyridin-3-yl | H | H | CF₂CF₃ | Br | CF₃ | CF₃ | 4-chloro-1H-pyrazol-1-yl |
| 2-66 | 2-chloropyridin-3-yl | H | H | SCF₃ | Br | CF₃ | CF₃ | 4-chloro-1H-pyrazol-1-yl |
| 2-67 | 2-chloropyridin-3-yl | H | H | S(O)CF₃ | Br | CF₃ | CF₃ | 4-chloro-1H-pyrazol-1-yl |
| 2-68 | 2-chloropyridin-3-yl | H | H | S(O)2CF₃ | Br | CF₃ | CF₃ | 4-chloro-1H-pyrazol-1-yl |
| 2-69 | 2-chloropyridin-3-yl | H | H | CH₃ | ethyl | CF₃ | phenyl | 4-chloro-1H-pyrazol-1-yl |
| 2-70 | 2-chlorop yridin-3-yl | H | H | Br | Br | CF₃ | phenyl | 4-chloro-1H-pyrazol-1-yl |
| 2-71 | 2-chloropyridin-3-yl | H | H | Br | Br | CF₃ | 2-chloro- | 4-chloro-1H-pyrazol-1-yl |
| | | | | | | | phenyl | |
| 2-72 | 2-chloropyridin-3-yl | H | H | Br | Br | CF₃ | 3-chloro- | 4-chloro-1H-pyrazol-1-yl |
| | | | | | | | phenyl | |
| 2-73 | 2-chloropyridin-3-yl | H | H | Br | Br | CF₃ | 4-chloro- | 4-chloro-1H-pyrazol-1-yl |
| | | | | | | | phenyl | |
| 2-74 | 2-chloropyridin-3-yl | H | H | Br | Br | CF₃ | 3,5-dichloro- | 4-chloro-1H-pyrazol-1-yl |
| | | | | | | | phenyl | |
| 2-75 | 2-chloropyridin-3-yl | H | H | Br | Br | CF₃ | 6-chloro- | 4-chloro-1H-pyrazol-1-yl |
| | | | | | | | pyridin-3-yl | |
| 2-76 | 2-chloropyridin-3-yl | H | H | CH₃ | CH₃ | CF₃ | CF₃ | 3-bromo-1H-pyrazol-1-yl |
| 2-77 | 2-chloropyridin-3-yl | H | H | CH₃ | ethyl | CF₃ | H | 4-bromo-1H-pyrazol-1-yl |
| 2-78 | unused number | | | | | | | |
| 2-79 | 2,2,2-trichloroethoxy | H | H | CH₃ | ethyl | CF₃ | H | 4-bromo-1H-pyrazol-1-yl |
| 2-80 | 2-chloropyridin-3-yl | H | H | CH₃ | CH₃ | CF₃ | CH₃ | 4-bromo-1H-pyrazol-1-yl |
| 2-81 | 2-chloropyridin-3-yl | H | H | CH₃ | ethyl | CF₃ | CH₃ | 4-bromo-1H-pyrazol-1-yl |
| 2-82 | 2-fluoropyridin-3-yl | H | H | Br | Br | CF₃ | CH₃ | 4-bromo-1H-pyrazol-1-yl |
| 2-83 | 2-chloropyridin-3-yl | H | H | Br | Br | CF₃ | CH₃ | 4-bromo-1H-pyrazol-1-yl |
| 2-84 | 2-chloropyridin-3-yl | H | H | CH₃ | CH₃ | CF₃ | CF₃ | 4-bromo-1H-pyrazol-1-yl |
| 2-85 | 2-chloropyridin-3-yl | H | F | CH₃ | CH₃ | CF₃ | CF₃ | 4-bromo-1H-pyrazol-1-yl |
| 2-86 | 2-chloropyridin-3-yl | F | H | CH₃ | CH₃ | CF₃ | CF₃ | 4-bromo-1H-pyrazol-1-yl |
| 2-87 | 2-chloropyridin-3-yl | H | H | CH₃ | ethyl | CF₃ | CF₃ | 4-bromo-1H-pyrazol-1-yl |
| 2-88 | 2-chloropyridin-3-yl | F | H | CH₃ | ethyl | CF₃ | CF₃ | 4-bromo-1H-pyrazol-1-yl |
| 2-89 | 2-chloropyridin-3-yl | H | F | CH₃ | ethyl | CF₃ | CF₃ | 4-bromo-1H-pyrazol-1-yl |
| 2-90 | 2-chloropyridin-3-yl | H | H | CH₃ | Br | CF₃ | CF₃ | 4-bromo-1H-pyrazol-1-yl |
| 2-91 | 2-chloropyridin-3-yl | H | H | Cl | Cl | CF₃ | CF₃ | 4-bromo-1H-pyrazol-1-yl |
| 2-92 | 2-chloropyridin-3-yl | H | H | Br | Br | CF₃ | CF₃ | 4-bromo-1H-pyrazol-1-yl |
| 2-93 | 2-chloropyridin-3-yl | H | H | I | I | CF₃ | CF₃ | 4-bromo-1H-pyrazol-1-yl |
| 2-94 | 2-chloropyridin-3-yl | H | H | SCF₃ | Br | CF₃ | CF₃ | 4-bromo-1H-pyrazol-1-yl |
| 2-95 | 2-chloropyridin-3-yl | H | H | CH₃ | CH₃ | CF₃ | CF₃ | 4-iodo-1H-pyrazol-1-yl |
| 2-96 | 2-chloropyridin-3-yl | H | H | CH₃ | CH₃ | CF₃ | CF₃ | 3-methyl-1H-pyrazol-1-yl |
| 2-97 | 2-chloropyridin-3-yl | H | H | CH₃ | CH₃ | CF₃ | CF₃ | 4-methyl-1H-pyrazol-1-yl |
| 2-98 | 2-fluoropyridin-3-yl | H | H | CH₃ | CH₃ | CF₃ | CF₃ | 3-(trifluoromethyl)-1H-pyrazol-1-yl |
| 2-99 | 2-chloropyridin-3-yl | H | H | Br | Br | CF₃ | phenyl | 3-(trifluoromethyl)-1H-pyrazol-1-yl |
| 2-100 | 2-chloropyridin-3-yl | H | H | Br | Br | CF₃ | phenyl | 3-(trifluoromethyl)-1H-pyrazol-1-yl |
| 2-101 | 2-chloropyridin-3-yl | H | H | Br | Br | CF₃ | 2-chloro- | 3-(trifluoromethyl)-1H-pyrazol-1-yl |
| | | | | | | | phenyl | |
| 2-102 | 2-chloropyridin-3-yl | H | H | Br | Br | CF₃ | 3-chloro- | 3-(trifluoromethyl)-1H-pyrazol-1-yl |
| | | | | | | | phenyl | |
| 2-103 | 2-fluoropyridin-3-yl | H | H | CH₃ | CH₃ | CF₃ | H | 4-(trifluoromethyl)-1H-pyrazol-1-yl |
| 2-104 | 2-chloropyridin-3-yl | H | H | CH₃ | CH₃ | CF₂CF₃ | H | 4-(trifluoromethyl)-1H-pyrazol-1-yl |
| 2-105 | 2-chloropyridin-3-yl | H | H | CH₃ | ethyl | CF₃ | CF₃ | 4-(trifluoromethyl)-1H-pyrazol-1-yl |
| 2-106 | 2-chloropyridin-3-yl | H | H | CH₃ | CH₃ | CF₃ | CF₃ | 3-(dichloromethyl)-1H-pyrazol-1-yl |
| 2-107 | 2-chloropyridin-3-yl | H | H | CH₃ | CH₃ | CF₃ | CF₃ | 3-(pentafluoroethyl)-1H-pyrazol-1-yl |
| 2-108 | 2-fluoropyridin-3-yl | H | H | CH₃ | CH₃ | CF₃ | CF₃ | 4-(trifluoromethyl)-1H-pyrazol-1-yl |
| 2-109 | 2-chloropyridin-3-yl | H | H | CH₃ | CH₃ | CF₃ | CF₃ | 4-(trifluoromethyl)-1H-pyrazol-1-yl |
| 2-110 | 2-chloropyridin-3-yl | H | H | CH₃ | CH₃ | CF₃ | CF₃ | 4-formyl-1H-pyrazol-1-yl |
| 2-111 | 2-chloropyridin-3-yl | H | H | CH₃ | CH₃ | CF₃ | CF₃ | 3-(dimethoxymethyl)-1H-pyrazol-1-yl |
| 2-112 | 2-chloropyridin-3-yl | H | H | CH₃ | CH₃ | CF₃ | CF₃ | 4-+(methoxyimino)methyl]- |
| | | | | | | | | 1H-pyrazol-1-yl |
| 2-113 | 2-chloropyridin-3-yl | H | H | CH₃ | CH₃ | CF₃ | CF₃ | 3-nitro-1H-pyrazol-1-yl |
| 2-114 | 2-chloropyridin-3-yl | H | H | CH₃ | CH₃ | CF₃ | CF₃ | 5-acetyl-1H-pyrazol-1-yl |
| 2-115 | 2-chloropyridin-3-yl | H | H | CH₃ | CH₃ | CF₃ | CF₃ | 3,4-dibromo-1H-pyrazol-1-yl |
| 2-116 | 2-chloropyridin-3-yl | H | H | CH₃ | CH₃ | CF₃ | CF₃ | 3,4-diiodo-1H-pyrazol-1-yl |
| 2-117 | 2-chloropyridin-3-yl | H | H | CH₃ | CH₃ | CF₃ | H | 3,5-dimethyl-1H-pyrazol-1-yl |
| 2-118 | 2-chloropyridin-3-yl | H | H | CH₃ | CH₃ | CF₃ | CF₃ | 4-chloro-3-(trifluoromethyl)-1H- |
| | | | | | | | | pyrazol-1-yl |
| 2-119 | 2-chloropyridin-3-yl | H | H | CH₃ | CH₃ | CF₃ | CF₃ | 4-iodo-3-(trifluoromethyl)-1H- |
| | | | | | | | | pyrazol-1-yl |
| 2-120 | 2-chloropyridin-3-yl | H | H | CH₃ | CH₃ | CF₃ | CF₃ | 4-chloro-3-(pentafluoroethyl)-1H- |
| | | | | | | | | pyrazol-1-yl |
| 2-121 | ethoxy | H | H | CH₃ | CH₃ | CF₃ | H | 3,5-bis(trifluoromethyl)-1H-pyrazol-1-yl |
| 2-122 | 2,2,2-trichloroethoxy | H | H | CH₃ | ethyl | CF₃ | H | 3,5-bis(trifluoromethyl)-1H-pyrazol-1-yl |
| 2-123 | 2-chloropyridin-3-yl | H | H | CH₃ | CH₃ | CF₃ | H | 3 ,5-bis(trifluoromethyl)-1H-pyrazol-1-yl |
| 2-124 | 2-fluorophenyl | H | H | CH₃ | CH₃ | CF₃ | CF₃ | 3 ,5-bis(trifluoromethyl)-1H-pyrazol-1-yl |
| 2-125 | 2-fluoropyridin-3-yl | H | H | CH₃ | CH₃ | CF₃ | CF₃ | 3,5-bis(trifluoromethyl)-1H-pyrazol-1-yl |
| 2-126 | 2-chloropyridin-3-yl | H | H | CH₃ | CH₃ | CF₃ | CF₃ | 4-(ethoxycarbonyl)-3-(trifluoromethyl)- |
| | | | | | | | | 1H-pyrazol-1-yl |
| 2-127 | 2-fluoropyridin-3-yl | H | H | CH₃ | CH₃ | CF₃ | CF₃ | 3,4,5-tribromo-1H-pyrazol-1-yl |
| 2-128 | 2-fluoropyridin-3-yl | H | H | CH₃ | ethyl | CF₃ | CF₃ | 3 ,4,5-tribromo-1H-pyrazol- I -yl |
| 2-129 | 2-fluoropyridin-3-yl | H | H | CH₃ | Br | CF₃ | CF₃ | 3,4,5-tribromo-1H-pyrazol-1-yl |
| 2-130 | 2-fluoropyridin-3-yl | H | H | Cl | Cl | CF₃ | CF₃ | 3,4,5-tribromo-1H-pyrazol-1-yl |
| 2-131 | 2-fluoropyridin-3-yl | H | H | Br | Br | CF₃ | CF₃ | 3,4,5-tribromo-1H-pyrazol-1-yl |
| 2-132 | 2-fluoropyridin-3-yl | H | H | I | I | CF₃ | CF₃ | 3,4,5-tribromo-1H-pyrazol-1-yl |
| 2-133 | 2-chloropyridin-3-yl | H | H | CH₃ | CH₃ | CF₃ | CF₃ | 3,4,5-tribromo-1H-pyrazol-1-yl |
| 2-134 | 2-chloropyridin-3-yl | H | H | CH₃ | ethyl | CF₃ | CF₃ | 3,4,5-tribromo-1H-pyrazol-1-yl |
| 2-135 | 2-chloropyridin-3-yl | H | H | CH₃ | Br | CF₃ | CF₃ | 3,4,5-tribromo-1H-pyrazol-1-yl |
| 2-136 | 2-chloropyridin-3-yl | H | H | Cl | Cl | CF₃ | CF₃ | 3,4,5-tribromo-1H-pyrazol-1-yl |
| 2-137 | 2-chloropyridin-3-yl | H | H | Br | Br | CF₃ | CF₃ | 3,4,5-tribromo-1H-pyrazol-1-yl |
| 2-138 | 2-chloropyridin-3-yl | H | H | I | I | CF₃ | CF₃ | 3,4,5-tribromo-1H-pyrazol-1-yl |
| 2-139 | 2-chloropyridin-3-yl | H | H | CH₃ | CH₃ | CF₃ | CF₃ | 3,4,5-triiodo-1H-pyrazol-1-yl |
| 2-140 | 2-chloropyridin-3-yl | H | H | CH₃ | CH₃ | CF₃ | H | 4-bromo-3,5-dimethyl-1H-pyrazol-1-yl |
| 2-141 | 2-chloropyridin-3-yl | H | H | CH₃ | CH₃ | CF₃ | CF₃ | 1H-imidazol-1-yl |
| 2-142 | 2-chloropyridin-3-yl | H | H | CH₃ | CH₃ | CF₃ | CF₃ | 4-(trifluoromethyl)imidazol-1-yl |
| 2-143 | 2-chloropyridin-3-yl | H | H | CH₃ | CH₃ | CF₃ | CF₃ | 4,5-dichloro-1H-imidazol-1-yl |
| 2-144 | 2-chloropyridin-3-yl | H | H | CH₃ | CH₃ | CF₃ | CF₃ | 2,4,5-tribromo-1H-imidazol-1-yl |
| 2-145 | 2-chloropyridin-3-yl | H | H | CH₃ | CH₃ | CF₃ | CF₃ | N₃ |
| 2-146 | 2-chloropyridin-3-yl | H | H | CH₃ | CH₃ | CF₃ | CF₃ | 1H-1,2,3-triazol-1-yl |
| 2-147 | 2-chloropyridin-3-yl | H | H | CH₃ | CH₃ | CF₃ | CF₃ | 2H-1,2,3-triazol-2-yl |
| 2-148 | 2-chloropyridin-3-yl | H | H | CH₃ | CH₃ | CF₃ | CF₃ | 1H-1,2,4-triazol-1-yl |
| 2-149 | 2-chloropyridin-3-yl | H | H | CH₃ | ethyl | CF₃ | CF₃ | 1H-1,2,4-triazol-1-yl |
| 2-150 | 2-fluorophenyl | H | H | CH₃ | ethyl | CF₃ | CF₃ | 1H-1,2,4-triazol-1-yl |
| 2-151 | 2-fluorophenyl | H | H | CH₃ | Br | CF₃ | CF₃ | 1H-1,2,4-triazol-1-yl |
| 2-152 | 2-fluoropyridin-3-yl | H | H | Br | Br | CF₃ | CF₃ | 1H-1,2,4-triazol-1-yl |
| 2-153 | 2-chloropyridin-3-yl | H | H | CH₃ | CH₃ | CF₃ | CF₃ | 3-(trifluoromethyl)-1H-1,2,4-triazol-1-yl |
| 2-154 | 2-chloropyridin-3-yl | H | H | CH₃ | CH₃ | CF₃ | CF₃ | 2H-tetrazol-2-yl |
| 2-155 | 2-chloropyridin-3-yl | H | H | CH₃ | CH₃ | CF₃ | CF₃ | 1H-indazol-1-yl |
| 2-156 | 2-chloropyridin-3-yl | H | H | CH₃ | ethyl | CF₃ | CF₃ | 1H-indazol-1-yl |
| 2-157 | unused number | | | | | | | |
| 2-158 | 2-chloropyridin-3-yl | H | H | CH₃ | ethyl | CF₃ | CF₃ | 3-chloro-1H-indazol-1-yl |
| 2-159 | 2-chloropyridin-3-yl | H | H | CH₃ | ethyl | CF₃ | CF₃ | 1H-benzimidazol-1-yl |
| 2-160 | 2-chloropyridin-3-yl | H | H | CH₃ | ethyl | CF₃ | CF₃ | 2-chloro-1H-benzimidazol-1-yl |
| 2-161 | 2-chloropyridin-3-yl | H | H | CH₃ | ethyl | CF₃ | CF₃ | 1H-benzotriazol-1-yl |
| 2-162 | 2-chloropyridin-3-yl | H | H | CH₃ | ethyl | CF₃ | CF₃ | 2H-benzotriazol-2-yl |
| 2-163 | 2-chloropyridin-3-yl | H | H | CH₃ | CH₃ | CF₃ | CF₃ | pyrrolidin-1-yl |
| 2-164 | 2-chloropyridin-3-yl | H | H | CH₃ | ethyl | CF₃ | CF₃ | pyrrolidin-1-yl |
| 2-165 | 2-chloropyridin-3-yl | H | H | CH₃ | ethyl | CF₃ | CF₃ | 2-methylpyrrolidin-1-yl |
| 2-166 | 2-chloropyridin-3-yl | H | H | CH₃ | ethyl | CF₃ | CF₃ | 3-chlorolpyrrolidin-1-yl |
| 2-167 | 2-chloropyridin-3-yl | H | H | CH₃ | ethyl | CF₃ | CF₃ | 2-(trifluoromethyl)pyrro lidin-1-yl |
| 2-168 | 2-chloropyridin-3-yl | H | H | CH₃ | ethyl | CF₃ | CF₃ | 2,5-dimethylpyrro lidin-1-yl |
| 2-169 | 2-chloropyridin-3-yl | H | H | CH₃ | ethyl | CF₃ | CF₃ | 3,3-dichlorolpyrrolidin-1-yl |
| 2-170 | 2-chloropyridin-3-yl | H | H | CH₃ | ethyl | CF₃ | CF₃ | 2,5-dihydro-1H-pyrrol-1-yl |
| 2-171 | 2-chloropyridin-3-yl | H | H | CH₃ | CH₃ | CF₃ | CF₃ | 3-(3,5-dichlorophenyl)-3-(trifluoro- |
| | | | | | | | | methyl)pyrrolidin-1-yl |
| 2-172 | 2-chloropyridin-3-yl | H | H | CH₃ | ethyl | CF₃ | CF₃ | piperidin-1-yl |
| 2-173 | 2-chloropyridin-3-yl | H | H | CH₃ | ethyl | CF₃ | CF₃ | 2-methylpiperidin-1-yl |
| 2-174 | 2-chloropyridin-3-yl | H | H | CH₃ | ethyl | CF₃ | CF₃ | 3-methylpiperidin-1-yl |
| 2-175 | 2-chloropyridin-3-yl | H | H | CH₃ | ethyl | CF₃ | CF₃ | 4-methylpiperidin-1-yl |
| 2-176 | 2-chloropyridin-3-yl | H | H | CH₃ | ethyl | CF₃ | CF₃ | 2-ethylpiperidin-1-yl |
| 2-177 | 2-chloropyridin-3-yl | H | H | CH₃ | ethyl | CF₃ | CF₃ | 2,6-dimethylpiperidin-1-yl |
| 2-178 | 2-chloropyridin-3-yl | H | H | CH₃ | ethyl | CF₃ | CF₃ | 3,3-dimethylpiperidin-1-yl |
| 2-179 | 2-chloropyridin-3-yl | H | H | CH₃ | ethyl | CF₃ | CF₃ | 3,5-dimethylpiperidin-1-yl |
| 2-180 | 2-chloropyridin-3-yl | H | H | CH₃ | ethyl | CF₃ | CF₃ | 3,6-dihydropyridin-1(2H)-yl |
| 2-181 | 2-chloropyridin-3-yl | H | H | CH₃ | ethyl | CF₃ | CF₃ | morpholin-4-yl |
| 2-182 | 2-chloropyridin-3-yl | H | H | CH₃ | ethyl | CF₃ | CF₃ | thiomorpholin-4-yl |
| 2-183 | 2-chloropyridin-3-yl | H | H | CH₃ | ethyl | CF₃ | CF₃ | 3-chloro-1H-1,2,4-triazol-1-yl |
| 2-184 | 2-chloropyridin-3-yl | H | H | CH₃ | CH₃ | CF₃ | CF₂CF₃ | 4-chloro-1H-pyrazol-1-yl |
| 2- 185 | 2-chloropyridin-3-yl | H | H | CH₃ | ethyl | CF₃ | CF₂CF₃ | 4-chloro-1H-pyrazol-1-yl |
| 2- 186 | 2-chloropyridin-3-yl | H | H | CH₃ | Br | CF₃ | CF₂CF₃ | 4-chloro-1H-pyrazol-1-yl |
| 2-187 | 2-chloropyridin-3-yl | H | H | Br | Br | CF₃ | CF₂CF₃ | 4-chloro-1H-pyrazol-1-yl |

TABLE 3

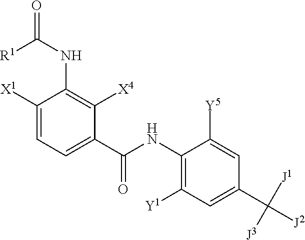

| | R1 | X1 | X4 | Y1 | Y5 | J1 | J2 | J3 |
|---|---|---|---|---|---|---|---|---|
| 3-1 | 2-chloropyridin-3-yl | H | H | CH3 | ethyl | CF3 | CF3 | methylsulfanyl |
| 3-2 | 2-chloropyridin-3-yl | H | H | Br | Br | CF3 | CF3 | methylsulfanyl |
| 3-3 | 2-chloropyridin-3-yl | H | H | CH3 | ethyl | CF3 | phenyl | methylsulfanyl |
| 3-4 | 2-chloropyridin-3-yl | H | H | CH3 | ethyl | CF3 | CF3 | ethylsulfanyl |
| 3-5 | 2-chloropyridin-3-yl | H | H | Br | Br | CF3 | CF3 | ethylsulfanyl |
| 3-6 | 2-chloropyridin-3-yl | H | H | CH3 | ethyl | CF3 | phenyl | ethylsulfanyl |
| 3-7 | 2-chloropyridin-3-yl | H | H | CH3 | ethyl | CF3 | CF3 | propylsulfanyl |
| 3-8 | 2-chloropyridin-3-yl | H | H | CH3 | ethyl | CF3 | CF3 | cyclopentylsulfanyl |
| 3-9 | 2-chloropyridin-3-yl | H | H | CH3 | ethyl | CF3 | CF3 | cyclohexylsulfanyl |
| 3-10 | 2-chloropyridin-3-yl | H | H | CH3 | ethyl | CF3 | CF3 | trifluoromethylsulfanyl |
| 3-11 | 2-chloropyridin-3-yl | H | H | CH3 | CH3 | CF3 | CF3 | 2,2,2-trifluoroethylsulfanyl |
| 3-12 | 2-chloropyridin-3-yl | H | H | CH3 | ethyl | CF3 | CF3 | 2,2,2-trifluoroethylsulfanyl |
| 3-13 | 2-chloropyridin-3-yl | H | H | CH3 | ethyl | CF3 | CF3 | 2-methyl-5-(trifluoromethyl)-2,5- |
| | | | | | | | | dihydro-1H-1,2,4-triazol-3-yl] |
| | | | | | | | | sulfanyl |
| 3-14 | 2-chloropyridin-3-yl | H | H | CH3 | ethyl | CF3 | CF3 | phenylsulfanyl |
| 3-15 | 2-chloropyridin-3-yl | H | H | CH3 | ethyl | CF3 | CF3 | 2-chlorophenylsulfanyl |
| 3-16 | 2-chloropyridin-3-yl | H | H | CH3 | ethyl | CF3 | CF3 | 3-chlorophenylsulfanyl |
| 3-17 | 2-chloropyridin-3-yl | H | H | CH3 | ethyl | CF3 | CF3 | 4-chlorophenylsulfanyl |

TABLE 4

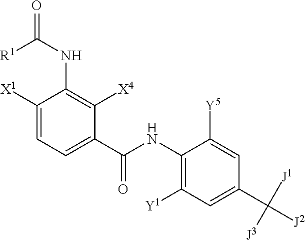

| | R1 | X1 | X4 | Y1 | Y5 | J1 | J2 | J3 |
|---|---|---|---|---|---|---|---|---|
| 4-1 | 2-chloropyridin-3-yl | H | H | CH3 | CH3 | CF3 | CF3 | 3-chloro-1H- |
| | | | | | | | | pyrrol-2-yl |
| 4-2 | 2-chloropyridin-3-yl | H | H | CH3 | CH3 | CF3 | CF3 | CN |
| 4-3 | 2-fluorophenyl | H | H | CH3 | ethyl | CF3 | CF3 | CN |
| 4-4 | 2-fluoropyridin-3-yl | H | H | CH3 | ethyl | CF3 | CF3 | CN |
| 4-5 | 2-chloropyridin-3-yl | H | H | CH3 | ethyl | CF3 | CF3 | CN |
| 4-6 | 2-chloropyridin-3-yl | H | H | CH3 | Br | CF3 | CF3 | CN |
| 4-7 | 2-chloropyridin-3-yl | H | H | Cl | Cl | CF3 | CF3 | CN |
| 4-8 | 2-chloropyridin-3-yl | H | H | Br | Br | CF3 | CF3 | CN |
| 4-9 | 2-chloropyridin-3-yl | H | H | I | I | CF3 | CF3 | CN |
| 4-10 | 2-chloropyridin-3-yl | H | H | CH3 | CH3 | CF3 | phenyl | CN |
| 4-11 | 2-chloropyridin-3-yl | H | H | CH3 | ethyl | CF3 | phenyl | CN |
| 4-12 | 2-chloropyridin-3-yl | H | H | CH3 | Br | CF3 | phenyl | CN |
| 4-13 | 2-chloropyridin-3-yl | H | H | Br | Br | CF3 | phenyl | CN |
| 4-14 | 2-chloropyridin-3-yl | H | H | CH3 | ethyl | CF3 | 2-chlorophenyl | CN |
| 4-15 | 2-chloropyridin-3-yl | H | H | CH3 | ethyl | CF3 | 3-chlorophenyl | CN |
| 4-16 | 2-chloropyridin-3-yl | H | H | CH3 | ethyl | CF3 | 4-chlorophenyl | CN |
| 4-17 | 2-chloropyridin-3-yl | H | H | CH3 | ethyl | CF3 | 3-(trifluoromethyl)phenyl | CN |
| 4-18 | 2-chloropyridin-3-yl | H | H | CH3 | ethyl | CF3 | 3,5-dichlorophenyl | CN |
| 4-19 | 2-chloropyridin-3-yl | H | H | CH3 | CH3 | CF3 | CF2CF3 | CN |

TABLE 5

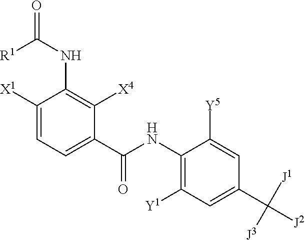

| | R1 | X1 | X4 | Y1 | Y5 | J1 | J2 | J3 |
|---|---|---|---|---|---|---|---|---|
| 5-1 | 2-chloropyridin-3-yl | H | H | Br | Br | CF3 | 3-chlorophenyl | OH |
| 5-2 | 2-chloropyridin-3-yl | H | H | Br | Br | CF3 | 3-chlorophenyl | Cl |
| 5-3 | 2-chloropyridin-3-yl | H | H | Br | Br | CF3 | 4-chlorophenyl | OH |
| 5-4 | 2-chloropyridin-3-yl | H | H | Br | Br | CF3 | 4-chlorophenyl | Cl |
| 5-5 | 2-chloropyridin-3-yl | H | H | Br | Br | CF3 | 4-chlorophenyl | F |
| 5-6 | 2-chloropyridin-3-yl | H | H | Br | Br | CF3 | 3-chlorophenyl | F |
| 5-7 | 2-chloropyridin-3-yl | H | H | Br | Br | CF3 | phenyl | H |

TABLE 6

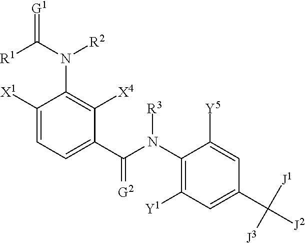

| | R1 | R2 | R3 | G1 | G2 | X¹ | X⁴ | Y1 | Y5 | J1 | J2 | J3 |
|---|---|---|---|---|---|---|---|---|---|---|---|---|
| 6-1 | 2-chloropyridin-3-yl | H | CH₃ | O | O | H | H | CH₃ | ethyl | CF₃ | CF₃ | 4-chloro-1H- |
| | | | | | | | | | | | | pyrazol-1-yl |
| 6-2 | 2-chloropyridin-3-yl | CH₃ | H | O | O | H | H | CH₃ | ethyl | CF₃ | CF₃ | 4-chloro-1H- |
| | | | | | | | | | | | | pyrazol-1-yl |
| 6-3 | 2-chloropyridin-3-yl | CH₃ | CH₃ | O | O | H | H | CH₃ | ethyl | CF₃ | CF₃ | 4-chloro-1H- |
| | | | | | | | | | | | | pyrazol-1-yl |
| 6-4 | phenyl | H | H | S | O | H | H | CH₃ | ethyl | CF₃ | CF₃ | 4-chloro-1H- |
| | | | | | | | | | | | | pyrazol-1-yl |
| 6-5 | phenyl | H | H | O | S | H | H | CH₃ | ethyl | CF₃ | CF₃ | 4-chloro-1H- |
| | | | | | | | | | | | | pyrazol-1-yl |
| 6-6 | phenyl | H | H | S | S | H | H | CH₃ | ethyl | CF₃ | CF₃ | 4-chloro-1H- |
| | | | | | | | | | | | | pyrazol-1-yl |

TABLE 7

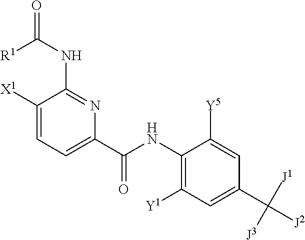

| | R1 | X1 | Y1 | Y5 | J1 | J2 | J3 |
|---|---|---|---|---|---|---|---|
| 7-1 | phenyl | H | CH3 | ethyl | CF3 | CF3 | 4-chloro-1H-pyrazol-1-yl |
| 7-2 | 2-fluorophenyl | H | CH3 | ethyl | CF3 | CF3 | 4-chloro-1H-pyrazol-1-yl |
| 7-3 | 2-fluoropyridin-3-yl | H | CH3 | ethyl | CF3 | CF3 | 4-chloro-1H-pyrazol-1-yl |
| 7-4 | 2-chloropyridin-3-yl | H | CH3 | CH3 | CF3 | CF3 | 4-chloro-1H-pyrazol-1-yl |
| 7-5 | 2-chloropyridin-3-yl | H | CH3 | ethyl | CF3 | CF3 | 4-chloro-1H-pyrazol-1-yl |
| 7-6 | 2-chloropyridin-3-yl | H | Br | Br | CF3 | CF3 | 4-chloro-1H-pyrazol-1-yl |
| 7-7 | 2-chloropyridin-3-yl | H | CH3 | ethyl | CF3 | CF3 | 4-bromo-1H-pyrazol-1-yl |
| 7-8 | 2-chloropyridin-3-yl | H | Br | Br | CF3 | CF3 | 4-bromo-1H-pyrazol-1-yl |
| 7-9 | 2-chloropyridin-3-yl | H | CH3 | ethyl | CF3 | CF3 | ethoxy |
| 7-10 | 2-chloropyridin-3-yl | H | CH3 | ethyl | CF3 | CF3 | 4-chlorophenoxy |
| 7-11 | 2-chloropyridin-3-yl | H | CH3 | ethyl | CF3 | CF3 | 4-bromophenoxy |
| 7-12 | 2-chloropyridin-3-yl | H | CH3 | ethyl | CF3 | CF2CF3 | ethoxy |
| 7-13 | 2-chloropyridin-3-yl | H | CH3 | ethyl | CF3 | CF2CF3 | 4-chlorophenoxy |
| 7-14 | 2-chloropyridin-3-yl | H | CH3 | ethyl | CF3 | CF2CF3 | 4-chloro-1H-pyrazol-1-yl |

TABLE 8

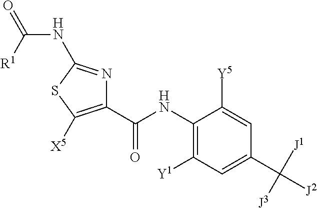

| | R1 | X⁵ | Y1 | Y5 | J1 | J2 | J3 |
|---|---|---|---|---|---|---|---|
| 8-1 | phenyl | H | CH₃ | ethyl | CF₃ | CF₃ | 4-chloro-1H-pyrazol-1-yl |
| 8-2 | 2-fluorophenyl | H | CH₃ | ethyl | CF₃ | CF₃ | 4-chloro-1H-pyrazol-1-yl |
| 8-3 | 4-chlorophenyl | H | CH₃ | ethyl | CF₃ | CF₃ | 4-chloro-1H-pyrazol-1-yl |
| 8-4 | 2,6-difluorophenyl | H | CH₃ | ethyl | CF₃ | CF₃ | 4-chloro-1H-pyrazol-1-y1 |
| 8-5 | 2,6-difluorophenyl | H | Br | Br | CF₃ | CF₃ | 4-chloro-1H-pyrazol-1-yl |
| 8-6 | 2,6-difluorophenyl | H | I | I | CF₃ | CF₃ | 4-chloro-1H-pyrazol-1-yl |
| 8-7 | 2-chloropyridin-3-yl | H | CH₃ | ethyl | CF₃ | CF₃ | 4-chloro-1H-pyrazol-1-yl |
| 8-8 | 2,6-difluorophenyl | H | CH₃ | ethyl | CF₃ | CF₃ | 4-bromo-1H-pyrazol-1-yl |
| 8-9 | 2,6-difluorophenyl | H | Br | Br | CF₃ | CF₃ | 4-bromo-1H-pyrazol-1-yl |
| 8-10a | 2-chloropyridin-3-yl | H | CH₃ | ethyl | CF₃ | CF₃ | ethoxy |
| 8-10b | 2-chloropyridin-3-yl | H | CH₃ | ethyl | CF₃ | CF₃ | 4-chlorophenoxy |
| 8-11 | 2-chloropyridin-3-yl | H | CH₃ | ethyl | CF₃ | CF₃ | 4-bromophenoxy |
| 8-12 | 2-chloropyridin-3-yl | H | CH₃ | ethyl | CF₃ | CF₂CF₃ | ethoxy |
| 8-13 | 2-chloropyridin-3-yl | H | CH₃ | ethyl | CF₃ | CF₂CF₃ | 4-chlorophenoxy |
| 8-14 | 2-chloropyridin-3-yl | H | CH₃ | ethyl | CF₃ | CF₂CF₃ | 4-chloro-1H-pyrazol-1-yl |

TABLE 9

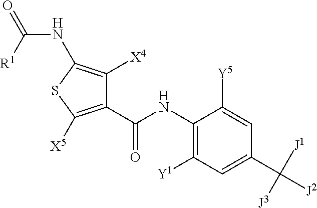

| | R1 | X⁴ | X⁵ | Y1 | Y5 | J1 | J2 | J3 |
|---|---|---|---|---|---|---|---|---|
| 9-1 | phenyl | H | H | CH₃ | ethyl | CF₃ | CF₃ | 4-chloro-1H-pyrazol-1-yl |
| 9-2 | 2-fluorophenyl | H | H | CH₃ | ethyl | CF₃ | CF₃ | 4-chloro-1H-pyrazol-1-yl |
| 9-3 | 4-chlorophenyl | H | H | CH₃ | ethyl | CF₃ | CF₃ | 4-chloro-1H-pyrazol-1-yl |
| 9-4 | 2,6-difluorophenyl | H | H | CH₃ | ethyl | CF₃ | CF₃ | 4-chloro-1H-pyrazol-1-yl |
| 9-5 | 2,6-difluorophenyl | H | H | Br | Br | CF₃ | CF₃ | 4-chloro-1H-pyrazol-1-yl |
| 9-6 | 2,6-difluorophenyl | H | H | I | I | CF₃ | CF₃ | 4-chloro-1H-pyrazol-1-yl |
| 9-7 | 2-chloropyridin-3-yl | H | H | CH₃ | ethyl | CF₃ | CF₃ | 4-chloro-1H-pyrazol-1-yl |
| 9-8 | 2,6-difluorophenyl | H | H | CH₃ | ethyl | CF₃ | CF₃ | 4-bromo-1H-pyrazol-1-yl |
| 9-9 | 2,6-difluorophenyl | H | H | Br | Br | CF₃ | CF₃ | 4-bromo-1H-pyrazol-1-yl |
| 9-10 | 2-chloropyridin-3-yl | H | H | CH₃ | ethyl | CF₃ | CF₃ | ethoxy |
| 9-11 | 2-chloropyridin-3-y1 | H | H | CH₃ | ethyl | CF₃ | CF₃ | 4-chlorophenoxy |
| 9-12 | 2-chloropyridin-3-y1 | H | H | CH₃ | ethyl | CF₃ | CF₃ | 4-bromophenoxy |
| 9-13 | 2-chloropyridin-3-y1 | H | H | CH₃ | ethyl | CF₃ | CF₂CF₃ | ethoxy |
| 9-14 | 2-chloropyridin-3-yl | H | H | CH₃ | ethyl | CF₃ | CF₂CF₃ | 4-chlorophenoxy |
| 9-15 | 2-chloropyridin-3-yl | H | H | CH₃ | ethyl | CF₃ | CF₂CF₃ | 4-chloro-1H-pyrazol-1-yl |

TABLE 10

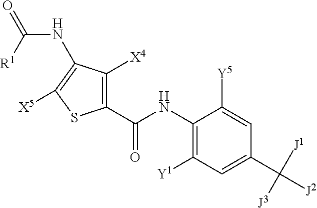

| | R1 | X4 | X⁵ | Y1 | Y5 | J1 | J2 | J3 |
|---|---|---|---|---|---|---|---|---|
| 10-1 | phenyl | H | H | CH₃ | ethyl | CF₃ | CF₃ | 4-chloro-1H-pyrazol-1-yl |
| 10-2 | 2-fluorophenyl | H | H | CH₃ | ethyl | CF₃ | CF₃ | 4-chloro-1H-pyrazol-1-yl |
| 10-3 | 4-chlorophenyl | H | H | CH₃ | ethyl | CF₃ | CF₃ | 4-chloro-1H-pyrazol-1-yl |
| 10-4 | 2,6-difluorophenyl | H | H | CH₃ | ethyl | CF₃ | CF₃ | 4-chloro-1H-pyrazol-1-yl |
| 10-5 | 2 ,6-difluorophenyl | H | H | Br | Br | CF₃ | CF₃ | 4-chloro-1H-pyrazol-1-yl |
| 10-6 | 2,6-difluorophenyl | H | H | I | I | CF₃ | CF₃ | 4-chloro-1H-pyrazol-1-yl |
| 10-7 | 2-chloropyridin-3-yl | H | H | CH₃ | ethyl | CF₃ | CF₃ | 4-chloro-1H-pyrazol-1-yl |
| 10-8 | 2,6-difluorophenyl | H | H | CH₃ | ethyl | CF₃ | CF₃ | 4-bromo-1H-pyrazol-1-yl |
| 10-9 | 2,6-difluorophenyl | H | H | Br | Br | CF₃ | CF₃ | 4-bromo-1H-pyrazol-1-yl |
| 10-10 | 2-chloropyridin-3-yl | H | H | CH₃ | ethyl | CF₃ | CF₃ | ethoxy |
| 10-11 | 2-chloropyridin-3-yl | H | H | CH₃ | ethyl | CF₃ | CF₃ | 4-chlorophenoxy |
| 10-12 | 2-chloropyridin-3-yl | H | H | CH₃ | ethyl | CF₃ | CF₃ | 4-bromophenoxy |
| 10-13 | 2-chloropyridin-3-yl | H | H | CH₃ | ethyl | CF₃ | CF₂CF₃ | ethoxy |
| 10-14 | 2-chloropyridin-3-yl | H | H | CH₃ | ethyl | CF₃ | CF₂CF₃ | 4-chlorophenoxy |
| 10-15 | 2-chloropyridin-3-yl | H | H | CH₃ | ethyl | CF₃ | CF₂CF₃ | 4-chloro-1H-pyrazol-1-yl |

TABLE 11

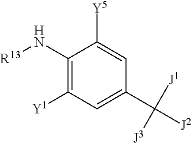

| | R13 | Y1 | Y5 | J1 | J2 | J3 |
|---|---|---|---|---|---|---|
| 11-1 | 3-nitrobenzoyl | CH₃ | CH₃ | CF₃ | H | OH |
| 11-2 | 3-aminobenzoyl | CH₃ | CH₃ | CF₃ | H | OH |
| 11-3 | 3-nitrobenzoyl | Br | Br | CF₃ | H | OH |
| 11-4 | 3-aminobenzoyl | Br | Br | CF₃ | H | OH |
| 11-5 | tert-butoxycarbonyl | H | CH₃ | CF₃ | CF₃ | OH |
| 11-6 | H | H | H | CF₃ | phenyl | OH |
| 11-7 | H | Br | Br | CF₃ | phenyl | OH |
| 11-8 | H | H | H | CF₃ | 2-chlorophenyl | OH |
| 11-9 | 3-nitrobenzoyl | CH₃ | CH₃ | CF₃ | 3-chlorophenyl | OH |
| 11-10 | 3-aminobenzoyl | CH₃ | CH₃ | CF₃ | 3-chlorophenyl | OH |
| 11-11 | 3-nitrobenzoyl | Br | Br | CF₃ | 4-chlorophenyl | OH |
| 11-12 | 3-aminobenzoyl | Br | Br | CF₃ | 4-chlorophenyl | OH |
| 11-13 | H | H | H | CF₃ | 3,5-dichlorophenyl | OH |
| 11-14 | H | H | H | CF₃ | 3-chlorophenyl | trimethylsilyloxy |
| 11-15 | H | Br | Br | CF₃ | 3-chlorophenyl | trimethylsilyloxy |
| 11-16 | H | H | H | CF₃ | 4-chlorophenyl | trimethylsilyloxy |
| 11-17 | H | Br | Br | CF₃ | 4-chlorophenyl | trimethylsilyloxy |
| 11-18 | tert-butoxycarbonyl | H | CH₃ | CF₃ | CF₃ | (methylsulfonyl)oxy |
| 11-19 | H | I | I | CF₃ | CF₃ | methoxy |
| 11-20 | 3-aminobenzoyl | I | I | CF₃ | CF₃ | methoxy |
| 11-21 | H | H | H | CF₃ | 3,5-dichlorophenyl | methoxy |
| 11-22 | H | I | I | CF₃ | CF₃ | ethoxy |
| 11-23 | 3-aminobenzoyl | I | I | CF₃ | CF₃ | ethoxy |
| 11-24 | H | I | I | CF₃ | CF₃ | 2-fluoroethoxy |
| 11-25 | 3-nitrobenzoyl | CH₃ | ethyl | CF₃ | CF₃ | 2,2,2-trifluoroethoxy |
| 11-26 | 3-aminobenzoyl | CH₃ | ethyl | CF₃ | CF₃ | 2,2,2-trifluoroethoxy |
| 11-27 | H | I | I | CF₃ | CF₃ | prop-2-en-1-yloxy |
| 11-28 | 3-aminobenzoyl | I | I | CF₃ | CF₃ | prop-2-en-1-yloxy |
| 11-29 | H | I | I | CF₃ | CF₃ | (3-methylbut-2-en-1-yl)oxy |
| 11-30 | H | I | I | CF₃ | CF₃ | 2-methoxyethoxy |
| 11-31 | 3-aminobenzoyl | I | I | CF3 | CF₃ | 2-methoxyethoxy |
| 11-32 | H | I | I | CF₃ | CF₃ | 2-(methylsulfanyl)ethoxy |
| 11-33 | 3-nitrobenzoyl | CH₃ | ethyl | CF₃ | CF₃ | phenoxy |
| 11-34 | 3-aminobenzoyl | CH₃ | ethyl | CF₃ | CF₃ | phenoxy |
| 11-35 | 3-nitrobenzoyl | CH₃ | ethyl | CF₃ | CF₃ | 2-chlorophenoxy |
| 11-36 | 3-aminobenzoyl | CH₃ | ethyl | CF₃ | CF₃ | 2-chlorophenoxy |
| 11-37 | 3-nitrobenzoyl | CH₃ | ethyl | CF₃ | CF₃ | 3-chlorophenoxy |
| 11-38 | 3-aminobenzoyl | CH₃ | ethyl | CF₃ | CF₃ | 3-chlorophenoxy |
| 11-39 | 3-nitrobenzoyl | CH₃ | ethyl | CF₃ | CF₃ | 4-chlorophenoxy |
| 11-40 | 3-aminobenzoyl | CH₃ | ethyl | CF₃ | CF₃ | 4-chlorophenoxy |
| 11-41 | 3-nitrobenzoyl | CH₃ | CH₃ | CF₃ | CF₃ | benzyloxy |
| 11-42 | 3-aminobenzoyl | CH₃ | CH₃ | CF₃ | CF₃ | benzyloxy |
| 11-43 | 3-nitrobenzoyl | CH₃ | CH₃ | CF₃ | CF₃ | (2-chlorobenzyl)oxy |
| 11-44 | 3-aminobenzoyl | CH₃ | CH₃ | CF₃ | CF₃ | (2-chlorobenzyl)oxy |
| 11-45 | 3-nitrobenzoyl | CH₃ | CH₃ | CF₃ | CF₃ | (4-chlorobenzyl)oxy |
| 11-46 | 3-aminobenzoyl | CH₃ | CH₃ | CF₃ | CF₃ | (4-chlorobenzyl)oxy |
| 11-47 | 3-nitrobenzoyl | CH₃ | ethyl | CF₃ | CF₃ | (4-chlorobenzyl)oxy |
| 11-48 | 3-aminobenzoyl | CH₃ | ethyl | CF₃ | CF₃ | (4-chlorobenzyl)oxy |
| 11-49 | 3-nitrobenzoyl | CH₃ | ethyl | CF₃ | CF₃ | (3-chlorobenzyl)oxy |
| 11-50 | 3-aminobenzoyl | CH₃ | ethyl | CF₃ | CF₃ | (3-chlorobenzyl)oxy |
| 11-51 | 3-nitrobenzoyl | CH₃ | ethyl | CF₃ | CF₃ | (propan-2-ylideneamino)oxy |
| 11-52 | 3-aminobenzoyl | CH₃ | ethyl | CF₃ | CF₃ | (propan-2-ylideneamino)oxy |
| 11-53 | 3-nitrobenzoyl | CH₃ | ethyl | CF₃ | CF₃ | (cyclopentylideneamino)oxy |
| 11-54 | 3-aminobenzoyl | CH₃ | ethyl | CF₃ | CF₃ | (cyclopentylideneamino)oxy |
| 11-55 | 3-nitrobenzoyl | CH₃ | ethyl | CF₃ | CF₃ | (cyclohexylideneamino)oxy |
| 11-56 | 3-aminobenzoyl | CH₃ | ethyl | CF₃ | CF₃ | (cyclohexylideneamino)oxy |
| 11-57 | 3-nitrobenzoyl | CH₃ | ethyl | CF₃ | CF₃ | ({phenylmethylidene-amino)oxy |
| 11-58 | 3-aminobenzoyl | CH₃ | ethyl | CF₃ | CF₃ | ({phenylmethylidene}-amino)oxy |
| 11-59 | 3-nitrobenzoyl | CH₃ | ethyl | CF₃ | CF₃ | {(1-phenylethylidene)-amino}oxy |
| 11-60 | 3-aminobenzoyl | CH₃ | ethyl | CF₃ | CF₃ | {(1-phenylethylidene)-amino}oxy |
| 11-61 | 3-nitrobenzoyl | CH₃ | ethyl | CF₃ | CF₃ | 1H-pyrrol-1-yl |
| 11-62 | 3-aminobenzoyl | CH₃ | ethyl | CF₃ | CF₃ | 1H-pyrrol-1-yl |
| 11-63 | 3-nitrobenzoyl | CH₃ | CH₃ | CF₃ | CF₃ | 1H-pyrazol-1-yl |
| 11-64 | 3-aminobenzoyl | CH₃ | CH₃ | CF₃ | CF₃ | 1H-pyrazol-1-yl |
| 11-65 | 3-nitrobenzoyl | CH₃ | CH₃ | CF₃ | H | 4-chloro-1H-pyrazol-1-yl |
| 11-66 | 3-aminobenzoyl | CH₃ | CH₃ | CF₃ | H | 4-chloro-1H-pyrazol-1-yl |
| 11-67 | 3-nitrobenzoyl | Br | Br | CF₃ | CH₂F | 4-chloro-1H-pyrazol-1-yl |
| 11-68 | 3-aminobenzoyl | Br | Br | CF₃ | CH₂F | 4-chloro-1H-pyrazol-1-yl |
| 11-69 | H | H | H | CF₃ | CF₃ | 4-chloro-1H-pyrazol-1-yl |
| 11-70 | tert-butoxycarbonyl | H | H | CF₃ | CF₃ | 4-chloro-1H-pyrazol-1-yl |
| 11-71 | 3-nitrobenzoyl | H | CH₃ | CF₃ | CF₃ | 4-chloro-1H-pyrazol-1-yl |
| 11-72 | 3-aminobenzoyl | H | CH₃ | CF₃ | CF₃ | 4-chloro-1H-pyrazol-1-yl |
| 11-73 | 3-nitrobenzoyl | CH₃ | CH₃ | CF₃ | CF₃ | 4-chloro-1H-pyrazol-1-yl |
| 11-74 | 3-aminobenzoyl | CH₃ | CH₃ | CF₃ | CF₃ | 4-chloro-1H-pyrazol-1-yl |
| 11-75 | 3-nitrobenzoyl | CH₃ | ethyl | CF₃ | CF₃ | 4-chloro-1H-pyrazol-1-yl |
| 11-76 | 3-aminobenzoyl | CH₃ | ethyl | CF₃ | CF₃ | 4-chloro-1H-pyrazol-1-yl |
| 11-77 | 3-nitrobenzoyl | CH₃ | Br | CF₃ | CF₃ | 4-chloro-1H-pyrazol-1-yl |
| 11-78 | 3-aminobenzoyl | CH₃ | Br | CF₃ | CF₃ | 4-chloro-1H-pyrazol-1-yl |
| 11-79 | 3-nitrobenzoyl | Cl | Cl | CF₃ | CF₃ | 4-chloro-1H-pyrazol-1-yl |
| 11-80 | 3-aminobenzoyl | Cl | Cl | CF₃ | CF₃ | 4-chloro-1H-pyrazol-1-yl |
| 11-81 | 3-nitrobenzoyl | Br | Br | CF₃ | CF₃ | 4-chloro-1H-pyrazol-1-yl |
| 11-82 | 3-aminobenzoyl | Br | Br | CF₃ | CF₃ | 4-chloro-1H-pyrazol-1-yl |
| 11-83 | H | Br | Br | CF₃ | 4-chlorophenyl | 4-chloro-1H-pyrazol-1-yl |
| 11-84 | 3-nitrobenzoyl | I | I | CF₃ | CF₃ | 4-chloro-1H-pyrazol-1-yl |
| 11-85 | 3-aminobenzoyl | I | I | CF₃ | CF₃ | 4-chloro-1H-pyrazol-1-yl |
| 11-86 | 3-nitrobenzoyl | CH₃ | CH₃ | CF₃ | H | 4-bromo-1H-pyrazol-1-yl |
| 11-87 | 3-aminobenzoyl | CH₃ | CH₃ | CF₃ | H | 4-bromo-1H-pyrazol-1-yl |
| 11-88 | 3-nitrobenzoyl | Br | Br | CF₃ | CH₃ | 4-bromo-1H-pyrazol-1-yl |
| 11-89 | 3-aminobenzoyl | Br | Br | CF₃ | CH₃ | 4-bromo-1H-pyrazol-1-yl |
| 11-90 | 3-nitrobenzoyl | CH₃ | CH₃ | CF₃ | CF₃ | 4-bromo-1H-pyrazol-1-yl |
| 11-91 | 3-aminobenzoyl | CH₃ | CH₃ | CF₃ | CF₃ | 4-bromo-1H-pyrazol-1-yl |
| 11-92 | 3-nitrobenzoyl | CH₃ | ethyl | CF₃ | CF₃ | 4-bromo-1H-pyrazol-1-yl |
| 11-93 | 3-aminobenzoyl | CH₃ | ethyl | CF₃ | CF₃ | 4-bromo-1H-pyrazol-1-yl |
| 11-94 | 3-nitrobenzoyl | CH₃ | CH₃ | CF₃ | CF₃ | 4-iodo-1H-pyrazol-1-yl |
| 11-95 | 3-aminobenzoyl | CH₃ | CH₃ | CF₃ | CF₃ | 4-iodo-1H-pyrazol-1-yl |
| 11-96 | 3-nitrobenzoyl | CH₃ | CH₃ | CF₃ | CF₃ | 3-methyl-1H-pyrazol-1-yl |
| 11-97 | 3-aminobenzoyl | CH₃ | CH₃ | CF₃ | CF₃ | 3-methyl-1H-pyrazol-1-yl |
| 11-98 | 3-nitrobenzoyl | CH₃ | CH₃ | CF₃ | CF₃ | 4-methyl-1H-pyrazol-1-yl |
| 11-99 | 3-aminobenzoyl | CH₃ | CH₃ | CF₃ | CF₃ | 4-methyl-1H-pyrazol-1-yl |
| 11-100 | H | H | H | CF₃ | phenyl | 3-(trifluoromethyl)-1H-pyrazol-1-yl |
| 11-101 | H | H | H | CF₃ | 2-chlorophenyl | 3-(trifluoromethyl)-1H-pyrazol-1-yl |
| 11-102 | tert-butoxycarbonyl | H | H | CF₃ | CF₃ | 3-(trifluoromethyl)-1H-pyrazol-1-yl |
| 11-103 | 3-nitrobenzoyl | CH₃ | CH₃ | CF₃ | CF₃ | 3-(trifluoromethyl)-1H-pyrazol-1-yl |
| 11-104 | 3-aminobenzoyl | CH₃ | CH₃ | CF₃ | CF₃ | 3-(trifluoromethyl)-1H-pyrazol-1-yl |
| 11-105 | H | Br | Br | CF₃ | phenyl | 3-(trifluoromethyl)-1H-pyrazol-1-yl |
| 11-106 | 3-nitrobenzoyl | Br | Br | CF₃ | phenyl | 3-(trifluoromethyl)-1H-pyrazol-1-yl |
| 11-107 | 3-aminobenzoyl | Br | Br | CF₃ | phenyl | 3-(trifluoromethyl)-1H-pyrazol-1-yl |
| 11-108 | 3-nitrobenzoyl | CH₃ | CH₃ | CF₃ | 2-chlorophenyl | 3-(trifluoromethyl)-1H-pyrazol-1-yl |
| 11-109 | 3-aminobenzoyl | CH₃ | CH₃ | CF₃ | 2-chlorophenyl | 3-(trifluoromethyl)-1H-pyrazol-1-yl |
| 11-110 | H | Br | Br | CF₃ | 2-chlorophenyl | 3-(trifluoromethyl)-1H-pyrazol-1-yl |
| 11-111 | 3-nitrobenzoyl | CH₃ | CH₃ | CF₃ | CF₃ | 4-(trifluoromethyl)-1H-pyrazol-1-yl |
| 11-112 | 3-aminobenzoyl | CH₃ | CH₃ | CF₃ | CF₃ | 4-(trifluoromethyl)-1H-pyrazol-1-yl |
| 11-113 | 3-nitrobenzoyl | CH₃ | ethyl | CF₃ | CF₃ | 4-(trifluoromethyl)-1H-pyrazol-1-yl |
| 11-114 | 3-aminobenzoyl | CH₃ | ethyl | CF₃ | CF₃ | 4-(trifluoromethyl)-1H-pyrazol-1-yl |
| 11-115 | 3-nitrobenzoyl | CH₃ | CH₃ | CF₃ | CF₃ | 3-(pentafluoroethyl)-1H-pyrazol-1-yl |
| 11-116 | 3-aminobenzoyl | CH₃ | CH₃ | CF₃ | CF₃ | 3-(pentafluoroethyl)-1H-pyrazol-1-yl |
| 11-117 | 3-nitrobenzoyl | CH₃ | CH₃ | CF₃ | CF₃ | 3-(dimethoxymethyl)-1H-pyrazol-1-yl |
| 11-118 | 3-aminobenzoyl | CH₃ | CH₃ | CF₃ | CF₃ | 3-(dimethoxymethyl)-1H-pyrazol-1-yl |
| 11-119 | 3-nitrobenzoyl | CH₃ | CH₃ | CF₃ | H | 4-cyano-1H-pyrazol-1-yl |
| 11-120 | 3-aminobenzoyl | CH₃ | CH₃ | CF₃ | H | 4-cyano-1H-pyrazol-1-yl |
| 11-121 | 3-nitrobenzoyl | CH₃ | CH₃ | CF₃ | CF₃ | 5-acetyl-1H-pyrazol-1-yl |
| 11-122 | 3-aminobenzoyl | CH₃ | CH₃ | CF₃ | CF₃ | 5-acetyl-1H-pyrazol-1-yl |
| 11-123 | 3-nitrobenzoyl | CH₃ | CH₃ | CF₃ | CF₃ | 3,4-dibromo-1H-pyrazol-1-yl |
| 11-124 | 3-aminobenzoyl | CH₃ | CH₃ | CF₃ | CF₃ | 3,4-dibromo-1H-pyrazol-1-yl |
| 11-125 | 3-nitrobenzoyl | CH₃ | CH₃ | CF₃ | H | 3,5-dimethyl-1H-pyrazol-1-yl |
| 11-126 | 3-aminobenzoyl | CH₃ | CH₃ | CF₃ | H | 3,5-dimethyl-1H-pyrazol-1-yl |
| 11-127 | 3-nitrobenzoyl | CH₃ | CH₃ | CF₃ | H | 3,5-bis(trifluoromethyl)-1H-pyrazol-1-yl |
| 11-128 | 3-aminobenzoyl | CH₃ | CH₃ | CF₃ | H | 3,5-bis(trifluoromethyl)-1H-pyrazol-1-yl |
| 11-129 | 3-nitrobenzoyl | CH₃ | CH₃ | CF₃ | CF₃ | 3,5-bis(trifluoromethyl)-1H-pyrazol-1-yl |
| 11-130 | 3-aminobenzoyl | CH₃ | CH₃ | CF₃ | CF₃ | 3,5-bis(trifluoromethyl)-1H-pyrazol-1-yl |
| 11-131 | 3-nitrobenzoyl | CH₃ | CH₃ | CF₃ | CF₃ | 4-chloro-3-(pentafluoroethyl)-1H-pyrazol-1-yl |
| 11-132 | 3-aminobenzoyl | CH₃ | CH₃ | CF₃ | CF₃ | 4-chloro-3-(pentafluoroethyl)-1H-pyrazol-1-yl |
| 11-133 | 3-nitrobenzoyl | CH₃ | CH₃ | CF₃ | CF₃ | 4-chloro-3-(trifluoromethyl)-1H-pyrazol-1-yl |
| 11-134 | 3-aminobenzoyl | CH₃ | CH₃ | CF₃ | CF₃ | 4-chloro-3-(trifluoromethyl)-1H-pyrazol-1-yl |
| 11-135 | 3-nitrobenzoyl | CH₃ | CH₃ | CF₃ | CF₃ | 4-iodo-3-(trifluoromethyl)-1H-pyrazol-1-yl |
| 11-136 | 3-aminobenzoyl | CH₃ | CH₃ | CF₃ | CF₃ | 4-iodo-3-(trifluoromethyl)-1H-pyrazol-1-yl |
| 11-137 | 3-nitrobenzoyl | CH₃ | CH₃ | CF₃ | CF₃ | 4-(ethoxycarbonyl)-3-(trifluoromethyl)-1H-pyrazol-1-yl |
| 11-138 | 3-aminobenzoyl | CH₃ | CH₃ | CF₃ | CF₃ | 4-(ethoxycarbonyl)-3-(trifluoromethyl)-1H-pyrazol-1-yl |
| 11-139 | 3-nitrobenzoyl | CH₃ | CH₃ | CF₃ | CF₃ | 3,4,5-tribromo-1H-pyrazol-1-yl |
| 11-140 | 3-aminobenzoyl | CH₃ | CH₃ | CF₃ | CF₃ | 3,4,5-tribromo-1H-pyrazol-1-yl |
| 11-141 | 3-nitrobenzoyl | CH₃ | ethyl | CF₃ | CF₃ | 3,4,5-tribromo-1H-pyrazol-1-yl |
| 11-142 | 3-aminobenzoyl | CH₃ | ethyl | CF₃ | CF₃ | 3,4,5-tribromo-1H-pyrazol-1-yl |
| 11-143 | 3-nitrobenzoyl | CH₃ | CH₃ | CF₃ | H | 4-bromo-3,5-dimethyl-1H-pyrazol-1-yl |
| 11-144 | 3-aminobenzoyl | CH₃ | CH₃ | CF₃ | H | 4-bromo-3,5-dimethyl-1H-pyrazol-1-yl |
| 11-145 | 3-nitrobenzoyl | CH₃ | CH₃ | CF₃ | CF₃ | 1H-imidazol-1-yl |
| 11-146 | 3-aminobenzoyl | CH₃ | CH₃ | CF₃ | CF₃ | 1H-imidazol-1-yl |
| 11-147 | 3-nitrobenzoyl | CH₃ | CH₃ | CF₃ | CF₃ | 2,4,5-tribromo-1H-imidazol-1-yl |
| 11-148 | 3-aminobenzoyl | CH₃ | CH₃ | CF₃ | CF₃ | 2,4,5-tribromo-1H-imidazol-1-yl |
| 11-149 | H | H | H | CF₃ | CF₃ | 1H-1,2,4-triazol-1-yl |
| 11-150 | H | H | CH₃ | CF₃ | CF₃ | 1H-1,2,4-triazol-1-yl |
| 11-151 | tert-butoxycarbonyl | H | CH₃ | CF₃ | CF₃ | 1H-1,2,4-triazol-1-yl |
| 11-152 | H | CH₃ | Br | CF₃ | CF₃ | 1H-1,2,4-triazol-1-yl |
| 11-153 | H | Br | Br | CF₃ | CF₃ | 1H-1,2,4-triazol-1-yl |
| 11-154 | 3-nitrobenzoyl | CH₃ | CH₃ | CF₃ | CF₃ | 1H-1,2,4-triazol-1-yl |
| 11-155 | 3-aminobenzoyl | CH₃ | CH₃ | CF₃ | CF₃ | 1H-1,2,4-triazol-1-yl |
| 11-156 | 3-nitrobenzoyl | CH₃ | ethyl | CF₃ | CF₃ | 1H-1,2,4-triazol-1-yl |
| 11-157 | 3-aminobenzoyl | CH₃ | ethyl | CF₃ | CF₃ | 1H-1,2,4-triazol-1-yl |
| 11-158 | 3-nitrobenzoyl | CH₃ | Br | CF₃ | CF₃ | 1H-1,2,4-triazol-1-yl |
| 11-159 | 3-aminobenzoyl | CH₃ | Br | CF₃ | CF₃ | 1H-1,2,4-triazol-1-yl |
| 11-160 | 3-nitrobenzoyl | Br | Br | CF₃ | CF₃ | 1H-1,2,4-triazol-1-yl |
| 11-161 | 3-aminobenzoyl | Br | Br | CF₃ | CF₃ | 1H-1,2,4-triazol-1-yl |
| 11-162 | 3-nitrobenzoyl | CH₃ | ethyl | CF₃ | CF₃ | 3-chloro-1H-1,2,4-triazol-1-yl |
| 11-163 | 3-aminobenzoyl | CH₃ | ethyl | CF₃ | CF₃ | 3-chloro-1H-1,2,4-triazol-1-yl |
| 11-164 | 3-nitrobenzoyl | CH₃ | CH₃ | CF₃ | CF₃ | 3-(trifluoromethyl)-1H-1,2,4-triazol-1-yl |
| 11-165 | 3-aminobenzoyl | CH₃ | CH₃ | CF₃ | CF₃ | 3-(trifluoromethyl)-1H-1,2,4-triazol-1-yl |
| 11-166 | 3-nitrobenzoyl | CH₃ | CH₃ | CF₃ | CF₃ | 2H-tetrazol-2-yl |
| 11-167 | 3-aminobenzoyl | CH₃ | CH₃ | CF₃ | CF₃ | 2H-tetrazol-2-yl |
| 11-168 | 3-nitrobenzoyl | CH₃ | ethyl | CF₃ | CF₃ | 3-chloro-1H-indazol-1-yl |
| 11-169 | 3-aminobenzoyl | CH₃ | ethyl | CF₃ | CF₃ | 3-chloro-1H-indazol-1-yl |
| 11-170 | 3-nitrobenzoyl | CH₃ | ethyl | CF₃ | CF₃ | 1H-benzimidazol-1-yl |
| 11-171 | 3-aminobenzoyl | CH₃ | ethyl | CF₃ | CF₃ | 1H-benzimidazol-1-yl |
| 11-172 | 3-nitrobenzoyl | CH₃ | ethyl | CF₃ | CF₃ | 2-chloro-1H-benzimidazol-1-yl |
| 11-173 | 3-aminobenzoyl | CH₃ | ethyl | CF₃ | CF₃ | 2-chloro-1H-benzimidazol-1-yl |
| 11-174 | 3-nitrobenzoyl | CH₃ | ethyl | CF₃ | CF₃ | 1H-benzotriazol-1-yl |
| 11-175 | 3-aminobenzoyl | CH₃ | ethyl | CF₃ | CF₃ | 1H-benzotriazol-1-yl |
| 11-176 | 3-nitrobenzoyl | CH₃ | ethyl | CF₃ | CF₃ | piperidin-1-yl |
| 11-177 | 3-aminobenzoyl | CH₃ | ethyl | CF₃ | CF₃ | piperidin-1-yl |
| 11-178 | 3-nitrobenzoyl | CH₃ | ethyl | CF₃ | CF₃ | pyrrolidin-1-yl |
| 11-179 | 3-aminobenzoyl | CH₃ | ethyl | CF₃ | CF₃ | pyrrolidin-1-yl |
| 11-180 | 3-nitrobenzoyl | CH₃ | ethyl | CF₃ | CF₃ | 3,6-dihydropyridin-1(2H)-yl |
| 11-181 | 3-aminobenzoyl | CH₃ | ethyl | CF₃ | CF₃ | 3,6-dihydropyridin-1(2H)-yl |
| 11-182 | 3-nitrobenzoyl | CH₃ | ethyl | CF₃ | CF₃ | morpholin-4-yl |
| 11-183 | 3-aminobenzoyl | CH₃ | ethyl | CF₃ | CF₃ | morpholin-4-yl |
| 11-184 | H | H | H | CF₃ | CF₃ | CN |
| 11-185 | tert-butoxycarbonyl | H | H | CF₃ | CF₃ | CN |
| 11-186 | H | Br | Br | CF₃ | CF₃ | CN |
| 11-187 | 3-nitrobenzoyl | Br | Br | CF₃ | CF₃ | CN |
| 11-188 | 3-aminobenzoyl | Br | Br | CF₃ | CF₃ | CN |
| 11-189 | 3-nitrobenzoyl | CH₃ | ethyl | CF₃ | CF₃ | CN |
| 11-190 | 3-aminobenzoyl | CH₃ | ethyl | CF₃ | CF₃ | CN |
| 11-191 | 3-nitrobenzoyl | CH₃ | ethyl | CF₃ | CF₃ | 3-(trifluoromethyl)phenoxy |
| 11-192 | 3-aminobenzoyl | CH₃ | ethyl | CF₃ | CF₃ | 3-(trifluoromethyl)phenoxy |
| 11-193 | 3-nitrobenzoyl | CH₃ | ethyl | CF₃ | CF₃ | 4-fluorophenoxy |
| 11-194 | 3-aminobenzoyl | CH₃ | ethyl | CF₃ | CF₃ | 4-fluorophenoxy |
| 11-195 | 3-nitrobenzoyl | CH₃ | ethyl | CF₃ | CF₃ | 4-bromophenoxy |
| 11-196 | 3-aminobenzoyl | CH₃ | ethyl | CF₃ | CF₃ | 4-bromophenoxy |
| 11-197 | 3-nitrobenzoyl | CH₃ | ethyl | CF₃ | CF₃ | 4-(trifluoromethyl)phenoxy |
| 11-198 | 3-aminobenzoyl | CH₃ | ethyl | CF₃ | CF₃ | 4-(trifluoromethyl)phenoxy |
| 11-199 | 3-nitrobenzoyl | CH₃ | ethyl | CF₃ | CF₃ | 4-methylphenoxy |
| 11-200 | 3-aminobenzoyl | CH₃ | ethyl | CF₃ | CF₃ | 4-methylphenoxy |
| 11-201 | 3-nitrobenzoyl | CH₃ | ethyl | CF₃ | CF₃ | 4-methoxylphenoxy |
| 11-202 | 3-aminobenzoyl | CH₃ | ethyl | CF₃ | CF₃ | 4-methoxylphenoxy |
| 11-203 | 3-nitrobenzoyl | CH₃ | ethyl | CF₃ | CF₃ | [2-(trifluoromethyl)pyridin-4-yl]oxy |
| 11-204 | 3-aminobenzoyl | CH₃ | ethyl | CF₃ | CF₃ | [2-(trifluoromethyl)pyridin-4-yl]oxy |
| 11-205 | 3-nitrobenzoyl | CH₃ | ethyl | CF₃ | CF₃ | [2-(pentafluoroethyl)pyridin-4-yl]oxy |
| 11-206 | 3-aminobenzoyl | CH₃ | ethyl | CF₃ | CF₃ | [2-(pentafluoroethyl)pyridin-4-yl]oxy |
| 11-207 | 3-nitrobenzoyl | CH₃ | ethyl | CF₃ | CF₃ | (pyridin-4-yl)oxy |
| 11-208 | 3-aminobenzoyl | CH₃ | ethyl | CF₃ | CF₃ | (pyridin-4-yl)oxy |
| 11-209 | 3-nitrobenzoyl | CH₃ | ethyl | CF₃ | CF₃ | (2-phenylethyl)oxy |
| 11-210 | 3-aminobenzoyl | CH₃ | ethyl | CF₃ | CF₃ | (2-phenylethyl)oxy |
| 11-211 | 3-nitrobenzoyl | CH₃ | ethyl | CF₃ | CF₃ | [1-methyl-3-(trifluoromethyl)-1H-pyrazol-5-yl]oxy |
| 11-212 | 3-aminobenzoyl | CH₃ | ethyl | CF₃ | CF₃ | [1-methyl-3-(trifluoromethyl)-1H-pyrazol-5-yl]oxy |
| 11-213 | 3-nitrobenzoyl | CH₃ | ethyl | CF₃ | CF₃ | {[(4-chlorophenyl)methylidene]amino}oxy |
| 11-214 | 3-aminobenzoyl | CH₃ | ethyl | CF₃ | CF₃ | {[(4-chlorophenyl)methylidene]amino}oxy |
| 11-215 | 3-nitrobenzoyl | CH₃ | ethyl | CF₃ | CF₃ | cyclopropylmethoxy |
| 11-216 | 3-aminobenzoyl | CH₃ | ethyl | CF₃ | CF₃ | cyclopropylmethoxy |
| 11-217 | 3-nitrobenzoyl | CH₃ | ethyl | CF₃ | CF₃ | {[(4-methylphenyl)methylidene]amino}oxy |
| 11-218 | 3-aminobenzoyl | CH₃ | ethyl | CF₃ | CF₃ | {[(4-methylphenyl)methylidene]amino}oxy |
| 11-219 | 3-nitrobenzoyl | CH₃ | ethyl | CF₃ | CF₃ | {[(4-methoxyphenyl)methylidene]amino}oxy |
| 11-220 | 3-aminobenzoyl | CH₃ | ethyl | CF₃ | CF₃ | {[(4-methoxyphenyl)methylidene]amino}oxy |
| 11-221 | 3-nitrobenzoyl | CH₃ | ethyl | CF₃ | CF₃ | 3,4-dichlorophenoxy |
| 11-222 | 3-aminobenzoyl | CH₃ | ethyl | CF₃ | CF₃ | 3,4-dichlorophenoxy |
| 11-223 | 3-nitrobenzoyl | CH₃ | ethyl | CF₃ | CF₃ | 4-iodophenoxy |
| 11-224 | 3-aminobenzoyl | CH₃ | ethyl | CF₃ | CF₃ | 4-iodophenoxy |
| 11-225 | 3-nitrobenzoyl | CH₃ | ethyl | CF₃ | CF₃ | 4-(trifluoromethoxy)phenyl |
| 11-226 | 3-aminobenzoyl | CH₃ | ethyl | CF₃ | CF₃ | 4-(trifluoromethoxy)phenyl |
| 11-227 | 3-nitrobenzoyl | CH₃ | ethyl | CF₃ | CF₃ | 4-ethylphenoxy |
| 11-228 | 3-aminobenzoyl | CH₃ | ethyl | CF₃ | CF₃ | 4-ethylphenoxy |
| 11-229 | 3-nitrobenzoyl | CH₃ | ethyl | CF₃ | CF₃ | 4-butylphenoxy |
| 11-230 | 3-aminobenzoyl | CH₃ | ethyl | CF₃ | CF₃ | 4-butylphenoxy |
| 11-231 | 3-nitrobenzoyl | CH₃ | CH₃ | CF₃ | CF₃ | 4-chlorophenoxy |
| 11-232 | 3-aminobenzoyl | CH₃ | CH₃ | CF₃ | CF₃ | 4-chlorophenoxy |
| 11-233 | 3-nitrobenzoyl | CH₃ | CH₃ | CF₃ | CF₃ | 4-bromophenoxy |
| 11-234 | 3-aminobenzoyl | CH₃ | CH₃ | CF₃ | CF₃ | 4-bromophenoxy |
| 11-235 | 3-nitrobenzoyl | CH₃ | ethyl | CF₃ | CF₃ | 4-(trifluoromethylthio)phenyl |
| 11-236 | 3-aminobenzoyl | CH₃ | ethyl | CF₃ | CF₃ | 4-(trifluoromethylthio)phenyl |
| 11-237 | 3-nitrobenzoyl | CH₃ | CH₃ | CF₃ | CF₃ | 3-(trifluoromethyl)phenoxy |
| 11-238 | 3-aminobenzoyl | CH₃ | CH₃ | CF₃ | CF₃ | 3-(trifluoromethyl)phenoxy |
| 11-239 | 3-nitrobenzoyl | CH₃ | ethyl | CF₃ | CF₃ | 4-cyanophenoxy |
| 11-240 | 3-aminobenzoyl | CH₃ | ethyl | CF₃ | CF₃ | 4-cyanophenoxy |
| 11-241 | 3-nitrobenzoyl | CH₃ | CH₃ | CF₃ | CF₃ | 4-cyanophenoxy |
| 11-242 | 3-aminobenzoyl | CH₃ | CH₃ | CF₃ | CF₃ | 4-cyanophenoxy |
| 11-243 | 3-nitrobenzoyl | CH₃ | ethyl | CF₃ | CF₃ | 4-acetamidophenoxy |
| 11-244 | 3-aminobenzoyl | CH₃ | ethyl | CF₃ | CF₃ | 4-acetamidophenoxy |
| 11-245 | 3-nitrobenzoyl | CH₃ | ethyl | CF₃ | CF₃ | (pyridin-2-yl)oxy |
| 11-246 | 3-aminobenzoyl | CH₃ | ethyl | CF₃ | CF₃ | (pyridin-2-yl)oxy |
| 11-247 | 3-nitrobenzoyl | CH₃ | ethyl | CF₃ | CF₃ | (pyridin-3-yl)oxy |
| 11-248 | 3-aminobenzoyl | CH₃ | ethyl | CF₃ | CF₃ | (pyridin-3-yl)oxy |
| 11-249 | 3-nitrobenzoyl | CH₃ | ethyl | CF₃ | CF₃ | (5-chloropyridin-2-yl)oxy |
| 11-250 | 3-aminobenzoyl | CH₃ | ethyl | CF₃ | CF₃ | (5-chloropyridin-2-yl)oxy |
| 11-251 | 3-nitrobenzoyl | CH₃ | ethyl | CF₃ | CF₃ | (6-chloropyridazin-3-yl)oxy |
| 11-252 | 3-aminobenzoyl | CH₃ | ethyl | CF₃ | CF₃ | (6-chloropyridazin-3-yl)oxy |
| 11-253 | 3-nitrobenzoyl | ethyl | ethyl | CF₃ | CF₃ | 4-chlorophenoxy |
| 11-254 | 3-aminobenzoyl | ethyl | ethyl | CF₃ | CF₃ | 4-chlorophenoxy |
| 11-255 | 3-nitrobenzoyl | ethyl | ethyl | CF₃ | CF₃ | 4-bromophenoxy |
| 11-256 | 3-aminobenzoyl | ethyl | ethyl | CF₃ | CF₃ | 4-bromophenoxy |
| 11-257 | 3-nitrobenzoyl | H | H | CF₃ | CF₃ | 4-chlorophenoxy |
| 11-258 | 3-aminobenzoyl | H | H | CF₃ | CF₃ | 4-chlorophenoxy |
| 11-259 | 3-nitrobenzoyl | H | H | CF₃ | CF₃ | 4-bromophenoxy |
| 11-260 | 3-aminobenzoyl | H | H | CF₃ | CF₃ | 4-bromophenoxy |
| 11-261 | 3-nitrobenzoyl | CH₃ | ethyl | CF₃ | CF₃ | (6-chloropyridin-3-yl)oxy |
| 11-262 | 3-aminobenzoyl | CH₃ | ethyl | CF₃ | CF₃ | (6-chloropyridin-3-yl)oxy |
| 11-263 | 3-nitrobenzoyl | CH₃ | CH₃ | CF₃ | CF₃ | (6-chloropyridin-3-yl)oxy |
| 11-264 | 3-aminobenzoyl | CH₃ | CH₃ | CF₃ | CF₃ | (6-chloropyridin-3-yl)oxy |
| 11-265 | 3-nitrobenzoyl | Br | Br | CF₃ | CF₃ | 4-chlorophenoxy |
| 11-266 | 3-aminobenzoyl | Br | Br | CF₃ | CF₃ | 4-chlorophenoxy |
| 11-267 | 3-nitrobenzoyl | CH₃ | H | CF₃ | CF₃ | 4-chlorophenoxy |
| 11-268 | 3-aminobenzoyl | CH₃ | H | CF₃ | CF₃ | 4-chlorophenoxy |
| 11-269 | 3-nitrobenzoyl | CH₃ | ethyl | CF₃ | CF₃ | 4-(dimethylamino)phenyl]methylidene}amino)oxy |
| 11-270 | 3-aminobenzoyl | CH₃ | ethyl | CF₃ | CF₃ | 4-(dimethylamino)phenyl]methylidene}amino)oxy |

TABLE 12

| No. | Physical properties (melting point [° C.] or NMR-data) |
|---|---|
| 1-1 | 128-141 |
| 1-2 | 1,5618 |
| 1-3 | 217-225 |
| 1-4 | 1,5257 |
| 1-5 | 1,5237 |
| 1-6 | 1,5032 |
| 1-7 | >250 |
| 1-18 | 1,546 |
| 1-19 | 1,542 |
| 1-20 | 175-176 |
| 1-21 | 115-122 |
| 1-22 | 213-217 |
| 1-23 | 236-238 |
| 1-24 | 157-161 |
| 1-25 | 144-154 |
| 1-26 | 1,5503 |
| 1-27 | 138-145 |
| 1-28 | 189 (dec.) |
| 1-40 | 1,5305 |
| 1-59 | 1,5515 |
| 1-60 | 140-145 |
| 1-61 | 1.52 |
| 1-62 | 1,542 |
| 1-63 | 106-116 |
| 1-64 | 118-122 |
| 1-65 | 1.52 |
| 1-66 | 1H-NMR (CDCl3) δ: 0.25-0.30 (2H, m), 0.59-0.66 (2H, m), 1.14-1.27 (4H, m), 2.31 (3H, s), 2.67 (2H, q), 3.42 (2H, d), 7.35-7.39 (3H, m), |
| | 7.49 (1H, dd), 7.70-7.83 (3H, m), 8.11 (1H, dd), 8.27 (1H, s), 8.48 (1H, dd), 8.66 (1H, s). |
| 1-79 | 186 |
| 1-80 | 1,5515 |
| 1-81 | 63-69 |
| 1-82 | 68-79 |
| 1-83 | 169-176 |
| 1-84 | 105-119 |
| 1-85 | 93-98 |
| 1-86 | 122-130 |
| 1-87 | 124-128 |
| 1-88 | 1,5057 |
| 1-89 | 1,5373 |
| 1-90 | 68-79 |
| 1-91 | 1,5443 |
| 1-92 | 1,5405 |
| 1-93 | 1,564 |
| 1-96 | 1,5472 |
| 1-97 | 212-216 |
| 1-98 | 1,5502 |
| 1-99 | 88-101 |
| 1-100 | 1,5453 |
| 1-101 | 1,5465 |
| 1-102 | 1,5298 |
| 1-103 | 136-140 |
| 1-104 | 78-89 |
| 1-105 | 1H-NMR (CDCl3) δ: 1.05 (3H, t), 2.22 (3H, s), 2.57 (2H, q), 6.85 (2H, d), 7.01 (1H, t), |
| | 7.13-7.18 (2H, m), 7.26 (1H, dd), 7.38 (2H, s), 7.63 (1H, d), |
| | 7.72 (1H, d), 7.93 (1H, dd), 8.05 (1H, s), 8.28 (1H, s), 8.36 (1H, dd), 9.09 (1H, s). |
| 1-107 | 1H-NMR (CDCl3) δ: 1.12 (3H, t), 2.33 (3H, s), 2.65 (2H, q), 6.61 (1H, d), 6.86-6.98 (2H, m), 7.59-7.35 (6H, m), 7.75 (1H, d), 7.84 (1H, d), |
| | 8.21 (1H, d), 8.33 (1H, s), 8.43 (1H, s), 8.54 (1H, dd). |
| 1-108 | 1H-NMR (CDCl3) δ: 1.11 (3H, t), 2.30 (3H, s), 2.64 (2H, q), 6.67 (1H, d), 6.98-7.11 (3H, m), 7.35-7.39 (3H, m), 7.49 (1H, t), |
| | 7.72 (1H, d), 7.85-7.80 (2H, m), 8.10 (1H, dd), 8.31 (1H, s), 8.48 (1H, dd), 8.76 (1H, s). |
| 1-109 | 1H-NMR (CDCl3) δ: 1.15 (3H, t), 2.34 (3H, s), 2.67 (2H, q), 6.81 (2H, d), 7.15 (2H, d), 7.39-7.44 (3H, m), 7.54 (1H, dd), 7.70 (1H, s), |
| | 7.76 (1H, d), 7.84 (1H, d), 8.19 (1H, dd), 8.33 (1H, s), 8.54-8.52 (2H, m). |
| 1-110 | 1H-NMR (CDCl3) δ: 1.12 (3H, t), 2.34 (3H, s), 2.66 (3H, q), 6.93-6.98 (2H, m), 7.25-7.45 (5H, m), 7.55 (1H, dd), 7.62 (1H, s), 7.76 (1H, d), |
| | 7.84 (1H, d), 8.21 (1H, dd), 8.32 (1H, s), 8.45 (1H, s), 8.54 (1H, dd). |
| 1-111 | 1H-NMR (CDCl3) δ: 2.33 (s, 6H), 6.66-6.76 (m, 1H), 7.19-7.89 (m, 8H), 8.12-8.26 (m, 1H), 8.31-8.61 (m, 4H) |
| 1-112 | 1H-NMR (CDCl3) δ: 1.12 (3H, t), 2.35 (3H, s), 2.66 (2H, q), 6.73 (1H, dd), 7.29 (1H, d), 7.32 (2H, s), 7.44 (1H, ddz), 7.56 (1H, dd), |
| | 7.62 (1H, s), 7.77 (1H, d), 7.82 (1H, d), 8.22 (1H, dd), 8.35 (1H, s), 8.41 (1H, s), 8.47 (1H, dz), 8.55 (1H, dd). |
| 1-114 | 1H-NMR (CDCl3) δ: 1.12 (3H, t), 2.27 (3H, s), 2.62 (2H, q), 4.66 (2H, s), 7.49-7.31 (9H, m), 7.68-7.83 (3H, m), 8.06 (1H, d), 8.27 |
| | (1H, s), 8.45 (1H, dd), 8.75 (1H, s). |
| 1-115 | 1H-NMR (CDCl3) δ: 1.11 (3H, t), 2.30 (3H, s), 2.63 (2H, q), 4.77 (2H, s), 7.40-7.28 (5H, m), 7.50 (1H, dd), 7.66-7.73 (4H, m), 7.84 (1H, d), |
| | 8.14 (1H, dd), 8.27 (1H, s), 8.50 (1H, dd), 8.56 (1H, s). |
| 1-116 | 1H-NMR (CDCl3) δ: 1.14 (3H, t), 2.30 (3H, s), 2.65 (2H, q), 4.64 (2H, s), 7.24-7.39 (7H, m), 7.50 (1H, dd), 7.68-7.73 (2H, m), 7.82 (1H, d), |
| | 8.14 (1H, dd), 8.29 (1H, s), 8.49 (1H, dd), 8.57 (1H, s). |
| 1-117 | 1H-NMR (CDCl3) δ: 1.17 (3H, t), 2.34 (3H, s), 2.67 (2H, q), 4.64 (2H, s), 7.31-7.39 (6H, m), 7.44 (1H, dd), 7.58-7.52 (2H, m), 7.75 (1H, d), |
| | 7.87 (1H, d), 8.23 (1H, d), 8.30 (1H, s), 8.38 (1H, s), 8.55 (1H, d). |
| 1-118 | 1H-NMR (CDCl3) δ: 1.90 (d, 3H), 2.29 (s, 6H), 7.23-7.91 (m, 8H), 8.04-8.14 (m, 1H), 8.22 (s, 1H), 8.43-8.52 (m, 1H), |
| | 8.64 (s, 1H) |
| 1-119 | 1H-NMR (CDCl3) δ: 1.20 (3H, t), 1.76-1.87 (4H, m), 2.32 (3H, s), 2.40 (2H, t), 2.64-2.71 (5H, m), 7.30 (2H, s), 7.41 (1H, dd), |
| | 7.51-7.56 (2H, m), 7.73 (1H, d), 7.90 (1H, d), 8.18-8.23 (2H, m), 8.45 (1H, br s), 8.53 (1H, d). |
| 1-123 | 1H-NMR (CDCl3) δ: 1.20 (3H, t), 1.74-1.89 (4H, m), 2.32 (3H, s), 2.40 (2H, t), 2.64-2.71 (4H, m), 7.30 (2H, s), 7.41 (1H, dd), 7.51-7.56 |
| | (2H, m), 7.73 (1H, d), 7.90 (1H, d), 8.20 (1H, d), 8.23 (1H, s), 8.45 (1H, s), 8.53 (1H, dd). |
| 1-124 | 1H-NMR (CDCl3) δ: 1.21 (3H, t), 1.61-1.76 (6H, m), 2.18-2.22 (2H, m), 2.32 (3H, s), 2.64-2.71 (4H, m), 7.28 (2H, s), 7.41 (1H, dd), |
| | 7.58-7.51 (2H, m), 7.73 (1H, d), 7.89 (1H, d), 8.19 (1H, d), 8.24 (1H, s), 8.47 (1H, s), 8.53 (1H, dd). |
| 1-125 | 1H-NMR (CDCl3) δ: 1.17 (2H, t), 2.30 (3H, s), 2.65 (2H, q), 7.33-7.72 (11H, m), 7.85 (1H, d), 8.13 (1H, d), 8.25 (1H, s), 8.40 (1H, s), |
| | 8.49-8.60 (2H, m). |
| 1-126 | 1H-NMR (CDCl3) δ: 1.17 (2H, t), 2.30 (3H, s), 2.47 (3H, s), 2.65 (2H, q), 7.40-7.62 (10H, m), 7.85 (1H, d), 8.12-8.24 (3H, m), 8.62-8.48 |
| | (3H, m). |
| 1-127 | 1H-NMR (CDCl3) δ: 2.30 (6H, s), 4.62 (2H, s), 7.26-7.41 (7H, m), 7.51 (1H, dd), 7.72-7.82 (3H, m), 8.14 (1H, d), 8.30 (1H, s), 8.50 (1H, d), |
| | 8.57 (1H, s). |
| 1-128 | 1H-NMR (CDCl3) δ: 1.16 (3H, t), 2.35 (3H, s), 2.68 (2H, q), 6.86-6.92 (4H, m), 7.41-7.46 (3H, m), 7.56 (1H, dd), 7.68 (1H, s), 7.77 (1H, d), |
| | 7.83 (1H, d), 8.21 (1H, dd), 8.33 (1H, s), 8.46 (1H, s), 8.55 (1H, dd). |
| 1-129 | 1H-NMR (CDCl3) δ: 1.15 (3H, t), 2.34 (3H, s), 2.67 (2H, q), 6.76 (2H, d), 7.30 (1H, d), 7.38-7.45 (3H, m), 7.56 (1H, dd), 7.63 (1H, s), |
| | 7.76 (1H, d), 7.84 (1H, d), 8.22 (1H, dd), 8.33 (1H, s), 8.43 (1H, s), 8.55 (1H, dd). |
| 1-130 | 1H-NMR (CDCl3) δ: 1.12 (3H, t), 2.33 (3H, s), 2.66 (2H, q), 6.95 (2H, d), 7.35-7.57 (6H, m), 7.75-7.77 (2H, m), 7.83 (1H, d), 8.18 (1H, d), |
| | 8.34 (1H, s), 8.53 (1H, dd), 8.58 (1H, s). |
| 1-131 | 1H-NMR (CDCl3) δ: 1.14 (3H, t), 2.35 (3H, s), 2.67 (2H, q), 6.89 (2H, d), 7.05 (2H, d), 7.38-7.46 (3H, m), 7.60-7.54 (2H, m), 7.77 (1H, d), |
| | 7.84 (1H, d), 8.23 (1H, d), 8.33 (1H, s), 8.41 (1H, s), 8.55 (1H, d). |
| 1-132 | 1H-NMR (CDCl3) δ: 1.11 (3H, t), 2.34 (3H, s), 2.65 (2H, q), 6.89 (2H, d), 7.33 (1H, s), 7.38 (1H, s), 7.42-7.50 (3H, m), 7.54-7.61 (2H, m), |
| | 7.77 (1H, d), 7.84 (1H, d), 8.23 (1H, d), 8.33 (1H, s), 8.40 (1H, s), 8.55 (1H, d). |
| 1-133 | 1H-NMR (CDCl3) δ: 1.14 (3H, t), 2.26 (3H, s), 2.32 (3H, s), 2.66 (2H, q) 6.77 (2H, d), 6.98 (2H, d), 7.38-7.43 (3H, m), 7.53 (1H, dd), |
| | 7.63 (1H, s), 7.74 (1H, d), 7.84 (1H, d), 8.18 (1H, d), 8.31 (1H, s), 8.52 (2H, d). |
| 1-134 | 1H-NMR (CDCl3) δ: 1.17 (3H, t), 2.35 (3H, s), 2.68 (2H, q), 3.74 (3H, s), 6.71 (2H, d), 6.84 (2H, d), 7.42-7.46 (3H, m), 7.54-7.59 (2H, m), |
| | 7.76 (1H, d), 7.86 (1H, d), 8.23 (1H, dd), 8.32 (1H, s), 8.40 (1H, s), 8.55 (1H, dd). |
| 1-138 | 1H-NMR (CDCl3) δ: 1.15 (3H, t), 2.26 (3H, s), 2.62 (2H, q), 7.27-7.36 (5H, m), 7.44-7.51 (3H, m), 7.67 (1H, d), 7.79-7.82 (2H, m), |
| | 8.04 (1H, dd), 8.24 (1H, s), 8.37 (1H, s), 8.44 (1H, dd), 8.80 (1H, s). |
| 1-142 | 1H-NMR (CDCl3) δ: 1.11 (3H, t), 2.34 (3H, s), 2.66 (2H, q), 6.79 (1H, dd), 7.27 (1H, d), 7.32 (2H, s), 7.43 (1H), 7.56 (1H, dd), 7.64 (1H, s), |
| | 7.76 (1H, d), 7.83 (1H, d), 8.21 (1H, dd), 8.34 (1H, s), 8.44 (1H, s), 8.50 (1H, d), 8.55 (1H, dd). |
| 1-143 | 1H-NMR (CDCl3) δ: 1.08 (3H, t), 2.29 (3H, s), 2.62 (2H, q), 6.72 (2H, d), 7.26-7.38 (6H, m), 7.49 (1H, dd), 7.73 (1H, d), 7.79 (1H, d), |
| | 8.07 (1H, d), 8.29-8.34 (2H, m), 8.46 (1H, d). |
| 1-144 | 172-176 |
| 1-145 | 1,537 |
| 1-146 | 1H-NMR (CDCl3) δ: 1.23 (13H, t), 2.36 (3H, s), 2.71 (2H, q), 3.99 (2H, q), 7.37-7.44 (3H, m), 7.58-7.53 (2H, m), 7.75 (1H, d), 7.87 (1H, d), |
| | 8.21 (1H, d), 8.31 (1H, s), 8.44 (1H, s), 8.54 (1H, dd). |
| 1-147 | 1H-NMR (CDCl3) δ: 1.11 (3H, t), 2.15 (3H, s), 2.54 (2H, q), 3.01 (2H, t), 3.79 (2H, t), 7.06 (2H, d), 7.24-7.39 (6H, m), 7.49 (1H, dd), |
| | 7.71-7.66 (2H, m), 7.82 (1H, d), 8.12 (1H, dd), 8.25 (1H, s), 8.49 (1H, dd), 8.60 (1H, s). |
| 1-148 | 1H-NMR (CDCl3) δ: 1.18 (3H, t), 2.37 (3H, s), 2.70 (2H, q), 3.88 (3H, s), 5.33 (1H, s), 7.33 (1H, s), 7.36 (1H, s), 7.43 (1H, dd), 7.56 |
| | (1H, dd), 7.71 (1H, s), 7.77 (1H, d), 7.84 (1H, d), 8.21 (1H, dd), 8.33 (1H, s), 8.48 (1H, s), 8.54 (1H, dd). |
| 1-149 | 1H-NMR (CDCl3) δ: 1.19 (3H, t), 2.33 (3H, s), 2.36 (3H, s), 2.68 (2H, q), 7.17 (2H, d), 7.36 (2H, s), 7.41-7.48 (3H, m), 7.53-7.58 (2H, m), |
| | 7.74 (1H, d), 7.89 (1H, d), 8.22 (1H, dd), 8.26 (1H, s), 8.36 (1H, s), 8.39 (1H, s), 8.55 (1H, dd). |
| 1-150 | 1H-NMR (CDCl3) δ: 1.15 (3H, t), 2.27 (3H, t), 2.63 (2H, q), 3.80 (3H, s), 6.87 (2H, d), 7.36-7.32 (3H, m), 7.51-7.44 (3H, m), 7.67 (1H, d), |
| | 7.75 (1H, s), 7.82 (1H, d), 8.07 (1H, dd), 8.23 (1H, s), 8.33 (1H, s), 8.46 (1H, dd), 8.70 (1H, s). |
| 1-151 | 1H-NMR (CDCl3) δ: 1.15 (3H, t), 2.34 (3H, s), 2.67 (2H, q), 6.64 (1H, dd), 7.11 (1H, d), 7.22 (1H, d), 7.37 (1H, s), 7.38 (1H, s), 7.43 |
| | (1H, dd), 7.55 (1H, dd), 7.67 (1H, s), 7.76 (1H, d), 7.83 (1H, d), 8.20 (1H, dd), 8.33 (1H, s), 8.47 (1H, s), |
| | 8.54 (1H, dd). |
| 1-152 | 1H-NMR (CDCl3) δ: 1.15 (3H, t), 2.35 (3H, s), 2.67 (2H, q), 6.64 (2H, d), 7.37 (1H, s), 7.39 (1H, s), 7.42-7.50 (3H, m), 7.59-7.54 (2H, m), |
| | 7.77 (1H, d), 7.85 (1H, d), 8.23 (1H, dd), 8.33 (1H, s), 8.39 (1H, s), 8.56 (1H, dd). |
| 1-153 | 1H-NMR (CDCl3) δ: 1.14-1.18 (6H, m), 2.34 (3H, s), 2.56 (2H, q), 2.66 (2H, q), 6.79 (2H, d), 7.00 (2H, d), 7.46-7.41 (3H, m), |
| | 7.58-7.53 (2H, m), 7.76 (1H, d), 7.86 (1H, d), 8.22 (1H, dd), 8.31 (1H, s), 8.41 (1H, s), 8.55 (1H, dd). |
| 1-154 | 1H-NMR (CDCl3) δ: 0.90 (3H, t), 1.14 (3H, t), 1.25-1.58 (4H, m), 2.34 (3H, s), 2.52 (2H, q), 2.66 (2H, q), 6.78 (2H, d), 6.98 (2H, d), |
| | 7.41-7.45 (3H, m), 7.53-7.58 (2H, m), 7.76 (1H, d), 7.86 (1H, d), 8.22 (1H, dd), 8.31 (1H, s), 8.41 (1H, s), |
| | 8.55 (1H, dd). |
| 1-160 | 1H-NMR (CDCl3) δ: 2.30 (6H, s), 6.75 (2H, d), 7.29 (2H, d), 7.35-7.40 (3H, m), 7.50 (1H, dd), 7.71-7.82 (3H, m), 8.11 (1H, dd), 8.31 |
| | (1H, s), 8.49 (1H, dd), 8.65 (1H, s). |
| 1-166 | 1H-NMR (CDCl3) δ: 2.31 (6H, s), 6.81 (2H, d), 7.14 (2H, d), 7.34-7.42 (3H, m), 7.50 (1H, dd), 7.70-7.87 (3H, m), 8.13 (1H, dd), 8.31 |
| | (1H, s), 8.49 (1H, dd), 8.69 (1H, s). |
| 1-167 | 1H-NMR (CDCl3) δ: 2.33 (6H, s), 6.92 (1H, d), 7.39-7.45 (3H, m), 7.39-7.45 (3H, m), 7.55 (1H, dd), 7.67 (1H, s), 7.77 (1H, d), 7.83 (1H, d), |
| | 8.21 (1H, dd), 8.33 (1H, s), 8.45 (1H, s), 8.54 (1H, dd). |
| 1-168 | 1H-NMR (CDCl3) δ: 1.07 (3H, t), 2.27 (3H, s), 2.62 (2H, q), 6.92 (2H, d), 7.35-7.29 (3H, m), 7.49-7.43 (3H, m), 7.70 (1H, d), 7.79 (1H, d), |
| | 7.97 (1H, s), 8.02 (1H, dd), 8.32 (1H, s), 8.43 (1H, dd), 8.91 (1H, s). |
| 1-169 | 1H-NMR (CDCl3) δ: 1.13 (3H, t), 2.09 (3H, s), 2.31 (3H, s), 2.64 (2H, q), 6.81 (2H, d), 7.26-7.29 (3H, m), 7.39-7.43 (3H, m), 7.53 (1H, dd), |
| | 7.68 (1H, s), 7.74 (1H, d), 7.87 (1H, d), 8.17 (1H, d), 8.29 (1H, s), 8.56-8.51 (2H, m). |
| 1-170 | 1H-NMR (DMSO-d6) δ: 1.07 (3H, t), 2.51 (3H, s), 2.63 (2H, q), 3.73 (2H, br s), 6.58 (2H, d), 6.70 (2H, d), 7.41 (1H, s), 7.44 (1H, s), |
| | 7.60-7.53 (2H, m), 7.79 (1H, d), 7.93 (1H, d), 8.11 (1H, dd), 8.31 (1H, s), 8.55 (1H, dd), 10.00 (1H, s), |
| | 10.88 (1H, s). |
| 1-171 | 1H-NMR (CDCl3) δ: 1.01 (3H, t), 2.20 (3H, s), 2.53 (2H, q), 6.93 (1H, ddd), 7.01 (1H, d), 7.16 (1H, s), 7.22 (1H, s), 7.33 (1H, dd), 7.45 |
| | (1H, dd), 7.70-7.63 (3H, m), 7.81 (1H, d), 7.92 (1H, dd), 8.05 (1H, dd), 8.21 (1H, s), 8.45 (1H, dd), |
| | 8.70 (1H, s). |
| 1-172 | 1H-NMR (CDCl3) δ: 1.07 (3H, t), 2.25 (3H, s), 2.60 (2H, q), 7.10-7.11 (2H, m), 7.28-7.46 (4H, m), 7.70 (1H, d), 7.78 (1H, d), 7.97 (1H, d), |
| | 8.13 (1H, s), 8.21-8.23 (2H, m), 8.31 (1H, s), 8.41 (1H, dd), 9.26 (1H, d). |
| 1-173 | 1H-NMR (CDCl3) δ: 1.07 (3H, t), 2.25 (3H, s), 2.59 (2H, q), 6.99 (1H, d), 7.16 (1H, s), 7.20 (1H, s), 7.39 (1H, dd), 7.50 (1H, dd), 7.55 |
| | (1H, s), 7.64 (1H, dd), 7.69 (1H, d), 7.84 (1H, d), 7.90 (1H, d), 8.14 (1H, dd), 8.25 (1H, s), 8.52-8.49 |
| | (2H, m). |
| 1-175 | 1H-NMR (CDCl3) δ: 1.16 (3H, t), 2.28 (3H, s), 2.63 (2H, q), 6.81 (1H, d), 7.14 (2H, s), 7.24 (1H, d), 7.39 (1H, dd), 7.48-7.55 (2H, m), 7.70 |
| | (1H, d), 7.90 (1H, d), 8.14-8.20 (2H, m), 8.52-8.50 (2H, m). |
| 1-176 | 1H-NMR (CDCl3) δ: 1.12 (6H, t), 2.63 (4H, q), 6.80 (2H, d), 7.13 (2H, d), 7.33 (1H, dd), 7.39 (2H, s), 7.46 (1H, dd), 7.69 (1H, d), |
| | 7.81-7.78 (2H, m), 8.06 (1H, dd), 8.30 (1H, s), 8.45 (1H, dd), 8.80 (1H, s). |
| 1-177 | 1H-NMR (CDCl3) δ: 1.16 (6H, t), 2.68 (4H, q), 6.75 (2H, d), 7.30 (2H, d), 7.40-7.45 (3H, m), 7.54-7.58 (2H, m), 7.75 (1H, d), 7.86 (1H, d), |
| | 8.22 (1H, dd), 8.32 (1H, s), 8.42 (1H, s), 8.54 (1H, dd). |
| 1-178 | 1H-NMR (CDCl3) δ: 6.78 (2H, d), 7.13 (2H, d), 7.43 (1H, dd), 7.54 (1H, dd), 7.63 (2H, d), 7.70 (1H, d), 7.84-7.77 (3H, m), 8.14 (1H, s), |
| | 8.21 (1H, dd), 8.25 (1H, s), 8.43 (1H, s), 8.54 (1H, dd). |
| 1-179 | 1H-NMR (CDCl3) δ: 6.73 (2H, d), 7.28 (2H, dd), 7.43 (1H, dd), 7.54 (1H, dd), 7.62 (2H, d), 7.71 (1H, d), 7.77-7.84 (3H, m), 8.12 (1H, s), |
| | 8.22 (1H, dd), 8.25 (1H, s), 8.41 (1H, s), 8.54 (1H, dd). |
| 1-180 | 1H-NMR (CDCl3) δ: 2.32 (6H, t), 6.94 (2H, d), 7.33 (2H, s), 7.42 (1H, dd), 7.49-7.62 (4H, m), 7.76 (1H, d), 7.82 (1H, d), 8.20 (1H, dd), |
| | 8.35 (1H, s), 8.46 (1H, br s), 8.54 (1H, dd). |
| 1-181 | 1H-NMR (CDCl3) δ: 1.17 (3H, t), 2.35 (3H, s), 2.68 (2H, q), 7.08 (1H, dd), 7.14 (1H, d), 7.37 (1H, s), 7.39 (1H, s), 7.44 (1H, dd), |
| | 7.54-7.60 (2H, m), 7.76 (1H, d), 7.87 (1H, d), 8.09 (1H, d), 8.23 (1H, dd), 8.32 (1H, s), 8.39 (1H, s), |
| | 8.55 (1H, dd). |
| 1-182 | 1H-NMR (CDCl3) δ: 2.35 (6H, s), 7.08 (1H, dd), 7.14 (1H, d), 7.38 (2H, s), 7.44 (1H, dd), 7.52-7.60 (2H, m), 7.77 (1H, d), 7.86 (1H, d), |
| | 8.09 (1H, d), 8.24 (1H, dd), 8.32 (1H, s), 8.38 (1H, s), 8.56 (1H, dd). |
| 1-197 | 1H-NMR (CDCl3) δ: 6.76 (2H, d), 7.19 (2H, d), 7.28 (1H, s), 7.40-7.48 (2H, m), 7.66 (1H, d), 7.72 (2H, s), 7.79 (1H, d), 8.04 (1H, s), 8.17 |
| | (1H, dd), 8.21 (1H, s), 8.53 (1H, dd). |
| 1-201 | 1H-NMR (CDCl3) δ: 2.41 (3H, s), 6.79 (2H, d), 7.14 (2H, d), 7.43 (1H, dd), 7.48 (1H, s), 7.52-7.58 (3H, m), 7.71 (1H, d), 7.84-7.89 |
| | (2H, m), 8.21-8.32 (2H, m), 8.42 (1H, s), 8.53 (1H, dd). |
| 2-1 | 1H-NMR (CDCl3) δ: 1.18 (3H, t), 2.32 (3H, s), 2.66 (2H, q), 6.27 (2H, dd), 6.81 (2H, br s), 7.23 (2H, s), 7.42 (1H, dd), 7.61-7.52 (2H, m), |
| | 7.75 (1H, d), 7.86 (1H, d), 8.20 (1H, dd), 8.28 (1H, d), 8.47 (1H, s), 8.53 (1H, dd). |
| 2-2 | 1H-NMR (CDCl3) δ: 2.49 (s, 6H), 6.32-6.37 (m, 1H), 6.54-6.60 (m, 1H), 7.58-7.80 (m, 5H), 7.94 (d, 1H), 8.08-8.25 (m, 2H), |
| | 8.47-8.68 (m, 2H), 9.33 (s, 1H), 9.97 (s, 1H) |
| 2-3 | 1H-NMR (CDCl3) δ: 2.33 (s, 6H), 6.24-6.32 (m, 1H), 6.69 (s, 1H), 6.83 (s, 1H), 7.21 (s, 2H), 7.36-7.61 (m, 3H), 7.75 (d, 1H), 7.86 (d, 1H), |
| | 8.13-8.22 (m, 1H), 8.30 (s, 1H), 8.44-8.57 (m, 2H) |
| 2-4 | 1H-NMR (CDCl3) δ: 2.25 (s, 6H), 6.47-6.60 (m, 1H), 7.10 (s, 2H), 7.30-7.82 (m, 7H), 8.03-8.24 (m, 2H), 8.41-8.52 (m, 1H), |
| | 8.60 (s, 1H) |
| 2-5 | 1H-NMR (CDCl3) δ: 2.31 (s, 6H), 7.35-7.77 (m, 6H), 7.85 (d, 1H), 8.15 (d, 1H), 8.29 (s, 1H), 8.47-8.61 |
| | (m, 2H) |
| 2-6 | 1H-NMR (CDCl3) δ: 2.32 (s, 6H), 6.36 (d, 1H), 7.17 (s, 2H), 7.37-7.61 (m, 4H), 7.73 (d, 1H), 7.86 (d, 1H), 8.20 (d, 1H), 8.29 (s, 1H), 8.43 |
| | (s, 1H), 8.50-8.58 (s, 1H) |
| 2-7 | 1H-NMR (CDCl3) δ: 2.32 (s, 6H), 6.36 (d, 1H), 7.17 (s, 2H), 7.37-7.61 (m, 4H), 7.73 (d, 1H), 7.86 (d, 1H), 8.20 (d, 1H), 8.29 (s, 1H), 8.43 |
| | (s, 1H), 8.50-8.58 (s, 1H) |
| 2-8 | 1H-NMR (CDCl3) δ: 1.29 (3H, t), 2.21 (6H, s), 4.20 (2H, q), 5.81 (1H, q), 7.14-7.17 (3H, m), 7.33 (1H, t), 7.58-7.53 (4H, m), 7.81 (1H, s), |
| | 7.96 (1H, s). |
| 2-12 | 1H-NMR (CDCl3) δ: 2.26 (6H, s), 5.82 (1H, q), 7.14-7.21 (3H, m), 7.30 (1H, t), 7.44 (1H, t), 7.50-7.58 (3H, m), 7.69 (1H, d), 7.85-7.82 |
| | (2H, m), 8.10 (1H, dd), 8.28 (1H, s), 8.64 (1H, d). |
| 2-13 | 1H-NMR (CDCl3) δ: 2.25 (6H, d), 5.81 (1H, q), 7.20 (2H, s), 7.36 (1H, dd), 7.44-7.58 (3H, m), 7.73-7.68 (2H, m), 7.81 (1H, d), 8.09 |
| | (1H, dd), 8.25 (1H, s), 8.48 (1H, dd), 8.65 (1H, s). |
| 2-24 | 1H-NMR (CDCl3) δ: 5.20-5.35 (2H, m), 7.34-7.40 (3H, m), 7.49-7.52 (1H, m), 7.61 (1H, s), 7.63 (1H, s), 7.75 (1H, d), 7.90 (1H, d), |
| | 8.01 (1H, s), 8.09-8.11 (1H, m), 8.25 (1H, s), 8.47-8.48 (1H, m), 8.70 (1H, s) |
| 2-27 | 1H-NMR (acetone-d6) δ: 1.15 (3H, t), 2.34 (3H, s), 2.74 (2H, q), 7.24 (2H, d), 7.50-7.62 (4H, m), 7.80 (1H, d), 7.84 (1H, d), 7.95 (1H, s), |
| | 8.03-8.06 (2H, m), 8.13 (1H, dz), 8.46 (1H, s), 9.25 (1H, s), 9.73 (1H, s). |
| 2-28 | 1H-NMR (CDCl3) δ: 2.38 (3H, s), 7.16 (1H, dd), 7.24-7.31 (3H, m), 7.43 (1H, t), 7.48-7.55 (2H, m), 7.64 (2H, dd), 7.83 (1H, d), |
| | 8.04-8.16 (3H, m), 8.28 (1H, br s), 8.69 (1H, d). |
| 2-30 | 1H-NMR (CDCl3) δ: 1.09 (3H, t), 2.22 (3H, s), 2.60 (2H, q), 7.06-7.13 (3H, m), 7.24 (1H, t), 7.38 (1H, t), 7.44-7.52 (2H, m), 7.63 (1H, s), |
| | 7.68 (1H, d), 7.79 (1H, d), 8.02 (1H, t), 8.12 (1H, s), 8.30 (1H, s), 8.67 (1H, d). |
| 2-31 | 1H-NMR (CDCl3) δ: 2.38 (3H, s), 7.15-7.33 (3H, m), 7.57-7.46 (4H, m), 7.68 (1H, s), 7.74 (1H), 7.90 (1H, d), 8.01-8.15 (2H, m), 8.31 |
| | (1H, s), 8.67 (1H, d). |
| 2-32 | 1H-NMR (CDCl3) δ: 7.21 (1H, dd), 7.33 (1H, t), 7.43 (2H, s), 7.50-7.59 (2H, m), 7.63 (1H, s), 7.69 (1H, s), 7.76 (1H, d), 8.04-7.89 (2H, m), |
| | 8.16 (1H, t), 8.32 (1H, t), 8.64 (1H, d). |
| 2-34 | 1H-NMR (CDCl3) δ: 7.19 (1H, dd), 7.31 (1H, t), 7.48-7.58 (2H, m), 7.63 (1H, s), 7.69 (1H, s), 7.78 (1H, d), 7.85 (2H, s), 7.94 (1H, d), |
| | 8.17-8.11 (2H, m), 8.32 (1H, s), 8.66 (1H, d). |
| 2-35 | 1H-NMR (acetone-d6) δ: 1.20 (3H, t), 2.40 (3H, s), 2.80 (2H, q), 7.30 (2H, d), 7.43 (1H, td), 7.68-7.57 (2H, m), 7.83-7.89 (3H, m), |
| | 7.95 (1H, d), 8.01 (1H, s), 8.18 (1H, d), 8.50 (1H, s), 9.31 (1H, s), 9.84 (1H, s). |
| 2-36 | 1H-NMR (acetone-d6) δ: 1.15 (4H, t), 2.34 (3H, s), 2.74 (2H, q), 7.23-7.31 (4H, m), 7.52 (1H, t), 7.79-7.84 (2H, m), 7.95 (1H, s), |
| | 8.10-8.15 (3H, m), 8.43 (1H, s), 9.24 (1H, s), 9.75 (1H, s). |
| 2-37 | 1H-NMR (CDCl3) δ: 1.17 (3H, t), 2.30 (3H, s), 2.66 (2H, q), 7.15 (2H, s), 7.21-7.28 (1H, m), 7.33-7.53 (3H, m), 7.87-7.67 (5H, m), |
| | 8.30 (1H, s), 8.52 (1H, d). |
| 2-38 | 1H-NMR (CDCl3) δ: 1.15 (3H, t), 2.29 (3H, s), 2.65 (2H, q), 6.90-7.06 (2H, m), 7.15 (2H, s), 7.44-7.52 (2H, m), 7.67-7.72 (2H, m), |
| | 7.89-7.83 (2H, m), 8.14 (1H, dd), 8.29 (1H, s), 8.57 (1H, d). |
| 2-39 | 1H-NMR (CDCl3) δ: 1.18 (3H, t), 2.31 (3H, s), 2.67 (2H, q), 7.16-7.26 (4H, m), 7.53-7.48 (2H, m), 7.68-7.88 (5H, m), 8.30 (1H, s), |
| | 8.66 (1H, d). |
| 2-40 | 1H-NMR (CDCl3) δ: 1.10 (3H, t), 2.22 (3H, s), 2.59 (2H, d), 6.91 (2H, t), 7.09 (2H, s), 7.30-7.47 (3H, m), 7.67-7.63 (2H, m), 7.78 (1H, d), |
| | 7.90 (1H, s), 8.25 (1H, s), 8.50 (1H, s). |
| 2-41 | 1H-NMR (CDCl3) δ: 2.36 (3H, s), 6.98 (2H, t), 7.24 (1H, s), 7.37-7.52 (3H, m), 7.57 (1H, s), 7.68 (1H, s), 7.74 (1H, d), 7.89-7.86 (2H, m), |
| | 8.16 (1H, s), 8.28 (1H, s). |
| 2-44 | 1H-NMR (CDCl3) δ: 1.16 (3H, t), 2.30 (3H, s), 2.66 (2H, q), 7.15 (2H, s), 7.39-7.53 (3H, m), 7.67-7.74 (2H, m), 7.85-7.88 (2H, m), |
| | 8.31 (1H, s), 8.38 (1H, dt), 8.62-8.56 (1H, m), 8.77 (1H, d). |
| 2-50 | 1H-NMR (CDCl3) δ: 2.37 (3H, s), 7.24-7.31 (3H, m), 7.44-7.53 (2H, m), 7.61-7.67 (2H, m), 7.87 (1H, d), 7.99 (1H, s), 8.06-8.10 (2H, m), |
| | 8.25 (1H, s), 8.39-8.36 (1H, m), 9.03 (1H, s). |
| 2-51 | 1H-NMR (CDCl3) δ: 2.29 (6H, s), 7.13 (2H, s), 7.35-7.37 (1H, m), 7.50-7.52 (2H, m), 7.68-7.71 (2H, m), 7.83-7.87 (2H, m), 8.08 |
| | (1H, d), 8.29 (1H, s), 8.46-8.48 (1H, m), 8.77 (1H, s) |
| 2-55 | 1H-NMR (CDCl3) δ: 1.11 (3H, t), 2.22 (3H, s), 2.60 (2H, q), 7.12 (2H, s), 7.26-7.31 (1H, m), 7.42 (1H, t), 7.54 (1H, s), 7.66-7.64 (2H, m), |
| | 7.76 (1H, d), 7.96 (1H, dd), 8.05 (1H, s), 8.26 (1H, s), 8.39 (1H, dd), 8.99 (1H, s). |
| 2-59 | 1H-NMR (CDCl3) δ: 2.34 (3H, s), 7.24 (1H, s), 7.33 (1H, dd), 7.51-7.46 (2H, m), 7.60 (1H, s), 7.67 (1H, s), 7.72 (1H, d), 7.86 (1H, d), |
| | 8.02-8.07 (2H, m), 8.27 (1H, s), 8.44 (1H, dd), 8.85 (1H, s). |
| 2-61 | 1H-NMR (CDCl3) δ: 7.35-7.42 (3H, m), 7.51 (1H, t), 7.66-7.76 (3H, m), 7.87 (1H, d), 8.12-8.06 (2H, m), 8.26 (1H, s), 8.47 (1H, dd), |
| | 8.67 (1H, s). |
| 2-62 | 1H-NMR (CDCl3) δ: 7.30-7.35 (1H, m), 7.47 (1H, dt), 7.61 (2H, s), 7.68-7.75 (3H, m), 7.86 (1H, d), 8.03 (1H, t), 8.24-8.26 (2H, m), |
| | 8.45-8.41 (1H, m), 8.86 (1H, s). |
| 2-63 | 1H-NMR (CDCl3) δ: 7.34-7.41 (1H, m), 7.54 (1H, t), 7.66-7.68 (2H, m), 7.78-7.92 (4H, m), 8.06-8.16 (2H, m), 8.30 (1H, s), |
| | 8.52-8.47 (1H, m), 8.60 (1H, s). |
| 2-72 | 1H-NMR (CDCl3) δ: 7.23-7.43 (m, 6H), 7.49-7.59 (m, 3H), 7.63-7.90 (m, 3H), 8.09-8.16 (m, 1H), 8.32-8.42 (m, 1H), |
| | 8.46-8.60 (m, 2H) |
| 2-73 | 1H-NMR (CDCl3) δ: 7.08 (d, 2H), 7.19-7.83 (m, 10H), 7.93 (d, 1H), 8.17-8.30 (m, 2H), 8.37 (s, 1H), 8.55 (d, 1H) |
| 2-76 | 1H-NMR (CDCl3) δ: 2.31 (s, 6H), 6.45 (d, 1H), 7.16 (s, 2H), 7.36-7.63 (m, 4H), 7.74 (d, 1H), 7.86 (d, 1H), 8.18 (d, 1H), 8.29 (s, 1H), |
| | 8.42 (m, 2H) |
| 2-77 | 1H-NMR (CDCl3) δ: 2.29 (6H, d), 5.84 (1H, q), 7.24 (2H, s), 7.43 (1H, dd), 7.51-7.61 (4H, m), 7.75 (1H, d), 7.84 (1H, d), 8.21 (1H, d), |
| | 8.28 (1H, s), 8.43 (1H, s), 8.54 (1H, dd). |
| 2-82 | 1H-NMR (CDCl3) δ: 2.16 (3H, s), 7.40-7.63 (6H, m), 7.76-7.78 (1H, m), 7.90-7.93 (1H, m), 8.08 (1H, s), 8.28-8.28 (1H, m), |
| | 8.38-8.39 (1H, m), 8.57-8.63 (1H, m), 8.77 (1H, d) |
| 2-83 | 1H-NMR (CDCl3) δ: 2.17 (3H, s), 7.34-7.40 (3H, m), 7.49-7.52 (1H, m), 7.60-7.62 (2H, m), 7.75 (1H, d), 7.90 (1H, d), 8.02 (1H, s), |
| | 8.08-8.11 (1H, m), 8.25 (1H, s), 8.47-8.48 (1H, m), 8.73 (1H, s) |
| 2-84 | 1H-NMR (CDCl3) δ: 2.36 (6H, s), 7.16 (2H, s), 7.43-7.61 (4H, m), 7.70 (1H, s), 7.75-7.76 (1H, m), 7.87-7.89 (1H, m), 8.26-8.32 |
| | (3H, m), 8.55-8.57 (1H, m) |
| 2-87 | 1H-NMR (CDCl3) δ: 1.15 (3H, t), 2.28 (3H, s), 2.64 (2H, q), 7.14 (2H, s), 7.36 (1H, dd), 7.49 (1H, t), 7.57 (1H, s), 7.74-7.70 (3H, m), |
| | 7.83 (1H, d), 8.09 (1H, d), 8.28 (1H, s), 8.47 (1H, dd), 8.68 (1H, s). |
| 2-98 | 1H-NMR (CDCl3) δ: 2.33 (6H, s), 6.68 (1H, d), 7.14 (2H, s), 7.43-7.48 (1H, m), 7.52-7.61 (3H, m), 7.75 (1H, d), 7.86 (1H, d), |
| | 8.34 (1H, s), 8.41-8.42 (1H, m), 8.65-8.69 (2H, m) |
| 2-95 | 1H-NMR (CDCl3) δ: 7.13 (s, 2H), 7.34-7.43 (m, 1H), 7.47-7.56 (m, 1H), 7.59 (s, 1H), 7.67-7.78 (m, 3H), 7.84 (d, 1H), 8.14 (d, 1H), |
| | 8.29 (s, 1H), 8.46-8.64 (m, 2H) |
| 2-96 | 1H-NMR (CDCl3) δ: 2.27 (s, 6H), 2.33 (s, 3H), 6.19 (d, 1H), 7.14 (s, 2H), 7.32-7.53 (m, 3H), 7.70 (d, 1H), 7.78-7.89 (m, 2H), 8.09 (d, 1H), |
| | 8.27 (s, 1H), 7.43-8.52 (m, 1H), 8.75 (s, 1H) |
| 2-97 | 1H-NMR (CDCl3) δ: 2.16 (s, 3H), 2.30 (s, 6H), 7.15 (s, 2H), 7.25-7.30 (m, 1H), 7.35-7.43 (m, 1H), 7.47-7.56 (m, 2H), 7.69-7.88 (m, 3H), |
| | 8.13 (d, 1H), 8.29 (s, 1H), 8.46-8.54 (m, 1H), 8.64 (s, 1H) |
| 2-99 | 1H-NMR (CDCl3) δ: 6.63 (1H, d), 7.15-7.20 (3H, m), 7.33 (2H, s), 7.41-7.58 (5H, m), 7.79 (1H, d), 7.89-7.95 (2H, m), 8.31 (1H, d), |
| | 8.40-8.42 (1H, m), 8.67-8.70 (2H, m) |
| 2-100 | 1H-NMR (CDCl3) δ: 6.62 (1H, d), 7.15 (2H, d), 7.20-7.20 (1H, m), 7.33 (2H, s), 7.40-7.58 (5H, m), 7.78 (1H, d), 7.86-7.92 (2H, m), |
| | 8.19-8.22 (1H, m), 8.27 (1H, s), 8.43-8.45 (1H, m), 8.52-8.53 (1H, m) |
| 2-101 | 1H-NMR (CDCl3) δ: 6.57 (d, 1H), 7.17 (s, 1H), 7.34-7.58 (m, 5H), 7.70-7.97 (m, 4H), 8.06 (s, 2H), 8.11-8.18 (m, 1H), 8.22 (s, 1H), |
| | 8.46-8.53 (m, 1H), 8.57 (s, 1H) |
| 2-102 | 1H-NMR (CDCl3) δ: 6.78 (d, 1H), 7.14-7.91 (m, 12H), 8.06-8.29 (m, 2H), 8.44-8.74 (m, 2H) |
| 2-103 | 1H-NMR (CDCl3) δ: 2.32 (6H, d), 5.90 (1H, q), 7.26 (2H, s), 7.43-7.47 (1H, m), 7.54 (1H, t), 7.65 (1H, s), 7.74-7.86 (4 H, m), 8.32 |
| | (1H, s), 8.41 (1H, d), 8.75-8.63 (2H, m). |
| 2-105 | 1H-NMR (CDCl3) δ: 1.14 (3H, t), 2.27 (3H, s), 2.64 (2H, q), 7.12 (2H, s), 7.33 (1H, dd), 7.47 (1H, t), 7.69 (1H, d), 7.94-7.82 (4H, m), |
| | 8.03 (1H, d), 8.29 (1H, s), 8.44 (1H, d), 8.86 (1H, s). |
| 2-106 | 1H-NMR (CDCl3) δ: 2.32 (s, 6H), 6.74 (d, 1H), 6.83 (s, 1H), 7.14 (s, 2H), 7.36-7.58 (m, 3H), 7.65-7.88 (m, 3H), 8.18 (d, 1H), 8.31 (s, 1H), |
| | 8.46-8.58 (m, 2H) |
| 2-107 | 1H-NMR (CDCl3) δ: 2.33 (s, 6H), 6.71 (d, 1H), 7.12 (s, 2H), 7.38-7.48 (m, 1H), 7.50-7.63 (m, 2H), 7.68-7.87 (m, 3H), 8.21 (d, 1H), 8.33 |
| | (s, 1H), 8.44 (s, 1H), 8.52-8.59 (m, 1H) |
| 2-108 | 1H-NMR (CDCl3) δ: 2.34 (6H, s), 7.14 (2H, s), 7.42-7.47 (1H, m), 7.52-7.55 (1H, m), 7.68 (1H, s), 7.75 (1H, d), 7.85 (2H, d), 7.94 (1H, s), |
| | 8.34 (1H, s), 8.40-8.41 (1H, m), 8.66-8.70 (2H, m) |
| 2-109 | 1H-NMR (CDCl3) δ: 2.25 (6H, s), 7.10 (2H, s), 7.28-7.30 (1H, m), 7.41-7.43 (1H, m), 7.69 (1H, d), 7.79 (1H, d), 7.91-7.94 (3H, m), |
| | 8.29-8.32 (2H, m), 8.38-8.39 (1H, m), 9.19 (1H, s) |
| 2-110 | 1H-NMR (CDCl3) δ: 2.33 (s, 6H), 5.93 (d, 1H), 7.13 (s, 2H), 7.39-7.48 (m, 1H), 7.51-7.60 (m, 2H), 7.66 (s, 1H), 7.72-7.87 (m, 2H), |
| | 8.14-8.24 (m, 1H), 8.33 (s, 1H), 8.45 (s, 1H), 8.50-8.58 (m, 1H), 10.07 (s, 1H) |
| 2-111 | 1H-NMR (CDCl3) δ: 2.34 (s, 6H), 3.40 (s, 6H), 5.45 (s, 1H), 6.51 (d, 1H), 7.15 (s, 2H), 7.40-7.61 (m, 4H), 7.77 (d, 1H), 7.86 (d, 1H), |
| | 8.19-8.26 (m, 1H), 8.30 (s, 1H), 8.38 (d, 1H), 8.52-8.62 (m, 1H) |
| 2-112 | 1H-NMR (CDCl3) δ: 2.30 (s, 6H), 3.97 (s, 3H), 6.77 (d, 1H), 7.13 (s, 2H), 7.35-7.44 (m, 1H), 7.47-7.60 (m, 2H), 7.64 (s, 1H), 7.72 (d, 1H), |
| | 7.85 (d, 1H), 8.10-8.20 (m, 2H), 8.28 (s, 1H), 8.46-8.60 (m, 2H) |
| 2-113 | 1H-NMR (CDCl3) δ: 2.33 (s, 6H), 7.14 (s, 2H), 7.37-7.46 (m, 1H), 7.50-7.64 (m, 2H), 7.76 (d, 1H), 7.85 (d, 1H), 8.15-8.36 (m, 4H), |
| | 8.44 (s, 1H), 8.50-8.57 (m, 1H) |
| 2-114 | 1H-NMR (CDCl3) δ: 2.34 (s, 6H), 2.63 (s, 3H), 6.89 (s, 1H), 7.14 (s, 2H), 7.39-7.49 (m, 2H), 7.52-7.66 (m, 2H), 7.76 (d, 1H), 7.83 (d, 1H), |
| | 8.17-8.27 (m, 1H), 8.34 (s, 1H), 8.42 (s, 1H), 8.50-8.61 (m, 1H) |
| 2-115 | 1H-NMR (CDCl3) δ: 2.33 (s, 6H), 7.17 (s, 2H), 7.36-7.45 (m, 1H), 7.46-7.58 (m, 2H), 7.64 (s, 1H), 7.74 (d, 1H), 7.85 (d, 1H), 8.14-8.20 (m, |
| | 1H), 8.31 (s, 1H), 8.47-8.58 (m, 2H) |
| 2-116 | 1H-NMR (CDCl3) δ: 2.32 (s, 6H), 7.15 (s, 2H), 7.35-7.46 (m, 2H), 7.49-7.59 (m, 1H), 7.65 (s, 1H), 7.75 (d, 1H), 7.86 (d, 1H), 8.13-8.20 |
| | (m, 1H), 8.30 (s, 1H), 8.46-8.60 (m, 2H) |
| 2-118 | 1H-NMR (CDCl3) δ: 2.36 (6H, s), 7.15 (2H, s), 7.43-7.46 (1H, m), 7.57-7.59 (3H, m), 7.77-7.84 (2H, m), 8.23-8.25 (1H, m), |
| | 8.35 (2H, d), 8.55-8.57 (1H, m) |
| 2-119 | 1H-NMR (CDCl3) δ: 2.31 (6H, s), 7.12 (2H, s), 7.36-7.42 (1H, m), 7.50-7.52 (1H, m), 7.64 (1H, s), 7.75-7.80 (2H, m), 7.94 (1H, s), |
| | 8.09-8.14 (1H, m), 8.33 (1H, s), 8.48-8.50 (1H, m), 8.69 (1H, s) |
| 2-120 | 1H-NMR (CDCl3) δ: 2.34 (6H, s), 7.12 (2H, s), 7.41-7.43 (1H, m), 7.53-7.56 (1H, m), 7.61 (1H, s), 7.70-7.85 (3H, m), 8.18-8.20 (1H, |
| | m), 8.33 (1H, s), 8.49-8.54 (2H, m) |
| 2-121 | 1H-NMR (CDCl3) δ: 1.33 (3H, t), 2.31 (6H, s), 4.25 (2H, q), 5.78 (1H, q), 6.86 (1H, s), 6.97 (1H, s), 7.41-7.46 (3H, m), 7.61-7.55 (3H, |
| | m), 8.01 (1H, s). |
| 2-123 | 1H-NMR (CDCl3) δ: 2.28 (6H, s), 5.79 (1H, q), 6.97 (1H, s), 7.35-7.50 (4H, m), 7.68-7.81 (3H, m), 8.11 (1H, d), 8.26 (1H, s), |
| | 8.47-8.59 (2H, m). |
| 2-124 | 1H-NMR (CDCl3) δ: 2.32 (6H, s), 7.11-7.36 (5H, m), 7.47-7.65 (2H, m), 7.74-7.84 (3H, m), 8.09-8.14 (1H, m), 8.38 |
| | (1H, s), 8.65 (1H, d) |
| 2-125 | 1H-NMR (CDCl3) δ: 2.35 (6H, s), 7.14 (1H, s), 7.23 (2H, s), 7.44-7.48 (1H, m), 7.55-7.57 (2H, m), 7.78 (1H, d), 7.86 |
| | (1H, d), 8.37 (1H, s), 8.42-8.43 (1H, m), 8.66-8.71 (2H, m) |
| 2-126 | 1H-NMR (CDCl3) δ: 1.37 (t, 3H), 2.35 (s, 6H), 4.34 (q, 2H), 7.13 (s, 2H), 7.40-7.48 (m, 1H), 7.52-7.61 (m, 1H), 7.66 (s, 1H), 7.77 (d, |
| | 1H), 7.84 (d, 1H), 8.07 (s, 1H), 8.19-8.27 (m, 1H), 8.34 (s, 1H), 8.41 (s, 1H), 8.52-8.58 (m, 1H) |
| 2-128 | 1H-NMR (CDCl3) δ: 1.19 (3H, t), 2.34 (3H, s), 2.68 (2H, q), 7.20 (1H, s), 7.24 (1H, s), 7.67-7.43 (3H, m), 7.76 (1H, d), 7.86 |
| | (1H, d), 8.36 (1H, s), 8.42 (1H, s), 8.62-8.75 (2H, m). |
| 2-133 | 1H-NMR (CDCl3) δ: 2.35 (s, 6H), 7.21 (s, 2H), 7.38-7.46 (m, 1H), 7.51-7.60 (m, 1H), 7.64 (s, 1H), 7.72-7.86 (m, 2H), 8.19-8.27 (m, |
| | 1H), 8.35 (s, 1H), 8.44 (s, 1H), 8.51-8.59 (m, 1H) |
| 2-134 | 1H-NMR (CDCl3) δ: 1.18 (3H, t), 2.34 (3H, s), 2.68 (2H, q), 7.19 (1H, s), 7.23 (1H, s), 7.42 (1H, dd), 7.54 (1H, t), 7.64 (1H, s), 7.75 |
| | (1H, d), 7.83 (1H, d), 8.18 (1H, d), 8.34 (1H, s), 8.54-8.52 (2H, m). |
| 2-139 | 1H-NMR (CDCl3) δ: 2.33 (s, 6H), 7.20 (s, 2H), 7.35-7.57 (m, 2H), 7.60-7.91 (m, 3H), 8.08-8.19 (m, 1H), 8.34 (s, 1H), |
| | 8.46-8.65 (m, 2H) |
| 2-141 | 1H-NMR (CDCl3) δ: 2.32 (6H, s), 6.42-6.43 (1H, m), 6.87-6.88 (1H, m), 7.14 (2H, s), 7.23-7.31 (6H, m), 7.53-7.54 (1H, |
| | m), 7.75 (1H, d) |
| 2-142 | 1H-NMR (CDCl3) δ: 2.35 (s, 6H), 7.17 (s, 2H), 7.36-7.59 (m, 3H), 7.70 (s, 1H), 7.76 (d, 1H), 7.83 (d, 1H), 8.19 (d, 1H), 8.35 (s, 1H), |
| | 8.46-8.59 (m, 2H) |
| 2-143 | 1H-NMR (acetone-d6) δ: 2.29 (s, 6H), 7.15 (2H), 7.41-7.58 (m, 3H), 7.76-7.88 (m, 1H), 7.95-8.08 (m, 2H), 8.34-8.55 (m, 2H), 9.24 (s, |
| | 1H), 9.91 (s, 1H) |
| 2-144 | 1H-NMR (CDCl3) δ: 2.32 (s, 6H), 7.20 (s, 2H), 7.35-7.57 (m, 2H), 7.66-7.88 (m, 3H), 8.09-8.20 (m, 1H), 8.33 (s, 1H), |
| | 8.47-8.54 (m, 1H), 8.61 (s, 1H) |
| 2-145 | 1H-NMR (CDCl3) δ: 2.34 (s, 6H), 7.36-7.45 (m, 3H), 7.49-7.61 (m, 2H), 7.73 (d, 1H), 7.85 (d, 1H), 8.14-8.20 (m, 1H), 8.28 (s, 1H), |
| | 8.44-8.55 (m, 2H) |
| 2-146 | 1H-NMR (CDCl3) δ: 2.31 (s, 6H), 7.06 (s, 2H), 7.36-7.45 (m, 1H), 7.49-7.58 (m, 1H), 7.66 (s, 1H), 7.68-7.90 (m, 4H), 8.13-8.23 (m, |
| | 1H), 8.32 (s, 1H), 8.49-8.59 (m, 2H) |
| 2-147 | 1H-NMR (CDCl3) δ: 2.29 (s, 6H), 7.00 (s, 2H), 7.36-7.44 (m, 1H), 7.48-7.57 (m, 1H), 7.67-7.73 (m, 2H), 7.83-7.93 (m, 3H), 8.14-8.27 (m, |
| | 2H), 8.46 (s, 1H), 8.49-8.57 (m, 1H) |
| 2-148 | 1H-NMR (CDCl3) δ: 2.33 (6H, s), 7.10 (2H, s), 7.42-7.44 (1H, m), 7.54-7.57 (1H, m), 7.71-7.84 (3H, m), 8.14 (1H, s), 8.21 (1H, d), |
| | 8.29-8.33 (2H, m), 8.47 (1H, s), 8.54-8.55 (1H, m) |
| 2-149 | 1H-NMR (CDCl3) δ: 1.13 (3H, t), 2.25 (3H, s), 2.62 (2H, q), 7.08 (2H, s), 7.33 (1H, dd), 7.45 (1H, t), 7.68 (1H, d), 7.77 (1H, d), |
| | 8.01-8.12 (3H, m), 8.24 (1H, s), 8.31 (1H, s), 8.43 (1H, dd), 8.98 (1H, s). |
| 2-150 | 1H-NMR (CDCl3) δ: 1.14 (3H, t), 2.29 (3H, s), 2.65 (2H, q), 7.08 (2H, s), 7.15-7.33 (3H, m), 7.46-7.57 (2H, m), 7.71 (1H, d), 7.78 |
| | (1H, d), 7.88 (1H, s), 8.10-8.15 (2H, m), 8.26 (1H, s), 8.33 (1H, s), 8.64 (1H, d). |
| 2-151 | 1H-NMR (CDCl3) δ: 2.41 (3H, s), 7.20-7.22 (2H, m), 7.34-7.36 (1H, m), 7.46 (1H, s), 7.54-7.59 (2H, m), 7.76 (1H, d), |
| | 7.89-7.91 (2H, m), 8.17-8.22 (2H, m), 8.36 (2H, s), 8.64 (1H, d) |
| 2-152 | 1H-NMR (CDCl3) δ: 7.40-7.45 (1H, m), 7.50-7.55 (3H, m), 7.79-7.86 (2H, m), 8.16 (1H, s), 8.35-8.40 (2H, m), 8.46 (2H, d), |
| | 8.56-8.63 (1H, m), 8.84 (1H, d) |
| 2-153 | 1H-NMR (CDCl3) δ: 2.35 (6H, s), 7.12 (2H, s), 7.41-7.44 (1H, m), 7.54-7.57 (1H, m), 7.72-7.84 (3H, m), 8.18-8.21 (1H, m), |
| | 8.31-8.35 (2H, m), 8.49-8.55 (2H, m) |
| 2-154 | 1H-NMR (CDCl3) δ: 2.34 (s, 6H), 7.04 (s, 2H), 7.39-7.62 (m, 2H), 7.66 (s, 1H), 7.72-7.86 (m, 2H), 8.16-8.26 (m, 1H), 8.35 (s, |
| | 1H), 8.40 (s, 1H), 8.50-8.61 (m, 1H), 8.77 (s, 1H) |
| 2-155 | 1H-NMR (CDCl3) δ: 2.26 (s, 6H), 6.35 (d, 1H), 7.03-7.54 (m, 6H), 7.59-8.12 (m, 5H), 8.20-8.52 (m, 3H), |
| | 8.85 (s, 1H) |
| 2-158 | 1H-NMR (CDCl3) δ: 0.94 (3H, t, J = 7.6 Hz), 2.18 (3H, s), 2.50 (2H, q, J = 7.5 Hz), 6.18 (1H, d, J = 8.4 Hz), 7.04-7.26 |
| | (5H, m), 7.38 (1H, t, J = 8.0 Hz), 7.62-7.60 (2H, m), 7.71 (1H, d, J = 8.1 Hz), 7.82 (1H, s), 7.95 (1H, d, J = 7.7 Hz), |
| | 8.26 (1H, s), |
| 2-159 | 1H-NMR (CDCl3) δ: 1.04 (3H, t), 2.30 (3H, s), 2.61 (2H, d), 6.30 (1H, d), 7.03 (1H, t), 7.16 (1H, s), 7.21-7.26 (3H, m), 7.39 |
| | (1H, dd), 7.53 (1H, t), 7.85-7.77 (4H, m), 8.15 (1H, d), 8.29 (1H, s), 8.38 (1H, s), 8.50 (1H, d), 8.76 (1H, s). |
| 2-161 | 1H-NMR (CDCl3) δ: 1.04 (3H, t), 2.31 (3H, s), 2.61 (2H, q), 6.45 (1H, d), 7.13 (1H, s), 7.27-7.43 (5H, m), 7.54 (1H, t), |
| | 7.75-7.84 (3H, m), 8.18-8.10 (2H, m), 8.38 (1H, s), 8.52 (1H, dd), 8.63 (1H, s). |
| 2-162 | 1H-NMR (CDCl3) δ: 1.12 (3H, t), 2.25 (3H, s), 2.62 (2H, q), 6.99 (2H, d), 7.37 (1H, dd), 7.47-7.53 (3H, m), 7.69 (2H, d), 7.97-7.86 |
| | (3H, m), 8.11 (1H, dd), 8.24 (1H, s), 8.49 (1H, dd), 8.66 (1H, s). |
| 2-163 | 1H-NMR (CDCl3) δ: 1.81 (bs, 4H), 2.31 (s, 6H), 2.95 (bs, 4H), 7.34-7.44 (m, 3H), 7.47-7.57 (m, 2H), 7.72 (d, 1H), 7.88 (d, 1H), 8.13-8.21 |
| | (m, 1H), 8.24 (s, 1H), 8.46 (s, 1H), 8.48-8.55 (m, 1H) |
| 2-164 | 1H-NMR (CDCl3) δ: 1.21 (3H, t), 1.78-1.84 (4H, m), 2.33 (3H, s), 2.68 (2H, q), 2.94-3.00 (4H, m), 7.41-7.47 (4H, m), 7.55 (1H, |
| | dd), 7.74 (1H, d), 7.90 (1H, d), 8.27-8.21 (2H, m), 8.35 (1H, s), 8.55 (1H, dd). |
| 2-171 | 1H-NMR (CDCl3) δ: 2.28 (s, 6H), 2.33-2.50 (m, 1H), 2.56-2.73 (m, 1H), 3.09-3.45 (m, 3H), 3.72 (d, 1H), 7.25 (s, 2H), 7.32 (s, 2H), |
| | 7.35-7.44 (m, 2H), 7.48-7.59 (m, 2H), 7.73 (d, 1H), 7.86 (d, 1H), 8.17 (d, 1H), 8.27 (s, 1H), 8.46-8.55 (m, 2H) |
| 2-172 | 1H-NMR (CDCl3) δ: 1.19 (3H, t, J = 12.9 Hz), 1.56 (6H, br s), 2.27 (3H, s), 2.63 (2H, q, J = 7.5 Hz), 2.80 (4H, br s), 7.34 (1H, |
| | dd, J = 7.7, 4.8 Hz), 7.45-7.52 (3H, m), 7.70-7.67 (2H, m), 7.84 (1H, d, J = 7.9 Hz), 8.06 (1H, dd, J = 7.6, 1.9 Hz), |
| | 8.24 (1 |
| 2-180 | 1H-NMR (CDCl3) δ: 1.22 (3H, t), 2.34 (3H, s), 2.68 (2H, q), 2.88 (2H, br s), 3.52 (2H, br s), 5.66 (1H, d), 5.81 (1H, d), 7.42-7.58 (5H, |
| | m), 7.74 (1H, d), 7.90 (1H, d), 8.22-8.35 (3H, m), 8.55 (1H, dd). |
| 2-181 | 1H-NMR (CDCl3) δ: 1.17 (3H, t), 2.26 (3H, s), 2.63 (2H, q), 2.89 (4H, br s), 3.71 (4H, br s), 7.32 (1H, dd), 7.41-7.52 (3H, m), |
| | 7.67 (1H, d), 7.85-7.79 (2H, m), 8.00 (1H, d), 8.26 (1H, s), 8.43 (1H, dd), 8.92 (1H, s). |
| 2-183 | 1H-NMR (CDCl3) δ: 1.16 (3H, t), 2.29 (3H, s), 2.66 (2H, q), 7.13 (2H, s), 7.37 (1H, dd), 7.50 (1H, dd), 7.71 (1H, d), 7.86-7.80 |
| | (2H, m), 8.09 (1H, dd), 8.16 (1H, s), 8.31 (1H, s), 8.47 (1H, dd), 8.74 (1H, s). |
| 4-1 | 1H-NMR (CDCl3) δ: 2.28 (s, 6H), 6.11 (bs, 1H), 6.46 (bs, 1H), 7.20 (s, 2H), 7.32-7.59 (m, 3H), 7.68 (d, 1H), 7.97 (d, 1H), 8.12 |
| | (d, 1H), 8.22 (s, 1H), 8.40-8.49 (m, 1H), 8.63 (bs, 1H), 8.74 (s, 1H) |
| 4-5 | 1H-NMR (CDCl3) δ: 1.25 (3H, t), 2.38 (3H, s), 2.73 (2H, q), 7.44 (1H, dd), 7.49 (2H, s), 7.54-7.59 (2H, m), 7.76 (1H, d), 7.86 |
| | (1H, d), 8.23 (1H, dd), 8.36 (2H, d), 8.55 (1H, dd). |
| 5-1 | 1H-NMR (CDCl3) δ: 7.27-7.42 (m, 5H), 7.47-7.58 (m, 2H), 7.63-7.76 (m, 4H), 7.99-8.18 (m, 3H), 8.35-8.42 (m, 1H), |
| | 9.01 (bs, 1H) |
| 5-2 | 1H-NMR (CDCl3) δ: 7.33-7.47 (m, 4H), 7.52-7.62 (m, 2H), 7.63 (s, 1H), 7.71 (s, 1H), 8.02-8.12 (m, 2H), 8.20-8.29 (m, 1H), 8.35 (s, 1H), |
| | 8.39 (s, 1H), 8.51-8.57 (m, 1H) |
| 5-3 | 1H-NMR (CDCl3) δ: 5.45 (s, 1H), 7.25-7.53 (m, 6H), 7.60-7.72 (m, 3H), 7.77 (s, 1H), 7.95 (d, 1H), 8.04 (s, 1H), 8.12 (d, 1H), 8.26-8.53 (m, |
| | 1H), 9.17 (s, 1H) |
| 5-4 | 1H-NMR (CDCl3) δ: 7.36-7.50 (m, 6H), 7.52-7.61 (m, 1H), 7.65 (s, 1H), 7.71 (s, 1H), 8.02-8.12 (m, 2H), 8.21-8.29 (m, 1H), |
| | 8.34-8.43 (m, 2H), 8.51-8.59 (m, 1H) |
| 5-5 | 1H-NMR (CDCl3) δ: 7.34-7.50 (m, 6H), 7.53-7.61 (m, 1H), 7.65 (s, 2H), 7.94 (d, 1H), 8.08 (d, 1H), 8.18-8.24 (m, 1H), 8.29 |
| | (s, 1H), 8.46 (s, 1H), 8.50-8.57 (m, 1H) |
| 5-6 | 1H-NMR (CDCl3) δ: 7.30-7.46 (m, 5H), 7.51-7.61 (m, 2H), 7.65 (s, 2H), 7.94 (d, 1H), 8.09 (d, 1H), 8.17-8.30 (m, 2H), 8.42 (s, 1H), |
| | 8.49-8.58 (m, 1H) |
| 5-7 | 1H-NMR (CDCl3) δ: 4.65 (q, 1H), 7.30-7.48 (m, 6H), 7.51-7.60 (m, 1H), 7.64 (s, 2H), 7.78 (d, 1H), 7.96 (d, 1H), 8.19-8.27 |
| | (m, 2H), 8.32 (s, 1H), 8.51-8.60 (m 1H) |
| 11-1 | 1H-NMR (DMSO-d6) δ: 2.21 (6H, s), 5.15-5.05 (1H, m), 6.82 (1H, d), 7.26 (2H, s), 7.86 (1H, t), 8.43-8.48 |
| | (2H, m), 8.81 (1H, t). |
| 11-3 | 1H-NMR (acetone-d6) δ: 5.40 (1H, q), 6.26 (1H, br s), 7.87-7.94 (3H, m), 8.47-8.52 (2H, m), 8.87 (1H, |
| | t), 9.95 (1H, s). |
| 11-5 | 1H-NMR (CDCl3) δ: 1.53 (9H, s), 2.29 (3H, s), 3.43 (1H, br s), 6.36 (1H, br s), 7.48-7.51 (2H, m), |
| | 7.99 (1H, d) |
| 11-6 | 1H-NMR (CDCl3) δ: 2.88 (1H, br s), 3.74 (2H, br s), 6.62 (2H, d), 7.23 (2H, d), 7.33-7.36 (3H, m), |
| | 7.49-7.50 (2H, m) |
| 11-7 | 1H-NMR (CDCl3) δ: 2.82 (s, 1H), 4.66 (s, 2H), 7.32-7.55 (m, 7H) |
| 11-8 | 1H-NMR (CDCl3) δ: 3.73 (bs, 2H), 3.83 (s, 1H), 6.62 (d, 2H), 7.11 (d, 2H), 7.28-7.42 (m, 3H), 7.72-7.84 (m, 1H) |
| 11-9 | 1H-NMR (CDCl3) δ: 3.18 (s, 1H), 7.30-7.46 (m, 2H), 7.54 (s, 1H), 7.62-7.82 (m, 4H), 8.30 (d, 1H), 8.46 (d, 1H), |
| | 8.78 (s, 1H) |
| 11-11 | 1H-NMR (CDCl3) δ: 3.15 (s, 1H), 7.40 (d, 2H), 7.45 (d, 2H), 7.65-7.79 (m, 3H), 8.30 (d, 1H), 8.46 (d, 1H), |
| | 8.77 (s, 1H) |
| 11-12 | 1H-NMR (CDCl3) δ: 4.01 (bs, 2H), 6.86 (d, 1H), 6.94 (s, 1H), 7.20-7.31 (m, 3H), 7.35 (d, 2H), 7.47 (d, 2H), 7.76 |
| | (s, 2H), 8.54 (s, 1H) |
| 11-13 | 1H-NMR (CDCl3) δ: 2.90 (s, 1H), 3.81 (bs, 2H), 6.05 (d, 2H), 7.21 (d, 2H), 7.33 (t, 1H), 7.38 (d, 2H) |
| 11-14 | 1H-NMR (CDCl3) δ: 3.79 (bs, 3H), 6.66 (d, 2H), 7.16 (d, 2H), 7.26-7.38 (m, 3H), 7.49 (s, 1H) |
| 11-15 | 1H-NMR (CDCl3) δ: 0.01 (s, 9H), 4.68 (bs, 2H), 7.24-7.48 (m, 6H) |
| 11-16 | 1H-NMR (CDCl3) δ: −0.06 (s, 9H), 3.73 (bs, 2H), 6.60 (d, 2H), 7.11 (d, 2H), 7.23-7.40 (m, 4H) |
| 11-17 | 1H-NMR (CDCl3) δ: 0.03 (s, 9H), 4.68 (bs, 2H), 7.35 (s, 4H), 7.37 (s, 2H) |
| 11-18 | 1H-NMR (CDCl3) δ: 1.53 (9H, s), 2.29 (3H, s), 3.25 (3H, s), 6.41 (1H, br s), 7.48-7.51 (2H, m), 8.10 (1H, d) |
| 11-19 | 57-59 |
| 11-20 | 189-191 |
| 11-21 | 1H-NMR (CDCl3) δ: 3.28 (s, 3H), 3.80 (bs, 2H), 6.66 (d, 2H), 7.12 (d, 2H), 7.31 (s, 3H) |
| 11-22 | 1,5475 |
| 11-23 | 183-185 |
| 11-24 | 1,5503 |
| 11-25 | 1H-NMR (CDCl3) δ: 1.24 (3H, t), 2.36 (3H, s), 2.70 (2H, q), 4.00 (2H, q), 7.39 (1H, s), 7.42 (1H, s), 7.53 (1H, s), 7.76 (1H, t), |
| | 8.30 (1H, d), 8.47 (1H, d), 8.76 (1H, t). |
| 11-26 | 1H-NMR (acetone-d6) δ: 1.18 (3H, t), 2.33 (3H, s), 2.74 (2H, q), 4.30 (2H, q), 4.87 (2H, br s), 6.85-6.89 (1H, m), 7.30-7.16 (3H, |
| | m), 7.42 (2H, s), 8.96 (1H, s). |
| 11-27 | 1,5535 |
| 11-28 | 159-161 |
| 11-29 | 1,5502 |
| 11-30 | 1,541 |
| 11-31 | 160-161 |
| 11-32 | 86-90 |
| 11-34 | 1H-NMR (CDCl3) δ: 1.12 (3H, t), 2.32 (3H, s), 2.64 (2H, q), 3.88 (2H, s), 6.87 (2H, d), 7.04 (1H, dd), 7.42-7.16 (8H, m). |
| 11-35 | 1H-NMR (CDCl3) δ: 1.10 (3H, t), 2.31 (3H, s), 2.63 (2H, q), 6.61 (1H, d), 6.91-7.01 (2H, m), 7.37-7.45 (3H, m), 7.72 (1H, |
| | dd), 7.99 (1H, s), 8.31 (1H, d), 8.44 (1H, d), 8.79 (1H, s). |
| 11-36 | 1H-NMR (CDCl3) δ: 1.12 (3H, t), 2.32 (3H, s), 2.63 (3H, q), 3.87 (2H, s), 6.62 (1H, d), 6.86-6.98 (4H, m), |
| | 7.48-7.16 (6H, m). |
| 11-37 | 1H-NMR (CDCl3) δ: 1.07 (3H, t), 2.24 (3H, s), 2.61 (3H, q), 6.71 (1H, d), 6.95-7.11 (4H, m), 7.63 (1H, dd), 8.29 (1H, d), |
| | 8.37 (1H, d), 8.83-8.75 (3H, m). |
| 11-38 | 1H-NMR (CDCl3) δ: 1.11 (3H, t), 2.30 (3H, s), 2.63 (2H, q), 3.85 (2H, s), 6.67 (1H, d), 6.86 (1H, d), 7.56-6.99 (9H, m). |
| 11-39 | 1H-NMR (CDCl3) δ: 1.16 (3H, t), 2.32 (3H, s), 2.66 (2H, q), 6.81 (2H, d), 7.16 (2H, d), 7.41-7.43 (2H, m), 7.60 (1H, s), 7.76 |
| | (1H, dd), 8.31 (1H, d), 8.46 (1H, d), 8.76 (1H, s). |
| 11-40 | 1H-NMR (CDCl3) δ: 1.14 (3H, t), 2.32 (3H, s), 2.65 (2H, q), 3.89 (2H, s), 6.73-6.90 (4H, m), 7.39-7.12 (7H, m). |
| 11-41 | 1H-NMR (CDCl3) δ: 1.10 (3H, t), 2.24 (3H, s), 2.61 (2H, q), 4.67 (2H, s), 7.30-7.40 (6H, m), 7.63 (1H, dd), 8.14 (1H, s), |
| | 8.25 (1H, d), 8.37 (1H, d), 8.76 (1H, s). |
| 11-42 | 1H-NMR (CDCl3) δ: 1.14 (3H, t), 2.30 (3H, s), 2.64 (2H, q), 3.85 (2H, s), 4.67 (2H, s), 6.86 (1H, d), 7.19-7.40 (11H, m). |
| 11-43 | 1H-NMR (CDCl3) δ: 1.14 (3H, t), 2.33 (3H, s), 2.65 (2H, q), 4.78 (2H, s), 7.47-7.27 (6H, m), 7.67-7.78 (2H, m), 8.29 (1H, d), |
| | 8.46 (1H, d), 8.75 (1H, s). |
| 11-44 | 1H-NMR (CDCl3) δ: 1.11 (3H, t), 2.31 (3H, s), 2.63 (2H, q), 3.86 (2H, s), 4.77 (2H, s), 6.87 (1H, d), 7.19-7.39 (9H, m), 7.68 (1H, dd). |
| 11-45 | 1H-NMR (CDCl3) δ: 2.32 (6H, s), 4.64 (2H, s), 7.40-7.31 (6H, m), 7.57 (1H, s), 7.74 (1H, dd), 8.30 (1H, d), 8.46 (1H, |
| | d), 8.76 (1H, s). |
| 11-46 | 1H-NMR (CDCl3) δ: 2.31 (6H, s), 4.63 (2H, s), 7.39-7.20 (11H, m). |
| 11-47 | 1H-NMR (CDCl3) δ: 1.16 (3H, t), 2.32 (3H, s), 2.66 (2H, q), 4.65 (2H, s), 7.22-7.40 (6H, m), 7.56 (1H, s), 7.74 (1H, dd), 8.29 |
| | (1H, d), 8.46 (1H, d), 8.75 (1H, s). |
| 11-48 | 1H-NMR (CDCl3) δ: 1.15 (3H, t), 2.30 (3H, s), 2.65 (2H, q), 3.86 (2H, s), 4.63 (2H, s), 6.88 (1H, dd), 7.39-7.19 (10H, m). |
| 11-49 | 1H-NMR (CDCl3) δ: 1.16 (3H, t), 2.33 (3H, s), 2.66 (2H, q), 4.65 (2H, s), 7.40-7.26 (6H, m), 7.54 (1H, s), 7.75 (1H, dd), |
| | 8.29 (1H, d), 8.46 (1H, d), 8.75 (1H, s). |
| 11-50 | 1H-NMR (CDCl3) δ: 1.14 (3H, t), 2.31 (3H, s), 2.65 (2H, q), 3.86 (2H, s), 4.64 (2H, s), 6.87 (1H, d), 7.19-7.39 (10H, m). |
| 11-51 | 1H-NMR (CDCl3) δ: 1.20 (3H), 2.08 (3H, s), 2.08 (3H, s), 2.32 (3H, s), 2.66 (2H, q), 7.30 (2H, s), 7.49 (1H, s), 7.73 (1H, dd), 8.27 |
| | (1H, d), 8.44 (1H, d), 8.74 (1H, dd). |
| 11-52 | 1H-NMR (CDCl3) δ: 1.17 (3H, t), 1.90 (3H, s), 2.07 (3H, s), 2.28 (3H, s), 2.64 (2H, q), 3.80 (2H, br s), 6.83 (1H, d), 7.29-7.17 |
| | (5H, m), 7.42 (1H, s). |
| 11-53 | 1H-NMR (CDCl3) δ: 1.21 (3H, t), 1.72-1.87 (4H, m), 2.33 (3H, s), 2.35-2.48 (2H, m), 2.63-2.71 (4H, m), 7.32 (2H, s), 7.42 (1H, s), |
| | 7.74 (1H, dd), 8.28 (1H, d), 8.45 (1H, d), 8.74 (1H, s). |
| 11-54 | 1H-NMR (CDCl3) δ: 1.15 (3H, t), 1.70-1.89 (4H, m), 2.25 (3H, s), 2.39 (2H, t), 2.58-2.69 (4H, m), 3.71 (2H, br s), 6.78-6.81 (1H, m), |
| | 7.18-7.28 (5H, m), 7.57-7.53 (1H, m). |
| 11-55 | 1H-NMR (CDCl3) δ: 1.20 (3H, t), 1.6H.73 (6H, m), 2.17-2.24 (2H, m), 2.31 (3H, s), 2.62-2.69 (4H, m), 7.30 (2H, s), 7.59 (1H, |
| | br s), 7.71 (1H, dd), 8.27 (1H, dd), 8.43 (1H, dd), 8.75 (1H, dd). |
| 11-56 | 1H-NMR (CDCl3) δ: 1.14-1.23 (3H, m), 1.61-1.76 (6H, m), 2.16-2.30 (5H, m), 2.59-2.69 (4H, m), 3.66 (2H, br s), 6.79-6.86 (1H, m), |
| | 7.17-7.29 (5H, m), 7.52-7.48 (1H, m). |
| 11-57 | 1H-NMR (CDCl3) δ: 1.17 (3H, t), 2.30 (3H, s), 2.65 (2H, q), 7.34-7.42 (5H, m), 7.57 (2H, dd), 7.67-7.73 (2H, m), 8.27 (1H, d), |
| | 8.40-8.43 (2H, m), 8.76 (1H, dd). |
| 11-58 | 1H-NMR (CDCl3) δ: 1.17 (3H, t), 2.30 (3H, s), 2.65 (2H, q), 3.84 (2H, br s), 6.85 (1H, d), 7.18-7.29 (3H, m), 7.31-7.44 (6H, m), |
| | 7.57 (2H, dd), 8.40 (1H, s). |
| 11-59 | 1H-NMR (CDCl3) δ: 1.19 (3H, t), 2.33 (3H, s), 2.47 (3H, s), 2.67 (2H, q), 7.34-7.39 (5H, m), 7.44 (1H, s), 7.60 (2H, dd), 7.74 |
| | (1H, dd), 8.28 (1H, d), 8.45 (1H, d), 8.74 (1H, s). |
| 11-60 | 1H-NMR (CDCl3) δ: 1.13 (3H, t), 2.26 (3H, s), 2.46 (3H, s), 2.62 (2H, q), 3.76 (2H, br s), 6.74 (1H, d), 7.17-7.20 (3H, m), |
| | 7.40-7.31 (5H, m), 7.61-7.51 (3H, m). |
| 11-61 | 1H-NMR (CDCl3) δ: 1.17 (3H, t), 2.30 (2H, s), 2.65 (2H, q), 6.28 (2H, dd), 6.80 (2H, s), 7.24 (2H, s), 7.79-7.71 (2H, m), 8.31 |
| | (1H, d), 8.45 (1H, d), 8.77 (1H, s). |
| 11-62 | 1H-NMR (CDCl3) δ: 1.16 (3H, t), 2.30 (3H, s), 2.65 (2H, q), 3.88 (2H, br s), 6.27 (2H, dd), 6.80 (2H, s), 6.86-6.90 (1H, m), |
| | 7.22-7.35 (6H, m). |
| 11-63 | 1H-NMR (CDCl3) δ: 2.32 (6H, s), 6.44-6.45 (1H, m), 7.15 (1H, s), 7.50-7.54 (1H, m), 7.70-7.81 (3H, m), 8.28-8.31 (1H, m), |
| | 8.43-8.45 (1H, m), 8.77 (1H, br s) |
| 11-64 | 1H-NMR (CDCl3) δ: 2.16 (6H, s), 6.43-6.44 (1H, m), 7.14 (2H, s), 7.37-7.54 (2H, m), 7.73-7.85 (3H, m), 8.12-8.15 (1H, m), 8.29 |
| | (1H, s), 8.50-8.51 (1H, m), 8.66 (1H, s) |
| 11-65 | 1H-NMR (CDCl3) δ: 2.31 (6H, s), 5.82 (1H, q), 7.25 (2H, s), 7.57-7.51 (3H, m), 7.73 (1H, t), 8.29 (1H, d), 8.45 (1H, d), 8.74 (1H, s). |
| 11-66 | 1H-NMR (CDCl3) δ: 2.30 (6H, d), 3.86 (2H, br s), 5.81 (1H, q), 6.85-6.88 (1H, m), 7.22-7.31 (6H, m), 7.53 (1H, s), 7.56 (1H, s). |
| 11-67 | 1H-NMR (CDCl3) δ: 5.23-5.36 (2H, m), 7.44 (2H, s), 7.59 (1H, s), 7.64-7.67 (2H, m), 7.75-7.78 (1H, m), 8.30-8.34 (1H, m), |
| | 8.47-8.50 (1H, m), 8.78-8.80 (1H, m) |
| 11-69 | 1H-NMR (CDCl3) δ: 6.71 (2H, d), 7.17 (2H, d), 7.45 (1H, s), 7.65 (1H, s) |
| 11-70 | 1H-NMR (CDCl3) δ: 1.50 (9H, d, J = 9.2 Hz), 4.95 (1H, br s), 6.77 (1H, br s), 7.43 (2H, d), 7.63 (2H, d) |
| 11-71 | 1H-NMR (CDCl3) δ: 2.41 (3H, s), 7.24-7.35 (2H, m), 7.52 (1H, s), 7.68 (1H, s), 7.73-7.82 (2H, m), 8.17 (1H, d), 8.25 (1H, d), 8.46 |
| | (1H, d), 8.72 (1H, s). |
| 11-72 | 1H-NMR (CDCl3) δ: 2.37 (3H, s), 3.88 (2H, br s), 6.85-6.89 (1H, m), 7.15 (1H, d), 7.21-7.32 (4H, m), 7.49 (1H, s), 7.67 (1H, s), 7.75 |
| | (1H, s), 8.28 (1H, d). |
| 11-73 | 1H-NMR (CDCl3) δ: 2.32 (6H, s), 7.16 (2H, s), 7.54 (1H, s), 7.67 (1H, s), 7.72-7.75 (1H, m), 7.87 (1H, s), 8.30-8.32 (1H, m), 8.43-8.46 |
| | (1H, m), 8.78 (1H, br s) |
| 11-74 | 1H-NMR (CDCl3) δ: 2.31 (6H, s), 6.87-6.89 (1H, m), 7.14 (2H, s), 7.23-7.28 (3H, m), 7.38 (1H, s), 7.52 (1H, s), 7.67 (1H, s) |
| 11-75 | 1H-NMR (CDCl3) δ: 1.19 (3H, t), 2.33 (3H, s), 2.68 (2H, q), 7.18 (2H, s), 7.55 (1H, s), 7.60 (1H, s), 7.68 (1H, s), 7.76 (1H, t), |
| | 8.30 (1H, d), 8.47 (1H, d), 8.76 (1H, s). |
| 11-76 | 1H-NMR (CDCl3) δ: 1.17 (3H, t), 2.31 (3H, s), 2.66 (2H, q), 6.86-6.90 (1H, m), 7.15 (2H, s), 7.20-7.31 (3H, m), 7.38 (1H, s), 7.53 |
| | (1H, s), 7.67 (1H, s). |
| 11-78 | 1H-NMR (CDCl3) δ: 2.38 (3H, s), 6.87-6.91 (1H, m), 7.24-7.30 (4H, m), 7.49 (1H, s), 7.57 (1H, s), 7.63 (1H, s), 7.68 (1H, s). |
| 11-79 | 1H-NMR (CDCl3) δ: 7.45 (2H, s), 7.67 (1H, s), 7.70 (1H, d), 7.74-7.79 (2H, m), 8.30-8.34 (1H, m), 8.51-8.47 (1H, m), 8.79 (1H, t). |
| 11-80 | 1H-NMR (CDCl3) δ: 6.88-6.92 (1H, m), 7.26-7.32 (3H, m), 7.42 (2H, s), 7.60 (1H, s), 7.62 (1H, s), 7.69 (1H, s). |
| 11-81 | 1H-NMR (CDCl3) δ: 7.65 (2H, s), 7.67 (1H, s), 7.70 (1H, s), 7.76 (1H, t), 7.91 (1H, s), 8.33 (1H, d), 8.48 (1H, d), 8.80 (1H, t). |
| 11-82 | 1H-NMR (CDCl3) δ: 6.88-6.92 (1H, m), 7.26-7.33 (3H, m), 7.65-7.62 (4H, m), 7.69 (1H, s). |
| 11-83 | 1H-NMR (CDCl3) δ: 4.80 (s, 2H), 6.98 (d, 2H), 7.11 (s, 2H), 7.17 (s, 1H), 7.38 (d, 2H), 7.65 (s, 1H) |
| 11-85 | 1H-NMR (CDCl3) δ: 6.89-6.93 (1H, m), 7.27-7.30 (3H, m), 7.70-7.63 (3H, m), 7.85 (2H, s). |
| 11-86 | 1H-NMR (CDCl3) δ: 2.32 (6H, s), 5.85 (1H, q), 7.25 (2H, s), 7.50-7.60 (3H, m), 7.74 (1H, dd), 8.29 (1H, d), 8.45 (1H, |
| | d, J = 8.1 Hz), 8.74 (1H, s). |
| 11-88 | 1H-NMR (CDCl3) δ: 2.20 (3H, s), 7.43 (2H, s), 7.59 (1H, s), 7.64 (1H, s), 7.74-7.77 (2H, m), 8.30-8.33 (1H, m), 8.46-8.49 (1H, |
| | m), 8.77-8.81 (1H, m) |
| 11-89 | 1H-NMR (CDCl3) δ: 2.18 (3H, s), 3.87 (2H, br s), 6.89-6.91 (1H, m), 7.26-7.29 (3H, m), 7.41 (2H, s), 7.56 (2H, s), 7.62 (1H, s) |
| 11-90 | 1H-NMR (CDCl3) δ: 2.33 (6H, s), 7.16 (2H, s), 7.57 (2H, s), 7.71-7.76 (2H, m), 8.06-8.06 (1H, m), 8.28-8.31 (1H, m), |
| | 8.44-8.47 (1H, m), 8.76 (1H, s) |
| 11-91 | 1H-NMR (CDCl3) δ: 2.31 (6H, s), 6.86-6.89 (1H, m), 7.14 (2H, s), 7.23-7.27 (1H, m), 7.44 (1H, s), 7.56 (1H, s), 7.70 (1H, s), |
| | 7.78 (1H, s), 8.07 (1H, br s) |
| 11-93 | 1H-NMR (CDCl3) δ: 1.17 (3H, t), 2.31 (3H, s), 2.66 (2H, q), 6.86-6.90 (1H, m), 7.15 (2H, s), 7.20-7.34 (4H, m), 7.56 (1H, s), 7.70 (1H, s). |
| 11-94 | 1H-NMR (CDCl3) δ: 2.34 (s, 6H), 7.16 (s, 2H), 7.50 (s, 1H), 7.60 (s, 1H), 7.70-7.82 (m, 2H), 8.30 (d, 1H), 8.47 (d, 1H), 8.76 (s, 1H) |
| 11-95 | 1H-NMR (CDCl3) δ: 2.31 (s, 6H), 3.5-4.0 (bs, 2H), 6.88 (d, 1H), 7.13 (s, 2H), 7.18-7.37 (m, 4H), 7.58 (s, 1H), 7.74 (s, 1H) |
| 11-96 | 1H-NMR (CDCl3) δ: 2.32 (s, 6H), 2.34 (s, 3H), 6.21 (d, 1H), 7.18 (s, 2H), 7.43 (d, 1H), 7.51 (s, 1H), 7.69-7.81 (m, 1H), 8.30 |
| | (d, 1H), 8.46 (d, 1H), 8.76 (s, 1H) |
| 11-97 | 1H-NMR (CDCl3) δ: 2.30 (s, 6H), 2.34 (s, 3H), 3.86 (bs, 2H), 6.19 (d, 1H), 6.87 (d, 1H), 7.15 (s, 2H), 7.18-7.35 (m, 4H), 7.40 (d, 1H) |
| 11-98 | 1H-NMR (CDCl3) δ: 2.12 (s, 3H), 2.32 (s, 6H), 7.18 (s, 2H), 7.29 (s, 1H), 7.48-7.59 (m, 2H), 7.71-7.80 (m, 1H), 8.30 (d, 1H), |
| | 8.46 (d, 1H), 8.76 (s, 1H) |
| 11-100 | 1H-NMR (CDCl3) δ: 3.86 (2H, br s), 6.53 (1H, d), 6.64 (2H, d), 6.89 (2H, d), 7.01 (2H, d), 7.11 (1H, s), 7.36-7.40 (3H, m) |
| 11-101 | 1H-NMR (CDCl3) δ: 3.84 (bs, 2H), 6.52 (d, 1H), 6.64 (d, 2H), 7.26-7.47 (m, 6H), 7.58-7.65 (m, 1H) |
| 11-102 | 1H-NMR (CDCl3) δ: 1.51 (9H, s), 6.66-6.69 (2H, m), 7.29 (2H, d), 7.46-7.49 (3H, m) |
| 11-104 | 1H-NMR (CDCl3) δ: 2.31 (6H, s), 6.67 (1H, d), 6.90-6.93 (1H, m), 7.12 (2H, s), 7.28-7.36 (3H, m), 7.55 (1H, s) |
| 11-105 | 1H-NMR (CDCl3) δ: 4.93 (2H, br s), 6.58 (1H, d), 7.04 (2H, d), 7.11 (2H, s), 7.17-7.17 (1H, m), 7.37-7.49 (3H, m) |
| 11-106 | 1H-NMR (CDCl3) δ: 6.63 (1H), 7.15-7.21 (2H, m), 7.35 (2H, s), 7.47-7.50 (3H, m), 7.74-7.77 (2H, m), 8.32 (1H, d), 8.46-8.49 |
| | (1H, m), 8.80-8.80 (1H, m) |
| 11-107 | 1H-NMR (CDCl3) δ: 6.62 (1H, d), 6.87-6.89 (1H, m), 7.14-7.18 (3H, m), 7.28-7.30 (5H, m), 7.47-7.50 (3H, m), 7.73 (1H, br s). |
| 11-108 | 1H-NMR (CDCl3) δ: 6.58 (d, 1H), 7.16 (d, 1H), 7.44-7.59 (m, 4H), 7.64-7.90 (m, 3H), 8.11 (s, 2H), 8.30 (d, 1H), 8.46 (d, 1H), 8.78 (s, 1H) |
| 11-109 | 1H-NMR (CDCl3) δ: 3.86 (bs, 2H), 6.57 (d, 1H), 6.82-6.91 (m, 1H), 7.18 (d, 1H), 7.22-7.32 (m, 2H), 7.44-7.56 (m, 4H), 7.61 (s, |
| | 1H), 7.75-7.86 (m, 1H), 8.05 (s, 2H), |
| 11-110 | 1H-NMR (CDCl3) δ: 4.75 (bs, 2H), 6.54 (d, 1H), 7.19 (d, 1H), 7.42-7.51 (m, 3H), 7.65-7.77 (m, 3H) |
| 11-111 | 1H-NMR (CDCl3) δ: 2.33 (6H, s), 7.15 (2H, s), 7.71-7.94 (3H, m), 8.30 (1H, d), 8.45 (1H, d), 8.76 (1H, br s) |
| 11-112 | 1H-NMR (CDCl3) δ: 2.32 (6H, s), 6.87-6.90 (1H, m), 7.12 (2H, s), 7.22-7.31 (3H, m), 7.84 (1H, s), 7.93 (1H, s) |
| 11-114 | 1H-NMR (CDCl3) δ: 1.16 (3H, t), 2.31 (3H, s), 2.66 (2H, q), 3.89 (2H, br s), 6.86-6.90 (1H, m), 7.13 (2H, s), 7.30-7.19 (3H, |
| | m), 7.39 (1H, s), 7.85 (1H, s), 7.93 (1H, s). |
| 11-115 | 1H-NMR (CDCl3) δ: 2.33 (s, 6H), 6.72 (d, 1H), 7.13 (s, 2H), 7.50-7.82 (m, 3H), 8.30 (d, 1H), 8.47 (d, 1H), 8.76 (s, 1H) |
| 11-116 | 1H-NMR (CDCl3) δ: 2.30 (s, 6H), 3.88 (bs, 2H), 6.71 (d, 1H), 6.88 (d, 1H), 7.10 (s, 2H), 7.18-7.33 (m, 3H), 7.41 (s, 1H), 7.60 (s, 1H) |
| 11-117 | 1H-NMR (CDCl3) δ: 2.31 (s, 6H), 3.41 (s, 6H), 5.45 (s, 1H), 6.52 (d, 1H), 7.16 (s, 2H), 7.45-7.56 (m, 2H), 7.69-7.80 (m, 1H), 8.30 |
| | (d, 1H), 8.46 (d, 1H), 8.76 (s, 1H) |
| 11-118 | 1H-NMR (CDCl3) δ: 2.29 (s, 6H), 3.41 (s, 6H), 3.87 (bs, 2H), 5.15 (s, 1H), 6.51 (d, 1H), 6.88 (d, 1H), 7.13 (s, 2H), 7.18-7.39 (m, |
| | 4H), 7.49 (d, 1H) |
| 11-119 | 1H-NMR (CDCl3) δ: 2.33 (6H, s), 5.92 (1H, q), 7.26 (2H, s), 7.57 (1H, s), 7.75 (1H, t), 7.90 (1H, s), 8.02 (1H, s), 8.30 (1H, d), 8.46 |
| | (1H, d), 8.75 (1H, s). |
| 11-121 | 1H-NMR (CDCl3) δ: 2.32 (s, 6H), 2.62 (s, 3H), 5.88 (d, 1H), 7.15 (s, 2H), 7.46 (d, 1H), 7.69-7.85 (m, 2H), 8.32 (d, 1H), 8.45 |
| | (d, 1H), 8.78 (s, 1H) |
| 11-122 | 1H-NMR (CDCl3) δ: 2.31 (s, 6H), 2.63 (s, 3H), 3.88 (s, 2H), 6.84-6.93 (m, 2H), 7.12 (s, 2H), 7.18-7.32 (m, 3H), 7.38 (s, 1H), 7.46 |
| | (s, 1H) |
| 11-123 | 1H-NMR (CDCl3) δ: 2.35 (s, 6H), 7.19 (s, 2H), 7.42-7.56 (m, 2H), 7.69-7.81 (m, 1H), 8.30 (d, 1H), 8.47 (d, 1H), 8.76 (s, 1H) |
| 11-124 | 1H-NMR (CDCl3) δ: 2.32 (s, 6H), 3.87 (bs, 2H), 6.89 (d, 1H), 7.16 (s, 2H), 7.19-7.38 (m, 4H), 7.46 (s, 1H) |
| 11-125 | 1H-NMR (CDCl3) δ: 2.25 (3H, s), 2.27 (3H, s), 2.29 (6H, s), 5.58 (1H, q), 5.88 (1H, s), 7.32 (2H, s), 7.53 (1H, s), 7.72 (1H, t), |
| | 8.28 (1H, d), 8.43 (1H, d), 8.74 (1H, s). |
| 11-127 | 1H-NMR (CDCl3) δ: 2.33 (6H, s), 5.80 (1H, q), 6.98 (1H, s), 7.46 (2H, s), 7.50 (1H, s), 7.74 (1H, t), 8.29 (1H, d), 8.45 |
| | (1H, d), 8.74 (1H, s). |
| 11-128 | 1H-NMR (CDCl3) δ: 2.30 (6H, s), 5.78 (1H, q), 6.84-6.88 (1H, m), 6.96 (1H, s), 7.19-7.33 (4H, m), 7.43 (2H, s). |
| 11-130 | 1H-NMR (CDCl3) δ: 2.32 (6H, s), 3.87 (2H, br s), 6.87-6.89 (1H, m), 7.13 (1H, s), 7.23-7.29 (6H, m), 7.38 (1H, br s) |
| 11-131 | 1H-NMR (CDCl3) δ: 2.35 (6H, s), 7.14 (2H, s), 7.64-7.74 (3H, m), 8.31 (1H, d), 8.48 (1H, d), 8.77 (1H, s) |
| 11-132 | 1H-NMR (CDCl3) δ: 2.33 (6H, s), 6.88-6.91 (1H, m), 7.11 (2H, s), 7.24-7.33 (4H, m), 7.59 (1H, s) |
| 11-133 | 1H-NMR (CDCl3) δ: 2.35 (6H, s), 7.16 (2H, s), 7.56-7.75 (3H, m), 8.31 (1H, d), 8.48 (1H, d), 8.76 (1H, s) |
| 11-134 | 1H-NMR (CDCl3) δ: 2.33 (6H, s), 5.27 (1H, br s), 6.88-6.90 (1H, m), 7.13 (2H, s), 7.24-7.33 (3H, m), 7.55 (1H, s) |
| 11-135 | 1H-NMR (CDCl3) δ: 2.34 (6H, s), 7.15 (2H, s), 7.63-7.79 (3H, m), 8.31 (1H, d), 8.47 (1H, d), 8.77 (1H, s) |
| 11-136 | 1H-NMR (CDCl3) δ: 2.33 (6H, s), 6.88-6.91 (1H, m), 7.13 (2H, s), 7.25-7.35 (3H, m), 7.61 (1H, s), 7.70-7.70 (1H, m). |
| 11-137 | 1H-NMR (CDCl3) δ: 1.37 (t, 3H), 2.35 (s, 6H), 4.35 (q, 2H), 7.15 (s, 2H), 7.56 (s, 1H), 7.17-7.82 (m, 1H), 8.09 (s, 1H), 8.31 (d, 1H), 8.48 |
| | (d, 1H), 8.77 (s, 1H) |
| 11-138 | 1H-NMR (CDCl3) δ: 1.36 (t, 3H), 2.33 (s, 6H), 3.88 (bs, 2H), 4.34 (q, 2H), 6.89 (d, 1H), 7.12 (s, 2H), 7.18-7.44 (m, 4H), |
| | 8.07 (s, 1H) |
| 11-139 | 1H-NMR (CDCl3) δ: 2.35 (s, 6H), 7.23 (s, 2H), 7.54 (s, 1H), 7.71-7.83 (m, 1H), 8.31 (d, 1H), 8.47 (d, 1H), 8.76 (s, 1H) |
| 11-140 | 1H-NMR (CDCl3) δ: 2.31 (s, 6H), 3.86 (bs, 2H), 6.87 (d, 1H), 7.14-7.31 (m, 5H), 7.49 (s, 1H) |
| 11-141 | 1H-NMR (CDCl3) δ: 1.19 (3H, t), 2.35 (3H, s), 2.68 (2H, q), 7.22 (2H, s), 7.56 (1H, s), 7.76 (1H, t), 8.31 (1H, d), 8.47 |
| | (1H, d), 8.76 (1H, s). |
| 11-142 | 1H-NMR (acetone-d6) δ: 1.13 (3H, t), 2.32 (3H, s), 2.71 (3H, q), 4.87 (2H, br s), 6.87 (1H, dd), 7.16-7.38 (5H, m), 9.00 (1H, s). |
| 11-143 | 1H-NMR (CDCl3) δ: 2.27 (6H, s), 2.30 (6H, s), 5.61 (1H, q), 7.31 (2H, s), 7.52 (1H, s), 7.73 (1H, t), 8.28 (1H, d), 8.44 (1H, |
| | d), 8.74 (1H, s). |
| 11-145 | 1H-NMR (CDCl3) δ: 2.34 (6H, s), 7.08 (1H, s), 7.18 (2H, s), 7.59 (1H, s), 7.75-7.77 (1H, m), 7.98 (1H, br s), 8.34 (1H, d), |
| | 8.45-8.48 (1H, m), 8.80 (1H, s) |
| 11-147 | 1H-NMR (CDCl3) δ: 2.34 (s, 6H), 7.23 (s, 2H), 7.66-7.86 (m, 2H), 8.31 (d, 1H), 8.45 (d, 1H), 8.77 (s, 1H) |
| 11-149 | 1H-NMR (CDCl3) δ: 4.01 (2H, br s), 6.68-6.71 (2H, m), 7.12 (2H, d), 8.12 (1H, s), 8.22 (1H, s) |
| 11-150 | 1H-NMR (CDCl3) δ: 2.17 (3H, s), 6.17 (2H, br s), 6.71 (1H, d), 7.00-7.03 (2H, m), 8.18 (1H, s), 8.32 |
| | (1H, s) |
| 11-151 | 1H-NMR (CDCl3) δ: 1.54 (9H, s), 2.27 (3H, s), 6.44 (1H, s), 7.08 (1H, s), 7.20 (1H, d), 8.10 (1H, d), |
| | 8.13 (1H, s), 8.22 (1H, s) |
| 11-152 | 1H-NMR (CDCl3) δ: 2.22 (3H, s), 4.41 (2H, br s), 6.92 (1H, s), 7.29 (1H, s), 8.13 (1H, s), 8.26 (1H, s) |
| 11-153 | 1H-NMR (CDCl3) δ: 4.94 (2H, br s), 7.33 (2H, s), 8.14 (1H, s), 8.32 (1H, s) |
| 11-154 | 1H-NMR (CDCl3) δ: 2.33 (6H, s), 7.12 (2H, s), 7.59 (1H, s), 7.74-7.77 (1H, m), 8.15 (1H, s), 8.29-8.31 (2H, m), |
| | 8.46-8.47 (1H, m), 8.76 (1H, s) |
| 11-156 | 1H-NMR (CDCl3) δ: 1.18 (3H, t), 2.32 (3H, s), 2.67 (2H, q), 7.13 (2H, s), 7.70-7.78 (2H, m), 8.15 (1H, s), 8.30-8.32 (2H, m), |
| | 8.47 (1H, d), 8.77 (1H, s). |
| 11-158 | 1H-NMR (CDCl3) δ: 2.41 (3H, s), 7.22 (1H, s), 7.47 (1H, s), 7.75-7.78 (2H, m), 8.16 (1H, s), 8.32 (1H, d), 8.38 (1H, s), |
| | 8.46-8.50 (1H, m), 8.78-8.81 (1H, m) |
| 11-159 | 1H-NMR (CDCl3) δ: 2.38 (3H, s), 3.85 (2H, br s), 6.88-6.91 (1H, m), 7.23-7.28 (4H, m), 7.44 (1H, s), 7.68 (1H, s), 8.15 |
| | (1H, s), 8.34 (1H, s) |
| 11-160 | 1H-NMR (CDCl3) δ: 7.16-7.19 (1H, m), 7.59 (2H, s), 7.76-7.79 (2H, m), 8.17 (1H, s), 8.32-8.33 (1H, m), 8.44-8.51 (2H, |
| | m), 8.80-8.80 (1H, m) |
| 11-161 | 1H-NMR (CDCl3) δ: 7.10-7.53 (7H, m), 8.14 (1H, s), 8.39 (1H, s) |
| 11-162 | 1H-NMR (CDCl3) δ: 1.19 (3H, t), 2.34 (3H, s), 2.68 (2H, q), 7.16 (2H, s), 7.61 (1H, s), 7.76 (1H, t), 8.19 (1H, s), 8.30 (1H, d), |
| | 8.47 (1H, d), 8.76 (1H, s). |
| 11-163 | 1H-NMR (CDCl3) δ: 1.18 (3H, t), 2.32 (3H, s), 2.67 (2H, q), 3.89 (2H, br s), 6.87-6.90 (1H, m), 7.13 (2H, s), 7.19-7.31 (3H, m), |
| | 7.38 (1H, s), 8.16 (1H, s). |
| 11-164 | 1H-NMR (CDCl3) δ: 2.31 (6H, s), 7.12 (2H, s), 7.70-7.73 (1H, m), 8.03 (1H, s), 8.27-8.31 (1H, m), 8.34 (1H, s), |
| | 8.42-8.45 (1H, m), 8.75 (1H, s) |
| 11-166 | 1H-NMR (CDCl3) δ: 2.29 (s, 6H), 6.94 (s, 1H), 7.63-7.81 (m, 2H), 7.63-7.81 (m, 2H), 8.29 (d, 1H), 8.44 (d, |
| | 1H), 8.77 (s, 1H) |
| 11-167 | 1H-NMR (CDCl3) δ: 2.32 (s, 6H), 3.89 (bs, 2H), 6.88 (d, 1H), 7.03 (s, 2H), 7.15 (m, 3H), 7.41 (s, 1H), 8.76 (s, 1H) |
| 11-168 | 1H-NMR (CDCl3) δ: 1.08 (3H, t), 2.33 (3H, s), 2.63 (2H, q), 6.30 (1H, d), 7.30-7.17 (3H, m), 7.38 (1H, s), 7.59 (1H, s), 7.71-7.79 |
| | (2H, m), 8.32 (1H, d), 8.47 (1H, d), 8.77 (1H, s). |
| 11-170 | 1H-NMR (CDCl3) δ: 1.05 (3H, t), 2.32 (3H, s), 2.61 (2H, q), 6.30 (1H, d), 7.03 (1H, t), 7.18-7.31 (3H, m), 7.74-7.82 (3H, |
| | m), 8.34-8.28 (2H, m), 8.46 (1H, d), 8.78 (1H, s). |
| 11-175 | 1H-NMR (acetone-d6) δ: 1.01 (3H, t), 2.31 (3H, s), 2.66 (2H, q), 4.87 (2H, br s), 6.48 (1H, d), 6.88 (1H, d), 7.16-7.51 (7H, m), |
| | 8.17 (1H, d), 9.04 (1H, s). |
| 11-176 | 1H-NMR (CDCl3) δ: 1.21 (3H, t), 1.55 (6H, br s), 2.31 (3H, s), 2.65 (2H, q), 2.80 (4H, br s), 7.44 (1H, s), 7.47 (1H, s), 7.54 |
| | (1H, s), 7.72 (1H, t), 8.27 (1H, d), 8.43 (1H, d), 8.73 (1H, s). |
| 11-178 | 1H-NMR (CDCl3) δ: 1.21 (3H, t), 1.82 (4H, br s), 2.32 (3H, s), 2.66 (2H, q), 2.96 (4H, br s), 7.43 (1H, s), 7.47 (2H, s), 7.74 |
| | (1H, t), 8.28 (1H, d), 8.45 (1H, d), 8.75 (1H, s). |
| 11-180 | 1H-NMR (CDCl3) δ: 1.22 (3H, t), 2.12 (2H, br s), 2.33 (3H, s), 2.67 (2H, q), 2.88 (2H, t), 3.51 (2H, br s), 5.67 (1H, d), 5.81 (1H, |
| | d), 7.60-7.46 (3H, m), 7.75 (1H, t), 8.29 (1H, d), 8.46 (1H, d), 8.75 (1H, s). |
| 11-181 | 1H-NMR (acetone-d6) δ: 1.16 (3H, t), 2.16 (2H, br s), 2.30 (3H, s), 2.69 (2H, q), 2.86 (2H, t), 3.50 (2H, br s), 4.86 (2H, br s), |
| | 5.70 (1H, d), 5.82 (1H, d), 6.84-6.88 (1H, m), 7.30-7.16 (3H, m), 7.47 (1H, s), 7.53 (1H, s), 8.85 (1H, s). |
| 11-182 | 1H-NMR (CDCl3) δ: 1.17 (3H, t), 2.26 (3H, s), 2.63 (2H, q), 2.89 (4H, br s), 3.71 (4H, br s), 7.31 (1H, dd), 7.41-7.52 (3H, |
| | m), 7.67 (1H, d), 7.85-7.80 (2H, m), 8.00 (1H, d), 8.26 (1H, s), 8.43 (1H, dd), 8.92 (1H, s). |
| 11-183 | 1H-NMR (acetone-d6) δ: 1.17 (3H, t), 2.30 (3H, s), 2.70 (2H, q), 2.88 (4H, br s), 3.70-3.73 (4H, m), 4.86 (2H, br s), 6.84-6.88 (1H, m), |
| | 7.30-7.15 (3H, m), 7.52 (1H, s), 7.58 (1H, s), 8.86 (1H, s). |
| 11-184 | 1H-NMR (CDCl3) δ: 3.38 (2H, br s), 6.73 (2H, d), 7.45 (2H, d). |
| 11-185 | 1H-NMR (CDCl3) δ: 1.53 (9H, s), 6.67 (1H, br s), 7.53 (2H, d), 7.62 (2H, d). |
| 11-186 | 1H-NMR (CDCl3) δ: 4.95 (2H, s), 7.69 (2H, s). |
| 11-187 | 1H-NMR (CDCl3) δ: 7.76-7.81 (2H, m), 8.01-8.03 (2H, m), 8.33 (1H, ddd), 8.50 (1H, ddd), 8.80 (1H, dd). |
| 11-188 | 1H-NMR (CDCl3) δ: 3.89 (2H, br s), 6.89-6.92 (1H, m), 7.28-7.33 (3H, m), 7.66 (1H, s), 7.97 (2H, s). |
| 11-189 | 1H-NMR (CDCl3) δ: 1.25 (3H, t), 2.38 (3H, s), 2.72 (2H, q), 7.51 (2H, s), 7.55 (1H, s), 7.76 (1H, t), 8.31 (1H, d), 8.47 (1H, |
| | d), 8.76 (1H, s). |
| 11-191 | 1H-NMR (CDCl3) δ: 1.13 (3H, t), 2.34 (3H, s), 2.65 (2H, q), 6.95 (1H, d), 7.22-7.34 (3H, m), 7.40-7.43 (2H, m), 7.51 (1H, s), 7.76 |
| | (1H, dd), 8.31 (1H, d), 8.47 (1H, d), 8.76 (1H |
| 11-192 | 1H-NMR (CDCl3) δ: 1.10 (3H, t), 2.32 (3H, s), 2.64 (2H, q), 3.86 (2H, s), 6.88-6.94 (2H, m), 7.60-7.20 (9H, m). |
| 11-193 | 1H-NMR (CDCl3) δ: 1.17 (3H, t), 2.35 (3H, s), 2.67 (2H, q), 6.84-6.92 (4H, m), 7.45-7.51 (3H, m), 7.76 (1H, dd), 8.31 (1H, d), |
| | 8.47 (1H, d), 8.75 (1H, s). |
| 11-194 | 1H-NMR (CDCl3) δ: 1.14 (3H, t), 2.33 (3H, s), 2.65 (2H, q), 3.88 (2H, s), 6.82-6.91 (5H, m), 7.42-7.20 (6H, m). |
| 11-195 | 1H-NMR (CDCl3) δ: 1.14 (3H, t), 2.32 (3H, s), 2.65 (2H, q), 6.76 (2H, d), 7.32-7.27 (2H, m), 7.41-7.40 (2H, m), 7.74 (2H, dd), |
| | 8.30 (1H, d), 8.45 (1H, d), 8.77 (1H, s). |
| 11-196 | 1H-NMR (CDCl3) δ: 1.12 (3H, t), 2.30 (3H, s), 2.63 (2H, q), 3.61 (2H, s), 6.76 (2H, d), 6.86 (1H, d), 7.15-7.55 (8H, m). |
| 11-197 | 1H-NMR (CDCl3) δ: 1.12 (3H, t), 2.33 (3H, s), 2.65 (2H, q), 6.95 (2H, d), 7.37 (1H, s), 7.41 (1H, s), 7.46 (2H, d), 7.66 (1H, s), 7.75 |
| | (1H, dd), 8.31 (1H, d), 8.46 (1H, d), 8.76 (1H, s). |
| 11-198 | 1H-NMR (CDCl3) δ: 1.11 (3H, t), 2.32 (3H, s), 2.64 (2H, q), 3.88 (2H, s), 6.88 (1H, d), 6.94 (2H, d), 7.20-7.47 (8H, m). |
| 11-199 | 1H-NMR (CDCl3) δ: 1.15 (3H, t), 2.26 (3H, s), 2.33 (3H, s), 2.66 (2H, q), 6.77 (2H, d), 6.98 (2H, d), 7.46 (2H, s), 7.55 (1H, s), |
| | 7.75 (1H, dd), 8.30 (1H, d), 8.46 (1H, d), 8.76 (1H, s). |
| 11-200 | 1H-NMR (CDCl3) δ: 1.11 (3H, t), 2.24 (3H, s), 2.28 (3H, s), 2.62 (2H, q), 3.62 (2H, br s), 6.77 (2H, d), 6.84 (1H, d), 6.96 (2H, d), |
| | 7.27-7.19 (3H, m), 7.42 (2H, s), 7.51 (1H, d). |
| 11-201 | 1H-NMR (CDCl3) δ: 1.17 (3H, t), 2.35 (3H, s), 2.67 (2H, q), 3.74 (3H, s), 6.72 (2H, d), 6.84 (2H, d), 7.49-7.50 (3H, m), 7.76 (1H, dd), |
| | 8.31 (1H, d), 8.47 (1H, d), 8.76 (1H, s). |
| 11-202 | 1H-NMR (CDCl3) δ: 1.15 (3H, t), 2.33 (3H, s), 2.66 (2H, q), 3.74 (3H, s), 3.84 (2H, br s), 6.71 (2H, d), 6.82-6.90 (3H, m), 7.31-7.20 (3H, |
| | m), 7.36 (1H, s), 7.45 (2H, s). |
| 11-203 | 1H-NMR (CDCl3) δ: 1.11 (3H, t), 2.34 (3H, s), 2.65 (2H, q), 6.75 (1H, dd), 7.26-7.29 (2H, m), 7.34 (1H, s), 7.63 (1H, s), 7.76 |
| | (1H, dd), 8.31 (1H, d), 8.48-8.46 (2H, m), 8.76 (1H, s). |
| 11-204 | 1H-NMR (CDCl3) δ: 1.09 (3H, t), 2.32 (3H, s), 2.64 (2H, q), 3.88 (2H, s), 6.74 (1H, d), 6.88 (1H, d), 7.39-7.20 (7H, |
| | m), 8.46 (1H, d). |
| 11-205 | 1H-NMR (CDCl3) δ: 1.11 (3H, t), 2.34 (3H, s), 2.65 (2H, q), 6.81 (1H, dd), 7.24-7.29 (2H, m), 7.34 (1H, s), 7.55 (1H, s), 7.76 |
| | (1H, dd), 8.31 (1H, d), 8.47 (1H, d), 8.51 (1H, d), 8.76 (1H, s). |
| 11-206 | 1H-NMR (CDCl3) δ: 1.09 (3H, t), 2.32 (3H, s), 2.63 (2H, q), 3.88 (2H, s), 6.80 (1H, dd), 6.88 (1H, dd), 7.31-7.20 (6H, m), |
| | 7.39 (1H, s), 8.49 (1H, d). |
| 11-207 | 1H-NMR (CDCl3) δ: 1.11 (3H, t), 2.33 (3H, s), 2.65 (2H, q), 6.73 (2H, d), 7.31 (1H, s), 7.35 (1H, s), 7.76 (1H, dd), 7.87 |
| | (1H, s), 8.37-8.31 (3H, m), 8.47 (1H, d), 8.77 (1H, s). |
| 11-208 | 1H-NMR (CDCl3) δ: 1.10 (3H, t), 2.31 (3H, s), 2.63 (2H, q), 3.88 (2H, s), 6.73 (2H, d), 6.88 (1H, dd), 7.20-7.31 (5H, m), |
| | 7.44 (1H, s), 8.38 (2H, d). |
| 11-211 | 1H-NMR (CDCl3) δ: 1.19 (3H, t), 2.37 (3H, s), 2.70 (2H, q), 3.88 (3H, s), 5.31 (1H, s), 7.36 (1H, s), 7.38 (1H, s), 7.54 (1H, s), |
| | 7.77 (1H, dd), 8.32 (1H, d), 8.48 (1H, d), 8.77 (1H, s). |
| 11-212 | 1H-NMR (CDCl3) δ: 1.16 (2H, t), 2.35 (3H, s), 2.68 (2H, q), 3.88 (3H, s), 3.90 (2H, s), 5.33 (1H, s), 6.89 (1H, d), |
| | 7.47-7.21 (6H, m). |
| 11-213 | 1H-NMR (CDCl3) δ: 1.19 (3H, t), 2.29 (3H, d), 2.66 (2H, q), 7.34-7.37 (4H, m), 7.51 (2H, d), 7.58 (1H, s), 7.73 (1H, dd), 8.29 |
| | (1H, d), 8.37 (1H, s), 8.45 (1H, dd), 8.75 (1H, dd). |
| 11-214 | 1H-NMR (CDCl3) δ: 1.17 (2H, t), 2.32 (3H, s), 2.66 (2H, q), 3.87 (2H, br s), 6.87 (1H, d), 7.37-7.19 (7H, m), 7.50-7.53 (3H, m), |
| | 8.36 (1H, s). |
| 11-215 | 1H-NMR (CDCl3) δ: 0.25-0.31 (2H, m), 0.60-0.67 (2H, m), 1.16-1.27 (4H, m), 2.34 (3H, s), 2.69 (2H, q), 3.44 (2H, d,), 7.40 (2H, s), |
| | 7.60 (1H, s), 7.74 (1H, dd), 8.30 (1H, d), 8.46 (1H, d), 8.76 (1H, s). |
| 11-216 | 1H-NMR (CDCl3) δ: 0.25-0.30 (2H, m), 0.59-0.65 (2H, m), 1.17-1.25 (4H, m), 2.33 (3H, s), 2.68 (2H, dd), 3.42 (2H, d), 3.87 (2H, |
| | br s), 6.88 (1H, dd), 7.20-7.36 (6H, m). |
| 11-217 | 1H-NMR (CDCl3) δ: 1.18 (3H, t), 2.29 (3H, s), 2.36 (3H, s), 2.65 (2H, q), 7.17 (2H, d), 7.38 (2H, s), 7.46 (2H, d), 7.54 (1H, br s), |
| | 7.71 (1H, dd), 8.27 (1H, d), 8.37 (1H, s), 8.43 (1H, d), 8.74 (1H, s). |
| 11-218 | 1H-NMR (CDCl3) δ: 1.17 (3H, t), 2.31 (3H, s), 2.36 (3H, s), 2.66 (2H, q), 6.87 (1H, d), 7.17 (2H, d), 7.21-7.31 (4H, m), 7.35 (2H, s), |
| | 7.46 (2H, d), 8.36 (1H, s). |
| 11-219 | 1H-NMR (CDCl3) δ: 1.19 (2H, t), 2.33 (3H, s), 2.67 (2H, q), 3.83 (3H, s), 6.89 (2H, d), 7.38 (2H, s), 7.47-7.53 (3H, m), 7.74 (1H, dd), |
| | 8.28 (1H, d), 8.34 (1H, s), 8.45 (1H, d), 8.74 (1H, s). |
| 11-220 | 1H-NMR (CDCl3) δ: 1.17 (2H, t), 2.31 (3H, s), 2.65 (2H, q), 3.82 (3H, s), 6.86-6.90 (3H, m), 7.38-7.18 (6H, m), 7.51 (2H, d), |
| | 8.33 (1H, s). |
| 11-221 | 1H-NMR (CDCl3) δ: 1.16 (3H, t), 2.35 (3H, s), 2.67 (2H, q), 6.65 (1H, dd), 7.10 (1H, d), 7.23 (1H, d), 7.39 (2H, s), 7.55 (1H, s), |
| | 7.76 (1H, dd), 8.31 (1H, d), 8.47 (1H, d), 8.76 (1H, s). |
| 11-222 | 1H-NMR (CDCl3) δ: 1.14 (3H, t), 2.33 (3H, s), 2.66 (2H, q), 3.88 (2H, s), 6.64 (1H, dd), 6.89 (1H, dd), 7.11 (1H, d), 7.20-7.32 |
| | (4H, m), 7.37-7.35 (3H, m). |
| 11-223 | 1H-NMR (CDCl3) δ: 1.15 (3H, t), 2.34 (3H, s), 2.66 (2H, q), 6.63 (2H, d), 7.39 (1H, s), 7.41 (1H, s), 7.47-7.53 (3H, m), 7.76 |
| | (1H, dd), 8.31 (1H, d), 8.47 (1H, d), 8.76 (1H, s). |
| 11-224 | 1H-NMR (CDCl3) δ: 1.13 (3H, t), 2.32 (3H, s), 2.65 (2H, q), 3.88 (2H, s), 6.63 (2H, d), 6.88 (1H, dd), 7.20-7.31 (3H, m), 7.36 |
| | (2H, s), 7.37 (1H, s), 7.48 (2H, d). |
| 11-225 | 1H-NMR (CDCl3) δ: 1.14 (3H, t), 2.35 (3H, s), 2.66 (2H, q), 6.88 (2H, d), 7.05 (2H, d), 7.40 (1H, s), 7.43 (1H, s), 7.53 (1H, s), 7.76 |
| | (1H, dd), 8.31 (1H, d), 8.46 (1H, dd), 8.75 (1H, d). |
| 11-226 | 1H-NMR (CDCl3) δ: 1.12 (3H, t), 2.33 (3H, s), 2.64 (2H, q), 3.89 (2H, s), 6.80 (1H, d), 6.88 (2H, d), 7.04 (2H, d), 7.20-7.31 |
| | (3H, m), 7.36 (2H, s), 7.40 (1H, s). |
| 11-227 | 1H-NMR (CDCl3) δ: 1.12-1.21 (6H, m), 2.33 (3H, s), 2.57 (2H, q), 2.65 (2H, q), 6.79 (2H, d), 7.01 (2H, d), 7.45 (1H, s), 7.46 (1H, s), |
| | 7.56 (1H, br s), 7.75 (1H, dd), 8.31 (1H, d), 8.46 (1H, d), 8.76 (1H, s). |
| 11-228 | 1H-NMR (CDCl3) δ: 1.10-1.20 (6H, m), 2.32 (3H, s), 2.56 (2H, q), 2.64 (2H, q), 3.87 (2H, s), 6.79 (2H, d), 6.88 (1H, d), 7.00 |
| | (2H, d), 7.20-7.35 (4H, m), 7.41 (1H, s), 7.43 (1H, s). |
| 11-229 | 1H-NMR (CDCl3) δ: 0.90 (3H, t), 1.14 (3H, t), 1.27-1.37 (2H, m), 1.48-1.59 (2H, m), 2.33 (3H, s), 2.49-2.56 (2H, m), |
| | 2.65 (2H, q), 6.78 (2H, d), 6.98 (2H, d), 7.44 (1H, s), 7.46 (1H, s), 7.57 (1H, s), 7.75 (1H, dd), 8.30 (1H, d), |
| | 8.46 (1H, dd), 8.76 (1H, dd). |
| 11-230 | 1H-NMR (CDCl3) δ: 0.91 (3H, t), 1.12 (3H, t), 1.26-1.37 (2H, m), 1.48-1.58 (2H, m), 2.32 (3H, s), 2.49-2.55 (2H, m), 2.64 |
| | (2H, q), 3.87 (2H, s), 6.78 (2H, d), 6.87 (1H, dd), 6.97 (2H, d), 7.20-7.31 (3H, m), 7.36 (1H, s), 7.40 (1H, s), |
| | 7.42 (1H, s). |
| 11-231 | 1H-NMR (CDCl3) δ: 2.34 (6H, s), 6.82 (2H, d), 7.16 (2H, d), 7.41 (2H, s), 7.56 (1H, s), 7.76 (1H, dd), 8.32 (1H, d), 8.47 |
| | (1H, d), 8.77 (1H, s). |
| 11-232 | 1H-NMR (CDCl3) δ: 2.32 (6H, s), 3.88 (2H, s), 6.82 (2H, d), 6.88 (1H, d), 7.15 (2H, d), 7.21-7.31 (3H, m), |
| | 7.41-7.36 (3H, m). |
| 11-233 | 1H-NMR (CDCl3) δ: 2.34 (6H, s), 6.76 (2H, d), 7.30 (2H, d), 7.41 (2H, s), 7.54 (1H, br s), 7.76 (1H, dd), 8.32 (1H, |
| | d), 8.47 (1H, d), 8.76 (1H, s). |
| 11-234 | 1H-NMR (CDCl3) δ: 2.32 (6H, s), 3.88 (2H, s), 6.76 (2H, d), 6.88 (1H, dd), 7.20-7.31 (5H, m), 7.38-7.37 (3H, m). |
| 11-235 | 1H-NMR (CDCl3) δ: 1.10 (3H, t), 2.34 (3H, s), 2.64 (2H, q), 6.85 (2H, d), 7.35 (1H, s), 7.41 (1H, s), 7.51 (2H, d), 7.62 (1H, s), 7.76 |
| | (1H, dd), 8.31 (1H, d), 8.47 (1H, d), 8.76 (1H, s). |
| 11-236 | 1H-NMR (CDCl3) δ: 1.09 (3H, t), 2.32 (3H, s), 2.63 (2H, q), 3.88 (2H, s), 6.89 (2H, d), 7.32-7.20 (5H, m), 7.37-7.40 (2H, |
| | m), 7.46 (2H, d). |
| 11-237 | 1H-NMR (CDCl3) δ: 2.34 (6H, s), 6.94 (1H, d), 7.27-7.35 (2H, m), 7.41 (2H, s), 7.51-7.61 (2H, m), 7.76 (1H, dd), 8.32 (1H, d), 8.47 |
| | (1H, d), 8.77 (1H, s). |
| 11-238 | 1H-NMR (CDCl3) δ: 2.32 (6H, s), 3.87 (2H, s), 6.87-6.94 (2H, m), 7.37-7.21 (7H, m), 7.38 (2H, s). |
| 11-239 | 1H-NMR (CDCl3) δ: 1.13 (3H, t), 2.34 (3H, s), 2.66 (2H, q), 6.94 (2H, d), 7.34 (1H, s), 7.38 (1H, s), 7.51 (2H, d), 7.58 |
| | (1H, br s), 7.76 (1H, dd), |
| | 8.31 (1H, d), 8.47 (1H, d), 8.76 (1H, s). |
| 11-240 | 1H-NMR (CDCl3) δ: 1.11 (3H, t), 2.32 (3H, s), 2.64 (2H, q), 3.88 (2H, s), 6.89 (1H, dd), 6.93 (2H, d), 7.37-7.20 |
| | (6H, m), 7.50 (2H, d). |
| 11-241 | 1H-NMR (CDCl3) δ: 2.33 (6H, s), 6.94 (2H, d), 7.35 (2H, s), 7.51 (2H, d), 7.70 (1H, s), 7.76 (1H, dd), 8.32 (1H, d), 8.46 |
| | (1H, d), 8.77 (1H, s). |
| 11-242 | 1H-NMR (CDCl3) δ: 2.31 (6H, s), 3.88 (2H, s), 6.88 (1H, dd), 6.94 (2H, d), 7.20-7.30 (3H, m), 7.32 (2H, s), 7.44 (1H, s), |
| | 7.50 (2H, d). |
| 11-243 | 1H-NMR (DMSO-d6) δ: 1.03 (3H, t), 2.00 (3H, s), 2.26 (3H, s), 2.61 (2H, q), 6.83 (2H, d), 7.38-7.47 (4H, m), 7.88 (1H, dd), |
| | 8.49-8.42 (2H, m), 8.81 (1H, s), 9.93 (1H, s), 10.32 (1H, s). |
| 11-244 | 1H-NMR (DMSO-d6) δ: 1.02 (3H, t), 2.00 (3H, s), 2.23 (3H, s), 2.59 (2H, q), 5.32 (2H, s), 6.75 (1H, dd), 6.83 (2H, d), 7.10-7.18 |
| | (3H, m), 7.35 (1H, s), 7.41 (1H, s), 7.45 (2H), 9.69 (1H, s), 9.93 (1H, s). |
| 11-245 | 1H-NMR (CDCl3) δ: 1.06 (3H, t), 2.28 (3H, s), 2.58 (2H, q), 6.96 (1H, dd), 7.03 (1H, d), 7.19 (1H, s), 7.28 (1H, s), 7.42 (1H, s), |
| | 7.76-7.66 (2H, m), 7.96 (1H, d), 8.27 (1H, d), 8.45 (1H, d), 8.72 (1H, s). |
| 11-246 | 1H-NMR (CDCl3) δ: 1.03 (3H, t), 2.25 (3H, s), 2.56 (2H, q), 3.84 (2H, s), 6.84 (1H, dd), 6.94 (1H, ddd), 7.01 (1H, dz), 7.16-7.32 |
| | (6H, m), 7.67 (1H, ddd), 7.97 (1H, dd). |
| 11-247 | 1H-NMR (CDCl3) δ: 1.15 (3H, t), 2.34 (3H, s), 2.66 (2H, q), 7.08-7.15 (2H, m), 7.41 (1H, s), 7.44 (1H, s), 7.58 (1H, s), 7.76 (1H, dd), |
| | 8.29-8.32 (3H, m), 8.47 (1H, d), 8.76 (1H, s). |
| 11-248 | 1H-NMR (CDCl3) δ: 1.03 (3H, t), 2.24 (3H, s), 2.56 (2H, q), 3.84 (2H, s), 6.84 (1H, dd), 6.94 (1H, ddd), 7.01 (1H, d), 7.28-7.16 (5H, |
| | m), 7.32 (1H, s), 7.67 (1H, ddd), 7.97 (1H, dd). |
| 11-249 | 1H-NMR (CDCl3) δ: 1.10 (3H, t), 2.28 (3H, s), 2.60 (2H, q), 7.01 (1H, d), 7.18 (1H, s), 7.22 (1H, s), 7.44 (1H, s), 7.66 (1H, dd), 7.73 |
| | (1H, dd), 7.90 (1H, d), 8.27 (1H, d), 8.45 (1H, dd), 8.73 (1H, d). |
| 11-250 | 1H-NMR (CDCl3) δ: 1.08 (3H, t), 2.26 (3H, s), 2.59 (2H, q), 3.85 (2H, s), 6.86 (1H, dd), 6.99 (1H, d), 7.14-7.29 (6H, m), 7.64 (1H, |
| | dd), 7.92 (1H, d). |
| 11-251 | 1H-NMR (CDCl3) δ: 1.12 (3H, t), 2.23 (3H, s), 2.59 (2H, q), 6.72 (1H, d), 7.14 (2H, s), 7.25 (1H, d), 7.66 (1H, dd), 8.04 (1H, s), |
| | 8.23 (1H, d), 8.39 (1H, dd), 8.72 (1H, d). |
| 11-252 | 1H-NMR (CDCl3) δ: 1.11 (3H, t), 2.23 (3H, s), 2.59 (2H, q), 3.87 (2H, s), 6.75-6.82 (2H, m), 7.13-7.26 (6H, m), 7.49 (1H, br s). |
| 11-253 | 1H-NMR (CDCl3) δ: 1.17 (6H, t), 2.67 (4H, q), 6.81 (2H, d), 7.15 (2H, d), 7.43 (2H, s), 7.51 (1H, s), 7.76 (1H, dd), 8.30 (1H, d), 8.47 |
| | (1H, dd), 8.75 (1H, d). |
| 11-254 | 1H-NMR (CDCl3) δ: 1.14 (6H, t), 2.65 (4H, q), 3.88 (2H, s), 6.81 (2H, d), 6.88 (1H, d), 7.14 (2H, d), 7.20-7.39 (6H, m). |
| 11-255 | 1H-NMR (CDCl3) δ: 1.16 (6H, t), 2.66 (4H, q), 6.75 (2H, d), 7.30 (2H, d), 7.42 (2H, s), 7.57 (1H, d), 7.75 (1H, dd), 8.30 |
| | (1H, dd), 8.46 (1H, dd), 8.76 (1H, dd). |
| 11-256 | 1H-NMR (CDCl3) δ: 1.15 (6H, t), 2.65 (4H, q), 3.88 (2H, s), 6.75 (2H, d), 6.88 (1H, dd), 7.18-7.33 (5H, m), 7.35 |
| | (1H, s), 7.39 (2H, s). |
| 11-257 | 1H-NMR (CDCl3) δ: 6.78 (2H, d), 7.14 (2H, d), 7.65-7.82 (5H, m), 8.07 (1H, s), 8.28 (1H, dd), 8.45 (1H, dd), 8.71 (1H, dd). |
| 11-258 | 1H-NMR (CDCl3) δ: 3.86 (2H, s), 6.78 (2H, d), 6.86 (1H, dd), 7.11-7.29 (5H, m), 7.61 (2H, d), 7.75 (2H, d), 7.90 (1H, s). |
| 11-259 | 1H-NMR (CDCl3) δ: 6.73 (2H, d), 7.28 (2H, d), 7.65 (2H, d), 7.74 (1H, dd), 7.81 (2H, d), 8.15 (1H, s), 8.28 (1H, dd), 8.44 |
| | (1H, dd), 8.72 (1H, dd). |
| 11-260 | 1H-NMR (CDCl3) δ: 3.87 (2H, s), 6.72 (2H, d), 6.86 (1H, dd), 7.15 (1H, dd), 7.20 (1H, dd), 7.24-7.29 (3H, m), 7.60 (2H, d), 7.75 |
| | (2H, d), 7.88 (1H, s). |
| 11-261 | 1H-NMR (CDCl3) δ: 1.17 (3H, t), 2.35 (3H, s), 2.67 (2H, q), 7.10 (1H, dd), 7.16 (1H, d), 7.40 (1H, s), 7.41 (1H, s), 7.51 (1H, s), |
| | 7.77 (1H, dd), 8.06 (1H, d), 8.30 (1H, d), 8.47 (1H, d), 8.76 (1H, s). |
| 11-262 | 1H-NMR (CDCl3) δ: 1.14 (3H, t), 2.32 (3H, s), 2.66 (2H, q), 3.88 (2H, s), 6.89 (1H, dd), 7.08 (1H, d), 7.14 (1H, dd), 7.20-7.31 (4H, m), |
| | 7.36 (1H, s), 7.37 (1H, s), 8.07 (1H, d). |
| 11-263 | 1H-NMR (CDCl3) δ: 2.34 (6H, s), 7.11 (1H, dd), 7.16 (1H, d), 7.40 (2H, s), 7.67 (1H, s), 7.75 (1H, dd), 8.06 (1H, d), 8.31 (1H, d), 8.46 |
| | (1H, d), 8.77 (1H, s). |
| 11-264 | 1H-NMR (CDCl3) δ: 2.32 (6H, s), 3.88 (2H, s), 6.89 (1H, dd), 7.09 (1H, dd), 7.14 (1H, d), 7.21-7.32 (4H, m), 7.36 (2H, s), 8.08 (1H, d). |
| 11-265 | 1H-NMR (CDCl3) δ: 6.76 (2H, d), 7.43 (2H, d), 7.69 (2H, s), 7.75 (1H, dd), 8.40 (1H, dd), 8.51 (1H, dd), 8.77 (1H, s), 9.03 (1H, dd). |
| 11-266 | 1H-NMR (CDCl3) δ: 3.79 (2H, br s), 6.76 (2H, d), 6.92-6.96 (1H, m), 7.31-7.17 (3H, m), 7.38 (2H, d), 7.47 (1H, dd), 7.69 (2H, s). |

### FORMULATION EXAMPLE 1

### A Granule Formulation

To a mixture comprising the compound of the present invention (Compound No. 1-1; 10 parts by weight), bentonite (montmorilonite; 30 parts by weight), talc (58 parts by weight) and lignin sulfonate (2 parts by weight), water (25 parts by weight) is added and the resulting mixture is kneaded well. By using an extrusive granulator, granules of 10 to 40 mesh are formed and a granule formulation is obtained after drying at 40 to 50° C.

### FORMULATION EXAMPLE 2

### Agranule Formulation

Clay mineral having a size distribution in the range of 0.2 to 2 mm (95 parts by weight) is added to a rotary mixer. By spraying the compound of the present invention (Compound No. 1-1; 5 parts by weight) together with a liquid diluent under rotation, the clay is moistened followed by drying at 40 to 50° C. to obtain a granule formulation.

### FORMULATION EXAMPLE 3

### An Emulsion

By mixing the compound of the present invention (Compound No. 1-1; 30 parts by weight), xylene (55 parts by weight), polyoxyethylenealkylphenyl ether (8 parts by weight) and calcium alkylbenzene sulfonate (7 parts by weight) with stirring, an emulsion is obtained.

### FORMULATION EXAMPLE 4

### A Wettable Agent

By mixing and pulverization of the compound of the present invention (Compound No. 1-1; 15 parts by weight), a mixture comprising white carbon (fine powders of hydrous non-crystalline silicon oxide) and powder clay (1:5 mixture; 80 parts by weight), and a condensate of sodium alkylnaphthalene sulfonate formalin (3 parts by weight) and sodium alkylbenzene sulfonate (2 parts by weight), a wettable agent is obtained.

### FORMULATION EXAMPLE 5

### Wettable Granules

The compound of the present invention (Compound No. 1-1; 20 parts by weight), lignin sodium sulfonate (30 parts by weight), bentonite (15 parts by weight) and calcined diatomite powder (35 parts by weight) are thoroughly mixed. After adding water thereto, the mixture is extruded through 0.3 mm screen followed by drying to obtain wettable granules.

### BIOLOGICAL EXAMPLES

Unless not mentioned otherwise, the test solutions were prepared as follows:

Containing as solvent: Dimethylformamide, 3 parts by weight; and as emulsifier: Polyoxyethylene alkyl phenyl ether, 1 part by weight

To prepare the test solution, 1 part by weight of an active compound is mixed with the above-mentioned amount of solvent containing the above-mentioned amount of emulsifier, and the mixture is diluted with water to the desired concentration.

### BIOLOGICAL TEST EXAMPLE 1

### Test Against Tobacco Cutworm (Spodoptera litura) Larvae

The leaves of sweet potato were immersed in the test solution at the appropriate concentration, and the leaves were dried in air. The leaves were then placed in a petri dish having a diameter of 9 cm, and ten third-instar larvae of tobacco cutworm were released therein. The petri dish was placed in a temperature-controlled chamber at 25° C. After 2 days and 4 days sweet potato leaves were additionally added. After 7 days, the number of dead larvae was counted to calculate the insecticidal activity. An insecticidal activity of 100% means that all larvae were killed, whereas an insecticidal activity of 0% means that no larva was killed. In the current test, the results of two petri dishes for each partition were averaged.

In the Biological Test Example 1, the compounds Nos.1-23, 1-24, 1-25, 1-26, 1-27, 1-28, 1-40, 1-59, 1-60, 1-63, 1-66, 1-79, 1-80, 1-83, 1-84, 1-85, 1-104, 1-108, 1-109, 1-110, 1-111, 1-112, 1-125, 1-128, 1-129, 1-130, 1-131, 1-138, 1-142, 1-146, 1-149, 1-150, 1-151, 1-152, 1-160, 1-166, 1-167, 1-168, 1-171, 1-173, 1-176, 1-177, 1-180, 1-181, 1-182, 1-201, 2-1, 2-2, 2-6, 2-24, 2-30, 2-31, 2-32, 2-33, 2-34, 2-35, 2-36, 2-37, 2-38, 2-39, 2-40, 2-41, 2-44, 2-51, 2-55, 2-59, 2-61, 2-62, 2-63, 2-76, 2-77, 2-82, 2-83, 2-84, 2-87, 2-95, 2-98, 2-99, 2-100, 2-103, 2-104, 2-105, 2-106, 2-107, 2-108, 2-109, 2-115, 2-116, 2-118, 2-120, 2-123, 2-124, 2-125, 2-128, 2-133, 2-134, 2-142, 2-143, 2-144, 2-145, 2-147, 2-149, 2-153, 2-154, 2-170, 2-180, 2-183, 4-5, and 11-26 showed an insecticidal activity of 100% at an active compound concentration of 100 ppm.

### BIOLOGICAL TEST EXAMPLE 2

### Test Against Two-Spotted Spider Mite (Tetranychus urticae)

50 to 100 adult two-spotted spider mites were inoculated onto the leaves of kidney beans at the two-true leaf stage, which plant had been cultivated in a pot having a diameter of 6 cm. After one day, test solution at the appropriate concentration was sprayed thereon in a sufficient amount using a spray gun. After the spraying, the plant pot was placed inside a greenhouse, and after 7 days, the acaricidal activity was calculated. An acaricidal activity of 100% means that all mites were killed, whereas an acaricidal activity of 0% means that no mite was killed.

In the Biological Test Example 2, the compound No. 1-27, 1-28, 1-40, 1-60, 1-61, 1-64, 1-66, 1-109, 2-1, 2-6, 2-24, 2-42, 2-51, 2-55, 2-62, 2-82, 2-83, 2-84, 2-145 and 4-5 showed an acaricidal activity rate of 100% at a concentration of 100 ppm.

### BIOLOGICAL TEST EXAMPLE 3

### Test Against Cucurbit Leaf Beetle (Aulacophora femoralis)

Cucumber leaves were immersed in the test solution at the appropriate concentration, and the leaves were dried in air. The leaves were then placed in a plastic cup containing sterilized black soil, and five second-instar larvae of cucurbit leaf beetle were released therein. The plastic cup was placed in a temperature-controlled chamber at 25° C. After 7 days, the number of dead larvae was counted, and thus the insecticidal activity was calculated. An insecticidal activity of 100% means that all beetles were killed, whereas an insecticidal activity of 0% means that no beetle was killed.

Compounds Nos. 1-25, 1-26, 1-27, 1-28, 1-40, 1-59, 1-60, 1-61, 1-66, 1-79, 1-83, 1-84, 1-85, 1-105, 1-108, 1-109, 1-128, 1-129, 1-130, 1-131, 1-138, 1-142, 1-146, 1-151, 1-152, 1-160, 1-166, 1-168, 1-171, 1-173, 1-176, 1-177, 1-182, 2-1, 2-2, 2-6, 2-7, 2-24, 2-31, 2-32, 2-33, 2-34, 2-41, 2-44, 2-55, 2-59, 2-61, 2-62, 2-63, 2-82, 2-83, 2-87, 2-100, 2-103, 2-104, 2-105, 2-108, 2-109, 2-116, 2-119, 2-128, 2-133, 2-134, 2-143, 2-144, 2-145, 2-147, 2-153, 2-180, 2-183 and 4-5 showed an insecticidal activity of 100% at an active compound concentration of 100 ppm.

### BIOLOGICAL TEST 4

### Phaedon Cochleariae—Test; (PHAECO Spray Application)

Solvent: 78.0 parts by weight of acetone and 1.5 parts by weight of dimethylformamide

Emulsifier: 0.5 parts by weight of alkylaryl polyglycolether

To produce a suitable preparation of active compound, 1 part by weight of active compound is mixed with the stated amount of solvent and emulsifier, and the concentrate is diluted with emulsifier-containing water to the desired concentration. Chinese cabbage (Brassica pekinesis) leaf-disks are sprayed with a preparation of the active ingredient of the desired concentration. Once dry, the leaf disks are infested with mustard beetle larvae (Phaedon cochleariae). After 7 days mortality in % is determined. 100% means that all beetle larvae have been killed and 0% means that none of the beetle larvae have been killed.

In this test compound No. 11-143 showed an activity of 100% at application rate of 500 g/ha:

### BIOLOGICAL TEST EXAMPLE 5

### Ctenocephalides felis—Test (CTECFE)

Solvent: dimethyl sulfoxide

To produce a suitable preparation of active compound, 10 mg of active compound are dissolved in 0.5 ml solvent, and the concentrate is diluted with cattle blood to the desired concentration.

Approximately 10 to 15 adult unfed (Ctenocepahlides felis) are placed in flea chambers. The blood chamber, are sealed with paraffin on the bottom are filled with cattle blood supplied with compound solution and placed on top of the flea chamber, so that the fleas are able to suck the blood. The blood chamber is heated to 37° C. whereas the flea chamber is kept at room temperature. After 2 days mortality in % is determined. 100% means that all the fleas have been killed; 0% means that none of the fleas have been killed.

In this test, compound no. 1-80 showed an activity of 80% at an application rate of 100 ppm; while the following compounds showed an activity of 90% at an application rate of 100 ppm: Ex no: 1-25, 1-27, 1-40, 2-77, 2-151. The Following compounds from the preparation examples showed an activity of 95% at application rate of 100 ppm: Ex no: 1-59, 2-84, 2-103, 2-148. The Following compounds showed an activity of 100% at application rate of 100 ppm: Ex no: 1-26, 1-60, 2-51, 2-104, 2-108, 2-109

### BIOLOGICAL TEST EXAMPLE 6

### Lucillia cuprina—Test

Solvent: dimethyl sulfoxide

To produce a suitable preparation of active compound, 10 mg of active compound are dissolved in 0.5 ml solvent, and the concentrate is diluted with water to the desired concentration. Approximately 20-30 (Lucilia cuprina larvae) are transferred into a test tube containing 1 cm³ of minced horse meat and 0.5 ml aqueous dilution of test compound. After 2 days mortality in % is determined. 100% means that all the larvae have been killed; 0% means that none of the larvae have been killed.

In this test the following compounds showed an activity of 90% at an application rate of 100 ppm: Ex no: 2-104, 2-120, 2-124; while the following compounds showed an activity of 100% at application rate of 100ppm Ex no: 1-6, 1-24, 1-25, 1-26, 1-27, 1-40, 1-59, 1-60, 1-61, 1-80, 11-114, 2-51, 2-84, 2-98, 2-100, 2-108, 2-109, 2-118, 2-119, 2-125, 2-151

### BIOLOGICAL TEST EXAMPLE 7

### Musca domestica—Test

Solvent: dimethyl sulfoxide

To produce a suitable preparation of active compound, 10 mg of active compound are dissolved in 0.5 ml solvent, and the concentrate is diluted with water to the desired concentration. Prior to the assay, a piece or kitchen sponge is soaked with a mixture of sugar and compound solution and placed into a container. 10 adults (Musca domestica) are placed into the container and closed with a perforated lid. After 2 days mortality in % is determined. 100% means that all the flies have been killed; 0% means that none of the flies have been killed.

In this test the following compounds from the preparation examples showed an activity of 80% at application rate of 100 ppm: Ex no: I-1-262, I-1-264, 1-25, 1-27, 2-104; while the following compounds showed an activity of 90% at an application rate of 100 ppm: Ex no: I-1-263, I-2-16, 1-24, 1-26. The following compounds showed an activity of 100% at an application rate of 100 ppm: Ex no: 1-40, 1-59, 1-60, 1-80

### BIOLOGICAL TEST EXAMPLE 8

### Boophilus microplus—Test (Injection)

Solvent: dimethyl sulfoxide

To produce a suitable preparation of active compound, 10 mg of active compound are dissolved in 0.5 ml solvent, and the concentrate is diluted with solvent to the desired concentration. Five adult engorged female ticks (Boophilus microplus) are injected with compound solution into the abdomen. Ticks are transferred into replica plates and incubated in a climate chamber for a period of time. Egg deposition of fertile eggs is monitored. After 7 days mortality in % is determined. 100% means that all eggs are infertile; 0% means that all eggs are fertile.

In this test compound no. 11-114 showed an activity of 80% at an application rate of 20 μg/animal; while compound no. 11-150 showed an activity of 90% at an application rate of 20 μg/animal. The following compounds showed an activity of 100% at an application rate of 20 μg/animal: Ex no: 1-6, 1-24, 1-25, 1-26, 1-27, 1-40, 1-59, 1-60, 1-61, 1-80, 2-51, 2-77, 2-84, 2-98, 2-100, 2-103, 2-104, 2-108, 2-109, 2-118, 2-119, 2-120, 2-124, 2-125, 2-148, 2-151, 2-153

The novel pesticidal amides of the present invention have an excellent pesticidal activity as shown in the above examples.

1. Compounds A compound of formula (I) 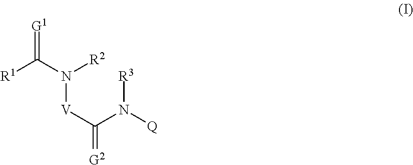
wherein:
R¹ represents hydrogen, or optionally substituted alkyl, haloalkyl, alkoxy, haloalkoxy, phenyl or a 5- or 6-membered heterocyclic group;
R² and R³ independently represent hydrogen; or optionally substituted alkyl, haloalkyl, alkylcarbonyl, haloalkylcarbonyl, alkoxycarbonyl or haloalkoxycarbonyl;
G¹ and G² independently represent oxygen or sulfur;
V is selected among the cyclic groups V1 to V5: 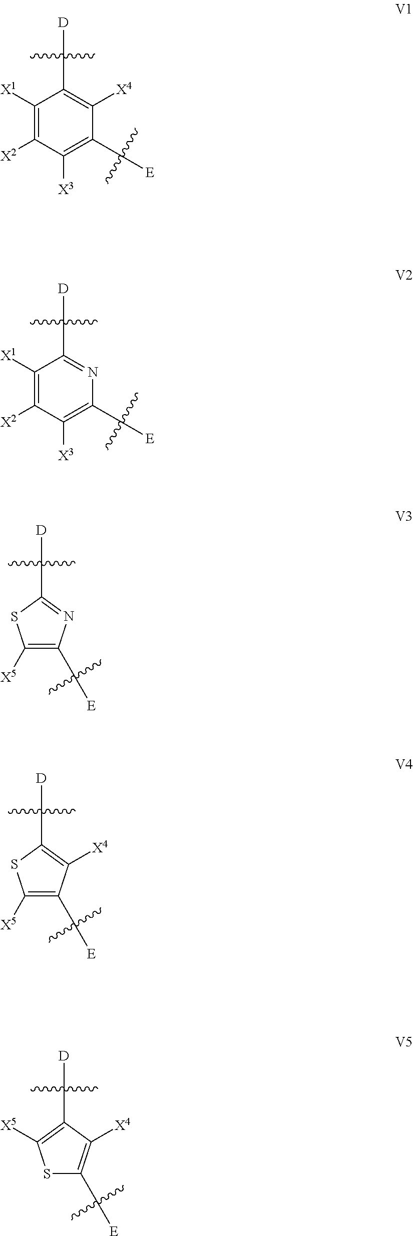
wherein
D stands for the bonding site to the nitrogen atom belonging to the moiety 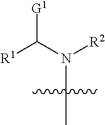
of formula (I), and
E stands for the bonding site to the carbon atom belonging to the moiety: 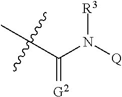
of formula (I); and
X¹ to X⁵ independently represent hydrogen, halogen, or optionally substituted alkyl, haloalkyl, alkoxy, haloalkoxy, or cyano or nitro;
Q is selected among the groups Q1 to Q7. 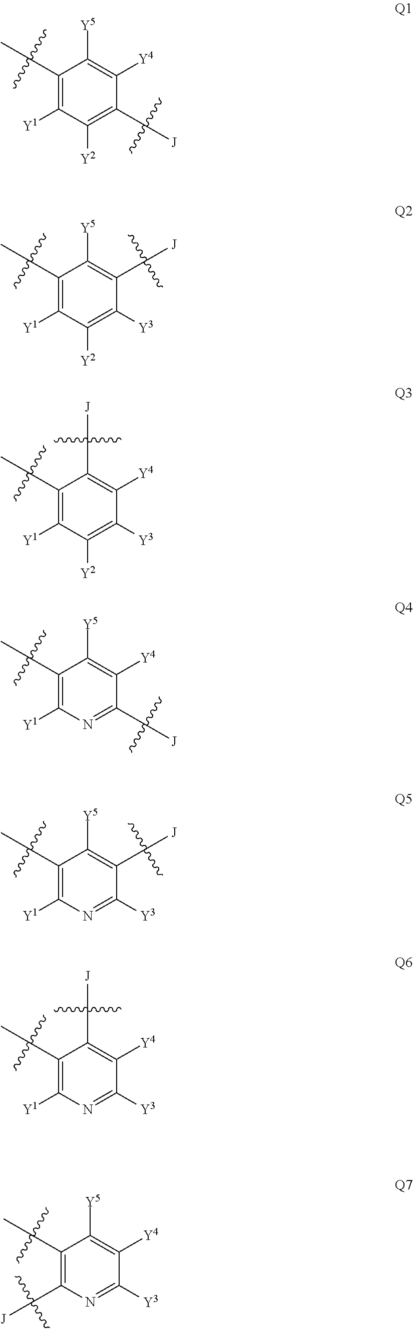
wherein
Y² to Y⁴ independently represent hydrogen, halogen, or optionally substituted alkyl, haloalkyl, alkoxy, haloalkoxy, or cyano or nitro;
Y¹ and Y⁵ independently represent halogen, or optionally substituted alkyl, haloalkyl, alkoxy, haloalkoxy, alkylthio, alkylsulfinyl, alkylsulfonyl, haloalkylthio, haloalkylsulfinyl, haloalkylsulfonyl, or cyano or nitro;
and
J represents a chemical grouping having the following formula: 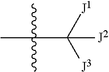
wherein
J¹ represents C₁-C₆ haloalkyl,
J² represents hydrogen, halogen, or optionally substituted alkyl, haloalkyl, phenyl or a heterocyclic group,
J³ represents hydroxyl, cyano, azide, halogen, or optionally substituted alkyl, haloalkyl, or OR⁴, SR⁵, NR⁶R⁷, N(R⁸)NR⁶R⁷, N(R⁸)OR⁶, an optionally substituted heterocyclic group, or a chemical group having the following formulae: 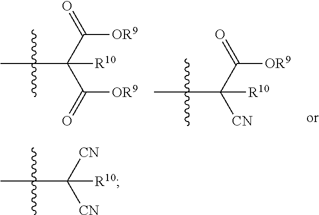
and wherein
R⁴ represents optionally substituted alkyl, haloalkyl, alkoxyalkyl, alkylthioalkyl, haloalkylthioalkyl alkenyl, alkynyl, cycloalkyl, cycloalkenyl, phenyl, aralkyl, iminyl, alkylcarbonyl, haloalkylcarbonyl, phenylcarbonyl, alkylsulfonyl, haloalkylsulfonyl, phenylsulfonyl, or a heterocyclic group, heterocyclic group-alkylene or a heterocyclic group-carbonyl,
R⁵ represents optionally substituted alkyl, haloalkyl, alkoxyalkyl, haloalkoxyalkyl, alkylthioalkyl, haloalkylthioalkyl, alkenyl, alkynyl, phenyl, aralkyl, or a 5- or 6-membered heterocyclic group comprising at least one hetero atom selected among from the group consisting of N, O and S, or a 5- or 6-membered heterocyclic group-alkylene comprising at least one hetero atom selected from the group consisting of N, O and S,
R⁶, R⁷ and R⁸ independently represent hydrogen, optionally substituted alkyl, haloalkyl, alkoxyalkyl, haloalkoxyalkyl, alkylthioalkyl, haloalkylthioalkyl, alkenyl, alkynyl, phenyl, aralkyl, alkylsulfonyl, haloalkylsulfonyl, phenylsulfonyl, alkylcarbonyl, haloalkylcarbonyl, phenylcarbonyl, a 5- or 6-membered heterocyclic group comprising at least one hetero atom selected from the group consisting of N, O and S, a 5- or 6-membered heterocyclic group-alkylene comprising at least one hetero atom selected from the group consisting of N, O and S, or a 5- or 6-membered heterocyclic group-carbonyl and comprising at least one hetero atom selected from the group consisting of N, O and S,
or
R⁶ and R⁷ may form a cyclic amino group together with the nitrogen atom to which they are bonded, and said cycle may comprise an oxygen atom, a sulfur atom or a carbonyl group;
R⁹ represents optionally substituted alkyl or haloalkyl; and
R¹⁰ represents hydrogen, optionally substituted alkyl or haloalkyl, preferably R⁹ represents optionally substituted C₁-C₆ or C₁-C₄ alkyl or C₁-C₆ or C₁-C4 haloalkyl;
under the proviso that J¹ and J² are not perfluoroalkyl and J³ is not hydroxy or halogen at the same time.

2. A compound according to claim 1, wherein
R¹ represents hydrogen, optionally substituted C₁-C₈ or C₁-C₄alkyl, C₁-C₈ or C₁-C₄ haloalkyl, C₁-C₈ or C₁-C₄ alkoxy , C₁-C₈ or C₁-C₄ haloalkoxy, phenyl or a 5-to 6-membered heterocyclic group comprising at least one heteroatom selected from the group consisting of N, O, and S;
R² and R³ independently represent hydrogen, optionally substituted C₁-C₆ or C₁-C₄ alkyl, C₁-C₆ or C₁-C₄ haloalkyl, C₂-C₇ or C₂-C₅ alkylcarbonyl, C₂-C₇ or C₂-C₅ haloalkylcarbonyl, C₂-C₇ or C₂-C₅ alkoxycarbonyl, or C₂-C₇ or C₂-C₂-C₅ haloalkoxycarbonyl;
G¹ and G² stand for oxygen;
X¹ to X⁵ independently represent hydrogen, halogen, optionally substituted C₁-C₆ or C₁-C₄alkyl, C₁-C₆ or C₁-C₄haloalkyl, C₁-C₆ or C₁-C₄alkoxy, C₁-C₆ or C₁-C₄haloalkoxy, or cyano or nitro;
Y² to Y⁴ independently represent hydrogen, halogen, optionally substituted C₁-C₆ or C₁-C₄alkyl, C₁-C₆ or C₁-C₄haloalkyl, C₁-C₆ or C₁-C₄alkoxy, C₁-C₆ or C₁-C₄haloalkoxy, cyano or nitro;
Y¹ and Y⁵ independently represent halogen, optionally substituted C₁-C₆ or C₁-C₄alkyl, C₁-C₆or C₁-C₄haloalkyl, C₁-C₆ or C₁-C₄alkoxy, C₁-C₆ or C₁-C₄haloalkoxy, C₁-C₆ or C₁-C₄alkylthio, C₁-C₆ or C₁-C₄alkylsulfinyl, C₁-C₆ or C₁-C₄alkylsulfonyl, C₁-C₆ or C₁-C₄haloalkylthio, C₁-C₆ or C₁-C₄haloalkylsulfinyl, C₁-C₆ or C₁-C₄haloalkylsulfonyl, or cyano or nitro;
and
J represents a chemical grouping having the following formula: 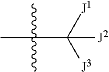
wherein
J¹ represents C₁-C₆ fluoroalkyl;
J² represents hydrogen, halogen, optionally substituted C₁-C₆ or C₁-C₄alkyl, C₁-C₆ or C₁-C₄haloalkyl, phenyl or a 5- or 6-membered heterocyclic group comprising at least one hetero atom selected from the group consisting of N, O and S; and
J³ represents hydroxyl, cyano, azide, halogen, or optionally substituted C₁-C₆ or C₁-C₄alkyl, or C₁-C₆ or C₁-C₄haloalkyl, or OR⁴, SR⁵, NR⁶R⁷, N(R⁸)NR⁶R⁷, N(R⁸)OR⁶, an optionally substituted heterocyclic group, or a chemical group having the following formulae: 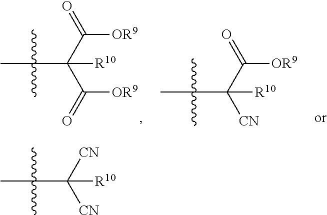
R⁴ represents optionally substituted C₁-C₆ or C₁-C₄alkyl, C₁-C₆ or C₁-C₄haloalkyl, C₁-C₆alkoxyC₁-C₆alkyl or C₁-C₄alkoxyC₁-C₄alkyl; C₁-C₆alkylthioC₁-C₆alkyl or C₁-C₄ alkylthioC₁-C₄alkyl, C₁-C₆haloalkylthioC₁-C₆alkyl or C₁-C₄haloalkylthioC₁-C₄alkyl, C₂-C₆alkenyl, C₂-C₆ or C₂-C₄alkynyl, C₃-C₁₀cycloalkyl, C₃-C₁₀cycloalkenyl, phenyl, C₇-C₁₂aralkyl or C₇-C₁₀ aralkyl, C₁-C₆iminyl or C₁-C₄iminyl, C₂-C₇ or C₂-C₅alkylcarbonyl, C₂-C₇ or C₂-C₅haloalkylcarbonyl, phenylcarbonyl, C₁-C₆or C₁-C₄ alkylsulfonyl, C₁-C₆ or C₁-C₄haloalkylsulfonyl, phenylsulfonyl, 5- to 6-membered heterocyclic group comprising at least one hetero atom selected from the group consisting of N, O and S, a 5- or 6-membered heterocyclic group-C₁-C₆ alkylene comprising at least one hetero atom selected from the group consisting of N, O and S, or a 5- or 6-membered heterocyclic group-carbonyl comprising at least one hetero atom selected from the group consisting of N, O and S;
R⁵ represents optionally substituted C₁-C₆ or C₁-C₄alkyl, optionally substituted C₁-C₆ or C₁-C₄haloalkyl, C₁-C₆alkoxyC₁-C₆alkyl or C₁-C₄alkoxyC₁-C₄alkyl, C₁-C₆haloalkoxyC₁-C₆ alkyl or C₁-C₄haloalkoxyC₁-C₄alkyl, C₁-C₆ or C₁-C₄alkyl substituted with optionally substituted C₁-C₆ alkylthio, such as C₁-C₄ alkylthioC₁-C₄alkyl, C₁-C₆ haloalkylthioC₁-C₆ alkyl or C₁-C₄ haloalkylthioC₁-C₄ alkyl; optionally substituted C₂-C₆ or C₂-C₄alkenyl, C₂-C₆ or C₂-C₄alkynyl, phenyl, C₇-C₁₂ or C₇-C₁₀aralkyl, a 5- or 6-membered heterocyclic group comprising at least one hetero atom selected from the group consisting of N, O and S, or a 5- or 6-membered heterocyclic group-C₁-C₆ alkylene comprising at least one hetero atom selected from the group consisting of N, O and S;
R⁶, R⁷ and R⁸ independently represent hydrogen, optionally substituted C₁-C₆ or C₁-C₄alkyl, C₁-C₆ or C₁-C₄haloalkyl, C₁-C₆ alkoxyC₁-C₆ alkyl or C₁-C₄ alkoxyC₁-C₄ alkyl C₁-C₆ haloalkoxyC₁-C₆ alkyl or C₁-C₄ haloalkoxyC₁-C₄ alkyl, C₁-C₆ alkylthioC₁-C₆ alkyl or C₁-C₄ alkylthioC₁-C₄ alkyl, C₁-C₆ haloalkylthioC₁-C₆ alkyl or C₁-C₄ haloalkylthioC₁-C₄ alkyl, C₂-C₆or C₂-C₄ alkenyl, C₂-C₆ or C₂-C₄ alkynyl, phenyl, C₇-C₁₂ or C₇-C₁₀ aralkyl, C₁-C₆ or C₁-C₄alkylsulfonyl, C₁-C₆ or C₁-C₄ haloalkylsulfonyl, phenylsulfonyl, C₂-C₇ or C₂-C₅alkylcarbonyl, C₂-C₇ or C₂-C₇ haloalkylcarbonyl, phenylcarbonyl, a 5- or 6-membered heterocyclic group comprising at least one hetero atom selected from the group consisting of N, O and S, a 5- or 6-membered heterocyclic group-C₁-C₆ alkylene comprising at least one hetero atom selected from the group consisting of N, O and S, or a 5- or 6-membered heterocyclic group-carbonyl comprising at least one hetero atom selected from the group consisting of N, O and S;
or
R⁶ and R⁷ may form a cyclic amino group together with the nitrogen atom to which they are bonded, preferably a 3- to 7-membered cyclic amino group, and said cycle may comprise an oxygen atom, a sulfur atom or a carbonyl group;
R⁹ represents optionally substituted alkyl or haloalkyl, preferably R⁹ represents C₁-C₆ or C₁-C₄ alkyl or C₁-C₆ or C₁-C₄ haloalkyl; and
R¹⁰ represents hydrogen, optionally substituted alkyl or haloalkyl, preferably R⁹ represents optionally substituted C₁-C₆ or C₁-C₄ alkyl or C₁-C₆ or C₁-C₄ haloalkyl.

3. A composition comprising at least one compound according to claim 1.

4. A method for controlling animal pests, comprising applying a compound according to claim 1 to animal pests, their habitat, or a combination thereof.

5-7. (canceled)

8. A compound of formula (II): 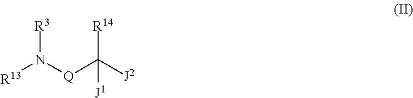
wherein
R¹³ represents hydrogen, C₁-C₆alkyloxycarbonyl, phenyloxycarbonyl, aralkyloxycarbonyl or the following group: 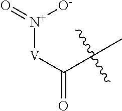
V is selected among the cyclic groups V1 to V5: 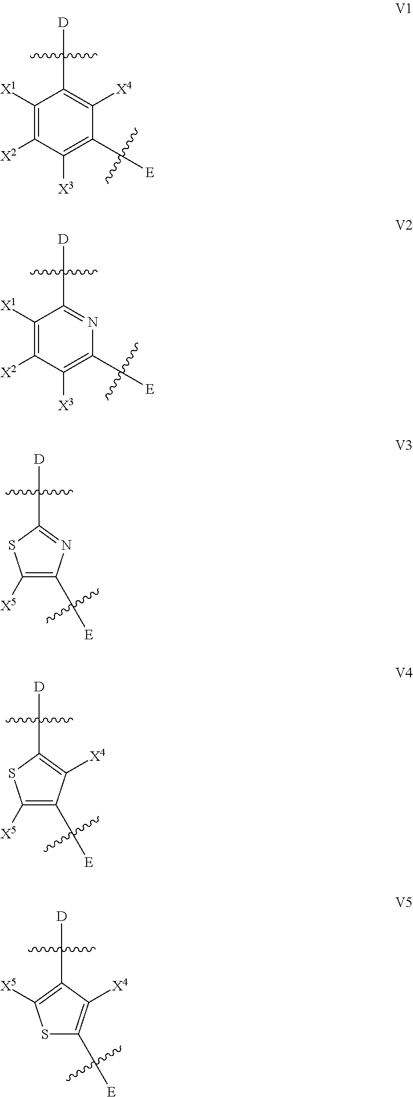
wherein X¹ to X⁵ independently represent hydrogen, halogen, or optionally substituted alkyl, haloalkyl, alkoxy, haloalkoxy, or cyano or nitro; provided that, in V, D represents a bonding site to the moiety: 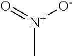
of the group, and E represents a bonding site to the moiety: 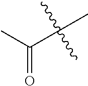
of the group;
or R¹³ represents the following group: 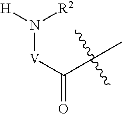
wherein V and X¹ to X⁴ are as defined above and R² represents hydrogen, or optionally substituted alkyl, haloalkyl, alkylcarbonyl, haloalkylcarbonyl, alkoxycarbonyl or haloalkoxycarbonyl;
provided that, in V, D represents a bonding site to the moiety: 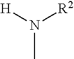
of the group, and E represents a bonding site to the moiety: 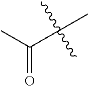
of the group:
R¹⁴ represents J³ or the following group:
—O-L¹;
wherein J³ represents hydroxyl, cyano, azide, halogen, or optionally substituted alkyl, haloalkyl, or OR⁴, SR⁵, NR⁶R⁷, N(R⁸)NR⁶R⁷, N(R⁸)OR⁶, an optionally substituted heterocyclic group, or a chemical group having the following formulae: 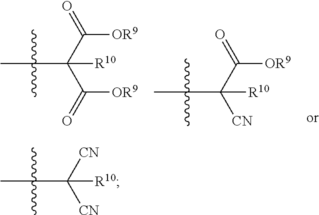
and wherein
R⁴ represents optionally substituted alkyl, haloalkyl, alkoxyalkyl, alkylthioalkyl, haloalkylthioalkyl alkenyl, alkynyl, cycloalkyl, cycloalkenyl, phenyl, aralkyl, iminyl, alkylcarbonyl, haloalkylcarbonyl, phenylcarbonyl, alkylsulfonyl, haloalkylsulfonyl, phenylsulfonyl, or a heterocyclic group, heterocyclic group-alkylene or a heterocyclic group-carbonyl,
R⁵ represents optionally substituted alkyl, haloalkyl, alkoxyalkyl, haloalkoxyalkyl, alkylthioalkyl, haloalkylthioalkyl, alkenyl, alkynyl, phenyl, aralkyl, or a 5- or 6-membered heterocyclic group comprising at least one hetero atom selected from the group consisting of N, O and S, or a 5- or 6-membered heterocyclic group-alkylene comprising at least one hetero atom selected from the group consisting of N, O and S,
R⁶, R⁷ and R⁸ independently represent hydrogen, optionally substituted alkyl, haloalkyl, alkoxyalkyl, haloalkoxyalkyl, alkylthioalkyl, haloalkylthioalkyl, alkenyl, alkynyl, phenyl, aralkyl, alkylsulfonyl, haloalkylsulfonyl, phenylsulfonyl, alkylcarbonyl, haloalkylcarbonyl, phenylcarbonyl, a 5- or 6-membered heterocyclic group comprising at least one hetero atom selected from the group consisting of N, O and S, a 5- or 6-membered heterocyclic group-alkylene comprising at least one hetero atom selected from the group consisting of N, O and S, or a 5- or 6-membered heterocyclic group-carbonyl and comprising at least one hetero atom selected from the group consisting of N, O and S,
or
R⁶ and R⁷ may form a cyclic amino group together with the nitrogen atom to which they are bonded, and said cycle may comprise an oxygen atom, a sulfur atom or a carbonyl group;
R⁹ represents optionally substituted alkyl or haloalkyl; and
R¹⁹ represents hydrogen. optionally substituted alkyl or haloalkyl, preferably R⁹ represents optionally substituted C₁-C₆ or C₁-C₄ alkyl or C₁-C₆ or C₁-C₄ haloalkyl;
wherein L¹ represents alkylsulfonyl or phenylsulfonyl;
R³ represents hydrogen, or optionally substituted alkyl, haloalkyl, alkylcarbonyl, haloalkylcarbonyl, alkoxycarbonyl or haloalkoxycarbonyl;
O is selected among the groups Q1 to Q7 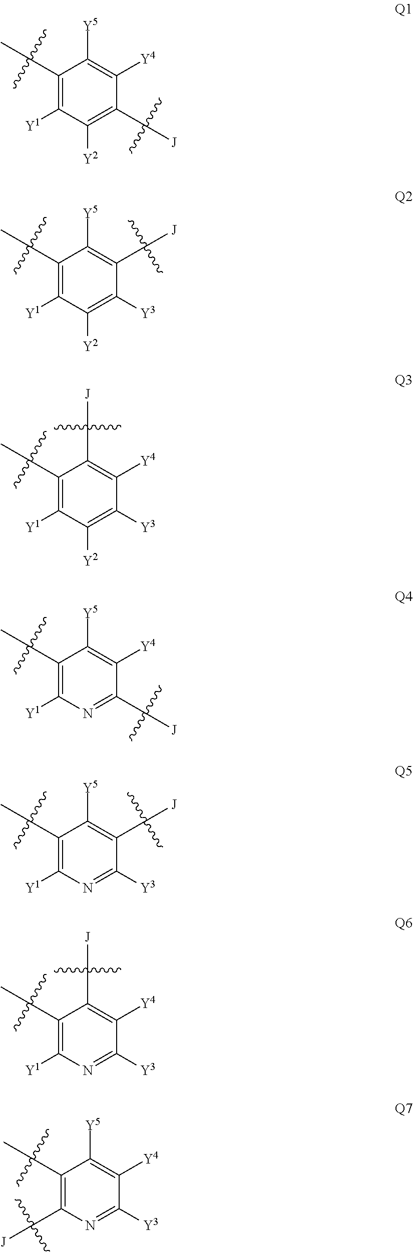
wherein
Y² to Y⁴ independently represent hydrogen, halogen, or optionally substituted alkyl, haloalkyl, alkoxy, haloalkoxy, or cyano or nitro
Y¹ and Y⁵ independently represent halogen, or optionally substituted alkyl, haloalkyl, alkoxy, haloalkoxy, alkylthio, alkylsulfinyl, alkylsulfonyl, haloalkylthio, haloalkylsulfinyl, haloalkylsulfonyl, or cyano or nitro;
and
J represents a bonding site to the moiety: 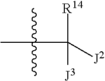
wherein
J¹ represents C₁-C₆ haloalkyl,
J² represents hydrogen, halogen. or optionally substituted alkyl, haloalkyl. phenyl or a heterocyclic group,
provided that J¹ and J² are not perfluoroalkyl and J³ is not a hydroxyl group or halogen.

9. A method according to claim 4, wherein the animal pest is an arthropod.

10. A method of treating seed of conventional or transgenic plants, comprising applying a compound according to claim 1 directly to the seed or their surroundings, habitat or storage space.

11. A method of preparing a composition, comprising mixing a compound of formula (I) according to claim 1 with an extender, a diluent, or combinations thereof.
